(19) United States
(12) Patent Application Publication
(10) Pub. No.: US 2004/0006205 A1
(43) Pub. Date: Jan. 8, 2004

(54) Therapeutic polypeptides, nucleic acids encoding same, and methods of use

(76) Inventors: Li Li, Branford, CT (US); Valerie Gerlach, Branford, CT (US); Xiaohong Liu, Lexington, MA (US); Charles E. Miller, Guilford, CT (US); Kimberly A. Spytek, New Haven, CT (US); Bryan D. Zerhusen, Branford, CT (US); Carol E. A. Pena, New Haven, CT (US); Suresh G. Shenoy, Branford, CT (US); Haihong Zhong, Guilford, CT (US); Glennda Smithson, Guilford, CT (US); Stacie J. Casman, North Haven, CT (US); Ferenc L. Boldog, North Haven, CT (US); Edward Z. Voss, Wallingford, CT (US); Corine A. Vernet, North Branford, CT (US); John R. MacDougall, Hamden, CT (US); Luca Rastelli, Guilford, CT (US); David W. Anderson, Branford, CT (US); Mei Zhong, Branford, CT (US); Peter S. Mezes, Old Lyme, CT (US); Katarzyna Furtak, Ansonia, CT (US); Meera Patturajan, Branford, CT (US); Catherine E. Burgess, Wethersfield, CT (US); Uriel M. Malyankar, Branford, CT (US); Richard A. Shimkets, Guilford, CT (US); Raymond J. Taupier JR., East Haven, CT (US); Schlomit Edinger, New Haven, CT (US); Ann Mazur, Bloomfield, CT (US)

Correspondence Address: Ivor R. Elrifi, Esq., MINTZ, LEVIN, COHN, FERRIS,, GLOVSKY and POPEO, P.C., One Financial Center, Boston, MA, 02111, US

(21) Appl. No.: 10/115,479

(22) Filed: Apr. 2, 2002

Related U.S. Application Data
(60) Provisional application No. 60/281,136, filed on Apr. 3, 2001.
(60) Provisional application No. 60/281,863, filed on Apr. 5, 2001.
(60) Provisional application No. 60/294,495, filed on May 30, 2001.
(60) Provisional application No. 60/281,906, filed on Apr. 5, 2001.
(60) Provisional application No. 60/282,934, filed on Apr. 10, 2001.
(60) Provisional application No. 60/294,801, filed on May 31, 2001.
(60) Provisional application No. 60/324,775, filed on Sep. 25, 2001.
(60) Provisional application No. 60/283,687, filed on Apr. 13, 2001.
(60) Provisional application No. 60/283,657, filed on Apr. 13, 2001.
(60) Provisional application No. 60/283,678, filed on Apr. 13, 2001.
(60) Provisional application No. 60/283,710, filed on Apr. 13, 2001.
(60) Provisional application No. 60/284,234, filed on Apr. 17, 2001.
(60) Provisional application No. 60/288,509, filed on May 3, 2001.
(60) Provisional application No. 60/309,216, filed on Jul. 31, 2001.
(60) Provisional application No. 60/285,325, filed on Apr. 19, 2001.
(60) Provisional application No. 60/285,609, filed on Apr. 20, 2001.
(60) Provisional application No. 60/285,748, filed on Apr. 23, 2001.
(60) Provisional application No. 60/333,900, filed on Nov. 28, 2001.
(60) Provisional application No. 60/285,890, filed on Apr. 23, 2001.
(60) Provisional application No. 60/286,068, filed on Apr. 24, 2001.
(60) Provisional application No. 60/287,213, filed on Apr. 27, 2001.

(51) Int. Cl. C07K 1/00; C07K 14/00; C07K 17/00

(52) U.S. Cl. 530/350

(57) ABSTRACT

Disclosed herein are nucleic acid sequences that encode G-coupled protein-receptor related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority to U.S.S. No. 60/281,136, filed on Apr. 3, 2001; U.S.S. No. 60/281,863, filed on Apr. 5, 2001; U.S.S. No. 60/281,906, filed on Apr. 5, 2001; U.S.S. No. 60/282,934, filed on Apr. 10, 2001; U.S.S. No. 60/283,657, filed on Apr. 13, 2001; U.S.S. No. 60/283,678, filed on Apr. 13, 2001; U.S.S. No. 60/283,687, filed on Apr. 13, 2001; U.S.S. No. 60/283,710, filed on Apr. 13, 2001; U.S.S. No. 60/284,234, filed on Apr. 17, 2001; U.S.S. No. 60/285,325, filed on Apr. 19, 2001; U.S.S. No. 60/285,609, filed on Apr. 20, 2001; U.S.S. No. 60/285,748, filed on Apr. 23, 2001; U.S.S. No. 60/285,890, filed on Apr. 23, 2001; U.S.S. No. 60/286,068, filed on Apr. 24, 2001; U.S.S. No. 60/287,213, filed on Apr. 27, 2001; U.S.S. No. 60/288,509, filed on May 3, 2001; U.S.S. No. 60/294,495, filed on May 30, 2001; U.S.S. No. 60/294,801, filed on May 31, 2001; U.S.S. No. 60/309,216, filed on Jul. 31, 2001; U.S.S. No. 60/324,775, filed on Sep. 25, 2001; and U.S.S. No. 60/333,900, filed on Nov. 28, 2001; each of which is incorporated by reference in its entirety.

### FIELD OF THE INVENTION

The present invention relates to novel polypeptides, and the nucleic acids encoding them, having properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND OF THE INVENTION

Eukaryotic cells are characterized by biochemical and physiological processes, which under normal conditions are exquisitely balanced to achieve the preservation and propagation of the cells. When such cells are components of multicellular organisms such as vertebrates or, more particularly, organisms such as mammals, the regulation of the biochemical and physiological processes involves intricate signaling pathways. Frequently, such signaling pathways include constituted of extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, such as two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including, by way of nonlimiting example, induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

### SUMMARY OF THE INVENTION

The invention is based in part upon the discovery of isolated polypeptides including amino acid sequences selected from mature forms of the amino acid sequences selected from the group consisting of SEQ ID NO:2n, wherein n is an integer between 1 and 45. The invention also is based in part upon variants of a mature form of the amino acid sequence selected from the group consisting of SEQ ID NO:2n, wherein n is an integer between 1 and 45, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO:2n, wherein n is an integer between 1 and 45. In another embodiment, the invention also comprises variants of the amino acid sequence selected from the group consisting of SEQ ID NO:2n, wherein n is an integer between 1 and 45 wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO:2n, wherein n is an integer between 1 and 45, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO:2n, wherein n is an integer between 1 and 45. These allelic variants include amino acid sequences that are the translations of nucleic acid sequences differing by a single nucleotide from nucleic acid sequences selected from the group consisting of SEQ ID NOS: 2n-1, wherein n is an integer between 1 and 45. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO:2n, wherein n is an integer between 1 and 45 and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO:2n, wherein n is an integer between 1 and 45 wherein said therapeutic is the polypeptide selected from this group.

In another embodiment, the invention comprises a method for determining the presence or amount of a polypeptide with an amino acid sequence selected from the group consisting of SEQ ID NO:2n, wherein n is an integer between 1 and 45 in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO:2n, wherein n is an integer between 1 and 45 in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO:2n, wherein n is an integer between 1 and 45, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO:2n, wherein n is an integer between 1 and 45, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method for screening for a modulator of activity or of latency or predisposition to a pathology associated with a polypeptide having an amino acid sequence selected from the group consisting of SEQ ID NO:2n, wherein n is an integer between 1 and 45, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of the invention. The recombinant test animal could express a test protein transgene or express the transgene under the control of a promoter at an increased level relative to a wild-type test animal The promoter may or may not b the native gene promoter of the transgene.

In another embodiment, the invention involves a method for modulating the activity of a polypeptide with an amino acid sequence selected from the group consisting of SEQ ID NO:2n, wherein n is an integer between 1 and 45, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO:2n, wherein n is an integer between 1 and 45, the method including administering the polypeptide to a subject in which such treatment or prevention is desired in an amount sufficient to treat or prevent the pathology in the subject. The subject could be human.

In another embodiment, the invention involves a method of treating a pathological state in a mammal, the method including administering to the mammal a polypeptide in an amount that is sufficient to alleviate the pathological state, wherein the polypeptide is a polypeptide having an amino acid sequence at least 95% identical to a polypeptide having the amino acid sequence selected from the group consisting of SEQ ID NO:2n, wherein n is an integer between 1 and 45 or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO:2n, wherein n is an integer between 1 and 45; a variant of a mature form of the amino acid sequence selected from the group consisting of SEQ ID NO:2n, wherein n is an integer between 1 and 45 wherein any amino acid in the mature form of the chosen sequence is changed to a different amino acid, provided that no more than 15% of the amino acid residues in the sequence of the mature form are so changed; the amino acid sequence selected from the group consisting of SEQ ID NO:2n, wherein n is an integer between 1 and 45; a variant of the amino acid sequence selected from the group consisting of SEQ ID NO:2n, wherein n is an integer between 1 and 45, in which any amino acid specified in the chosen sequence is changed to a different amino acid, provided that no more than 15% of the amino acid residues in the sequence are so changed; a nucleic acid fragment encoding at least a portion of a polypeptide comprising the amino acid sequence selected from the group consisting of SEQ ID NO:2n, wherein n is an integer between 1 and 45 or any variant of the polypeptide wherein any amino acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 45, wherein the nucleic acid molecule comprises the nucleotide sequence of a naturally occurring allelic nucleic acid variant.

In another embodiment, the invention involves an isolated nucleic acid molecule including a nucleic acid sequence encoding a polypeptide having an amino acid sequence selected from the group consisting of a mature form of the amino acid sequence given SEQ ID NO:2n, wherein n is an integer between 1 and 45 that encodes a variant polypeptide, wherein the variant polypeptide has the polypeptide sequence of a naturally occurring polypeptide variant.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 45, wherein the nucleic acid molecule differs by a single nucleotide from a nucleic acid sequence selected from the group consisting of SEQ ID NOS: 2n-1, wherein n is an integer between 1 and 45.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 45, wherein the nucleic acid molecule comprises a nucleotide sequence selected from the group consisting of the nucleotide sequence selected from the group consisting of SEQ ID NO:2n-1, wherein n is an integer between 1 and 45; a nucleotide sequence wherein one or more nucleotides in the nucleotide sequence selected from the group consisting of SEQ ID NO:2n-1, wherein n is an integer between 1 and 45 is changed from that selected from the group consisting of the chosen sequence to a different nucleotide provided that no more than 15% of the nucleotides are so changed; a nucleic acid fragment of the sequence selected from the group consisting of SEQ ID NO:2n-1, wherein n is an integer between 1 and 45; and a nucleic acid fragment wherein one or more nucleotides in the nucleotide sequence selected from the group consisting of SEQ ID NO:2n-1, wherein n is an integer between 1 and 45 is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 45, wherein the nucleic acid molecule hybridizes under stringent conditions to the nucleotide sequence selected from the group consisting of SEQ ID NO:2n-1, wherein n is an integer between 1 and 45, or a complement of the nucleotide sequence.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 45, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group consisting of a mature form of the amino acid sequence given SEQ ID NO:2n, wherein n is an integer between 1 and 45. This vector can have a promoter operably linked to the nucleic acid molecule. This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO:2n, wherein n is an integer between 1 and 45 in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO:2n, wherein n is an integer between 1 and 45 in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 1 provides a summary of the NOVX nucleic acids and their encoded polypeptides.

TABLE 1
Sequences and Corresponding SEQ ID Numbers

| | | SEQ ID | SEQ ID | |
|---|---|---|---|---|
| | | NO | NO | |
| NOVX | Internal | (nucleic | (amino | |
| Assignment | Identification | acid) | acid) | Homology |
| 1 | CG56908-02 | 1 | 2 | Prorelaxin H2 Precursor |
| 2a | CG59783-01 | 3 | 4 | CGI-67 |
| 2b | CG59783-02 | 5 | 6 | CGI-67 |
| 3 | CG59873-01 | 7 | 8 | Cystatin |
| 4 | CG89060-01 | 9 | 10 | Collagen Alpha 1(XIV) Chain Precursor |
| | | | | (Undulin) |
| 5 | CG89511_01 | 11 | 12 | Plasma Kallekrein |
| 6 | CG89614_02 | 13 | 14 | Neurophysin |
| 7 | CG90031-01 | 15 | 16 | Cathepsin L |
| 8 | CG90155-01 | 17 | 18 | Secreted Protein |
| 9a | CG90750-01 | 19 | 20 | High (Glycine + Tyrosine) Keratin |
| 9b | CG90750-02 | 21 | 22 | High (Glycine + Tyrosine) Keratin |
| 10 | CG91235-01 | 23 | 24 | Interleukin 8 |
| 11a | CG91657-01 | 25 | 26 | Brush Border 61.0 kDa Protein Precursor |
| 11b | CG91657-02 | 27 | 28 | Brush Border 61.0 kDa Protein Precursor |
| 12a | CG91678-01 | 29 | 30 | MMP-1 |
| 12b | 172557724 | 31 | 32 | MMP-1 |
| 12c | 172557764 | 33 | 34 | MMP-1 |
| 12d | 173877223 | 35 | 36 | MMP-1 |
| 12e | 172557827 | 37 | 38 | MMP-1 |
| 12f | CG91678-03 | 39 | 40 | MMP-1 |
| 13 | CG91698-01 | 41 | 42 | Heparanase |
| 14a | CG91708-01 | 43 | 44 | MMP-3 |
| 14b | CG91708-02 | 45 | 46 | MMP-3 |
| 14c | 240317953 | 47 | 48 | MMP-3 |
| 14d | 240317980 | 49 | 50 | MMP-3 |
| 15a | CG91729-01 | 51 | 52 | MMP-13 |
| 15b | CG91729-02 | 53 | 54 | MMP-13 |
| 16a | CG92489-01 | 55 | 56 | BCG-Induced Integral Membrane Protein |
| 16b | 228495688 | 57 | 58 | BCG-Induced Integral Membrane Protein |
| 16c | 228495693 | 59 | 60 | BCG-Induced Integral Membrane Protein |
| 16d | 228495682 | 61 | 62 | BCG-Induced Integral Membrane Protein |
| 17a | CG93008-01 | 63 | 64 | Prepro-Plasma Carboxypeptidase B |
| 17b | CG93008-02 | 65 | 66 | Prepro-Plasma Carboxypeptidase B |
| 17c | CG93008-03 | 67 | 68 | Prepro-Plasma Carboxypeptidase B |
| 17d | CG93008-04 | 69 | 70 | Prepro-Plasma Carboxypeptidase B |
| 18a | CG93252-01 | 71 | 72 | Procathepsin L |
| 18b | CG93252-02 | 73 | 74 | Procathepsin L |
| 18c | CG93252-03 | 75 | 76 | Procathepsin L |
| 19 | CG93285-01 | 77 | 78 | Matrix Metalloprotease |
| 20a | CG93387-01 | 79 | 80 | Fibropellin I Precursor |
| 20b | CG93387-02 | 81 | 82 | Fibropellin I Precursor |
| 21 | CG93702-01 | 83 | 84 | Interleukin Receptor |
| 22 | CG93792-01 | 85 | 86 | Properdin |
| 23 | CG94013-01 | 87 | 88 | Properdin |
| 24 | CG94442_01 | 89 | 90 | Carboxylesterase Precursor |

Table 1 indicates homology of NOVX nucleic acids to known protein families. Thus, the nucleic acids and polypeptides, antibodies and related compounds according to the invention corresponding to a NOVX as identified in column 1 of Table 1 will be useful in therapeutic and diagnostic applications implicated in, for example, pathologies and disorders associated with the known protein families identified in column 5 of Table 1.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table 1, the NOVX polypeptides of the present invention show homology to, and contain domains that are characteristic of, other members of such protein families. Details of the sequence relatedness and domain analysis for each NOVX are presented in Examples 1-24.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table 1.

The NOVX nucleic acids and polypeptides are also useful for detecting specific cell types. Details of the expression analysis for each NOVX are presented in Example 27. Accordingly, the NOVX nucleic acids, polypeptides, antibodies and related compounds according to the invention will have diagnostic and therapeutic applications in the detection of a variety of diseases with differential expression in normal vs. diseased tissues, e.g. a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

In one specific embodiment, the invention includes an isolated polypeptide comprising an amino acid sequence selected from the group consisting of: (a) a mature form of the amino acid sequence selected from the group consisting of SEQ ID NO:2n, wherein n is an integer between 1 and 45; (b) a variant of a mature form of the amino acid sequence selected from the group consisting of SEQ ID NO:2n, wherein n is an integer between 1 and 45, wherein any amino acid in the mature form is changed to a different amino acid, provided that no more than 15% of the amino acid residues in the sequence of the mature form are so changed; (c) an amino acid sequence selected from the group consisting of SEQ ID NO:2n, wherein n is an integer between 1 and 45; (d) a variant of the amino acid sequence selected from the group consisting of SEQ ID NO:2n, wherein n is an integer between 1 and 45 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO:2n, wherein n is an integer between 1 and 45; (b) a variant of a mature form of the amino acid sequence selected from the group consisting of SEQ ID NO:2n, wherein n is an integer between 1 and 45 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO:2n, wherein n is an integer between 1 and 45; (d) a variant of the amino acid sequence selected from the group consisting of SEQ ID NO:2n, wherein n is an integer between 1 and 45, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO:2n, wherein n is an integer between 1 and 45 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO:2n-1, wherein n is an integer between 1 and 45; (b) a nucleotide sequence wherein p one or more nucleotides in the nucleotide sequence selected from the group consisting of SEQ ID NO:2n-1, wherein n is an integer between 1 and 45 is changed from that selected from the group consisting of the chosen sequence to a different nucleotide provided that no more than 15% of the nucleotides are so changed; (c) a nucleic acid fragment of the sequence selected from the group consisting of SEQ ID NO:2n-1, wherein n is an integer between 1 and 45; and (d) a nucleic acid fragment wherein one or more nucleotides in the nucleotide sequence selected from the group consisting of SEQ ID NO:2n-1, wherein n is an integer between 1 and 45 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide, precursor form, or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (host cell) in which the gene product arises. Examples of such processing steps leading to a “mature” form of a polypeptide or protein include the cleavage of the N-terminal methionine residue encoded by the initiation codon of an ORF or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), and 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as used herein, is a nucleic acid which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb, 0.1 kb, or less of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, culture medium, or of chemical precursors or other chemicals.

A nucleic acid molecule of the invention, e.g., a nucleic acid molecule having the nucleotide sequence SEQ ID NOS: 2n-1, wherein n is an integer between 1 and 45, or a complement of this nucleotide sequence, can be isolated using standard molecular biology techniques and the sequence information provided herein. Using all or a portion of the nucleic acid sequence of SEQ ID NOS:2n-1, wherein n is an integer between 1 and 45, as a hybridization probe, NOVX molecules can be isolated using standard hybridization and cloning techniques (e.g., as described in Sambrook, et al., (eds.), Molecular Cloning: A Laboratory Manual 2^{nd} Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993).

A nucleic acid of the invention can be amplified using cDNA, mRNA or, alternatively, genomic DNA as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:2n-1, wherein n is an integer between 1 and 45, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:2n-1, wherein n is an integer between 1 and 45, or a portion of this nucleotide sequence (e.g., a fragment that can be used as a probe or primer or a fragment encoding a biologically-active portion of A NOVX polypeptide). A nucleic acid molecule that is complementary to the nucleotide sequence shown SEQ ID NOS:2n-1, wherein n is an integer between 1 and 45, is one that is sufficiently complementary to the nucleotide sequence shown SEQ ID NOS:2n-1, wherein n is an integer between 1 and 45, that it can hydrogen bond with few or no mismatches to the nucleotide sequence shown SEQ ID NOS:2n-1, wherein n is an integer between 1 and 45, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

“Fragments” provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, and are at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

“Derivatives” are nucleic acid sequences or amino acid sequences formed from the native compounds either directly, by modification, or by partial substitution. “Analogs” are nucleic acid sequences or amino acid sequences that have a structure similar to, but not identical to, the native compound, e.g. they differ from it in respect to certain components or side chains. Analogs may be synthetic or derived from a different evolutionary origin and may have a similar or opposite metabolic activity compared to wild type. Homologs are nucleic acid sequences or amino acid sequences of a particular gene that are derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions. See e.g. Ausubel, et al., Current Protocols in Molecular Biology, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for A NOVX polypeptide of species other than humans, including, but not limited to vertebrates, and thus can include, e.g., frog, mouse, rat, rabbit, dog, cat, cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding a human NOVX protein. Homologous nucleic acid sequences include those nucleic acid sequences that encode conservative amino acid substitutions (see below) in SEQ ID NOS:2n-1, wherein n is an integer between 1 and 45, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS:2n-1, wherein n is an integer between 1 and 45; or an anti-sense strand nucleotide sequence of SEQ ID NOS:2n-1, wherein n is an integer between 1 and 45; or of a naturally occurring mutant of SEQ ID NOS:2n-1, wherein n is an integer between 1 and 45.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express A NOVX protein, such as by measuring a level of A NOVX-encoding nucleic acid in a sample of cells from a subject e.g., detecting NOVX mRNA levels or determining whether a genomic NOVX gene has been mutated or deleted.

“A polypeptide having a biologically-active portion of A NOVX polypeptide” refers to polypeptides exhibiting activity similar, but not necessarily identical, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:2n-1, wherein n is an integer between 1 and 45, that encodes a polypeptide having A NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:2n-1, wherein n is an integer between 1 and 45, due to degeneracy of the genetic code and thus encode the same NOVX proteins as that encoded by the nucleotide sequences shown in SEQ ID NOS:2n-1, wherein n is an integer between 1 and 45. In another embodiment, an isolated nucleic acid molecule of the invention has a nucleotide sequence encoding a protein having an amino acid sequence shown in SEQ ID NOS:2n, wherein n is an integer between 1 and 45.

In addition to the human NOVX nucleotide sequences shown in SEQ ID NOS:2n-1, wherein n is an integer between 1 and 45, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding A NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:2n-1, wherein n is an integer between 1 and 45, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:2n-1, wherein n is an integer between 1 and 45. In another embodiment, the nucleic acid is at least 10, 25, 50, 100, 250, 500, 750, 1000, 1500, 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured sahnon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:2n-1, wherein n is an integer between 1 and 45, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:2n-1, wherein n is an integer between 1 and 45, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, Current Protocols in Molecular Biology, John Wiley & Sons, NY, and Kriegler, 1990; Gene Transfer and Expression, A Laboratory Manual, Stockton Press, NY.

In a third embodiment, a nucleic acid that is hybridizable to the nucleic acid molecule comprising the nucleotide sequences SEQ ID NOS:2n-1, wherein n is an integer between 1 and 45, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, Current Protocols in Molecular Biology, John Wiley & Sons, NY, and Kriegler, 1990, Gene Transfer and Expression, A Laboratory Manual, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:2n-1, wherein n is an integer between 1 and 45, thereby leading to changes in the amino acid sequences of the encoded NOVX proteins, without altering the functional ability of the NOVX proteins. For example, nucleotide substitutions leading to amino acid substitutions at “non-essential” amino acid residues can be made in the sequence SEQ ID NOS:2n, wherein n is an integer between 1 and 45. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well known within the art.

Another aspect of the invention pertains to nucleic acid molecules encoding NOVX proteins that contain changes in amino acid residues that are not essential for activity. Such NOVX proteins differ in amino acid sequence from SEQ ID NOS:2n-1, wherein n is an integer between 1 and 45, yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2n, wherein n is an integer between 1 and 45. Preferably, the protein encoded by the nucleic acid molecule is at least about 60% homologous to SEQ ID NOS:2n, wherein n is an integer between 1 and 45; more preferably at least about 70% homologous SEQ ID NOS:2n, wherein n is an integer between 1 and 45; still more preferably at least about 80% homologous to SEQ ID NOS:2n, wherein n is an integer between 1 and 45; even more preferably at least about 90% homologous to SEQ ID NOS:2n, wherein n is an integer between 1 and 45; and most preferably at least about 95% homologous to SEQ ID NOS:2n, wherein n is an integer between 1 and 45.

An isolated nucleic acid molecule encoding A NOVX protein homologous to the protein of SEQ ID NOS:2n, wherein n is an integer between 1 and 45, can be created by introducing one or more nucleotide substitutions, additions or deletions into the nucleotide sequence of SEQ ID NOS:2n-1, wherein n is an integer between 1 and 45, such that one or more amino acid substitutions, additions or deletions are introduced into the encoded protein.

Mutations can be introduced into SEQ ID NOS:2n-1, wherein n is an integer between 1 and 45, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g. alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of A NOVX coding sequence, such as by saturation mutagenesis, and the resultant mutants can be screened for NOVX biological activity to identify mutants that retain activity. Following mutagenesis SEQ ID NOS:2n-1, wherein n is an integer between 1 and 45, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and A NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:2n-1, wherein n is an integer between 1 and 45, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of A NOVX protein of SEQ ID NOS:2n, wherein n is an integer between 1 and 45, or antisense nucleic acids complementary to A NOVX nucleic acid sequence of SEQ ID NOS:2n-1, wherein n is an integer between 1 and 45, are additionally provided.

In one embodiment, an antisense nucleic acid molecule is antisense to a “coding region” of the coding strand of a nucleotide sequence encoding A NOVX protein. The term “coding region” refers to the region of the nucleotide sequence comprising codons, which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences, which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, beta-D-mannosylqueosine, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding A NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. A α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of A NOVX cDNA disclosed herein (i.e., SEQ ID NOS:2n-1, wherein n is an integer between 1 and 45). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S1 nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2n, wherein n is an integer between 1 and 45. The invention also includes a mutant or variant protein any of whose residues may be changed from the corresponding residues shown in SEQ ID NOS:2n, wherein n is an integer between 1 and 45, while still encoding a protein that maintains its NOVX activities and physiological functions, or a functional fragment thereof.

In general, A NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, A NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2n, wherein n is an integer between 1 and 45) that include fewer amino acids than the full-length NOVX proteins, and exhibit at least one activity of A NOVX protein. Typically, biologically-active portions comprise a domain or motif with at least one activity of the NOVX protein. A biologically-active portion of A NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2n, wherein n is an integer between 1 and 45. In other embodiments, the NOVX protein is substantially homologous to SEQ ID NOS:2n, wherein n is an integer between 1 and 45, and retains the functional activity of the protein of SEQ ID NOS:2n, wherein n is an integer between 1 and 45, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2n, wherein n is an integer between 1 and 45, and retains the functional activity of the NOVX proteins of SEQ ID NOS:2n, wherein n is an integer between 1 and 45.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”)

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:2n-1, wherein n is an integer between 1 and 45.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, A NOVX “chimeric protein” or “fusion protein” comprises A NOVX polypeptide operatively-linked to a non-NOVX polypeptide. An “NOVX polypeptide” refers to a polypeptide having an amino acid sequence corresponding to A NOVX protein SEQ ID NOS:2n, wherein n is an integer between 1 and 45, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within A NOVX fusion protein the NOVX polypeptide can correspond to all or a portion of A NOVX protein. In one embodiment, A NOVX fusion protein comprises at least one biologically active portion of A NOVX protein. In another embodiment, A NOVX fusion protein comprises at least two biologically active portions of A NOVX protein. In yet another embodiment, A NOVX fusion protein comprises at least three biologically active portions of A NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is A NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between A NOVX ligand and A NOVX protein on the surface of a cell, to thereby suppress NOVX-mediated signal transduction in vivo. The NOVX-immunoglobulin fusion proteins can be used to affect the bioavailability of A NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with A NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g. Ausubel, et al. (eds.) Current Protocols IN Molecular Biology, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX AGonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade, which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods, which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well known within the art. See, e.g., Narang, 1983. Tetrahedron 39: 3; Itakara, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of A NOVX protein. In one embodiment, a library of coding sequence fragments can be generated by treating a double stranded PCR fragment of A NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S1 nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

NOVX Antibodies

The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, Fab, Fab, and F(ab)₂ fragments, and an Fab expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NOs: 2n, wherein n is an integer between 1 and 45, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies and Cancer Therapy, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F(ab)₂ fragment produced by pepsin digestion of an antibody molecule; (ii) an Fab fragment generated by reducing the disulfide bridges of an F(ab)₂ fragment; (iii) an Fab fragment generated by the treatment of the antibody molecule with papain and a reducing agent and (iv) Fv fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹In, ⁹⁰Y, and ¹⁸⁶R.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, O-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding A NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g. NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (In Vitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:2n-1, wherein n is an integer between 1 and 45, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: Manipulating the Mouse Embryo, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of A NOVX gene into which a deletion, addition or substitution has been introduced to thereby alter, e.g., functionally disrupt, the NOVX gene. The NOVX gene can be a human gene (e.g., the cDNA of SEQ ID NOS:2n-1, wherein n is an integer between 1 and 45), but more preferably, is a non-human homologue of a human NOVX gene. For example, a mouse homologue of human NOVX gene of SEQ ID NOS:2n-1, wherein n is an integer between 1 and 45, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: Teratocarcinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., A NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in A NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays The invention provides a method (also referred to herein as a “screening assay”) for identifying modulators, ie., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of A NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to A NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with A NOVX protein, wherein determining the ability of the test compound to interact with A NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with A NOVX target molecule. As used herein, a “target molecule” is a molecule with which A NOVX protein binds or interacts in nature, for example, a molecule on the surface of a cell which expresses A NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or A NOVX protein or polypeptide of the invention. In one embodiment, A NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with A NOVX target molecule can be accomplished by one of the methods described above for determining direct binding. In one embodiment, determining the ability of the NOVX protein to bind to or interact with A NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising A NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting A NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with A NOVX protein, wherein determining the ability of the test compound to interact with A NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to A NOVX target molecule by one of the methods described above for determining direct binding. In an alternative embodiment, determining the ability of the test compound to modulate the activity of NOVX protein can be accomplished by determining the ability of the NOVX protein further modulate A NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with A NOVX protein, wherein determining the ability of the test compound to interact with A NOVX protein comprises determining the ability of the NOVX protein to preferentially bind to or modulate the activity of A NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS(N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming A NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:2n-1, wherein n is an integer between 1 and 45, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., Human Chromosomes: A Manual OF Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:2n-1, wherein n is an integer between 1 and 45, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in A NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:2n-1, wherein n is an integer between 1 and 45, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in A NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding A NOVX-protein, or the misexpression of the NOVX gene. For example, such genetic lesions can be detected by ascertaining the existence of at least one of: (i) a deletion of one or more nucleotides from A NOVX gene; (ii) an addition of one or more nucleotides to A NOVX gene; (iii) a substitution of one or more nucleotides of A NOVX gene, (iv) a chromosomal rearrangement of A NOVX gene; (v) an alteration in the level of a messenger RNA transcript of A NOVX gene, (vi) aberrant modification of A NOVX gene, such as of the methylation pattern of the genomic DNA, (vii) the presence of a non-wild-type splicing pattern of a messenger RNA transcript of A NOVX gene, (viii) a non-wild-type level of A NOVX protein, (ix) allelic loss of A NOVX gene, and (x) inappropriate post-translational modification of A NOVX protein. As described herein, there are a large number of assay techniques known in the art which can be used for detecting lesions in A NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to A NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qp Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in A NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on A NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g. Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving A NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome Pregnancy Zone Protein Precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with A NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of A NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, A NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of A NOVX protein, a peptide, A NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of A NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering A NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

The NOV1 clone was analyzed, and the nucleotide and encoded polypeptide sequences are shown in Table 1A.

TABLE 1A
NOV1 Sequence Analysis

| | | |
|---|---|---|
| | SEQ ID NO:1 | 558 bp |
| NOV1, | ATGCCTCGCCTGTTTTTTTTCCACCTGCTAGAATTCTGTTTACTACTGAACCAATTTT | |
| CG56908-02 | DNACCAGAGCAGTCGCGGCCAAATGGAAGGACGATGTTATTAAATTATGCGGCCGCGAATT | |
| Sequence | AGTTCGCGCGCAGATTGCCATTTGCGGCATGAGCACCTGGAGCAAAAGGTCTCTGAGC | |
| | CAGGAAGATGCTCCTCAGACACCTAGACCAGTGGCAGAAATTGTGCCATCCTTCATCA | |
| | ACAAAGATACAGAAACCATAAATATGATGTCAGAATTTGTTGCTAATTTGCCACAGGA | |
| | GCTGAAGTTAACCCTGTCTGAGATGCAGCCAGCATTACCACAGCTACAACAACATGTA | |
| | CCTGTATTAAAAGATTCCAGTCTTCTCTTTGAAGAATTTAAGAAACTTATTCGCAATA | |
| | GACAAAGTGAAGCCGCAGACAGCAGTCCTTCAGAATTAAAATACTTAGGCTTGGATAC | |
| | TCATTCTCGAAAAAAGAGACAACTCTACAGTGCATTGGCTAATAAATGTTGCCATGTT | |
| | GGTTGTACCAAAAGATCTCTTGCTAGATTTTGCTGA | |
| | ORF Start: ATG at 1 | ORF Stop: TGA at 556 |
| | SEQ ID NO:2 | 185 aa MW at 21128.4 kD |

| | |
|---|---|
| NOV1, | MPRLFFFHLLEFCLLLNQFSRAVAAKWKDDVIKLCGRELVRAQIAICGMSTWSKRSLS |
| CG56908-02 | ProteinQEDAPQTPRPVAEIVPSFINKDTETINMMSEFVANLPQELKLTLSEMQPALPQLQQHV |
| Sequence | PVLKDSSLLFEEFKKLIRNRQSEAADSSPSELKYLGLDTHSRKKRQLYSALANKCCHV |
| | GCTKRSLARFC |

Further analysis of the NOV1 protein yielded the following properties shown in Table 1B.

TABLE 1B
Protein Sequence Properties NOV1

| | |
|---|---|
| PSort | 0.4712 probability located in mitochondrial matrix space; 0.3000 probability located in |
| analysis: | nucleus; 0.1737 probability located in mitochondrial inner membrane; 0.1737 probability |
| | located in mitochondrial intermembrane space |
| SignalP | Cleavage site between residues 25 and 26 |
| analysis: | |

A search of the NOV1 protein against the Geneseq database, a proprietary database that contains sequences published in patents and patent publication, yielded several homologous proteins shown in Table 1C.

TABLE 1C
Geneseq Results for NOV1

| | | NOV1 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Regionc | Value |
| AAP94621 | Amino acid sequence of human | 1 . . . 185 | 178/185 (96%) | 1e−99 |
| | preprorelaxin H2 - Homo sapiens, 185 aa. | 1 . . . 185 | 180/185 (97%) | |
| | [EP303033-A, 15 Feb. 1989] | | | |
| AAP40108 | Sequence of human preprorelaxin H2 - H2, | 1 . . . 185 | 177/185 (95%) | 6e−99 |
| | 185 aa. [EP112149-A, 27 Jun. 1984] | 1 . . . 185 | 179/185 (96%) | |
| AAP40155 | Sequence of human preprorelaxin - Homo | 1 . . . 185 | 159/185 (85%) | 3e−89 |
| | sapiens, 185 aa. [EP101309-A, | 1 . . . 185 | 171/185 (91%) | |
| | 22 Feb. 1984] | | | |
| AAP40154 | Sequence of human preprorelaxin - Homo | 1 . . . 185 | 159/185 (85%) | 3e−89 |
| | sapiens, 185 aa. [EP101309-A, | 1 . . . 185 | 171/185 (91%) | |
| | 22 Feb. 1984] | | | |
| AAP94622 | Amino acid sequence of human | 1 . . . 185 | 157/185 (84%) | 2e−87 |
| | preprorelaxin H1 - Homo sapiens, 185 aa. | 1 . . . 185 | 169/185 (90%) | |
| | [EP303033-A, 15 Feb. 1989] | | | |

In a BLAST search of public sequence databases, the NOV1 protein was found to have homology to the proteins shown in the BLASTP data in Table 1D.

TABLE 1D
Public BLASTP Results for NOV1

| | | NOV1 | | |
|---|---|---|---|---|
| Protein | | Residues/ | Identities/ | |
| Accession | | Match | Similarities for the | Expect |
| Number | Protein/Organism/Length | Residues | Matched Portion | Value |
| P04090 | Prorelaxin H2 precursor - Homo | 1 . . . 185 | 178/185 (96%) | 4e−99 |
| | sapiens (Human), 185 aa. | 1 . . . 185 | 180/185 (97%) | |
| P04808 | Prorelaxin H1 precursor - Homo | 1 . . . 185 | 159/185 (85%) | 8e−89 |
| | sapiens (Human), 185 aa. | 1 . . . 185 | 171/185 (91%) | |
| P51455 | Prorelaxin H2 precursor - Pan | 20 . . . 185 | 160/166 (96%) | 1e−87 |
| | troglodytes (Chimpanzee), 166 aa | 1 . . . 166 | 162/166 (97%) | |
| | (fragment). | | | |
| P19884 | Prorelaxin precursor - Macaca mulatta | 1 . . . 185 | 154/185 (83%) | 2e−85 |
| | (Rhesus macaque), 185 aa. | 1 . . . 185 | 165/185 (88%) | |
| P51454 | Prorelaxin H1 precursor - Pan | 20 . . . 185 | 137/166 (82%) | 3e−74 |
| | troglodytes (Chimpanzee), 166 aa | 1 . . . 166 | 148/166 (88%) | |
| | (fragment). | | | |

PFam analysis predicts that the NOV1 protein contains the domains shown in the Table 1E.

TABLE 1E
Domain Analysis of NOV1

| | | Identities/ | |
|---|---|---|---|
| | NOV1 | Similarities for | Expect |
| Pfam Domain | Match Region | the Matched Region | Value |
| DUF38: domain 1 of 1 | 6 . . . 33 | 11/40 (28%) | 2.2 |
| | | 20/40 (50%) | |
| Insulin: domain 1 of 1 | 32 . . . 185 | 59/160 (37%) | 4.2e−49 |
| | | 128/160 (80%) | |

### Example 2

The NOV2 clone was analyzed, and the nucleotide and encoded polypeptide sequences are shown in Table 2A.

TABLE 2A
NOV2 Sequence Analysis

| | SEQ ID NO:3 | 1055 bp |
|---|---|---|
| NOV2a, | GCCCGCGACTCGGAGCACCCCACCCCTCCCCTGCCGGGCCAGGCCGGGCGGCGTTGTT | |
| CG59783-01 DNA | GGCGGGGGCCCCGGTGGAGGCCCGGCCCGGGCGGCGCCCGCCATGAACGGGCTGTCGC | |
| Sequence | TGAGTGAGCTCTGCTGCCTCTTCTGCTGCCCTCCCTGCCCCGGCCGCATCGCTGCCAA | |
| | GCTCGCCTTCCTGCCGCCGGAGGCCACCTACTCCCTGGTGCCTGAGCCCGAGCTGGGG | |
| | CGCTGGAAGCTGCACCTGACGGAGCGTGCCGACTTCCAGTACAGCCAGCGCGAGCTGG | |
| | ACACCATCGAGGTCTTCCCCACCAAGAGCGCCCGCGGCAACCGTGTCTCCTGCATGTA | |
| | TGTTCGCTGCGTGCCTGGTGCCAGGTACACGGTCCTCTTCTCGCACGGCAATGCCGTG | |
| | GACCTGGGCCAGATGAGCAGCTTCTACATTGGCCTGGGCTCCCGCCTCCACTGCAACA | |
| | TCTTCACCTACGACTCCTCCGGCTACGGTGCCAGCTCGGGCAGGCCTTCCGAGAGGAA | |
| | CCTCTATGCCGACATCGACGCCACCTGGCAGGCCCTGCGCACCAGGTACGGCATCAGC | |
| | CCGGACAGCATCATCCTGTACGGGCAGAGCATCGGCACGGTGCCCACCATGGACCTGG | |
| | CCTCGCGCTACGAGTGTGCCGCGGTGGTGCTGCACTCGCCGCTCACCTCGGGCATGCG | |
| | CGTCGCCTTCCGCGACACCAAGAAGACCTACTGCTTCGACGCCTTCCCTAACATCGAG | |
| | AAGGTGTCCAAGATCACGTCTCCCGTGCTCATCATCCACGGCAGGGAGGACGAGGTGA | |
| | TCGACTTCTCGCACGGGCTGGCGCTCTACGAGCGCTGCCCCAAGGCGGTGGAGCCGCT | |
| | GTGGGTGGAGGGCGCCGGGCACAACGACATCGAGCTCTACAGCCAGTACCTGGAGCGC | |
| | CTGCGTCGCTTCATCTCCCAGGAGCTGCCCAGCCAGCGCGCCTAGCGGCGGCCCCAAC | |
| | CAGCCGGACCTCAGCAATAAGGCGGCCCCCGGACCTCACCCCGCGCCGGCCCCCACCC | |
| | AGGGGCTGCAT | |
| | ORF Start: ATG at 101 | ORF Stop: TAG at 971 |
| | SEQ ID NO:4 | 290 aa MW at 32472.6 kD |
| NOV2a, | MNGLSLSELCCLFCCPPCPGRIAAKLAFLPPEATYSLVPEPELGRWKLHLTERADFQY | |
| CG59783-01 Protein | SQRELDTIEVFPTKSARGNRVSCMYVRCVPGARYTVLFSHGNAVDLGQMSSFYIGLGS | |
| Sequence | RLHCNIFTYDSSGYGASSGRPSERNLYADIDATWQALRTRYGISPDSIILYGQSIGTV | |
| | PTMDLASRYECAAVVLHSPLTSGMRVAFRDTKKTYCFDAFPNIEKVSKITSPVLIIHG | |
| | REDEVIDFSHGLALYERCPKAVEPLWVEGAGHNDIELYSQYLERLRRFISQELPSQRA | |
| | SEQ ID NO:5 | 976 bp |
| NOV2b, | CCATGAACGGGCTGTCGCTGAGTGAGCTCTGCTGCCTCTTCTGCTGCCCGCCCTGCCC | |
| CG59783-02 DNA | CGGCCGCATCGCTGCCAAGCTCGCCTTCCTGCCGCCGGAGGCCACCTACTCCCTGGTG | |
| Sequence | CCTGAGCCCGAACCGGGGCCTGGTGGGGCCGGGGCCGCCCCCTTGGGGACCCTGAGAG | |
| | CCTCCTCGGGCGCACCCGGGCGCTGGAAGCTGCACCTGACGGAGCGTGCCGACTTCCA | |
| | GTACAGCCAGCGCGAGCTGGACACCATCGAGGTCTTCCCCACCAAGAGCGCCCGCGGC | |
| | AACCGCGTCTCCTGCATGTATGTTCGCTGCGTGCCTGGTGCCAGGTACACGGTCCTCT | |
| | TCTCGCACGGCAATGCCGTGGACCTGGGCCAGATGAGCAGCTTCTACATTGGCCTGGG | |
| | CTCCCGCCTCCACTGCAACATCTTCTCCTACGACTACTCCGGCTACGGTGCCAGCTCG | |
| | GGCAGGCCTTCCGAGAGGAACCTCTATGCCGACATCGACGCCGCCTGGCAGGCCCTGC | |
| | GCACCAGGTACGGCATCAGCCCGGACAGCATCATCCTGTACGGGCAGAGCATCGGCAC | |
| | GGTGCCCACCGTGGACCTGGCCTCGCGCTACGAGTGTGCCGCGGTGGTGCTGCACTCG | |
| | CCGCTCACCTCGGGCATGCGCGTCGCCTTCCCCGACACCAAGAAGACCTACTGCTTCG | |
| | ACGCCTTCCCTAACATCGAGAAGGTGTCCAAGATCACGTCTCCCGTGCTCATCATCCA | |
| | CGGCACGGAGGACGAGGTGATCGACTTCTCGCACGGGCTGGCGCTCCACGAGCGCTGC | |
| | CCCAAGGCGGTGGAGCCGCTGTGGGTGGAGGGCGCCGGGCACAACGACATCGAGCTCT | |
| | ACAGCCAGTACCTGGAGCGCCTGCGTCGCTTCATCTCCCAGGAGCTGCCCAGCCAGCG | |
| | CGCCTAGCGGCGGCCCCAACCGGCCGGACCTCAGCAATAAGGCGGCCC | |
| | ORF Start: ATG at 3 | ORF Stop: TAG at 933 |
| | SEQ ID NO:6 | 310 aa MW at 33963.2 kD |

| | |
|---|---|
| NOV2b, | MNGLSLSELCCLFCCPPCPGRIAAKLAFLPPEATYSLVPEPEPGPGGAGAAPLGTLRA |
| CG59783-02 Protein | SSGAPGRWKLHLTERADFQYSQRELDTIEVFPTKSARGNRVSCMYVRCVPGARYTVLF |
| Sequence | SHGNAVDLGQMSSFYIGLGSRLHCNIFSYDYSGYGASSGRPSERNLYADIDAAWQALR |
| | TRYGISPDSIILYGQSIGTVPTVDLASRYECAAVVLHSPLTSGMRVAFPDTKKTYCFD |
| | AFPNIEKVSKITSPVLIIHGTEDEVIDFSHGLALHERCPKAVEPLWVEGAGHNDIELY |
| | SQYLERLRRFISQELPSQRA |

Sequence comparison of the above protein sequences yields the following sequence relationships shown in Table 2B.

TABLE 2B
Comparison of NOV2a against NOV2b.

| | NOV2a Residues/ | Identities/Similarities |
|---|---|---|
| Protein Sequence | Match Residues | for the Matched Region |
| NOV2b | 20 . . . 290 | 249/291 (85%) |
| | 20 . . . 310 | 251/291 (85%) |

Further analysis of the NOV2a protein yielded the following properties shown in Table 2C.

TABLE 2C
Protein Sequence Properties NOV2a

| | |
|---|---|
| PSort | 0.3700 probability located in outside; 0.1674 probability located in microbody |
| analysis: | (peroxisome); 0.1000 probability located in endoplasmic reticulum (membrane); 0.1000 |
| | probability located in endoplasmic reticulum (lumen) |
| SignalP | Cleavage site between residues 21 and 22 |
| analysis: | |

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM93226 | Human polypeptide, SEQ ID NO:2641 - | 1 . . . 290 | 283/310 (91%) | e−164 |
| | Homo sapiens, 310 aa. [EP1130094-A2, | 1 . . . 310 | 285/310 (91%) | |
| | 05 Sep. 2001] | | | |
| ABG27979 | Novel human diagnostic protein #27970 - | 1 . . . 290 | 273/310 (88%) | e−154 |
| | Homo sapiens, 403 aa. | 96 . . . 403 | 275/310 (88%) | |
| | [WO200175067-A2, 11 Oct. 2001] | | | |
| ABG27979 | Novel human diagnostic protein #27970 - | 1 . . . 290 | 273/310 (88%) | e−154 |
| | Homo sapiens, 403 aa. | 96 . . . 403 | 275/310 (88%) | |
| | [WO200175067-A2, 11 Oct. 2001] | | | |
| ABG18429 | Novel human diagnostic protein #18420 - | 1 . . . 290 | 215/349 (61%) | 5e−99 |
| | Homo sapiens, 344 aa. | 3 . . . 344 | 226/349 (64%) | |
| | [WO200175067-A2, 11 Oct. 2001] | | | |
| ABG18429 | Novel human diagnostic protein #18420 - | 1 . . . 290 | 215/349 (61%) | 5e−99 |
| | Homo sapiens, 344 aa. | 3 . . . 344 | 226/349 (64%) | |
| | [WO200175067-A2, 11 Oct 2001] | | | |

In a BLAST search of public sequence databases, the NOV2a protein was found to have homology to the proteins shown in the BLASTP data in Table 2E.

TABLE 2E
Public BLASTP Results for NOV2a

| | | NOV2a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q96GS6 | UNKNOWN (PROTEIN FOR | 1 . . . 290 | 283/310 (91%) | e—164 |
| | MGC:14860) - Homo sapiens | 1 . . . 310 | 285/310 (91%) | |
| | (Human), 310 aa. | | | |
| Q99JW1 | SIMILAR TO CGI-67 PROTEIN - | 1 . . . 290 | 267/310 (86%) | e−156 |
| | Mus musculus (Mouse), 310 aa. | 1 . . . 310 | 278/310 (89%) | |
| AAH18511 | HYPOTHETICAL 34.3 KDA | 1 . . . 287 | 227/312 (72%) | e−134 |
| | PROTEIN - Mus musculus (Mouse), | 1 . . . 312 | 261/312 (82%) | |
| | 313 aa. | | | |
| Q9Y377 | CGI-67 PROTEIN - Homo sapiens | 1 . . . 285 | 216/285 (75%) | e−133 |
| | (Human), 293 aa. | 1 . . . 285 | 256/285 (89%) | |
| Q9BWL0 | SIMILAR TO CGI-67 PROTEIN - | 1 . . . 215 | 208/235 (88%) | e−118 |
| | Homo sapiens (Human), 236 aa. | 1 . . . 235 | 210/235 (88%) | |

PFam analysis predicts that the NOV2a protein contains the domains shown in the Table 2F.

TABLE 2F
Domain Analysis of NOV2a

| | | Identities/ | |
|---|---|---|---|
| | NOV2a | Similarities | Expect |
| Pfam Domain | Match Region | for the Matched Region | Value |
| abhydrolase_2: | 79 . . . 285 | 42/255 (16%) | 0.11 |
| domain 1 of 1 | | 139/255 (55%) | |

### Example 3

The NOV3 clone was analyzed, and the nucleotide and encoded polypeptide sequences are shown in Table 3A.

TABLE 3A
NOV3 Sequence Analysis

| | SEQ ID NO:7 | 468 bp |
|---|---|---|
| NOV3, | TGCTTCCTGTGCCCTGCGCCATGTGGAGTCTGCCGCCGAGCAGGGCTCTGTCCTGTGC | |
| CG59873-01 | DNAGCCACTGCTGCTTCTCTTCAGCTTCCAGTTCCTGGTTACCTATGCTTGGCGTTTCCAA | |
| Sequence | GAGGAAGAGGAGTGGAATGACCAAAAACAAATTGCTGTTTATCTCCCTCCCACCCTGG | |
| | GCCTGTCCTGGCTTCCTGGAAGGAGCAGGGTTATGATAAGATGACATTCTCCATGAAT | |
| | CTGCAACTGGGCAGAACCATGTGTGGGAAATTTGAAGATGACATTGACAACTGCCCTT | |
| | TTCAAGAGAGCCCAGAGCTGAACAATACCTGCACCTGCTTCTTCACCATTGGAATAGA | |
| | GCCCTGGAGGACACGGTTTGACCTCTGGAACAAGACGTGCTCAGGCGGGCATTCCTGA | |
| | GTGG | |
| | ORF Start: ATG at 21 | ORF Stop: TGA at 462 |
| | SEQ ID NO:8 | 147 aa MW at 17315.6 kD |

| | |
|---|---|
| NOV3, | MWSLPPSRALSCAPLLLLFSFQFLVTYAWRFQEEEEWNDQKQIAVYLPPTLEFAVYTF |
| CG59873-01 Protein | NKQSKDWYAYKLVPVLASWKEQGYDKMTFSMNLQLGRTMCGKFEDDIDNCPFQESPEL |
| Sequence | NNTCTCFFTIGIEPWRTRFDLWNKTCSGGHS |

Further analysis of the NOV3 protein yielded the following properties shown in Table 3B.

TABLE 3B
Protein Sequence Properties NOV3

| | |
|---|---|
| PSort | 0.7475 probability located in outside; 0.3200 probability |
| analysis: | located in microbody (peroxisome); 0.1900 probability |
| | located in lysosome (lumen); 0.1000 probability located |
| | in endoplasmic reticulum (membrane) |
| SignalP | Cleavage site between residues 29 and 30 |
| analysis: | |

A search of the NOV3 protein against the Geneseq database, a proprietary database that contains sequences published in patents and patent publication, yielded several homologous proteins shown in Table 3C.

TABLE 3C
Geneseq Results for NOV3

| | | NOV3 | | |
|---|---|---|---|---|
| | | Residues/ | Identities/ | |
| Geneseq | Protein/Organism/Length [Patent # | Match | Similarities for the | Expect |
| Identifier | Date] | Residues | Matched Region | Value |
| AAG67508 | Amino acid sequence of a human secreted | 1 . . . 147 | 147/147 (100%) | 8e−89 |
| | polypeptide - Homo sapiens, 148 aa. | 2 . . . 148 | 147/147 (100%) | |
| | [WO200166690-A2, 13-SEP-2001] | | | |
| AAG67507 | Amino acid sequence of a human secreted | 1 . . . 118 | 118/118 (100%) | 4e−68 |
| | polypeptide - Homo sapiens, 159 aa. | 2 . . . 119 | 118/118 (100%) | |
| | [WO200166690-A2, 13-SEP-2001] | | | |
| AAY53771 | A human cystatin-related protein, | 1 . . . 145 | 89/145 (61%) | 5e−46 |
| | designated testatin - Homo sapiens, 147 aa. | 1 . . . 145 | 102/145 (69%) | |
| | [WO9958565-A1, 18-NOV-1999] | | | |
| AAG67506 | Amino acid sequence of a human secreted | 1 . . . 145 | 88/145 (60%) | 7e−45 |
| | polypeptide - Homo sapiens, 148 aa. | 2 . . . 146 | 101/145 (68%) | |
| | [WO200166690-A2, 13-SEP-2001] | | | |
| AAB87597 | Human PRO3543 - Homo sapiens, 147 aa. | 1 . . . 145 | 88/145 (60%) | 7e−45 |
| | [WO200116318-A2, 08-MAR-2001] | 1 . . . 145 | 101/145 (68%) | |

In a BLAST search of public sequence databases, the NOV3 protein was found to have homology to the proteins shown in the BLASTP data in Table 3D.

TABLE 3D
Public BLASTP Results for NOV3

| | | NOV3 | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9H4G1 | Cystatin 9-like precursor - Homo | 1 . . . 145 | 88/145 (60%) | 2e−44 |
| | sapiens (Human), 147 aa. | 1 . . . 145 | 101/145 (68%) | |
| CAC05423 | BA218C14.3 PROTEIN - Homo | 8 . . . 147 | 81/145 (55%) | 3e−37 |
| | sapiens (Human), 152 aa. | 8 . . . 152 | 100/145 (68%) | |
| Q9Z0H6 | Cystatin 9 precursor (Testatin) - Mus | 8 . . . 143 | 63/136 (46%) | 2e−28 |
| | musculus (Mouse), 137 aa. | 8 . . . 137 | 87/136 (63%) | |
| Q9D264 | 9230104L09RIK PROTEIN - Mus | 9 . . . 145 | 50/137 (36%) | 2e−13 |
| | musculus (Mouse), 133 aa. | 2 . . . 131 | 70/137 (50%) | |
| Q9DAN8 | 1700006F03RIK PROTEIN - Mus | 50 . . . 142 | 34/93 (36%) | 5e−13 |
| | musculus (Mouse), 128 aa. | 36 . . . 125 | 57/93 (60%) | |

PFam analysis predicts that the NOV3 protein contains the domains shown in the Table 3E.

TABLE 3E
Domain Analysis of NOV3

| | NOV3 | Identities/ | |
|---|---|---|---|
| Pfam | Match | Similarities | |
| Domain | Region | for the Matched Region | Expect Value |
| cystatin: | 49 . . . 142 | 28/97 (29%) | 8.4e−07 |
| domain 1 of 1 | | 62/97 (64%) | |

### Example 4

The NOV4 clone was analyzed, and the nucleotide and encoded polypeptide sequences are shown in Table 4A.

TABLE 4A
NOV4 Sequence Analysis

| | SEQ ID NO:9 | 5538 bp |
|---|---|---|
| NOV4, | GGCGCGGAGAGCTCCCAACCTGGGCTGGAACCTTGCCCAGCACAGGTGGCTGCTACAC | |
| CG89060-01 DNA | CCCATGTAAAAAGCGGAAAATAAAATGAAGATTTTCCAGCGCAAGATGCGGTACTGGT | |
| Sequence | TGCTTCCACCTTTTTTGGCAATTGTTTATTTCTGCACCATTGTCCAAGGTCAAGTGGC | |
| | TCCACCCACAAGGTTAAGATATAATGTAATATCTCATGACAGTATACAGATTTCATGG | |
| | AAGGCTCCAAGAGGGAAATTTGGTGGTTACAAACTTCTTGTGACTCCAACTTCAGGTG | |
| | GAAAAACTAACCAGCTGAATCTGCAGAACACTGCAACTAAAGCAATTATTCAAGGCCT | |
| | TATGCCAGACCAGAATTACACAGTTCAAATTATTGCATACAATAAAGATAAAGAAAGC | |
| | AAGCCAGCTCAAGGCCAATTCAGAATTAAAGATTTAGAAAAAAGAAAGGATCCAAAGC | |
| | CCAGAGTCAAAGTTGTGGACAGAGGAAATGGGAGTAGACCATCTTCACCAGAAGAAGT | |
| | GAAATTTGTCTGTCAAACTCCAGCAATTGCTGACATTGTAATCCTGGTCGATGGTTCA | |
| | TGGAGTATTGGAAGATTCAACTTCAGACTGGTTCGGCATTTCTTGGAAAACCTGGTTA | |
| | CGGCATTCGATGTGGGCTCAGAGAAGACACGAATTGGTCTTGCACAGTATAGTGGTGA | |
| | CCCCAGAATAGAATGGCACTTGAATGCATTTAGCACAAAAGATGAAGTGATTGAAGCT | |
| | GTCCGAAACCTCCCATATAAAGGAGGAAATACACTAACAGGTCTTGCTTTGAACTACA | |
| | TTTTTGAAAATAGCTTCAAACCAGAAGCAGGATCAAGGACTGGAGTATCCAAAATTGG | |
| | CATTTTAATCACAGATGGAAAATCCCAAGATGACATTATTCCACCATCTAGAAATCTT | |
| | CGTGAGTCTGGTGTAGAACTGTTTGCCATAGGGGTGAAAAACGCGGATGTGAATGAGC | |
| | TGCAGGAGATCGCCTCTGAACCAGACAGCACTCATGTGTACAATGTTGCCGAATTCGA | |
| | TCTGATGCACACAGTTGTGGAGAGTCTGACCAGGACTCTCTGCTCTAGAGTGGAAGAA | |
| | CAGGACAGAGAAATTAAAGCCTCAGCCCATGCCATCACTGGGCCGCCTACGGAGTTGA | |
| | TTACTTCTGAAGTCACTGCCAGAAGCTTTATGGTTAACTGGACTCATGCCCCAGGAAA | |
| | TGTGGAAAAATACAGAGTTGTGTATTATCCTACCAGGGGTGGAAAACCAGACGAGGTG | |
| | GTGGTAGATGGAACTGTATCTTCCACAGTGTTGAAAAACTTGATGTCTTTAACTGAAT | |
| | ATCAGATAGCAGTCTTTGCAATCTATGCCCACACTGCTAGTGAAGGCCTACGGGGAAC | |
| | TGAAACTACACTTGCTTTACCGATGGCTTCTGACCTTCTACTGTACGACGTGACTGAG | |
| | AACAGCATGCGAGTCAAATGGGATGCAGTGCCTGGGGCCTCAGGTTACCTGATCCTTT | |
| | ATGCTCCTCTAACAGAGGGCCTGGCTGGGGATGAAAAAGAGATGAAAATTGGAGAGAC | |
| | CCACACAGATATTGAATTGAGTGGGTTGTTGCCCAATACAGAATACACAGTCACAGTT | |
| | TATGCCATGTTTGGAGAAGAGGCCAGTGATCCTGTTACGGGACAAGAAACAACATTGG | |
| | CTTTAAGTCCACCAAGAAACCTGAGAATCTCCAATGTTGGCTCTAACAGTGCTCGATT | |
| | AACCTGGGACCCAACTTCAAGACAGATCCATGGTTATCGAATTGTATATAACAATGCA | |
| | GATGGGACTGAAATCAATGAGGTTGAAGTCGATCCTATTACTACCTTCCCTCTGAAGG | |
| | GCTTGACACCTCTCACAGAGTATACTATTGCTATTTTCTCCATCTATGATGAAGGACA | |
| | GTCAGAGCCTCTGACTGGAGTTTTTACCACCGAGGAAGTTCCAGCCCAGCAATACTTA | |
| | GAAATTGATGAGGTGACGACAGACAGTTTTAGGGTGACCTGGCATCCCCTCTCAGCTG | |
| | ATGAAGGGCTACACAAATTGATGTGGATTCCAGTCTATGGGGGGAAGACTGAGGAGGT | |
| | TGTCCTGAAAGAAGAGCAGGACTCACATGTTATTGAAGGCCTGGAGCCCGGTACGGAG | |
| | TATGAAGTTTCACTATTGGCCGTACTTGATGATGGAAGCGAGAGTGAGGTGGTGACTG | |
| | CTGTCGGGACCACACTTGACAGTTTTTGGACAGAACCAGCTACAACCATAGTGCCTAC | |
| | CACATCTGTGACTTCAGTTTTCCAGACGGGAATCAGAAACCTAGTTGTAGGTGATGAA | |
| | ACTACTTCTAGCCTGCGGGTAAAATGGGACATTTCTGACAGCGATGTGCAGCAGTTTA | |
| | GGGTGACCTACATGACAGCTCAAGGGGACCCTGAGGAAGAAGTCATAGGAACGGTTAT | |
| | GGTGCCTGGAAGCCAGAACAACCTCCTTCTGAAGCCTCTGCTTCCTGATACTGAATAC | |
| | AAAGTCACAGTGACTCCCATCTACACGGATGGCGAAGGCGTCAGCGTCTCCGCTCCTG | |
| | GAAAAACCTTACCATCCTCGGGGCCCCAGAACTTGCGGGTGTCCGAGGAATGGTATAA | |
| | CCGGTTGCGCATTACGTGGGACCCCCCATCTTCCCCGGTGAAAGGCTATAGAATTGTC | |
| | TACAAACCTGTCAGTGTTCCTGGTCCAACACTGGAAACGTTTGTGGGAGCTGACATTA | |
| | ACACCATCCTTATCACAAACCTCCTCAGCGGAATGGACTACAATGTGAAGATATTTGC | |
| | CTCCCAGGCCTCAGGCTTCAGCGACGCCCTGACAGGCATGGTGAAAACATTGTTCTTG | |
| | GGTGTTACCAATCTCCAAGCCAAACATGTTGAAATGACCAGCTTGTGTGCCCACTGGC | |
| | AGGTACATCGCCATGCCACAGCCTATAGGGTTGTTATAGAATCCCTCCAGGATAGGCA | |
| | AAAGCAAGAATCCACTGTGAGTGGAGGGACAACCAGGCATTGCTTCTATGGACTTCAG | |
| | CCTGATTCTGAATATAAAATCAGTGTTTATACAAAGCTCCAGGAGATTGAAGGACCTA | |
| | GTGTGAGCATAATGGAAAAAACACAATCACTTCCTACACGACCACCAACTTTTCCTCC | |
| | AACCATTCCACCAGCAAAAGAAGTATGTAAGGCGGCCAAGGCTGACCTGGTATTTATG | |
| | GTGGATGGATCCTGGAGCATTGGAGATGAAAATTTCAATAAGATCATCAGCTTTCTAT | |
| | ACAGCACTGTTGGAGCCCTGAACAAGATTGGCACAGATGGAACCCAAGTTGCAATGGT | |
| | TCAGTTCACTGATGATCCCAGAACAGAATTTAAACTAAATGCTTACAAAACCAAAGAG | |
| | ACTCTTCTTGATGCAATTAAACACATTTCATACAAAGGAGGAAATACAAAAACAGGAA | |
| | AAGCAATTAAGTATGTTCGAGATACCTTGTTCACTGCAGAGTCAGGTACAAGAAGGGG | |
| | CATCCCAAAGGTTATCGTGGTTATAACTGATGGAAGATCACAAGATGATGTGAACAAA | |
| | ATCTCCAGGGAGATGCAATTAGATGGCTATAGCATTTTTGCAATTGGTGTGGCCGATG | |
| | CAGATTACTCGGAGTTGGTTAGCATTGGCAGTAAGCCCAGCGCACGCCATGTCTTCTT | |
| | TGTGGATGACTTTGACGCCTTTAAGAAAATCGAAGATGAGTTAATTACTTTTGTCTGC | |
| | GAAACAGCATCAGCAACCTGTCCAGTGGTACACAAGGATGGCATTGATCTTGCAGGAT | |
| | TTAAGATGATGGAAATGTTTGGTTTGGTTGAAAAAGATTTTTCATCAGTGGAAGGGGT | |
| | TTCTATGGAGCCTGGTACCTTCAATGTGTTTCCATGTTACCAACTCCATAAAGATGCC | |
| | CTGGTTTCCCAGCCAACCAGGTACTTGCACCCAGAAGGATTGCCCTCCGACTACACAA | |
| | TCAGTTTTCTATTCCGGATTCTTCCTGACACTCCACAGGAGCCATTTGCTCTTTGGGA | |
| | GATTTTAAATAAAAATTCTGACCCATTGGTTGGGGTTATTCTAGACAATGGTGGGAAA | |
| | ACTCTAACATATTTCAACTATGACCAGAGTGGGGATTTTCAAACTGTTACTTTCGAAG | |
| | GACCTGAAATTAGGAAAATTTTTTATGGAAGCTTTCACAAGCTACACATTGTTGTCAG | |
| | TGAGGCTTTGGTCAAAGTGGTTATTGACTGCAAGCAAGTGGGTGAGAAGGCAATGAAC | |
| | GCATCAGCTAATATCACGTCAGATGGTGTAGAAGTGCTAGGGAAAATGGTTCGATCAA | |
| | GAGGACCAGGTGGAAACTCTGCACCGTTCCAGTTACAGATGTTTGATATTGTTTGCTC | |
| | CACATCATGGGCCAATACAGACAAATGCTGTGAACTTCCAGGCCTGAGAGATGATGAG | |
| | TCTTGCCCAGACCTTCCCCATTCCTGCTCCTGTTCTGAAACCAATGAAGTGGCTCTGG | |
| | GACCAGCGGGCCCACCAGGTGGTCCAGGACTCCGAGGACCAAAGGGCCAGCAAGGTGA | |
| | ACCGGGTCCAAAGGGACCAGATGGCCCTCGGGGTGAAATTGGTCTGCCAGGACCTCAG | |
| | GGTCCACCTGGACCTCAAGGACCAAGTGGTCTGTCCATTCAAGGAATGCCCGGAATGC | |
| | CAGGAGAAAAAGGAGAGAAAGGAGATACTGGCCTTCCAGGTCCACAGGGTATCCCAGG | |
| | AGGCGTTGGTTCACCAGGACGTGATGGCTCACCAGGCCAGAGGGGCCTTCCGGGAAAG | |
| | GATGGATCCTCGGGACCTCCAGGACCACCAGGGCCAATAGGCATTCCTGGCACCCCTG | |
| | GAGTCCCAGGGATCACAGGAAGCATGGGACCGCAAGGCGCCCTGGGACCACCTGGTGT | |
| | CCCTGGAGCAAAGGGGGAACGAGGAGAGCGGGGTGACCTGCAGTCTCAAGCCATGGTG | |
| | AGATCAGTGGCGCGTCAAGTATGCGAACAGCTCATCCAGAGTCACATGGCCAGGTACA | |
| | CTGCCATCCTCAACCAGATTCCCAGCCACTCCTCATCCATCCGGACTGTCCAAGGGCC | |
| | TCCTGGGGAGCCTGGGAGGCCAGGCTCACCTGGAGCCCCTGGTGAACAAGGACCCCCA | |
| | GGCACACCAGGCTTCCCCGGAAATGCAGGCGTGCCAGGGACCCCAGGAGAACGAGGTC | |
| | TAACTGGTATCAAAGGAGAAAAAGGAAATCCAGGCGTTGGAACCCAAGGTCCAAGAGG | |
| | CCCCCCTGGACCAGCAGGACCTTCAGGGGAGAGTCGGCCTGGCAGCCCTGGGCCCCCT | |
| | GGCTCTCCTGGACCAAGAGGCCCACCAGGTCATCTGGGGGTTCCTGGACCCCAAGGTC | |
| | CTTCTGGCCAGCCTGGATATTGTGACCCCTCATCATGTTCTGCCTATGGTGTGAGAGA | |
| | TCTGATCCCCTACAATGATTACCAGCACTGAAGTGGAAATCCTCCACTCTGGTTCCAT | |
| | TGGCCCCAGACATTTAGCTGTGGATACAGAACTGTCCTGTCAACCACCACCACCACCA | |
| | AGCCCCTGCCCCTAACAATGGACACTCT | |
| | ORF Start: ATG at 83 | ORF Stop: TGA at 5423 |
| | SEQ ID NO:10 | 1780 aa MW at 191924.0 kD |

| | |
|---|---|
| NOV4, | MKIFQRKMRYWLLPPFLAIVYFCTIVQGQVAPPTRLRYNVISHDSIQISWKAPRGKFG |
| CG89060-01 Protein | GYKLLVTPTSGGKTNQLNLQNTATKAIIQGLMPDQNYTVQIIAYNKDKESKPAQGQFR |
| Sequence | IKDLEKRKDPKPRVKVVDRGNGSRPSSPEEVKFVCQTPAIADIVILVDGSWSIGRFNF |
| | RLVRHFLENLVTAFDVGSEKTRIGLAQYSGDPRIEWHLNAFSTKDEVIEAVRNLPYKG |
| | GNTLTGLALNYIFENSFKPEAGSRTGVSKIGILITDGKSQDDIIPPSRNLRESGVELF |
| | AIGVKNADVNELQEIASEPDSTHVYNVAEFDLMHTVVESLTRTLCSRVEEQDREIKAS |
| | AHAITGPPTELITSEVTARSFMVNWTHAPGNVEKYRVVYYPTRGGKPDEVVVDGTVSS |
| | TVLKNLMSLTEYQIAVFAIYAHTASEGLRGTETTLALPMASDLLLYDVTENSMRVKWD |
| | AVPGASGYLILYAPLTEGLAGDEKEMKIGETHTDIELSGLLPNTEYTVTVYAMFGEEA |
| | SDPVTGQETTLALSPPRNLRISNVGSNSARLTWDPTSRQIHGYRIVYNNADGTEINEV |
| | EVDPITTFPLKGLTPLTEYTIAIFSIYDEGQSEPLTGVFTTEEVPAQQYLEIDEVTTD |
| | SFRVTWHPLSADEGLHKLMWIPVYGGKTEEVVLKEEQDSHVIEGLEPGTEYEVSLLAV |
| | LDDGSESEVVTAVGTTLDSFWTEPATTIVPTTSVTSVFQTGIRNLVVGDETTSSLRVK |
| | WDISDSDVQQFRVTYMTAQGDPEEEVIGTVMVPGSQNNLLLKPLLPDTEYKVTVTPIY |
| | TDGEGVSVSAPGKTLPSSGPQNLRVSEEWYNRLRITWDPPSSPVKGYRIVYKPVSVPG |
| | PTLETFVGADINTILITNLLSGMDYNVKIFASQASGFSDALTGMVKTLFLGVTNLQAK |
| | HVEMTSLCAHWQVHRHATAYRVVIESLQDRQKQESTVSGGTTRHCFYGLQPDSEYKIS |
| | VYTKLQEIEGPSVSIMEKTQSLPTRPPTFPPTIPPAKEVCKAAKADLVFMVDGSWSIG |
| | DENFNKIISFLYSTVGALNKIGTDGTQVAMVQFTDDPRTEFKLNAYKTKETLLDAIKH |
| | ISYKGGNTKTGKAIKYVRDTLFTAESGTRRGIPKVIVVITDGRSQDDVNKISREMQLD |
| | GYSIFAIGVADADYSELVSIGSKPSARHVFFVDDFDAFKKIEDELITFVCETASATCP |
| | VVHKDGIDLAGFKMMEMFGLVEKDFSSVEGVSMEPGTFNVFPCYQLHKDALVSQPTRY |
| | LHPEGLPSDYTISFLFRILPDTPQEPFALWEILNKNSDPLVGVILDNGGKTLTYFNYD |
| | QSGDFQTVTFEGPEIRKIFYGSFHKLHIVVSEALVKVVIDCKQVGEKAMNASANITSD |
| | GVEVLGKMVRSRGPGGNSAPFQLQMFDIVCSTSWANTDKCCELPGLRDDESCPDLPHS |
| | CSCSETNEVALGPAGPPGGPGLRGPKGQQGEPGPKGPDGPRGEIGLPGPQGPPGPQGP |
| | SGLSIQGMPGMPGEKGEKGDTGLPGPQGIPGGVGSPGRDGSPGQRGLPGKDGSSGPPG |
| | PPGPIGIPGTPGVPGITGSMGPQGALGPPGVPGAKGERGERGDLQSQAMVRSVARQVC |
| | EQLIQSHMARYTAILNQIPSHSSSIRTVQGPPGEPGRPGSPGAPGEQGPPGTPGFPGN |
| | AGVPGTPGERGLTGIKGEKGNPGVGTQGPRGPPGPAGPSGESRPGSPGPPGSPGPRGP |
| | PGHLGVPGPQGPSGQPGYCDPSSCSAYGVRDLIPYNDYQH |

Further analysis of the NOV4 protein yielded the following properties shown in Table 4B.

TABLE 4B
Protein Sequence Properties NOV4

| | |
|---|---|
| PSort | 0.5804 probability located in outside; 0.4449 probability |
| analysis: | located in lysosome (lumen); 0.1273 probability located in |
| | microbody (peroxisome); 0.1000 probability located in |
| | endoplasmic reticulum (membrane) |
| SignalP | Cleavage site between residues 29 and 30 |
| analysis: | |

A search of the NOV4 protein against the Geneseq database, a proprietary database that contains sequences published in patents and patent publication, yielded several homologous proteins shown in Table 4C.

TABLE 4C
Geneseq Results for NOV4

| | | NOV4 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAB27229 | Human EXMAD-7 SEQ ID NO:7 - Homo | 1002 . . . 1770 | 768/769 (99%) | 0.0 |
| | sapiens, 795 aa. [WO200068380-A2, | 1 . . . 769 | 768/769 (99%) | |
| | 16-NOV-2000] | | | |
| AAU27790 | Human full-length polypeptide sequence | 328 . . . 1776 | 656/1469 (44%) | 0.0 |
| | #115 - Homo sapiens, 3118 aa. | 1627 . . . 3055 | 901/1469 (60%) | |
| | [WO200164834-A2, 07-SEP-2001] | | | |
| AAG73916 | Human colon cancer antigen protein SEQ | 1223 . . . 1776 | 303/554 (54%) | 0.0 |
| | ID NO:4680 - Homo sapiens, 561 aa. | 12 . . . 553 | 378/554 (67%) | |
| | [WO200122920-A2, 05-APR-2001] | | | |
| AAM39822 | Human polypeptide SEQ ID NO:2967 - | 1582 . . . 1770 | 189/189 (100%) | e−113 |
| | Homo sapiens, 250 aa. | 36 . . . 224 | 189/189 (100%) | |
| | [WO200153312-A1, 26-JUL-2001] | | | |
| AAY08304 | Human collagen IX alpha-1 chain protein - | 1217 . . . 1757 | 191/576 (33%) | 4e−77 |
| | Homo sapiens, 921 aa. | 44 . . . 589 | 264/576 (45%) | |
| | [WO9921011-A1, 29-APR-1999] | | | |

In a BLAST search of public sequence databases, the NOV4 protein was found to have homology to the proteins shown in the BLASTP data in Table 4D.

TABLE 4D
Public BLASTP Results for NOV4

| | | NOV4 | | |
|---|---|---|---|---|
| Protein | | Residues/ | Identities/ | |
| Accession | | Match | Similarities for the | Expect |
| Number | Protein/Organism/Length | Residues | Matched Portion | Value |
| S31212 | collagen alpha 1(XIV) chain precursor, | 16 . . . 1779 | 1349/1793 (75%) | 0.0 |
| | short form - chicken, 1857 aa. | 15 . . . 1802 | 1542/1793 (85%) | |
| P32018 | Collagen alpha 1(XIV) chain precursor | 16 . . . 1779 | 1349/1793 (75%) | 0.0 |
| | (Undulin) - Gallus gallus (Chicken), | 15 . . . 1802 | 1542/1793 (85%) | |
| | 1888 aa. | | | |
| A45974 | collagen alpha 1(XIV) chain precursor, | 149 . . . 1779 | 1252/1664 (75%) | 0.0 |
| | short form 2 - chicken, 1747 aa. | 33 . . . 1692 | 1424/1664 (85%) | |
| Q05707 | UNDULIN 1 (MATRIX | 188 . . . 1024 | 834/837 (99%) | 0.0 |
| | GLYCOPROTEIN) - Homo sapiens | 1 . . . 837 | 835/837 (99%) | |
| | (Human), 843 aa (fragment). | | | |
| O00261 | COLLAGEN TYPE XIV - Homo | 1026 . . . 1780 | 754/755 (99%) | 0.0 |
| | sapiens (Human), 755 aa (fragment). | 1 . . . 755 | 754/755 (99%) | |

PFam analysis predicts that the NOV4 protein contains the domains shown in the Table 4E.

TABLE 4E
Domain Analysis of NOV4

| | | Identities/ | |
|---|---|---|---|
| | NOV4 | Similarities | |
| | Match | for the | Expect |
| Pfam Domain | Region | Matched Region | Value |
| fn3: domain 1 of 8 | 30 . . . 108 | 26/84 (31%) | 1.1e−15 |
| | | 65/84 (77%) | |
| vwa: domain 1 of 2 | 158 . . . 330 | 86/201 (43%) | 6.8e−64 |
| | | 148/201 (74%) | |
| fn3: domain 2 of 8 | 353 . . . 431 | 27/84 (32%) | 5e−15 |
| | | 59/84 (70%) | |
| fn3: domain 3 of 8 | 443 . . . 523 | 26/87 (30%) | 8.3e−09 |
| | | 54/87 (62%) | |
| fn3: domain 4 of 8 | 535 . . . 615 | 28/85 (33%) | 4.7e−17 |
| | | 66/85 (78%) | |
| fn3: domain 5 of 8 | 624 . . . 703 | 26/84 (31%) | 1.6e−08 |
| | | 57/84 (68%) | |
| fn3: domain 6 of 8 | 735 . . . 817 | 24/87 (28%) | 1.3e−06 |
| | | 60/87 (69%) | |
| E6: domain 1 of 1 | 866 . . . 886 | 9/21 (43%) | 8.7 |
| | | 16/21 (76%) | |
| fn3: domain 7 of 8 | 828 . . . 908 | 24/86 (28%) | 8.2e−15 |
| | | 58/86 (67%) | |
| fn3: domain 8 of 8 | 918 . . . 996 | 24/85 (28%) | 0.0018 |
| | | 54/85 (64%) | |
| vwa: domain 2 of 2 | 1032 . . . 1205 | 83/201 (41%) | 3.7e−71 |
| | | 155/201 (77%) | |
| TSPN: domain 1 of 1 | 1229 . . . 1424 | 62/222 (28%) | 5.2e−70 |
| | | 183/222 (82%) | |
| Collagen: domain 1 of 4 | 1460 . . . 1518 | 32/60 (53%) | 0.00028 |
| | | 46/60 (77%) | |
| Collagen: domain 2 of 4 | 1545 . . . 1604 | 33/60 (55%) | 1.5e−10 |
| | | 46/60 (77%) | |
| Collagen: domain 3 of 4 | 1646 . . . 1704 | 29/60 (48%) | 0.0001 |
| | | 42/60 (70%) | |
| Collagen: domain 4 of 4 | 1705 . . . 1762 | 33/60 (55%) | 0.0019 |
| | | 46/60 (77%) | |

### Example 5

The NOV5 clone was analyzed, and the nucleotide and encoded polypeptide sequences are shown in Table 5A.

TABLE 5A
NOV5 Sequence Analysis

| | SEQ ID NO:11 | 677 bp |
|---|---|---|
| NOV5, | ATGTGGGTCCCGGTTGTCTTCCTCACCCTGTCCGTGACGTGGATTGGTGCTGCGCCCC | |
| CG89511-01 DNA | TCATCCTGTCTCGGATTGTGGGAGGCTGGGAGTGCGAGAAGCATTCCCAACCCTGGCA | |
| Sequence | GGTGCTTGTGGCCTCTCGTGGCAGGGCAGTCTGCGGCGGTGTTCTGGTGCACCCCCAG | |
| | TGGGTCCTCACAGCTGCCCACTGCATCAGGAAGCCAGGTGATGACTCCAGCCACGACC | |
| | TCATGCTGCTCCGCCTGTCAGAGCCTGCCGAGCTCACGGATGCTGTGAAGGTCATGGA | |
| | CCTGCCCACCCAGGAGCCAGCACTGGGGACCACCTGCTACGCCTCAGGCTGGGGCAGC | |
| | ATTGAACCAGAGGAGTTCTTGACCCCAAAGAAACTTCAGTGTGTGGACCTCCATGTTA | |
| | TTTCCAATGACGTGTGTGCGCAAGTTCACCCTCAGAAGGTGACCAAGTTCATGCTGTG | |
| | TGCTGGACGCTGGACAGGGGGCAAAAGCACCTGCTGGGGTGATTCTGGGGGCCCACTT | |
| | GTCTGTAATGGTGTGCTTCAAGGTATCACGTCATGGGGCAGTGAACCATGTGCCCTGC | |
| | CCGAAAGGCCTTCCCTGTACACCAAGGTGGTGCATTACCGGAAGTGGATCAAGGACAC | |
| | CATCGTGGCCAACCCCTGAGCACCCCTATCAACCCCCTA | |
| | ORF Start: ATG at 1 | ORF Stop: TGA at 655 |
| | SEQ ID NO:12 | 218 aa MW at 23823.5 kD |

| | |
|---|---|
| NOV5, | MWVPVVFLTLSVTWIGAAPLILSRIVGGWECEKHSQPWQVLVASRGRAVCGGVLVHPQ |
| CG89511-01 | ProteinWVLTAAHCIRKPGDDSSHDLMLLRLSEPAELTDAVKVMDLPTQEPALGTTCYASGWGS |
| Sequence | IEPEEFLTPKKLQCVDLHVISNDVCAQVHPQKVTKFMLCAGRWTGGKSTCWGDSGGPL |
| | VCNGVLQGITSWGSEPCALPERPSLYTKVVHYRKWIKDTIVANP |

Further analysis of the NOV5 protein yielded the following properties shown in Table 5B.

TABLE 5B
Protein Sequence Properties NOV5

| | |
|---|---|
| PSort | 0.7236 probability located in outside; 0.1000 probability |
| analysis: | located in endoplasmic reticulum (membrane); 0.1000 |
| | probability located in endoplasmic reticulum (lumen); 0.1000 |
| | probability located in lysosome (lumen) |
| SignalP | Cleavage site between residues 18 and 19 |
| analysis: | |

A search of the NOV5 protein against the Geneseq database, a proprietary database that contains sequences published in patents and patent publication, yielded several homologous proteins shown in Table 5C.

TABLE 5C
Geneseq Results for NOV5

| | | NOV5 | Identities/ | |
|---|---|---|---|---|
| | | Residues/ | Similarities for | |
| Geneseq | | Match | the Matched | Expect |
| Identifier | Protein/Organism/Length [Patent #, Date] | Residues | Region | Value |
| AAB74830 | Prostate tumour antigen amino acid | 1 . . . 218 | 216/261 (82%) | e−124 |
| | sequence for a fusion protein - Homo | 8 . . . 268 | 217/261 (82%) | |
| | sapiens, 1079 aa. [WO200125272-A2, | | | |
| | 12-APR-2001] | | | |
| AAB74821 | Prostate tumour antigen amino acid | 1 . . . 218 | 216/261 (82%) | e−124 |
| | sequence for PSA - Homo sapiens, 261 aa. | 1 . . . 261 | 217/261 (82%) | |
| | [WO200125272-A2, 12-APR-2001] | | | |
| AAB19819 | Prostate specific antigen specific to benign | 25 . . . 218 | 192/237 (81%) | e−109 |
| | prostatic hyperplasia - Homo sapiens, 237 | 1 . . . 237 | 193/237 (81%) | |
| | aa. [WO200067030-A1, 09-NOV-2000] | | | |
| AAB19818 | Prostate specific antigen elevated in benign | 25 . . . 218 | 192/237 (81%) | e−109 |
| | prostatic hyperplasia - Homo sapiens, 237 | 1 . . . 237 | 193/237 (81%) | |
| | aa. [WO200066718-A1, 09-NOV-2000] | | | |
| AAG03734 | Human secreted protein, SEQ ID NO:7815 - | 1 . . . 174 | 168/174 (96%) | 1e−98 |
| | Homo sapiens, 234 aa. [EP1033401-A2, | 1 . . . 174 | 168/174 (96%) | |
| | 06-SEP-2000] | | | |

In a BLAST search of public sequence databases, the NOV5 protein was found to have homology to the proteins shown in the BLASTP data in Table SD.

TABLE 5D
Public BLASTP Results for NOV5

| | | NOV5 | Identities/ | |
|---|---|---|---|---|
| Protein | | Residues/ | Similarities for | |
| Accession | | Match | the Matched | Expect |
| Number | Protein/Organism/Length | Residues | Portion | Value |
| P07288 | Prostate specific antigen precursor (EC | 1 . . . 218 | 216/261 (82%) | e−124 |
| | 3.4.21.77) (PSA) (Gamma- seminoprotein) | 1 . . . 261 | 217/261 (82%) | |
| | (Kallikrein 3) (Semenogelase) (Seminin) | | | |
| | (P-30 antigen) - Homo sapiens | | | |
| | (Human), 261 aa. | | | |
| AAA59995 | APS PROTEIN PRECURSOR - Homo | 5 . . . 218 | 212/257 (82%) | e−120 |
| | sapiens (Human), 257 aa (fragment). | 1 . . . 257 | 213/257 (82%) | |
| P33619 | Prostate specific antigen precursor (EC | 1 . . . 218 | 199/261 (76%) | e−113 |
| | 3.4.21.35) (PSA) (Gamma- seminoprotein) | 1 . . . 261 | 207/261 (79%) | |
| | (Kallikrein 3) - Macaca mulatta | | | |
| | (Rhesus macaque), 261 aa. | | | |
| P20151 | Glandular kallikrein 2 precursor (EC | 1 . . . 218 | 172/261 (65%) | 3e−98 |
| | 3.4.21.35) (Tissue kallikrein) (Prostate) | 1 . . . 261 | 191/261 (72%) | |
| | (hGK-1) - Homo sapiens (Human), | | | |
| | 261 aa. | | | |
| QO7277 | PRE-PRO-PROTEIN FOR KALLIKREIN | 1 . . . 217 | 122/217 (56%) | 9e−67 |
| | (EC 3.4.21.35) - Homo sapiens | 1 . . . 194 | 142/217 (65%) | |
| | (Human), 195 aa. | | | |

PFam analysis predicts that the NOV5 protein contains the domains shown in the Table 5E.

TABLE 5E
Domain Analysis of NOV5

| | | Identities/ | |
|---|---|---|---|
| | NOV5 | Similarities for | Expect |
| Pfam Domain | Match Region | the Matched Region | Value |
| trypsin: domain 1 of 2 | 25 . . . 68 | 23/51 (45%) | 6.2e−18 |
| | | 38/51 (75%) | |
| trypsin: domain 2 of 2 | 75 . . . 210 | 59/156 (38%) | 1.2e−53 |
| | | 116/156 (74%) | |

### Example 6

The NOV6 clone was analyzed, and the nucleotide and encoded polypeptide sequences are shown in Table 6A.

TABLE 6A
NOV6 Sequence Analysis

| | | |
|---|---|---|
| | SEQ ID NO:13 | 515 bp |
| NOV6, | GCCTGACACCATGCTGCCCGCCTGCTTCCTCGGCCTACTGGCCTTCTCCTCCGCGTGC | |
| CG89614-02 DNA | TACTTCCAGAACTGCCCGAGGGGCGGCAAGAGGGCCATGTCCGACCTGGAGCTGAGAC | |
| Sequence | AGTGCCTCCCCTGCGGCCCCGGGGGCAAAGGCCGCTGCTTCGGGCCCAGCATTTGCTG | |
| | CGCGGACGAGCTGGGCTGCTTCGTGGGCACGGCTGAGGCGCTGCGCTGCCAGGAGGAG | |
| | AACTACCTGCCGTCGCCCTGCCAGTCCGGCCAGAAGGCGTGCGGGAGCGGGGGCCGCT | |
| | GCGCCGCCTTCGGCGTTTGCTGCAACGACGAGAGCTGCGTGACCGAGTCCGAGTGCCG | |
| | CGAGGGCTTTCACCGCCGCGCCCGCGCCAGCGACCGGAGCAACGCCACGCAACTGGAC | |
| | AGGCCGGCCGGGGCCTTGCTGCTGCGGCTGGTGCAGCTGGCCGGGGCGCCCGAGCCCT | |
| | TTGAGCCCGCCCAGCCCGACGCCTACTGAGCCCCGCGCTCGCCCCACCGGC | |
| | ORF Start: ATG at 11 | ORF Stop: TGA at 491 |
| | SEQ ID NO:14 | 160 aa MW at 16969.0 kD |
| NOV6, | MLPACFLGLLAFSSACYFQNCPRGGKRAMSDLELRQCLPCGPGGKGRCFGPSICCADE | |
| CG89614-02 Protein | LGCFVGTAEALRCQEENYLPSPCQSGQKACGSGGRCAAFGVCCNDESCVTESECREGF | |
| Sequence | HRRARASDRSNATQLDRPAGALLLRLVQLAGAPEPFEPAQPDAY | |

Further analysis of the NOV6 protein yielded the following properties shown in Table 6B.

TABLE 6B
Protein Sequence Properties NOV6

| | |
|---|---|
| PSort | 0.4753 probability located in outside; 0.1000 probability located in endoplasmic reticulum |
| analysis: | (membrane); 0.1000 probability located in endoplasmic reticulum (lumen); 0.1000 |
| | probability located in lysosome (lumen) |
| SignalP | Cleavage site between residues 16 and 17 |
| analysis: | |

A search of the NOV6 protein against the Geneseq database, a proprietary database that contains sequences published in patents and patent publication, yielded several homologous proteins shown in Table 6C.

TABLE 6C
Geneseq Results for NOV6

| | | NOV6 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB50995 | Human PRO1710 protein - Homo | 2 . . . 112 | 85/111 (76%) | 9e−52 |
| | sapiens, 125 aa. [WO200073445-A2, | 6 . . . 116 | 95/111 (85%) | |
| | 07 Dec. 2000] | | | |
| AAB24086 | Human PRO1710 pro-oxytocin protein | 2 . . . 112 | 85/111 (76%) | 9e−52 |
| | sequence SEQ ID NO:73 - Homo | 6 . . . 116 | 95/111 (85%) | |
| | sapiens, 125 aa. [WO200053755-A2, | | | |
| | 14 Sep. 2000] | | | |
| AAB24085 | Human PRO1710 mature oxytocin protein | 16 . . . 112 | 76/97 (78%) | 1e−46 |
| | sequence SEQ ID NO:73 - Homo | 1 . . . 97 | 85/97 (87%) | |
| | sapiens, 106 aa. [WO200053755-A2, | | | |
| | 14 Sep. 2000] | | | |
| AAB39235 | Gene 4 human secreted protein homologous | 54 . . . 97 | 39/44 (88%) | 8e−19 |
| | amino acid sequence #115 - | 1 . . . 44 | 41/44 (92%) | |
| | Callithrix jacchus, 44 aa. | | | |
| | [WO200056754-A1, 28 Sep. 2000] | | | |
| AAR08000 | Neurophysin I/II and pro-pressophysin | 22 . . . 49 | 27/28 (96%) | 2e−09 |
| | peptide antigen - Homo sapiens, | 1 . . . 28 | 27/28 (96%) | |
| | 28 aa.[EP399257-A, 28 Nov. 1990] | | | |

In a BLAST search of public sequence databases, the NOV6 protein was found to have homology to the proteins shown in the BLASTP data in Table 6D.

TABLE 6D
Public BLASTP Results for NOV6

| | | NOV6 | Identities/ | |
|---|---|---|---|---|
| Protein | | Residues/ | Similarities for | |
| Accession | | Match | the Matched | Expect |
| Number | Protein/Organism/Length | Residues | Portion | Value |
| P01185 | Vasopressin-neurophysin 2-copeptin | 1 . . . 160 | 158/160 (98%) | 4e−94 |
| | precursor [Contains: Arg- vasopressin; | 5 . . . 164 | 158/160 (98%) | |
| | Neurophysin 2 (Neurophysin-II); Copeptin] - | | | |
| | Homo sapiens (Human), 164 aa. | | | |
| O14935 | VASOPRESSIN - Homo sapiens (Human), | 1 . . . 160 | 156/160 (97%) | 3e−92 |
| | 164 aa. | 5 . . . 164 | 156/160 (97%) | |
| P01183 | Vasopressin-neurophysin 2-copeptin | 2 . . . 160 | 144/161 (89%) | 8e−84 |
| | precursor [Contains: Arg- vasopressin; | 6 . . . 166 | 148/161 (91%) | |
| | Neurophysin 2 (Neurophysin-I/-III); | | | |
| | Copeptin] - Sus scrofa (Pig), 166 aa. | | | |
| P01180 | Vasopressin-neurophysin 2-copeptin | 2 . . . 160 | 143/161 (88%) | 2e−83 |
| | precursor [Contains: Arg- vasopressin; | 6 . . . 166 | 147/161 (90%) | |
| | Neurophysin 2 (Neurophysin-II); Copeptin] - | | | |
| | Bos taurus (Bovine), 166 aa. | | | |
| P35455 | Vasopressin-neurophysin 2-copeptin | 2 . . . 160 | 130/159 (81%) | 6e−76 |
| | precursor [Contains: Arg-vasopressin; | 10 . . . 168 | 138/159 (86%) | |
| | Neurophysin 2 (Neurophysin-I); Copeptin] - | | | |
| | Mus musculus (Mouse), 168 aa. | | | |

PFam analysis predicts that the NOV6 protein contains the domains shown in the Table 6E.

TABLE 6E
Domain Analysis of NOV6

| | | Identities/ | |
|---|---|---|---|
| | NOV6 | Similarities for | Expect |
| Pfam Domain | Match Region | the Matched Region | Value |
| hormone4: domain 1 of 1 | 16 . . . 24 | 7/9 (78%) | 0.34 |
| | | 9/9 (100%) | |
| hormone5: domain 1 of 1 | 35 . . . 112 | 57/79 (72%) | 3.4e−46 |
| | | 75/79 (95%) | |

### Example 7

The NOV7 clone was analyzed, and the nucleotide and encoded polypeptide sequences are shown in Table 7A.

TABLE 7A
NOV7 Sequence Analysis

| | SEQ ID NO:15 | 1134 bp |
|---|---|---|
| NOV7, | TGGCCAGGCCCAGCTGTGGCCGGACAGGGACTGGAAGAGAGGACGCGGTCGAGTAGGT | |
| CG90031-01 DNA | GTGCACCAGCCCTGGCAACGAGAGCGTCTACCCCGAACTCTGCTGGCCTTGAGGTTTT | |
| Sequence | AAAACATGAATCCTTCACTCCTCCTGGCTGCCTTTTTCCTGGGAATTGCCTCAGCTGC | |
| | TCTAACATTTGACCACAGTTTAGACGCACAATGGACCAAGTGGAAGGCGATGCACAAC | |
| | AGATTATACGGCATGAATGAAGAAGGATGGAGGAGAGCAGTGTGGGAGAAGAACATGA | |
| | AGATGATTGAACTGCACAATCAGGAATACAGGGAAGGGAAACACAGCTTCACAATGGC | |
| | CATGAACGCCTTTGGAGACATGACCAGTGAAGAATTCAGGCAGGTGATGAATGGTTTT | |
| | CAATACCAGAAGCACAGGAAGGGGAAACAGTTCCAGGAACGCCTGCTTCTTGAGATCC | |
| | CCACATCTGTGGACTGGAGAGAGAAAGGCTACATGACTCCTGTGAAGGATCAGGGTCA | |
| | GTGTGGCTCTTGTTGGGCTTTTAGTGCAACTGGTGCTCTGGAAGGGCAGATGTTCTGG | |
| | AAAACAGGCAAACTTATCTCACTGAATGAGCAGAATCTGGTAGACTGCTCTGGGCCTC | |
| | AAGGCAATGAGGGCTGCAATGGTGACTTCATGGATAATCCCTTCCGGTATGTTCAGGA | |
| | GAACGGAGGCCTGGACTCTGAGGCATCCTATCCATATGAAGGAAAGGTTAAAACCTGT | |
| | AGGTACAATCCCAAGTATTCTGCTGCTAATGACACTGGTTTTGTGGACATCCCTTCAC | |
| | GGGAGAAGGACCTGGCGAAGGCAGTGGCAACTGTGGGGCCCATCTCTGTTGCTGTTGG | |
| | TGCAAGCCATGTCTTCTTCCAGTTCTATAAAAAAGGAATTTATTTTGAGCCACGCTGT | |
| | GACCCTGAAGGCCTGGATCATGCTATGCTGGTGGTTGGCTACAGCTATGAAGGAGCAA | |
| | ACTCAGATAACAATAAATATTGGCTGGTGAAGAACAGCTGGGGTAAAAACTGGGGCAT | |
| | GGATGGCTACATAAAGATGGCCAAAGACCGGAGGAACAACTGTGGAATTGCCACAGCA | |
| | GCCAGCTACCCCACTGTGTGAGCTGATGGATG | |
| | ORF Start: ATG at 122 | ORF Stop: TGA at 1121 |
| | SEQ ID NO:16 | 333 aa MW at 37753.3 kD |

| | |
|---|---|
| NOV7, | MNPSLLLAAFFLGIASAALTFDHSLDAQWTKWKAMHNRLYGMNEEGWRRAVWEKNMKM |
| CG90031-01 Protein | IELHNQEYREGKHSFTMAMNAFGDMTSEEFRQVMNGFQYQKHRKGKQFQERLLLEIPT |
| Sequence | SVDWREKGYMTPVKDQGQCGSCWAFSATGALEGQMFWKTGKLISLNEQNLVDCSGPQG |
| | NEGCNGDFMDNPFRYVQENGGLDSEASYPYEGKVKTCRYNPKYSAANDTGFVDIPSRE |
| | KDLAKAVATVGPISVAVGASHVFFQFYKKGIYFEPRCDPEGLDHAMLVVGYSYEGANS |
| | DNNKYWLVKNSWGKNWGMDGYIKMAKDRRNNCGIATAASYPTV |

Further analysis of the NOV7 protein yielded the following properties shown in Table 7B.

TABLE 7B
Protein Sequence Properties NOV7

| | |
|---|---|
| PSort | 0.8200 probability located in outside; 0.1846 probability located in microbody |
| analysis: | peroxisome); 0.1000 probability located in endoplasmic reticulum (membrane); 0.1000 |
| | probability located in endoplasmic reticulum (lumen) |
| SignalP | Cleavage site between residues 18 and 19 |
| analysis: | |

A search of the NOV7 protein against the Geneseq database, a proprietary database that contains sequences published in patents and patent publication, yielded several homologous proteins shown in Table 7C.

TABLE 7C
Geneseq Results for NOV7

| | | NOV7 | Identities/ | |
|---|---|---|---|---|
| | | Residues/ | Similarities for | |
| Geneseq | Protein/Organism/Length | Match | the Matched | Expect |
| Identifier | [Patent #,Date] | Residues | Region | Value |
| AAW47031 | Human procathepsin L - Homo sapiens, | 1 . . . 333 | 271/333 (81%) | e−167 |
| | 333 aa. [US5710014-A, 20 Jan. 1998] | 1 . . . 333 | 294/333 (87%) | |
| AAM93531 | Human polypeptide, SEQ ID NO:3271 - | 1 . . . 333 | 270/333 (81%) | e−166 |
| | Homo sapiens, 333 aa. [EP1130094-A2, | 1 . . . 333 | 293/333 (87%) | |
| | 05 Sep. 2001] | | | |
| AAR28829 | Human procathepsin L - Homo sapiens, | 1 . . . 333 | 270/333 (81%) | e−165 |
| | 333 aa. [WO9219756-A, 12 Nov. 1992] | 1 . . . 333 | 293/333 (87%) | |
| AAP82094 | pHu-16 sequence encoded human | 1 . . . 333 | 265/333 (79%) | e−164 |
| | procathepsin L - Homo sapiens, 333 aa. | 1 . . . 333 | 293/333 (87%) | |
| | [USN7154692-N, 11 Feb. 1988] | | | |
| AAU12177 | Human PRO305 polypeptide sequence - | 1 . . . 333 | 240/334 (71%) | e−144 |
| | Homo sapiens, 334 aa. | 1 . . . 334 | 274/334 (81%) | |
| | [WO200140466-A2, 07 Jun. 2001] | | | |

In a BLAST search of public sequence databases, the NOV7 protein was found to have homology to the proteins shown in the BLAST? data in Table 7D.

TABLE 7D
Public BLASTP Results for NOV7

| | | NOV7 | Identities/ | |
|---|---|---|---|---|
| Protein | | Residues/ | Similarities for | |
| Accession | | Match | the Matched | Expect |
| Number | Protein/Organism/Length | Residues | Portion | Value |
| P07711 | Cathepsin L precursor (EC 3.4.22.15) | 1 . . . 333 | 271/333 (81%) | e−66 |
| | (Major excreted protein) (MEP) - Homo | 1 . . . 333 | 294/333 (87%) | |
| | sapiens (Human), 333 aa. | | | |
| Q96QJ0 | SIMILAR TO CATHEPSIN L - Homo | 1 . . . 333 | 270/333 (81%) | e−166 |
| | sapiens (Human), 333 aa. | 1 . . . 333 | 294/333 (88%) | |
| Q9GKL8 | CYSTEINE PROTEASE - Cercopithecus | 1 . . . 333 | 263/333 (78%) | e−162 |
| | aethiops (Green monkey) (Grivet), 333 aa. | 1 . . . 333 | 289/333 (85%) | |
| Q9GL24 | CATHEPSIN L (EC 3.4.22.15) - Canis | 1 . . . 333 | 254/334 (76%) | e−154 |
| | familiaris (Dog), 333 aa. | 1 . . . 333 | 283/334 (84%) | |
| Q28944 | Cathepsin L precursor (EC 3.4.22.15) - | 1 . . . 333 | 245/334 (73%) | e−151 |
| | Sus scrofa (Pig), 334 aa. | 1 . . . 334 | 281/334 (83%) | |

PFam analysis predicts that the NOV7 protein contains the domains shown in the Table 7E.

TABLE 7E
Domain Analysis of NOV7

| | | Identities/ | |
|---|---|---|---|
| | | Similarities | |
| Pfam Domain | NOV7 Match Region | for the Matched Region | Expect Value |
| Peptidase_C1: domain 1 of 1 | 114 . . . 332 | 125/337 (37%) | 8.7e−120 |
| | | 197/337 (58%) | |

### Example 8

The NOV8 clone was analyzed, and the nucleotide and encoded polypeptide sequences are shown in Table 8A.

TABLE 8A
NOV8 Sequence Analysis

| | SEQ ID NO:17 | 793 bp |
|---|---|---|
| NOV8, | TAAATTCGCGGCCGCGTCGACCTCCTCATGGTCGTGACGACGCGTTCTCGTAAGGACA | |
| CG90155-01 DNA | AGCTTGACGCCGAGGTGCATGCCGGTGAAGGCACCCCCGGGGATGTCATCGTGCTGCG | |
| Sequence | GTTTTCCGGAGCCATGGCGAAGCGTCCTGCCTCAGTTATCCTTCCGCTGCTACTGTCG | |
| | GACTCCCCCGTCATTGCGTGGTGGCCCTTCTCCGGCCCTGACAACCTCGCCTCGGACC | |
| | CCATCGGAGCCCTTGCGGACCGCCGCATCACCGACTCGGCAGCTGACAAAGATCCGTG | |
| | CAAAGCCCTCATACGCCGTGCGGCTCACCTAACCGAGGGTGACTCCGACCTGTGTTGG | |
| | GCTCGCACCACCAGCTGGAGAGCCCTAGCTGCAGCAGCTTTGGATCAACATCCAGCGA | |
| | CCGTCAAGTTCGCTCGGGTAGAGTCAGCCGCCGGTAATGCGCCGGCGATGCTGCTGGC | |
| | AGCCTGGCTAGGATTGCGTCTCGGCGTCCCGGTCGAGCGGGTGACAACCGACGCGCCC | |
| | GGCATCTCCGCGATCGTCATGTCGACCTCAGGTGGTGACATCGAGATACGCCGTCGCA | |
| | GCGGCAGATACGCCGTCTACCGGATCCCGGGAGAACCAGCGCGCGGAGTAGCCCTGGA | |
| | CCGTCGTGAGGTACAGATGCTCATCGGTGAGGAGCTTCGTCGGCTCGGCCCCGACAAG | |
| | GTGTTCACCGCTGTCATGGCTGAAATTCACGATGGGGCGGGCCGAATCTCATTGACAA | |
| | ATGATAGGGATGAGTCATGACAAGCCGACGCCCCTCGTG | |
| | ORF Start: ATG at 28 | ORF Stop: TGA at 772 |
| | SEQ ID NO:18 | 248 aa MW at 26579.9 kD |

| | |
|---|---|
| NOV8, | MVVTTRSRKDKLDAEVHAGEGTPGDVIVLRFSGAMAKRPASVILPLLLSDSPVIAWWP |
| CG90155-01 Protein | FSGPDNLASDPIGALADRRITDSAADKDPCKALIRRAAHLTEGDSDLCWARTTSWRAL |
| Sequence | AAAALDQHPATVKFARVESAAGNAPAMLLAAWLGLRLGVPVERVTTDAPGISAIVMST |
| | SGGDIEIRRRSGRYAVYRIPGEPARGVALDRREVQMLIGEELRRLGPDKVFTAVMAEI |
| | HDGAGRISLTNDRDES |

Further analysis of the NOV8 protein yielded the following properties shown in Table 8B.

TABLE 8B
Protein Sequence Properties NOV8

| | |
|---|---|
| PSort | 0.4500 probability located in cytoplasm; 0.3000 probability located in microbody |
| analysis: | (peroxisome); 0.2377 probability located in lysosome (lumen); 0.1000 probability located |
| | in mitochondrial matrix space |
| SignalP | Cleavage site between residues 56 and 57 |
| analysis: | |

A search of the NOV8 protein against the Geneseq database, a proprietary database that contains sequences published in patents and patent publication, yielded several homologous proteins shown in Table 8C.

TABLE 8C
Geneseq Results for NOV8

| | | NOV8 | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAU48672 | Propionibacterium acnes immunogenic | 1 . . . 248 | 245/248 (98%) | e−138 |
| | protein #9568 - Propionibacterium acnes, | 66 . . . 313 | 247/248 (98%) | |
| | 313 aa. [WO200181581-A2, | | | |
| | 01-NOV-2001] | | | |
| AAU48672 | Propionibacterium acnes immunogenic | 1 . . . 248 | 245/248 (98%) | e−138 |
| | protein #9568 - Propionibacterium acnes, | 66 . . . 313 | 247/248 (98%) | |
| | 313 aa. [WO200181581-A2, | | | |
| | 01-NOV-2001] | | | |
| AAB41505 | Human ORFX ORF1269 polypeptide | 5 . . . 173 | 169/169 (100%) | 2e−93 |
| | sequence SEQ ID NO:2538 - Homo | 1 . . . 169 | 169/169 (100%) | |
| | sapiens, 169 aa. [WO200058473-A2, | | | |
| | 05-OCT-2000] | | | |
| ABB53105 | Human ORF11 protein - Homo sapiens, 144 | 9 . . . 152 | 144/144 (100%) | 2e−79 |
| | aa. [WO200177155-A2, 18-OCT-2001] | 1 . . . 144 | 144/144 (100%) | |
| ABB53189 | Human ORF95 protein - Homo sapiens, 144 | 9 . . . 152 | 142/144 (98%) | 8e−78 |
| | aa. [WO200177155-A2, 18-OCT-2001] | 1 . . . 144 | 143/144 (98%) | |

In a BLAST search of public sequence databases, the NOV8 protein was found to have homology to the proteins shown in the BLASTP data in Table 8D.

TABLE 8D
Public BLASTP Results for NOV8

| | | NOV8 | Identities/ | |
|---|---|---|---|---|
| Protein | | Residues/ | Similarities for | |
| Accession | | Match | the Matched | Expect |
| Number | Protein/Organism/Length | Residues | Portion | Value |
| O88016 | HYPOTHETICAL 33.9 KDA PROTEIN - | 9 . . . 229 | 104/222 (46%) | 3e−50 |
| | Streptomyces coelicolor, 311 aa. | 78 . . . 299 | 136/222 (60%) | |
| Q9XAB8 | HYPOTHETICAL 37.7 KDA PROTEIN - | 5 . . . 229 | 105/226 (46%) | 3e−48 |
| | Streptomyces coelicolor, 351 aa. | 77 . . . 299 | 134/226 (58%) | |
| CAC26326 | SEQUENCE 79 FROM PATENT | 1 . . . 222 | 89/238 (37%) | 3e−33 |
| | WO0100804 - Corynebacterium | 66 . . . 301 | 130/238 (54%) | |
| | glutamicum (Brevibacterium flavum), 319 | | | |
| | aa. | | | |
| AAK45756 | OXPPCYCLE PROTEIN OPCA - | 1 . . . 232 | 87/238 (36%) | 2e−31 |
| | Mycobacterium tuberculosis CDC1551, | 63 . . . 297 | 126/238 (52%) | |
| | 303 aa. | | | |
| O06813 | HYPOTHETICAL 32.7 KDA PROTEIN - | 1 . . . 232 | 86/238 (36%) | 2e−30 |
| | Mycobacterium tuberculosis, 303 aa. | 63 . . . 297 | 125/238 (52%) | |

PFam analysis predicts that the NOV8 protein contains the domains shown in the Table 8E.

TABLE 8E
Domain Analysis of NOV8

| | | | |
|---|---|---|---|
| | | Identities/ | |
| | | Similarities | |
| | NOV8 Match | for the Matched | |
| Pfam Domain | Region | Region | Expect Value |

No Significant Known Matches Found

### Example 9

The NOV9 clone was analyzed, and the nucleotide and encoded polypeptide sequences are shown in Table 9A.

TABLE 9A
NOV9 Sequence Analysis

| | SEQ ID NO:19 | 438 bp |
|---|---|---|
| NOV9a, | CCCTGTACGGGAAGAGACCTTCATTAACACTTGGGTAACTTACCCTTCACAATCCATC | |
| CG90750-01 DNA | TAAATCCTTCTCAATTGCTGCCACCATGACTCGTTACTTCTGCTGTGGAAGCTACTTC | |
| Sequence | CCAGGATACCCTATTTATGGGACCAACTTCCATGGGACCTTCAGAGCCACCCCCTTGA | |
| | ACTGTGTTGTGCCTCTGGGCTCTCCCCTGAACTATGGCTGTGGATGCAATGGCTACAG | |
| | CTCCCTGGGCTACAGCTTTGGTGGTAGCAACATCAACAACCTGGGCGGCTGCTATGGT | |
| | GGTAGCTTCTATAGGCCATGGGGCTCTGGCTCTGGCTTTGGCTACAGCACCTACTGAT | |
| | GGACCAATGGCTCCAGTGACTACAGGACTCTCAATTAATTCTCTGCACAGAACAACCT | |
| | GAAGAGCAATGACTGTCTTCCTACCTTCCCAT | |
| | ORF Start: ATG at 84 | ORF Stop: TGA at 345 |
| | SEQ ID NO:20 | 87 aa MW at 9288.2 kD |
| NOV9a, | MTRYFCCGSYFPGYPIYGTNFHGTFRATPLNCVVPLGSPLNYGCGCNGYSSLGYSFGG | |
| C690750-01 Protein | SNINNLGGCYGGSFYRPWGSGSGFGYSTY | |
| Sequence | | |
| | SEQ ID NO:21 | 358 bp |
| NOV9b, | ACCCTTCACAATCCATCTAAATCCTTCTCAATTGCTGCCACCATGACTCGTTACTTCT | |
| CG90750-02 DNA | GCTGTGGAAGCTACTTCCCAGGATACCCTATCTATGGGACCAACTTCCACGGGACCTT | |
| Sequence | CAGAGCCACCCCCTTGAACTGTGTTGTGCCTCTGGGCTCTCCCCTGAACTATGGCTGT | |
| | GGATGCAATGGCTACAGCCCCCTGGGCTACAGCTTTGGTGGTAGCAACAGCAACAACC | |
| | TGGGAGGCTGCTATGGTGGTAGCTTCTATAGGCCATGGGGCTCTGGCTCTGGCTTTGG | |
| | CTACAGCACCTACTGATGGACCAATGGCTCCAGTGACTACAGGACTCTCAATTAATTC | |
| | TCTGCACAGA | |
| | ORF Start: ATG at 43 | ORF Stop: TGA at 304 |
| | SEQ ID NO:22 | 87 aa MW at 9272.2 kD |

| | |
|---|---|
| NOV9b, | MTRYFCCGSYFPGYPIYGTNFHGTFRATPLNCVVPLGSPLNYGCGCNGYSPLGYSFGG |
| CG90750-02 Protein | SNSNNLGGCYGGSFYRPWGSGSGFGYSTY |
| Sequence | |

Sequence comparison of the above protein sequences yields the following sequence relationships shown in Table 9B.

TABLE 9B
Comparison of NOV9a against NOV9b.

| | | Identities/ |
|---|---|---|
| | NOV9a Residues/ | Similarities for the Matched |
| Protein Sequence | Match Residues | Region |
| NOV9b | 1 . . . 87 | 66/87 (75%) |
| | 1 . . . 87 | 66/87 (75%) |

Further analysis of the NOV9a protein yielded the following properties shown in Table 9C.

TABLE 9C
Protein Sequence Properties NOV9a

| | |
|---|---|
| PSort | 0.6400 probability located in microbody (peroxisome); 0.4500 probability located in |
| analysis: | cytoplasm; 0.3060 probability located in lysosome (lumen); 0.1000 probability located in |
| | mitochondrial matrix space |
| SignalP | No Known Signal Sequence Predicted |
| analysis: | |

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities for | |
| Geneseq | | Match | the Matched | Expect |
| Identifier | Protein/Organism/Length [Patent #, Date] | Residues | Region | Value |
| AAB81935 | Marmoset vitamin D response element | 8 . . . 84 | 29/77 (37%) | 0.004 |
| | binding protein #2 - Saguinus oedipus, 341 | 269 . . . 335 | 34/77 (43%) | |
| | aa. [WO200121649-A2, 29-MAR-2001] | | | |
| AAG75147 | Human colon cancer antigen protein SEQ ID | 8 . . . 84 | 29/77 (37%) | 0.004 |
| | NO:5911 - Homo sapiens, 212 aa. | 140 . . . 206 | 34/77 (43%) | |
| | [WO200122920-A2, 05-APR-2001] | | | |
| AAB57093 | Human prostate cancer antigen protein | 8 . . . 84 | 29/77 (37%) | 0.004 |
| | sequence SEQ ID NO:1671 - Homo sapiens, | 146 . . . 212 | 34/77 (43%) | |
| | 218 aa. [WO200055174-A1, 21-SEP-2000] | | | |
| AAW54362 | Heterogeneous nuclear ribonucleoproteins | 8 . . . 84 | 29/77 (37%) | 0.004 |
| | A2/B1 - Homo sapiens, 353 aa. | 281 . . . 347 | 34/77 (43%) | |
| | [WO9810291-A1, 12-MAR-1998] | | | |
| AAW50921 | Amino acid sequence of a heterogenous | 8 . . . 84 | 29/77 (37%) | 0.004 |
| | ribonucleotide protein - Homo sapiens, 353 | 281 . . . 347 | 34/77 (43%) | |
| | aa. [WO9814469-A2, 09-APR-1998] | | | |

In a BLAST search of public sequence databases, the NOV9a protein was found to have homology to the proteins shown in the BLASTP data in Table 9E.

TABLE 9E
Public BLASTP Results for NOV9a

| | | NOV9a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q28580 | HGT-C2 HIGH-(GLYCINE + TYROSINE) | 1 . . . 87 | 75/87 (86%) | 9e−42 |
| | (HGT) KERATIN - Ovis aries (Sheep), 85 | 1 . . . 85 | 78/87 (89%) | |
| | aa. | | | |
| Q9D3I6 | 5430433J05RIK PROTEIN - Mus musculus | 1 . . . 87 | 69/88 (78%) | 9e−38 |
| | (Mouse), 87 aa. | 1 . . . 87 | 75/88 (84%) | |
| Q22168 | T04F8.8 PROTEIN - Caenorhabditis | 7 . . . 84 | 30/78 (38%) | 8e−05 |
| | elegans, 165 aa. | 18 . . . 89 | 37/78 (46%) | |
| Q925H7 | KERATIN-ASSOCIATED PROTEIN 16.4 - | 40 . . . 87 | 20/50 (40%) | 0.011 |
| | Mus musculus (Mouse), 84 aa. | 35 . . . 83 | 28/50 (56%) | |
| Q9TTV2 | VITAMIN D RESPONSE ELEMENT | 8 . . . 84 | 29/77 (37%) | 0.011 |
| | BINDING PROTEIN - Saguinus oedipus | 269 . . . 335 | 34/77 (43%) | |
| | (Cotton-top tamarin), 341 aa. | | | |

PFam analysis predicts that the NOV9a protein contains the domains shown in the Table 9F.

TABLE 9F
Domain Analysis of NOV9a

| | | | |
|---|---|---|---|
| | | Identities/ | |
| | | Similarities | |
| | NOV9a Match | for the Matched | |
| Pfam Domain | Region | Region | Expect Value |

No Significant Known Matches Found

### Example 10

The NOV10 clone was analyzed, and the nucleotide and encoded polypeptide sequences are shown in Table 10A.

TABLE 10A
NOV10 Sequence Analysis

| | SEQ ID NO:23 | 385 bp |
|---|---|---|
| NOV10, | ACTGGAAAGAAACAATCCAGTGTAAATATGACTTCTAAGCTGGCTGTTGCTCTACTGC | |
| CG91235-01 DNA | TTTCTTGGCAGTTGCATGCTTTCTCTATGTTCACTGCTTCCATTGTGCCAAGTATTAG | |
| Sequence | TACAGTACCACAATGCCAGTGCATGAGGACACATTTTATACCTTTGCATCCCAAATTT | |
| | ATTAAAGAACTCAGAATTATTCAGAGTGGATTATATTATAAAAATTCAGAAATCATAG | |
| | TCAGACTGAAAGATGGGAAATTAATTTGTTTGGATCCTGAGGCTACATGGGTGATGAC | |
| | TAACTATTATCAAAGAGATTATGGACAGGTATAATTAATGCCAAAAATTATCATATTC | |
| | ACTTTCTTTTTCTCTTTCTTTTCTTTTAATTAAGGAT | |
| | ORF Start: ATG at 28 | ORF Stop: TAA at 322 |
| | SEQ ID NO:24 | 98 aa MW at 11337.3 kD |

| | |
|---|---|
| NOV10, | MTSKLAVALLLSWQLHAFSMFTASIVPSISTVPQCQCMRTHFIPLHPKFIKELRIIQS |
| CG91235-01 Protein | GLYYKNSEIIVRLKDGKLICLDPEATWVMTNYYQRDYGQV |
| Sequence | |

Further analysis of the NOV10 protein yielded the properties shown in Table 10B.

TABLE 10B
Protein Sequence Properties NOV10

| | |
|---|---|
| PSort | 0.3703 probability located in outside; 0.1748 probability located in microbody |
| analysis: | (peroxisome); 0.1000 probability located in endoplasmic reticulum (membrane); 0.1000 |
| | probability located in endoplasmic reticulum (lumen) |
| SignalP | Cleavage site between residues 20 and 21 |
| analysis: | |

A search of the NOV10 protein against the Geneseq database, a proprietary database that contains sequences published in patents and patent publication, yielded several homologous proteins shown in Table 10C.

TABLE 10C
Geneseq Results for NOV10

| | | NOV10 | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG66022 | Human interleukin (IL)-8 polypeptide - | 1 . . . 86 | 43/86 (50%) | 1e—18 |
| | Homo sapiens, 99 aa. | 1 . . . 85 | 64/86 (74%) | |
| | [WO200183499-A2, 08 Nov. 2001] | | | |
| AAB90797 | Human shear stress-response protein SEQ | 1 . . . 86 | 43/86 (50%) | 1e—18 |
| | ID NO:94 - Homo sapiens, 99 aa. | 1 . . . 85 | 64/86 (74%) | |
| | [WO200125427-A1, 12 APR. 2001] | | | |
| AAB07714 | Amino acid sequence of porcine | 1 . . . 86 | 45/86 (52%) | le—18 |
| | interleukin-8 (IL-8) - Sus sp, 103 aa. | 1 . . . 85 | 60/86 (69%) | |
| | [WO200042069-A1, 20 Jul. 2000] | | | |
| AAB15792 | Human chemokine IL-8 SEQ ID NO:23 - | 1 . . . 86 | 43/86 (50%) | 1e—18 |
| | Homo sapiens, 99 aa. | 1 . . . 85 | 64/86 (74%) | |
| | [WO200042071-A2, 20 Jul. 2000] | | | |
| AAW96711 | Interluekin-8 (IL-8) protein - Homo | 1 . . . 86 | 43/86 (50%) | 1e—18 |
| | sapiens, 99 aa. [US5871723-A, | 1 . . . 85 | 64/86 (74%) | |
| | 16 Feb. 1999] | | | |

In a BLAST search of public sequence databases, the NOV10 protein was found to have homology to the proteins shown in the BLASTP data in Table 10D.

TABLE 10D
Public BLASTP Results for NOV10

| | | NOV10 | Identities/ | |
|---|---|---|---|---|
| Protein | | Residues/ | Similarities for | |
| Accession | | Match | the Matched | Expect |
| Number | Protein/Organism/Length | Residues | Portion | Value |
| P36925 | Interleukin-8 precursor (IL-8) - Ovis aries | 1 . . . 86 | 48/86 (55%) | 2e—20 |
| | (Sheep), 101 aa. | 1 . . . 85 | 67/86 (77%) | |
| P19874 | Interleukin-8 precursor (IL-8) (Neutrophil | 1 . . . 86 | 46/86 (53%) | 2e—19 |
| | attractant/activation protein-1) (NAP-1) | 1 . . . 85 | 64/86 (73%) | |
| | (Permeability factor 1) (RPF1) - Oryctolagus | | | |
| | cuniculus (Rabbit), 101 aa. | | | |
| P79255 | Interleukin-8 precursor (IL-8) - Bos taurus | 1 . . . 86 | 46/86 (53%) | 2e—19 |
| | (Bovine), 101 aa. | 1 . . . 85 | 66/86 (76%) | |
| P26894 | Interleukin-8 precursor (IL-8) (Alveolar | 1 . . . 86 | 46/86 (53%) | 5e—19 |
| | macrophage chemotactic factor I) (AMCF-I) - | 1 . . . 85 | 63/86 (72%) | |
| | Sus scrofa (Pig), 103 aa. | | | |
| JN0841 | interleukin-8 - dog, 95 aa. | 1 . . . 86 | 45/86 (52%) | 7e—19 |
| | | 1 . . . 85 | 65/86 (75%) | |

PFam analysis predicts that the NOV10 protein contains the domains shown in the Table 10E.

TABLE 10E
Domain Analysis of NOV10

| | | Identities/ | |
|---|---|---|---|
| | NOV10 | Similarities for | Expect |
| Pfam Domain | Match Region | the Matched Region | Value |
| IL8: domain 1 of 1 | 26 . . . 86 | 24/62 (39%) | 2.9e—13 |
| | | 45/62 (73%) | |

### Example 11

The NOV11 clone was analyzed, and the nucleotide and encoded polypeptide sequences are shown in Table 11A.

TABLE 11A
NOV11 Sequence Analysis

| | SEQ ID NO:25 | 1766 bp |
|---|---|---|
| NOV11a, | TAGCTCGCCAGAGAGTCTATGTATGGGATTGAACAATCTGTAAACTAAAGGATCCTAA | |
| CG91657-01 DNA | TCATGAAAATAAGTATGATAAATTATAAGTCACTATTGGCACTGTTGTTTATATTAGC | |
| Sequence | CTCCTGGATCATTTTTACAGTTTTCCAGAACTCCATTTCAAAGGTTTGGTCTGCTCTA | |
| | AACTTATCCATCTCCCTCCATTACTGGAACAACTCCACAAAGTCCTTATTCCCTAAAA | |
| | CACCACTGATATCATTAAAGCCACTAACAGAGACTGAACTCAGAATAAAGGAAATCAT | |
| | AGAGAAACTAGATCAGCAGATCCCACCCAGACCTTTCACCCACGTGAACACCACCACC | |
| | AGCGCCACACATAGCACAGCCACCATCCTCAACCCTCGAGATACGTACTGCAGGGGAG | |
| | ACCAGCTGCACATCCTGCTGGAGGTGAGGGACCACTTGGGACGCAGGAAGCAATATGG | |
| | CGGGGATTTCCTGAGGGCCAGGATGTCTTCCCCAGCGCTGATGGCAGGTGCTTCAGGA | |
| | AAGGTGACTGACTTCAACAACGGCACCTACCTGGTCAGCTTCACTCTGTTCTGGGAGG | |
| | GCCAGGTCTCTCTGTCTGTGCTGCTCATCCACCCCAGTGAAGGGGTGTCAGCTCTCTG | |
| | GAGTGCAAGGAACCAAGGCTATGACAGGGTGATCTTCACTGGCCAGTTTGTCAATGGC | |
| | ACTTCCCAAGTCCACTCTGAATGTGGCCTGATCCTAAACACAAATGCTGAATTGTGCC | |
| | AGTACCTGGACAACAGAGACCAAGAAGGCTTCTACTGTGTGAGGCCTCAACACATGCC | |
| | CTGTGCTGCACTCACTCACATGTATTCTAAGAACAAGAAAGTTTCTTATCTTAGCAAA | |
| | CAAGAAAAGAGCCTCTTTGAAAGGTCAAATGTGGGTGTAGAGATTATGGAAAAATTCA | |
| | ATACAATTAGTGTCTCCAAATGCAACAAAGAAACAGTTGCAATGAAAGAGAAATGCAA | |
| | GTTTGGAATGACATCCACAATCCCCAGTGGGCATGTCTGGAGAAACACATGGAATCCT | |
| | GTCTCCTGTAGTTTGGCTACAGTCAAAATGAAGGAATGCCTGAGAGGAAAACTCATAT | |
| | ACCTAATGGGAGATTCCACGATCCGCCAGTGGATGGAATACTTCAAAGCCAGTATCAA | |
| | CACACTGAAGTCAGTGGATCTGCATGAATCTGGAAAATTGCAACACCAGCTTGCTGTG | |
| | GATTTGGATAGGAACATCAACATCCAGTGGCAAAAATATTGTTATCCCTTGATAGGAT | |
| | CAATGACCTATTCAGTCAAAGAGATGGAGTACCTCACCCGGGCCATTGACAGAACTGG | |
| | AGGAGAAAAAAATACTGTCATTGTTATTTCCCTGGGCCAGCATTTCAGACCCTTTCCC | |
| | ATTGATGTTTTTATCCGAAGGGCCCTCAATGTCCACAAAGCCATTCAGCATCTTCTTC | |
| | TGAGAAGCCCAGACACTATGGTTATCATCAAAACAGAAAACATCAGGGAGATGTACAA | |
| | TGATGCAGAAAGATTTAGTGACTTTCATGGTTACATTCAATATCTCATCATAAAGGAC | |
| | ATTTTCCAGGATCTCAGTGTGAGTATCATTGATGCCTGGGATATAACAATTGCATATG | |
| | GCACAAATAATGTACACCCACCTCAACATGTAGTCGGAAATCAGATTAATATATTATT | |
| | AAACTATATTTGTTAAATAACACAAAAGTCTGAAATTCATTCACTTAAGTAAAAAAAT | |
| | TTATTGACTGTCTACTAGCAGGCCAG | |
| | ORF Start: ATG at 61 | ORF Stop: TAA at 1696 |
| | SEQ ID NO:26 | 545 aa MW at 62347.3 kD |
| NOV11a, | MKISMINYKSLLALLFILASWIIFTVFQNSISKVWSALNLSISLHYWNNSTKSLFPKT | |
| CG91657-01 Protein | PLISLKPLTETELRIKEIIEKLDQQIPPRPFTHVNTTTSATHSTATILNPRDTYCRGD | |
| Sequence | QLHILLEVRDHLGRRKQYGGDFLRARMSSPALMAGASGKVTDFNNGTYLVSFTLFWEG | |
| | QVSLSVLLIHPSEGVSALWSARNQGYDRVIFTGQFVNGTSQVHSECGLILNTNAELCQ | |
| | YLDNRDQEGFYCVRPQHMPCAALTHMYSKNKKVSYLSKQEKSLFERSNVGVEIMEKFN | |
| | TISVSKCNKETVAMKEKCKFGMTSTIPSGHVWRNTWNPVSCSLATVKMKECLRGKLIY | |
| | LMGDSTIRQWMEYFKASINTLKSVDLHESGKLQHQLAVDLDRNINIQWQKYCYPLIGS | |
| | MTYSVKEMEYLTRAIDRTGGEKNTVIVISLGQHFRPFPIDVFIRRALNVHKAIQHLLL | |
| | RSPDTMVIIKTENIREMYNDAERFSDFHGYIQYLIIKDIFQDLSVSIIDAWDITIAYG | |
| | TNNVHPPQHVVGNQINILLNYIC | |
| | SEQ ID NO:27 | 1763 bp |
| NOV11b, | TAGCTCGCCAGAGAGTCTATGTATGGGATTGAACAATCTGTAAACTAAAGGATCCTAA | |
| CG91657-02 DNA | TCATGAAAATAAGTATGATAAATTATAAGTCACTATTGGCACTGTTGTTTATATTAGC | |
| Sequence | CTCCTGGATCATTTTTACAGTTTTCCAGAACTCCACAAAGGTTTGGTCTGCTCTAAAC | |
| | TTATCCATCTCCCTCCATTACTGGAACAACTCCACAAAGTCCTTATTCCCTAAAACAC | |
| | CACTGATATCATTAAAGCCACTAACAGAGACTGAACTCAGAATAAAGGAAATCATAGA | |
| | GAAACTAGATCAGCAGATCCCACCCAGACCTTTCACCCACGTGAACACCACCACCAGC | |
| | GCCACACATAGCACAGCCACCATCCTCAACCCTCGAGATACGTACTGCAGGGGAGACC | |
| | AGCTGCACATCCTGCTGGAGGTGAGGGACCACTTGGGACGCAGGAAGCAATATGGCGG | |
| | GGATTTCCTGAGGGCCAGGATGTCTTCCCCAGCGCTGATGGCAGGTGCTTCAGGAAAG | |
| | GTGACTGACTTCAACAACGGCACCTACCTGGTCAGCTTCACTCTGTTCTGGGAGGGCC | |
| | AGGTCTCTCTGTCTCTGCTGCTCATCCACCCCAGTGAAGGGGTGTCAGCTCTCTGGAG | |
| | TGCAAGGAACCAAGGCTATGACAGGGTGATCTTCACTGGCCAGTTTGTCAATGGCACT | |
| | TCCCAAGTCCACTCTGAATGTGGCCTGATCCTAAACACAAATGCTGAATTGTGCCAGT | |
| | ACCTGGACAACAGAGACCAAGAAGGCTTCTACTGTGTGAGGCCTCAACACATGCCCTG | |
| | TGCTGCACTCACTCACATGTATTCTAAGAACAAGAAAGTTTCTTATCTTAGCAAACAA | |
| | GAAAAGAGCCTCTTTGAAAGGTCAAATGTGGGTGTAGAGATTATGGAAAAATTCAATA | |
| | CAATTAGTGTCTCCAAATGCAACAAAGAAACAGTTGCAATGAAAGAGAAATGCAAGTT | |
| | TGGAATGACATCCACAATCCCCAGTGGGCATGTCTGGAGAAACACATGGAATCCTGTC | |
| | TCCTGTAGTTTGGCTACAGTCAAAATGAAGGAATGCCTGAGAGGAAAACTCATATACC | |
| | TAATGGGAGATTCCACGATCCGCCAGTGGATGGAATACTTCAAAGCCAGTATCAACAC | |
| | ACTGAAGTCAGTGGATCTGCATGAATCTGGAAAATTGCAACACCAGCTTGCTGTGGAT | |
| | TTGGATAGGAACATCAACATCCAGTGGCAAAAATATTGTTATCCCTTGATAGGATCAA | |
| | TGACCTATTCAGTCAAAGAGATGGAGTACCTCACCCGGGCCATTGACAGAACTGGAGG | |
| | AGAAAAAAATACTGTCATTGTTATTTCCCTGGGCCAGCATTTCAGACCCTTTCCCATT | |
| | GATGTTTTTATCCGAAGGGCCCTCAATGTCCACAAAGCCATTCAGCATCTTCTTCTGA | |
| | GAAGCCCAGACACTATGGTTATCATCAAAACAGAAAACATCAGGGAGATGTACAATGA | |
| | TGCAGAAAGATTTAGTGACTTTCATGGTTACATTCAATATCTCATCATAAAGGACATT | |
| | TTCCAGGATCTCAGTGTGAGTATCATTGATGCCTGGGATATAACAATTGCATATGGCA | |
| | CAAATAATGTACACCCACCTCAACATGTAGTCGGAAATCAGATTAATATATTATTAAA | |
| | CTATATTTGTTAAATAACACAAAAGTCTGAAATTCATTCACTTAAGTAAAAAAATTTA | |
| | TTGACTGTCTACTAGCAGGCCAG | |
| | ORF Start: ATG at 61 | ORF Stop: TAA at 1693 |
| | SEQ ID NO:28 | 544 aa MW at 62262.2 kD |

| | |
|---|---|
| NOV11b, | MKISMINYKSLLALLFILASWIIFTVFQNSTKVWSALNLSISLHYWNNSTKSLFPKTP |
| CG91657-02 Protein | LISLKPLTETELRIKEIIEKLDQQIPPRPFTHVNTTTSATHSTATILNPRDTYCRGDQ |
| Sequence | LHILLEVRDHLGRRKQYGGDFLRARMSSPALMAGASGKVTDFNNGTYLVSFTLFWEGQ |
| | VSLSLLLIHPSEGVSALWSARNQGYDRVIFTGQFVNGTSQVHSECGLILNTNAELCQY |
| | LDNRDQEGFYCVRPQHMPCAALTHMYSKNKKVSYLSKQEKSLFERSNVGVEIMEKFNT |
| | ISVSKCNKETVANKEKCKFGMTSTIPSGHVWRNTWNPVSCSLATVKMKECLRGKLIYL |
| | MGDSTIRQWMEYFKASINTLKSVDLHESGKLQHQLAVDLDRNINIQWQKYCYPLIGSM |
| | TYSVKEMEYLTRAIDRTGGEKNTVIVISLGQHFRPFPIDVFIRRALNVHKAIQHLLLR |
| | SPDTMVIIKTENIREMYNDAERFSDFHGYIQYLIIKDIFQDLSVSIIDAWDITIAYGT |
| | NNVHPPQHVVGNQINILLNYIC |

Sequence comparison of the above protein sequences yields the following sequence relationships shown in Table 11B.

TABLE 11B
Comparison of NOV11a against NOV11b.

| | NOV11a Residues/ | Identities/Similarities |
|---|---|---|
| Protein Sequence | Match Residues | for the Matched Region |
| NOV11b | 1 . . . 545 | 527/545 (96%) |
| | 1 . . . 544 | 529/545 (96%) |

Further analysis of the NOV11a protein yielded the following properties shown in Table 11C.

TABLE 11C
Protein Sequence Properties NOV11a

| | |
|---|---|
| Psort | 0.8200 probability located in outside; 0.4496 probability located in lysosome (lumen); |
| analysis: | 0.1000 probability located in endoplasmic reticulum (membrane); 0.1000 probability |
| | located in endoplasmic reticulum (lumen) |
| SignalP | Cleavage site between residues 28 and 29 |
| analysis: | |

A search of the NOV11a protein against the Geneseq database, a proprietary database that contains sequences published in patents and patent publication, yielded several homologous proteins shown in Table 11D.

TABLE 11D
Geneseq Results for NOV11a

| | | NOV11a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG27904 | Novel human diagnostic protein #27895 - | 29 . . . 545 | 360/520 (69%) | 0.0 |
| | Homo sapiens, 590 aa. | 72 . . . 590 | 425/520 (81%) | |
| | [WO200175067-A2, 11 Oct. 2001] | | | |
| ABG27904 | Novel human diagnostic protein #27895 - | 29 . . . 545 | 360/520 (69%) | 0.0 |
| | Homo sapiens, 590 aa. | 72 . . . 590 | 425/520 (81%) | |
| | [WO200175067-A2, 11 Oct. 2001] | | | |
| ABG12444 | Novel human diagnostic protein #12435 - | 110 . . . 508 | 296/399 (74%) | e—160 |
| | Homo sapiens, 378 aa. | 1 . . . 330 | 308/399 (77%) | |
| | [WO200175067-A2, 11 Oct. 2001] | | | |
| AAB74709 | Human membrane associated protein | 1 . . . 278 | 275/278 (98%) | e—159 |
| | MEMAP-15 - Homo sapiens, 277 aa. | 1 . . . 277 | 277/278 (98%) | |
| | [WO200112662-A2, 22 Feb. 2001] | | | |
| AAM92506 | Human digestive system antigen SEQ ID | 299 . . . 541 | 235/243 (96%) | e—137 |
| | NO:1855 - Homo sapiens, 262 aa. | 13 . . . 255 | 236/243 (96%) | |
| | [WO200155314-A2, 02 Aug. 2001] | | | |

In a BLAST search of public sequence databases, the NOV11a protein was found to have homology to the proteins shown in the BLASTP data in Table 11E.

TABLE 11E
Public BLASTP Results for NOV11a

| | | NOV11a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q05004 | Brush border 61.9 kDa protein precursor - | 12 . . . 545 | 338/537 (62%) | 0.0 |
| | Oryctolagus cuniculus (Rabbit), 540 aa. | 6 . . . 540 | 417/537 (76%) | |
| Q9CX72 | 4432416J03RIK PROTEIN - Mus musculus | 9 . . . 545 | 298/541 (55%) | e—170 |
| | (Mouse), 558 aa. | 21 . . . 558 | 381/541 (70%) | |
| Q96DL1 | CDNA FLJ25224 FIS, CLONE STM00905 - | 9 . . . 297 | 206/289 (71%) | e—113 |
| | Homo sapiens (Human), 365 aa. | 21 . . . 308 | 229/289 (78%) | |
| Q9NXP5 | CDNA FLJ20127 FIS, CLONE COL06176 - | 286 . . . 428 | 142/143 (99%) | 4e—80 |
| | Homo sapiens (Human), 160 aa. | 1 . . . 143 | 142/143 (99%) | |
| Q969Y0 | CDNA FLJ30102 FIS, CLONE | 76 . . . 545 | 161/484 (33%) | le—71 |
| | BNGH41000137, WEAKLY SIMILAR TO | 81 . . . 555 | 269/484 (55%) | |
| | BRUSH BORDER 61.9 KDA PROTEIN | | | |
| | PRECURSOR (UNKNOWN) (PROTEIN FOR | | | |
| | MGC:15606) - Homo sapiens (Human), 559 aa. | | | |

PFam analysis predicts that the NOV11a protein contains the domains shown in the Table 11F.

TABLE 11F
Domain Analysis of NOV11a

| | | Identities/ | |
|---|---|---|---|
| | NOV11a | Similarities for | Expect |
| Pfam Domain | Match Region | the Matched Region | Value |
| Filamin: domain 1 of 1 | 105 . . . 187 | 23/104 (22%) | 5.8 |
| | | 48/104 (46%) | |

### Example 12

The NOV12 clone was analyzed, and the nucleotide and encoded polypeptide sequences are shown in Table 12A.

TABLE 12A
NOV12 Sequence Analysis

| | SEQ ID NO:29 | 1973 bp |
|---|---|---|
| NOV12a, | GGGATATTGGAGTAGCAAGAGGCTGGGAAGCCATCACTTACCTTGCACTGAGAAAGAA | |
| CG91678-01 DNA | GACAAAGGCCAGTATGCACAGCTTTCCTCCACTGCTGCTGCTGCTGTTCTGGGGTGTG | |
| Sequence | GTGTCTCACAGCTTCCCAGCGACTCTAGAAACACAAGAGCAAGATGTGGACTTAGTCC | |
| | AGAAATACCTGGAAAAATACTACAACCTGAAGAATGATGGGAGGCAAGTTGAAAAGCG | |
| | GAGAAATAGTGGCCCAGTGGTTGAAAAATTGAAGCAAATGCAGGAATTCTTTGGGCTG | |
| | AAAGTGACTGGGAAACCAGATGCTGAAACCCTGAAGGTGATGAAGCAGCCCAGATGTG | |
| | GAGTGCCTGATGTGGCTCAGTTTGTCCTCACTGAGGGGAACCCTCGCTGGGAGCAAAC | |
| | ACATCTGACCTACAGGATTGAAAATTACACGCCAGATTTGCCAAGAGCAGATGTGGAC | |
| | CATGCCATTGAGAAAGCCTTCCAACTCTGGAGTAATGTCACACCTCTGACATTCACCA | |
| | AGGTCTCTGAGGGTCAAGCAGACATCATGATATCTTTTGTCAGGGGAGATCATCGGGA | |
| | CAACTCTCCTTTTGATGGACCTGGAGGAAATCTTGCTCATGCTTTTCAACCAGGCCCA | |
| | GGTATTGGAGGGGATGCTCATTTTGATGAAGATGAAAGGTGGACCAACAATTTCAGAG | |
| | AGTACAACTTACATCGTGTTGCGGCTCATGAACTCGGCCATTCTCTTGGACTCTCCCA | |
| | TTCTACTGATATCGGGGCTTTGATGTACCCTAGCTACACCTTCAGTGGTGATGTTCAG | |
| | CTAGCTCAGGATGACATTGATGGCATCCAAGCCATATATGGACGTTCCCAAAATCCTG | |
| | TCCAGCCCATCGGCCCACAAACCCCAAAAGCGTGTGACAGTAAGCTAACCTTTGATGC | |
| | TATAACTACGATTCGGGGAGAAGTGATGTTCTTTAAAGACAGATTCTACATGCGCACA | |
| | AATCCCTTCTACCCGGAAGTTGAGCTCAATTTCATTTCTGTTTTCTGGCCACAACTGC | |
| | CAAATGGGCTTGAAGCTGCTTACGAATTTGCCGACAGAGATGAAGTCCGGTTTTTCAA | |
| | AGGGAATAAGTACTGGGCTGTTCAGGGACAGAATGTGCTACACGGATACCCCAAGGAC | |
| | ATCTACAGCTCCTTTGGCTTCCCTAGAACTGTGAAGCATATCGATGCTGCTCTTTCTG | |
| | AGGAAAACACTGGAAAAACCTACTTCTTTGTTGCTAACAAATACTGGAGGTATGATGA | |
| | ATATAAACGATCTATGGATCCAGGTTATCCCAAAATGATAGCACATGACTTTCCTGGA | |
| | ATTGGCCACAAAGTTGATGCAGTTTTCATGAAAGATGGATTTTTCTATTTCTTTCATG | |
| | GAACAAGACAATACAAATTTGATCCTAAAACGAAGAGAATTTTGACTCTCCAGAAAGC | |
| | TAATAGCTGGTTCAACTGCAGGAAAAATTGAACATTACTAATTTGAATGGAAAACACA | |
| | TGGTGTGAGTCCAAAGAAGGTGTTTTCCTGAAGAACTGTCTATTTTCTCAGTCATTTT | |
| | TAACCTCTAGAGTCACTGATACACAGAATATAATCTTATTTATACCTCAGTTTGCATA | |
| | TTTTTTTACTATTTAGAATGTAGCCCTTTTTGTACTGATATAATTTAGTTCCACAAAT | |
| | GGTGGGTACAAAAAGTCAAGTTTGTGGCTTATGGATTCATATAGGCCAGAGTTGCAAA | |
| | GATCTTTTCCAGAGTATGCAACTCTGACGTTGATCCCAGAGAGCAGCTTCAGTGACAA | |
| | ACATATCCTTTCAAGACAGAAAGAGACAGGAGACATGAGTCTTTGCCGGAGGAAAAGC | |
| | AGCTCAAGAACACATGTGCAGTCACTGGTGTCACCCTGGATAGGCAAGGGATAACTCT | |
| | TCTAACACAAAATAAGTGTTTTATGTTTGGAATAAAGTCAACCTTGTTTCTACTGTTT | |
| | T | |
| | ORF Start: ATG at 72 | ORF Stop: TGA at 1479 |
| | SEQ ID NO:30 | 469 aa MW at 54006.5 kD |
| NOV12a, | MHSFPPLLLLLFWGVVSHSFPATLETQEQDVDLVQKYLEKYYNLKNDGRQVEKRRNSG | |
| CG91678-01 Protein | PVVEKLKQMQEFFGLKVTGKPDAETLKVMKQPRCGVPDVAQFVLTEGNPRWEQTHLTY | |
| Sequence | RIENYTPDLPRADVDHAIEKAFQLWSNVTPLTFTKVSEGQADIMISFVRGDHRDNSPF | |
| | DGPGGNLAHAFQPGPGIGGDAHFDEDERWTNNFREYNLHRVAAHELGHSLGLSHSTDI | |
| | GALMYPSYTFSGDVQLAQDDIDGIQAIYGRSQNPVQPIGPQTPKACDSKLTFDAITTI | |
| | RGEVMFFKDRFYMRTNPFYPEVELNFISVFWPQLPNGLEAAYEFADRDEVRFFKGNKY | |
| | WAVQGQNVLHGYPKDIYSSFGFPRTVKHIDAALSEENTGKTYFFVANKYWRYDEYKRS | |
| | MDPGYPKMIAHDFPGIGHKVDAVFMKDGFFYFFHGTRQYKFDPKTKRILTLQKANSWF | |
| | NCRKN | |
| | SEQ ID NO:31 | 1362 bp |
| NOV12b, | GGTACCTTCCCAGCGACTCTAGAAACACAAGAGCAAGATGTGGACTTAGTCCAGAAAT | |
| 172557724 DNA | ACCTGGAAAAATACTACAACCTGAAGAATGATGGGAGGCAAGTTGAAAAGCGGAGAAA | |
| Sequence | TAGTGGCCCAGTGGTTGAAAAATTGAAGCAAATGCAGGAATTCTTTGGGCTGAAAGTG | |
| | ACTGGGAAACCAGATGCTGAAACCCTGAAGGTGATGAAGCAGCCCAGATGTGGAGTGC | |
| | CTGATGTGGCTCAGTTTGTCCTCACTGAGGGAAACCCTCGCTGGGAGCAAACACATCT | |
| | GACCTACAGGATTGAAAATTACACGCCAGATTTGCCAAGAGCAGATGTGGACCATGCC | |
| | ATTGAGAAAGCCTTCCAACTCTGGAGTAATGTCACACCTCTGACATTCACCAAGGTCT | |
| | CTGAGGGTCAAGCAGACATCATGATATCTTTTGTCAGGGGAGATCATCGGGACAACTC | |
| | TCCTTTTGATGGACCTGGAGGAAATCTTGCTCATGCTTTTCAACCAGGCCCAGGTATT | |
| | GGAGGGGATGCTCATTTTGATGAAGATGAAAGGTGGACCAACAATTTCAGAGAGTACA | |
| | ACTTACATCGTGTTGCGGCTCATGAACTCGGCCATTCTCTTGGACTCTCCCATTCTAC | |
| | TGATATCGGGGCTTTGATGTACCCTAGCTACACCTTCAGTGGTGATGTTCAGCTAGCT | |
| | CAGGATGACATTGATGGCATCCAAGCCATATATGGACGTTCCCAAAATCCTGTCCAGC | |
| | CCATCGGCCCACAAACCCCAAAAGCGTGTGACAGTAAGCTAACCTTTGATGCTATAAC | |
| | TACGATTCGGGGAGAAGTGATGTTCTTTAAAGACAGATTCTACATGCGCACAAATCCC | |
| | TTCTACCCGGAAGTTGAGCTCAATTTCATTTCTGTTTTCTGGCCACAACTGCCAAATG | |
| | GGCTTGAAGCTGCTTACGAATTTGCCGACAGAGATGAAGTCCGGTTTTTCAAAGGGAA | |
| | TAAGTACTGGGCTGTTCAGGGACAGAATGTGCTACACGGATACCCCAAGGACATCTAC | |
| | AGCTCCTTTGGCTTCCCTAGAACTGTGAAGCATATCGATGCTGCTCTTTCTGAGGAAA | |
| | ACACTGGAAAAACCTACTTCTTTGTTGCTAACAAATACTGGAGGTATGATGAATATAA | |
| | ACGATCTATGGATCCAGGTTATCCCAAAATGATAGCACATGACTTTCCTGGAATTGGC | |
| | CACAAAGTTGATGCAGTTTTCATGAAAGATGGATTTTTCTATTTCTTTCATGGAACAA | |
| | GACAATACAAATTTGATCCTAAAACGAAGAGAATTTTGACTCTCCAGAAAGCTAATAG | |
| | CTGGTTCAACTGCAGGAAAAATCTCGAG | |
| | ORF Start: at 1 | ORF Stop: end of sequence |
| | SEQ ID NO:32 | 454 aa MW at 52244.3 kD |
| NOV12b, | GTFPATLETQEQDVDLVQKYLEKYYNLKNDGRQVEKRRNSGPVVEKLKQMQEFFGLKV | |
| 172557724 Protein | TGKPDAETLKVMKQPRCGVPDVAQFVLTEGNPRWEQTHLTYRIENYTPDLPRADVDHA | |
| Sequence | IEKAFQLWSNVTPLTFTKVSEGQADIMISFVRGDHRDNSPFDGPGGNLAHAFQPGPGI | |
| | GGDAHFDEDERWTNNFREYNLHRVAAHELGHSLGLSHSTDIGALMYPSYTFSGDVQLA | |
| | QDDIDGIQAIYGRSQNPVQPIGPQTPKACDSKLTFDAITTIRGEVMFFKDRFYMRTNP | |
| | FYPEVELNFISVFWPQLPNGLEAAYEFADRDEVRFFKGNKYWAVQGQNVLHGYPKDIY | |
| | SSFGFPRTVKHIDAALSEENTGKTYFFVANKYWRYDEYKRSMDPGYPKMIAHDFPGIG | |
| | HKVDAVFMKDGFFYFFHGTRQYKFDPKTKRILTLQKANSWFNCRKDJLE | |
| | SEQ ID NO:33 | 1362 bp |
| NOV12c, | GGTACCTTCCCAGCGACTCTAGAAACACAAGAGCAAGATGTGGACTTAGTCCAGAAAT | |
| 172557764 DNA | ACCTGGAAAAATACTACAACCTGAAGAATGATGGGAGGCAAGTTGAAAAGCGGAGAAA | |
| Sequence | TAGTGGCCCAGTGGTTGAAAAATTGAAGCAAATGCAGGAATTCTTTGGGCTGAAAGTG | |
| | ACTGGGAAACCAGATGCTGAAACCCTGAAGGTGATGAAGCAGCCCAGATGTGGAGTGC | |
| | CTGATGTGGCTCAGTTTGTCCTCACTGAGGGGAACCCTCGCTGGGAGCAAACACATCT | |
| | GACCTACAGGATTGAAAATTACACGCCAGATTTGCCAAGAGCAGATGTGGACCATGCC | |
| | ATTGAGAAAGCCTTCCAACTCTGGAGTAATGTCACACCTCTGACATTCACCAAGGTCT | |
| | CTGAGGGTCAAGCAGACATCATGATATCTTTTGTCAGGGGAGATCATCGGGACAACTC | |
| | TCCTTTTGATGGACCTGGAGGAAATCTTGCTCATGCTTTTCAACCAGGCCCAGGTATT | |
| | GGAGGGGATGCTCATTTTGATGAAGATGAAAGGTGGACCAACAATTTCAGAGAGTACA | |
| | ACTTACATCGTGTTGCGGCTCATGAACTCGGCCATTCTCTTGGACTCTCCCATTCTAC | |
| | TGATATCGGGGCTTTGATGTACCCTAGCTACACCTTCAGTGGTGATGTTCAGCTAGCT | |
| | CAGGATGACATTGATGGCATCCAAGCCATATATGGACGTTCCCAAAATCCTGTCCAGC | |
| | CCATCGGCCCACAAACCCCAAAAGCGTGTGACAGTAAGCTAACCTTTGATGCTATAAC | |
| | TACGATTCGGGGAGAAGTGATGTTCTTTAAAGACAGATTCTACATGCGCACAAATCCC | |
| | TTCTACCCGGAAGTTGAGCTCAATTTCATTTCTGTTTTCTGGTCACAACTGCCAAATG | |
| | GGCTTGAAGCTGCTTACGAATTTGCCGACAGAGATGAAGTCCGGTTTTTCAAAGGGAA | |
| | TAAGTACTGGGCTGTTCAGGGACAGAATGTGCTACACGGATACCCCAAGGACATCTAC | |
| | AGCTCCTTTGGCTTCCCTAGAACTGTGAAGCATATCGATGCTGCTCTTTCTGAGGAAA | |
| | ACACTGGAAAAACCTACTTCTTTGTTGCTAACAAATACTGGAGGTATGATGAATATAA | |
| | ACGATCTATGGATCCAGGTTATCCCAAAATGATAGCACATGACTTTCCTGGAATTGGC | |
| | CACAAAGTTGATGCAGTTTTCATGAAAGATGGATTTTTCTATTTCTTTCATGGAACAA | |
| | GACAATACAAATTTGATCCTAAAACGAAGAGAATTTTGACTCTCCAGAAAGCTAATAG | |
| | CTGGTTCAACTGCAGGAAAAATCTCGAG | |
| | ORF Start: at 1 | ORF Stop: end of sequence |
| | SEQ ID NO:34 | 454 aa MW at 52234.3 kD |
| NOV12c, | GTFPATLETQEQDVDLVQKYLEKYYNLKNDGRQVEKRRNSGPVVEKLKQMQEFFGLKV | |
| 172557764 Protein | TGKPDAETLKVMKQPRCGVPDVAQFVLTEGNPRWEQTHLTYRIENYTPDLPRADVDHA | |
| Sequence | IEKAFQLWSNVTPLTFTKVSEGQADIMISFVRGDHRDNSPFDGPGGNLAHAFQPGPGI | |
| | GGDAHFDEDERWTNNFREYNLHRVAAHELGHSLGLSHSTDIGALMYPSYTFSGDVQLA | |
| | QDDIDGIQAIYGRSQNPVQPIGPQTPKACDSKLTFDAITTIRGEVMFFKDRFYMRTNP | |
| | FYPEVELNFISVFWSQLPNGLEAAYEFADRDEVRFFKGNKYWAVQGQNVLHGYPKDIY | |
| | SSFGFPRTVKHIDAALSEENTGKTYFFVANKYWRYDEYKRSMDPGYPKMIAHDFPGIG | |
| | HKVDAVFMKDGFFYFFHGTRQYKFDPKTKRILTLQKANSWFNCRKNLE | |
| | SEQ ID NO:35 | 1362 bp |
| NOV12d, | GGCACCTTCCCAGCGACTCTAGAAACACAAGAGCAAGATGTGGACTTAGTCCAGAAAT | |
| 173877223 DNA | ACCTGGAAAAATACTACAACCTGAAGAATGATGGGAGGCAAGTTGAAAAGCGGAGAAA | |
| Sequence | TAGTGGCCCAGTGGTTGAAAAATTGAAGCAAATGCAGGAATTCTTTGGGCTGAAAGTG | |
| | ACTGGGAAACCAGATGCTGAAACCCTGAAGGTGATGAAGCAGCCCAGATGTGGAGTGC | |
| | CTGATGTGGCTCAGTTTGTCCTCACTGAGGGGAACCCTCGCTGGGAGCAAACACATCT | |
| | GACCTACAGGATTGAAAATTACACGCCAGATTTGCCAAGAGCAGATGTGGACCATGCC | |
| | ATTGAGAAAGCCTTCCAACTCTGGAGTAGTGTCACACCTCTGACATTCACCAAGGTCT | |
| | CTGAGGGTCAAGCAGACATCATGATATCTTTTGTCAGGGGAGGTCATCGGGACAACTC | |
| | TCCTTTTGATGGACCTGGAGGAAATCTTGCTCATGCTTTTCAACCAGGCCCAGGTATT | |
| | GGAGGGGATGCTCATTTTGATGAAGATGAAAGGTGGACCAACAATTTCAGAGAGTACA | |
| | ACTTACATCGTGTTGCGGCTCATGAACTCGGCCATTCTCTTGGACTCTCCCATTCTAC | |
| | TGATATCGGGGCTTTGATGTACCCTAGCTACACCTTCAGTGGTGATGTTCAGCTAGCT | |
| | CAGGATGACATTGATGGCATCCAAGCCATATATGGACGTTCCCAAAATCCTGTCCAGC | |
| | CCATCGGCCCACAAACCCCAAAAGCGTGTGGCAGTAAGCTAACCTTTGATGCTATAAC | |
| | TACGATTCGGGGAGAAGTGATGTTCTTTAAAGACAGATTCTACATGCGCACAAATCCC | |
| | TTCTACCCGGAAGTTGAGCTCAATTTCATTTCTGTTTTCTGGCCACAACTGCCAAATG | |
| | GGCTTGAAGCTGCTTACGAATTTGCCGACAGAGATGAAGTCCGGTTTTTCAAAGGGAA | |
| | TAAGTACTGGGCTGTTCAGGGACAGAATGTGCTACACGGATACCCCAAGGACATCTAC | |
| | AGCTCCTTTGGCTTCCCTAGAACTGTGAAGCATATCGATGCTGCTCTTTCTGAGGAAA | |
| | ACACTGGAAAAACCTACTTCTTTGTTGCTAACAAATACTGGAGGTATGATGAATATAA | |
| | ACGATCTATGGATCCAGGTTATCCCAAAATGATAGCACATGACTTTCCTGGAATTGGC | |
| | CACAAAGTTGATGCAGTTTTCATGAAAGATGGATTTTTCTATTTCTTTCATGGAACAA | |
| | GACAATACAAATTTGATCCTAAAACGAAGAGAATTTTGACTCTCCAGAAAGCTAATAG | |
| | CTGGTTCAACTGCAGGAAAAATCTCGAG | |
| | ORF Start: at 1 | ORF Stop: end of sequence |
| | SEQ ID NO:36 | 454 aa MW at 52101.2 kD |
| NOV12d, | GTFPATLETQEQDVDLVQKYLEKYYNLKNDGRQVEKRRNSGPVVEKLKQMQEFFGLKV | |
| 173877223 Protein | TGKPDAETLKVMKQPRCGVPDVAQFVLTEGNPRWEQTHLTYRIENYTPDLPRADVDHA | |
| Sequence | IEKAFQLWSSVTPLTFTKVSEGQADIMISFVRGGHRDNSpFDGPGGNLAHAFQPGPGI | |
| | GGDAHFDEDERWTNNFREYNLHRVAAHELGHSLGLSHSTDIGALMYPSYTFSGDVQLA | |
| | QDDIDGIQAIYGRSQNPVQPIGPQTPKACGSKLTFDAITTIRGEVMFFKDRFYMRTNP | |
| | FYPEVELNFISVFWPQLPNGLEAAYEFADRDEVRFFKGNKYWAVQGQNVLHGYPKDIY | |
| | SSFGFPRTVKHIDAALSEENTGKTYFFVANKYWRYDEYKRSMDPGYPKMIAHDFPGIG | |
| | HKVDAVFMKDGFFYFFHGTRQYKFDPKTKRILTLQKANSWFNCRKNLE | |
| | SEQ ID NO:37 | 1362 bp |
| NOV12e, | GGTACCTTCCCAGCGACTCTAGAAACACAAGAGCAAGATGTGGACTTAGTCCAGAAAT | |
| 172557827 DNA | ACCTGGAAAAATACTACAACCTGAAGAATGATGGGAGGCAAGTTGAAAAGCGGAGAAA | |
| Sequence | TAGTGGCCCAGTGGTTGAAAAATTGAAGCAAATGCAGGAATTCTTTGGGCTGAAAGTG | |
| | ACTGGGAAACCAGATGCTGAAACCCTGAAGGTGATGAAGCAGCCCAGATGTGGAGTGC | |
| | CTGATGTGGCTCAGTTTGTCCTCACTGAGGGGAACCCTCGCTGGGAGCAAACACATCT | |
| | GACCTACAGGATTGAAAATTACACGCCAGATTTGCCAAGAGCAGATGTGGACCATGCC | |
| | ATTGAGAAAGCCTTCCAACTCTGGAGTAATGTCACACCTCTGACATTCACCAAGGTCT | |
| | CTGAGGGTCAAGCAGACATCATGATATCTTTTGTCAGGGGAGATCATCGGGACAACTC | |
| | TCCTTTTGATGGACCTGGAGGAAATCTTGCTCATGCTTTTCAACCAGGCCCAGGTATT | |
| | GGAGGGGATGCTCATTTTGATGAAGATGAAAGGTGGACCAACAATTTCAGAGAGTACA | |
| | ACTTACATCGTGTTGCGGCTCATGAACTCGGCCATTCTCTTGGACTCTCCCATTCTAC | |
| | TGATATCGGGGCTTTGATGTACCCTAGCTACACCTTCAGTGGTGATGTTCAGCTAGCT | |
| | CAGGATGACATTGATGGCATCCAAGCCATATATGGACGTTCCCAAAATCCTGTCCAGC | |
| | CCATCGGCCCACAAACCCCAAAAGCGTGTGACAGTAAGCTAACCTTTGATGCTATAAC | |
| | TACGATTCGGGGAGAAGTGATGTTCTTTAAAGACAGATTCTACATGCGCACAAATCCC | |
| | TTCTACCCGGAAGTTGAGCTCAATTTCATTTCTGTTTTCTGGCCACAACTGCCAAATG | |
| | GGCTTGAAGCTGCTTACGAATTTGCCGACAGAGATGAAGTCCGGTTTTTCAAAGGGAA | |
| | TAAGTACTGGGCTGTTCAGGGACAGAATGTGCTACACGGATACCCCAAGGACATCTAC | |
| | AGCTCCTTTGGCTTCCCTAGAACTGTGAAGCATATCGATGCTGCTCTTTCTGAGGAAA | |
| | ACACTGGAAAAACCTACTTCTTTGTTGCTAACAAATACTGGAGGTATGATGAATATAA | |
| | ACGATCTATGGATCCAGGTTATCCCAAAATGATAGCACATGACTTTCCTGGAATTGGC | |
| | CACAAAGTTGATGCAGTTTTCATGAAAGATGGATTTTTCTATTTCTTTCATGGAACAA | |
| | GACAATACAAATTTGATCCTAAAACGAAGAGAATTTTGACTCTCCAGAAAGCTAATAG | |
| | CTGGTTCAACTGCAGGAAAAATCTCGAG | |
| | ORF Start: at 1 | ORF Stop: end of sequence |
| | SEQ ID NO:38 | 454 aa MW at 52244.3 kD |
| NOV12e, | GTFPATLETQEQDVDLVQKYLEKYYNL1NDGRQVEKRRNSGPVVEKLKQMQEFFGLKV | |
| 172557827 Protein | TGKPDAETLKVMKQPRCGVPDVAQFVLTEGNPRWEQTNLTYRIENYTPDLPRADVDNA | |
| Sequence | IEKAFQLWSNVTPLTFTKVSEGQADIMISFVRGDHRDNSPFDGFGGNLAHAFQPGPGI | |
| | GGDAHFDEDERWTNNFREYNLHRVAAHELGHSLGLSHSTDIGALMYPSYTFSGDVQLA | |
| | QDDIDGIQAIYGRSQNPVQPIGPQTPKACDSKLTFDAITTIRGEVMFFKDRFYMRTNP | |
| | FYPEVELNFISVFWPQLPNGLEAAYEFADRDEVRFFKGNKYWAVQGQNVLHGYPKDIY | |
| | SSFGFPRTVKHIDAALSEENTGKTYFFVANKYWRYDEYKRSMDPGYPKMIAHDFPGIG | |
| | HKVDAVFMKDGFFYFFHGTRQYKFDFKTKRILTLQKANSWFNCRKNLE | |
| | SEQ ID NO:39 | 1452 bp |
| NOV12f, | TCACTTACCTTGCACTGAGAAAGAAGACAAAGGCCAGTATGCACAGCTTTCCTCCACT | |
| CG91678-03 DNA | GCTGCTGCTGCTGTTCTGGGGTGTGGTGTCTCACAGCTTCCCAGCGACTCTAGAAACA | |
| Sequence | CGAGAGCAAGATGTGGACTTAGTCCAGAAATACCTGGAAAAATACTACAACCTGAAGA | |
| | ATGATGGGAGGCAAGTTGAAAAGCGGAGAAATAGTGGCCCAGTGGTTGAAAAATTGAA | |
| | GCAAATGCAGGAATTCTTTGGGCTGAAAGTGACTGGGAAACCAGATGCTGAAACCCTG | |
| | AAGGTGATGAAGCAGCCCAGATGTGGAGTGCCTGATGTGGCTCAGTTTGTCCTCACTG | |
| | AGGGAAACCCTCGCTGGGAGCAAACACATCTGACCTACAGGATTGAAAATTACACGCC | |
| | AGATTTGCCAAGAGCAGATGTGGACCATGCCATTGAGAAAGCCTTCCAACTCTGGAGT | |
| | AATGTCACACCTCTGACATTCACCAAGGTCTCTGAGGGTCAAGCAGACATCATGATAT | |
| | CTTTTGTCAGGGGAGATCATCGGGACAACTCTCCTTTTGATGGACCTGGAGGAAATCT | |
| | TGCTCATGCTTTTCAACCAGGCCCAGGTATTGGAGGGGATGCTCATTTTGATGAAGAT | |
| | GAAAGGTGGACCAACAATTTCAGAGAGTACAACTTACATCGTGTTGCGGCTCATGAAC | |
| | TCGGCCATTCTCTTGGACTCTCCCATTCTACTGATATCGGGGCTTTGATGTACCCTAG | |
| | CTACACCTTCAGTGGTGATGTTCGGCTAGCTCAGGATGACATTGATGGCATCCAAGCC | |
| | ATATATGGACGTTCCCAAAATCCTGTCCAGCCCATCGGCCCACAAACCCCAAAAGCGT | |
| | GTGACAGTAAGCTAACCTTTGATGCTATAACTACGATTCGGGGAGAAGTGATGTTCTT | |
| | TAAAGACAGATTCTACATGCGCACAAATCCCTTCTACCCGGAAGTTGAGCTCAATTTC | |
| | ATTTCTGTTTTCTGGCCACAACTGCCAAATGGGCTTGAAGCTGCTTACGAATTTGCCG | |
| | ACAGAGATGAAGTCCGGTTTTTCAAAGGGAATAAGTACTGGGCTGTTCAGGGACAGAA | |
| | TGTGCTACACGGATACCCCAAGGACATCTACAGCTCCTTTGGCTTCCCTAGAACTGTG | |
| | AAGCATATCGATGCTGCTCTTTCTGAGGAAAACACTGGAAAAACCTACTTCTTTGTTG | |
| | CTAACAAATACTGGAGGTATGATGAATATAAACGATCTATGGATCCAGGTTATCCCAA | |
| | AATGATAGCACATGACTTTCCTGGAATTGGCCACAAAGTTGATGCAGTTTTCATGAAA | |
| | GATGGATTTTTCTATTTCTTTCATGGAACAAGACAATACAAATTTGATCCTAAAACGA | |
| | AGAGAATTTTGACTCTCCAGAAAGCTAATAGCTGGTTCAACTGCAGGAAAAATTGAAC | |
| | AT | |
| | ORF Start: ATG at 39 | ORF Stop: TGA at 1446 |
| | SEQ ID NO:40 | 469 aa MW at 54062.6 kD |

| | |
|---|---|
| NOV12f, | MHSFPPLLLLLFWGVVSHSFPATLETREQDVDLVQKYLEKYYNLKNDGRQVEKRRNSG |
| CG91678-03 Protein | PVVEKLKQMQEFFGLKVTGKPDAETLKVMKQPRCGVPDVAQFVLTEGNPRWEQTHLTY |
| Sequence | RIENYTPDLPRADVDHAIEKAFQLWSNVTPLTFTKVSEGQADIMISFVRGDHRDNSPF |
| | DGPGGNLAHAFQPGPGIGGDAHFDEDERWTNNFREYNLHRVAAHELGHSLGLSHSTDI |
| | GALMYPSYTFSGDVRLAQDDIDGIQAIYGRSQNPVQPIGPQTPKACDSKLTFDAITTI |
| | RGEVMFFKDRFYMRTNPFYPEVELNFISVFWPQLPNGLEAAYEFADRDEVRFFKGNKY |
| | WAVQGQNVLHGYPKDIYSSFGFPRTVKHIDAALSEENTGKTYFFVANKYWRYDEYKRS |
| | MDPGYPKMIAHDFPGIGHKVDAVFMKDGFFYFFHGTRQYKFDPKTKRILTLQKANSWF |
| | NCRKN |

Sequence comparison of the above protein sequences yields the following sequence relationships shown in Table 12B.

TABLE 12B
Comparison of NOV12a against NOV12b through NOV12f.

| | NOV12a Residues/ | Identities/Similarities |
|---|---|---|
| Protein Sequence | Match Residues | for the Matched Region |
| NOV12b | 19 . . . 469 | 450/451 (99%) |
| | 2 . . . 452 | 451/451 (99%) |
| NOV12c | 19 . . . 469 | 449/451 (99%) |
| | 2 . . . 452 | 450/451 (99%) |
| NOV12d | 19 . . . 469 | 447/451 (99%) |
| | 2 . . . 452 | 449/451 (99%) |
| NOV12e | 19 . . . 469 | 450/451 (99%) |
| | 2 . . . 452 | 451/451 (99%) |
| NOV12f | 1 . . . 469 | 467/469 (99%) |
| | 1 . . . 469 | 469/469 (99%) |

Further analysis of the NOV12a protein yielded the following properties shown in Table 12C.

TABLE 12C
Protein Sequence Properties NOV12a

| | |
|---|---|
| PSort | 0.5411 probability located in lysosome (lumen); 0.3700 probability located in outside; |
| analysis: | 0.3404 probability located in microbody (peroxisome); 0.1000 probability located in |
| | endoplasmic reticulum (membrane) |
| SignalP | Cleavage site between residues 20 and 21 |
| analysis: | |

A search of the NOV12a protein against the Geneseq database, a proprietary database that contains sequences published in patents and patent publication, yielded several homologous proteins shown in Table 12D.

TABLE 12D
Geneseq Results for NOV12a

| | | | Identifies/ | |
|---|---|---|---|---|
| Geneseq | Protein/Organism/Length | NOV12a Residues/ | Similarities for the | Expect |
| Identifier | [Patent #, Date] | Match Residues | Matched Region | Value |
| AAG75509 | Human colon cancer antigen protein SEQ | 1 . . . 469 | 469/469 (100%) | 0.0 |
| | ID NO:6273 - Homo sapiens, 496 aa. | 28 . . . 496 | 469/469 (100%) | |
| | [WO200122920-A2, 05-APR-2001] | | | |
| AAB84606 | Amino acid sequence of matrix | 1 . . . 469 | 469/469 (100%) | 0.0 |
| | metalloproteinase collagenase 1 - Homo | 1 . . . 469 | 469/469 (100%) | |
| | sapiens, 469 aa. [WO200149309-A2, | | | |
| | 12-JUL-2001] | | | |
| AAE10415 | Human matrix metalloprotinase-1 | 1 . . . 469 | 469/469 (100%) | 0.0 |
| | (MMP-1) protein - Homo sapiens, 469 aa. | 1 . . . 469 | 469/469 (100%) | |
| | [WO200166766-A2, 13-SEP-2001] | | | |
| AAP70611 | Sequence encoded by human skin | 1 . . . 469 | 467/469 (99%) | 0.0 |
| | collagenase cDNA - Homo sapiens, 469 aa. | 1 . . . 469 | 467/469 (99%) | |
| | [GB2182665-A, 20-MAY-1987] | | | |
| AAP93628 | Sequence of human interstitial | 20 . . . 469 | 448/450 (99%) | 0.0 |
| | procollagenase - Homo sapiens, 457 aa. | 8 . . . 457 | 448/450 (99%) | |
| | [GB2209526-A, 17-MAY-1989] | | | |

In a BLAST search of public sequence databases, the NOV12a protein was found to have homology to the proteins shown in the BLASTP data in Table 12E.

TABLE 12E
Public BLASTP Results for NOV12a

| Protein | | | Identities/ | |
|---|---|---|---|---|
| Accession | | NOV12a Residues/ | Similarities for | Expect |
| Number | Protein/Organism/Length | Match Residues | the Matched Portion | Value |
| P03956 | Interstitial collagenase precursor (EC | 1 . . . 469 | 469/469 (100%) | 0.0 |
| | 3.4.24.7) (Matrix metalloproteinase-1) | 1 . . . 469 | 469/469 (100%) | |
| | (MMP-1) (Fibroblast collagenase) - Homo | | | |
| | sapiens (Human), 469 aa. | | | |
| Q9XSZ5 | Interstitial collagenase precursor (EC | 6 . . . 469 | 404/465 (86%) | 0.0 |
| | 3.4.24.7) (Matrix metalloproteinase-1) | 5 . . . 469 | 435/465 (92%) | |
| | (MMP-1) - Equus caballus (Horse), 469 aa. | | | |
| P13943 | Interstitial collagenase precursor (EC | 6 . . . 469 | 403/464 (86%) | 0.0 |
| | 3.4.24.7) (Matrix metalloproteinase-1) | 5 . . . 468 | 428/464 (91%) | |
| | (MMP-1) - Oryctolagus cuniculus (Rabbit), | | | |
| | 468 aa. | | | |
| P28053 | Interstitial collagenase precursor (EC | 6 . . . 469 | 396/465 (85%) | 0.0 |
| | 3.4.24.7) (Matrix metalloproteinase-1) | 5 . . . 469 | 426/465 (91%) | |
| | (MMP-1) (Fibroblast collagenase) - Bos | | | |
| | taurus (Bovine), 469 aa. | | | |
| P21692 | Interstitial collagenase precursor (EC 3.4.24.7) | 7 . . . 469 | 396/464 (85%) | 0.0 |
| | (Matrix metalloproteinase-1) (MMP-1) - Sus | 6 . . . 469 | 429/464 (92%) | |
| | scrofa (Pig), 469 aa. | | | |

PFam analysis predicts that the NOV12a protein contains the domains shown in the Table 12F.

TABLE 12F
Domain Analysis of NOV12a

| | | Identities/ | |
|---|---|---|---|
| | | Similarities | |
| | NOV12a | for the | |
| Pfam Domain | Match Region | Matched Region | Expect Value |
| PG_binding_1: | 27 . . . 91 | 15/73 (21%) | 0.5 |
| domain 1 of 1 | | 46/73 (63%) | |
| Peptidase_M10: | 37 . . . 204 | 113/171 (66%) | 5.9e−121 |
| domain 1 of 1 | | 164/171 (96%) | |
| Astacin: domain | 107 . . . 264 | 38/236 (16%) | 0.3 |
| 1 of 1 | | 104/236 (44%) | |
| hemopexin: domain | 284 . . . 326 | 16/50 (32%) | 1.3e−09 |
| 1 of 4 | | 33/50 (66%) | |
| hemopexin: domain | 328 . . . 372 | 20/50 (40%) | 8.1e−13 |
| 2 of 4 | | 36/50 (72%) | |
| hemopexin: domain | 377 . . . 424 | 24/50 (48%) | 3.1e−21 |
| 3 of 4 | | 44/50 (88%) | |
| hemopexin: domain | 426 . . . 466 | 13/50 (26%) | 4.7e−07 |
| 4 of 4 | | 32/50 (64%) | |

The NOV13 clone was analyzed, and the nucleotide and encoded polypeptide sequences are shown in Table 13A.

TABLE 13A
NOV13 Sequence Analysis

| | SEQ ID NO:41 | 1669 bp |
|---|---|---|
| NOV13, | ATGCTGCTGCGCTCGAAGCCTGCGCTGCCGCCGCCGCTGCTGATGCTGCTGCTCCTGG | |
| CG91698-01 DNA | GGCCGCTGGGTCCCCTCTCCCCTGGCGCCCTGCCCCGACCTGCGCAAGCACAGCAGGA | |
| Sequence | CGTCGTGGACCTGGACTTCTTCACCCAGGAGCCGCTGCACCTGGTGAGCCCCTCGTTC | |
| | CTGTCCGTCACCATTGACGCCAACCTGGCCACGGACCCGCGGTTCCTCATCCTCCTGG | |
| | GTTCTCCAAAGCTTCGTACCTTGGCCAGAGGCTTGTCTCCTGCGTACCTGAGGTTTGG | |
| | TGGCACCAAGACAGACTTCCTAATTTTCGATCCCAAGAAGGAATCAACCTTTGAAGAG | |
| | AGAAGTTACTGGCAATCTCAAGTCAACCAGGATATTTGCAAATATGGATCCATCCCTC | |
| | CTGATGTGGAGGAGAAGTTACGGTTGGAATGGCCCTACCAGGAGCAATTGCTACTCCG | |
| | AGAACACTACCAGAAAAAGTTCAAGAACAGCACCTACTCAAGAAGCTCTGTAGATGTG | |
| | CTATACACTTTTGCAAACTGCTCAGGACTGGACTTGATCTTTGGCCTAAATGCGTTAT | |
| | TAAGAACAGCAGATTTGCAGTGGAACAGTTCTAATGCTCAGTTGCTCCTGGACTACTG | |
| | CTCTTCCAAGGGGTATAACATTTCTTGGGAACTAGGCAATGAACCTAACAGTTTCCTT | |
| | AAGAAGGCTGATATTTTCATCAATGGGTCGCAGTTAGGAGAAGATTTTATTCAATTGC | |
| | ATAAACTTCTAAGAAAGTCCACCTTCAAAAATGCAAAACTCTATGGTCCTGATGTTGG | |
| | TCAGCCTCGAAGAAAGACGGCTAAGATGCTGAAGAGCTTCCTGAAGGCTGGTGGAGAA | |
| | GTGATTGATTCAGTTACATGGCATCACTACTATTTGAATGGACGGACTGCTACCAGGG | |
| | AAGATTTTCTAAACCCTGATGTATTGGACATTTTTATTTCATCTGTGCAAAAAGTTTT | |
| | CCAGGTGGTTGAGAGCACCAGGCCTGGCAAGAAGGTCTGGTTAGGAGAAAGAAGCTCT | |
| | GCATATGGAGGCGGAGCGCCCTTGCTATCCGACACCTTTGCAGCTGGCTTTATGTGGC | |
| | TGGATAAATTGGGCCTGTCAGCCCGAATGGGAATAGAAGTGGTGATGAGGCAAGTATT | |
| | CTTTGGAGCAGGAAACTACCATTTAGTGGATGAAAACTTCGATCCTTTACCTGATTAT | |
| | TGGCTATCTCTTCTGTTCAAGAAATTGGTGGGCACCAAGGTGTTAATGGCAAGCGTGC | |
| | AAGGTTCAAAGAGAAGGAAGCTTCGAGTATACCTTCATTGCACAAACACTGACAATCC | |
| | AAGGTATAAAGAAGGAGATTTAACTCTGTATGCCATAAACCTCCATAACGTCACCAAG | |
| | TACTTGCGGTTACCCTATCCTTTTTCTAACAAGCAAGTGGATAAATACCTTCTAAGAC | |
| | CTTTGGGACCTCATGGATTACTTTCCAAATCTGTCCAACTCAATGGTCTAACTCTAAA | |
| | GATGGTGGATGATCAAACCTTGCCACCTTTAATGGAAAAACCTCTCCGGCCAGGAAGT | |
| | TCACTGGGCTTGCCAGCTTTCTCATATAGTTTTTTTGTGATAAGAAATGCCAAAGTTG | |
| | CTGCTTGCATCTGAAAATAAAATATACTAGTCCTGACACTGAAAA | |
| | ORF Start: ATG at 1 | ORF Stop: TGA at 1636 |
| | SEQ ID NO:42 | 545 aa MW at 61417.3 kD |

| | |
|---|---|
| NOV13, | MLLRSKPALPPPLLMLLLLGPLGPLSPGALPRPAQAQQDVVDLDFFTQEPLHLVSPSF |
| CG91698-01 Protein | LSVTIDANLATDPRFLILLGSPKLRTLARGLSPAYLRFGGTKTDFLIFDPKKESTFEE |
| Sequence | RSYWQSQVNQDICKYGSIPPDVEEKLRLEWPYQEQLLLREHYQKKFKNSTYSRSSVDV |
| | LYTFANCSGLDLIFGLNALLRTADLQWNSSNAQLLLDYCSSKGYNISWELGNEPNSFL |
| | KKADIFINGSQLGEDFIQLHKLLRKSTFKNAKLYGPDVGQPRRKTAKMLKSFLKAGGE |
| | VIDSVTWHHYYLNGRTATREDFLNPDVLDIFISSVQKVFQVVESTRPGKKVWLGETSS |
| | AYGGGAPLLSDTFAAGFMWLDKLGLSARMGIEVVMRQVFFGAGNYHLVDENFDPLPDY |
| | WLSLLFKKLVGTKVLMASVQGSKRRKLRVYLHCTNTDNPRYKEGDLTLYAINLHNVTK |
| | YLRLPYPFSNKQVDKYLLRPLGPHGLLSKSVQLNGLTLKMVDDQTLPPLMEKPLRPGS |
| | SLGLPAFSYSFFVIRNAKVAACI |

Further analysis of the NOV13 protein yielded the following properties shown in Table 13B.

TABLE 13B
Protein Sequence Properties NOV13

| | |
|---|---|
| PSort | 0.4669 probability located in lysosome (lumen); |
| analysis: | 0.3894 probability located in outside; |
| | 0.2239 probability located in microbody (peroxisome); |
| | 0.1000 probability located in |
| | endoplasmic reticulum (membrane) |
| SignalP | Cleavage site between residues 37 and 38 |
| analysis: | |

A search of the NOV13 protein against the Geneseq database, a proprietary database that contains sequences published in patents and patent publication, yielded several homologous proteins shown in Table 13C.

TABLE 13C
Geneseq Results for NOV13

| | | | Identities/ | |
|---|---|---|---|---|
| Geneseq | Protein/Organism/Length | NOV13 Residues/ | Similarities for the | |
| Identifier | [Patent #, Date] | Match Residues | Matched Region | Expect Value |
| AAB86206 | Human heparanase inhibitor protein - | 1 . . . 545 | 543/545 (99%) | 0.0 |
| | Homo sapiens, 543 aa. [DE19955803-A1, | 1 . . . 543 | 543/545 (99%) | |
| | 23-MAY-2001] | | | |
| AAY17082 | Human heparanase enzyme - Homo sapiens, | 1 . . . 545 | 543/545 (99%) | 0.0 |
| | 543 aa. [WO9921975-A1, 06-MAY-1999] | 1 . . . 543 | 543/545 (99%) | |
| AAY30124 | A human protein with heparanase activity - | 1 . . . 545 | 543/545 (99%) | 0.0 |
| | Homo sapiens, 588 aa. [WO9940207-A1, | 46 . . . 588 | 543/545 (99%) | |
| | 12-AUG-1999] | | | |
| AAY97635 | Human heparanase protein sequence - | 1 . . . 545 | 542/545 (99%) | 0.0 |
| | Homo sapiens, 543 aa. [WO200100643-A2, | 1 . . . 543 | 543/545 (99%) | |
| | 04-JAN-2001] | | | |
| AAY52990 | Human heparanase protein sequence - | 1 . . . 545 | 542/545 (99%) | 0.0 |
| | Homo sapiens, 543 aa. [WO9957153-A1, | 1 . . . 543 | 543/545 (99%) | |
| | 11-NOV-1999] | | | |

In a BLAST search of public sequence databases, the NOV13 protein was found to have homology to the proteins shown in the BLASTP data in Table 13D.

TABLE 13D
Public BLASTP Results for NOV13

| Protein | | | Identities/ | |
|---|---|---|---|---|
| Accession | | NOV13 Residues/ | Similarities for the | |
| Number | Protein/Organism/Length | Match Residues | Matched Portion | Expect Value |
| Q9UL39 | HEPARANASE - Homo sapiens | 1 . . . 545 | 545/545 (100%) | 0.0 |
| | (Human), 545 aa. | 1 . . . 545 | 545/545 (100%) | |
| Q9Y251 | HEPARANASE - Homo sapiens | 1 . . . 545 | 543/545 (99%) | 0.0 |
| | (Human), 543 aa. | 1 . . . 543 | 543/545 (99%) | |
| CAC39726 | SEQUENCE 89 FROM PATENT | 1 . . . 545 | 541/545 (99%) | 0.0 |
| | EP1067182 - Homo sapiens (Human), | 1 . . . 543 | 542/545 (99%) | |
| | 543 aa. | | | |
| CAC10140 | SEQUENCE 14 FROM PATENT | 1 . . . 525 | 523/525 (99%) | 0.0 |
| | EP1032656 - Homo sapiens (Human), | 1 . . . 523 | 523/525 (99%) | |
| | 532 aa. | | | |
| Q9MYY0 | HEPARANASE - Bos taurus (Bovine), | 1 . . . 545 | 437/546 (80%) | 0.0 |
| | 545 aa. | 1 . . . 545 | 471/546 (86%) | |

PFam analysis predicts that the NOV13 protein contains the domains shown in the Table 13E.

TABLE 13E
Domain Analysis of NOV13

| | | | |
|---|---|---|---|
| | NOV13 | Identities/Similarities | Expect |
| Pfam Domain | Match Region | for the Matched Region | Value |

No Significant Known Matches Found

### Example 14

The NOV14 clone was analyzed, and the nucleotide and encoded polypeptide sequences are shown in Table 14A.

TABLE 14A
NOV14 Sequence Analysis

| | SEQ ID NO:43 | 1821 bp |
|---|---|---|
| NOV14a, | ACAAGGAGGCAGGCAAGACAGCAAGGCATAGAGACAACATAGAGCTAAGTAAAGCCAG | |
| CG91708-01 DNA | TGGAAATGAAGAGTCTTCCAATCCTACTGTTGCTGTGCGTGGCAGTTTGCTCAGCCTA | |
| Sequence | TCCATTGGATGGAGCTGCAAGGGGTGAGGACACCAGCATGAACCTTGTTCAGAAATAT | |
| | CTAGAAAACTACTACGACCTCAAAAAAGATGTGAAACAGTTTGTTAGGAGAAAGGACA | |
| | GTGGTCCTGTTGTTAAAAAAATCCGAGAAATGCAGAAGTTCCTTGGATTGGAGGTGAC | |
| | GGGGAAGCTGGACTCCGACACTCTGGAGGTGATGCGCAAGCCCAGGTGTGGAGTTCCT | |
| | GATGTTGGTCACTTCAGAACCTTTCCTGGCATCCCGAAGTGGAGGAAAACCCACCTTA | |
| | CATACAGGATTGTGAATTATACACCAGATTTGCCAAAAGATGCTGTTGATTCTGCTGT | |
| | TGAGAAAGCTCTGAAAGTCTGGGAAGAGGTGACTCCACTCACATTCTCCAGGCTGTAT | |
| | GAAGGAGAGGCTGATATAATGATCTCTTTTGCAGTTAGAGAACATGGAGACTTTTACC | |
| | CTTTTGATGGACCTGGAAATGTTTTGGCCCATGCCTATGCCCCTGGGCCAGGGATTAA | |
| | TGGAGATGCCCACTTTGATGATGATGAACAATGGACAAAGGATACAACAGGGACCAAT | |
| | TTATTTCTCGTTGCTGCTCATGAAATTGGCCACTCCCTGGGTCTCTTTCACTCAGCCA | |
| | ACACTGAAGCTTTGATGTACCCACTCTATCACTCACTCACAGACCTGACTCGGTTCCG | |
| | CCTGTCTCAAGATGATATAAATGGCATTCAGTCCCTCTATGGACCTCCCCCTGACTCC | |
| | CCTGAGACCCCCCTGGTACCCACGGAACCTGTCCCTCCAGAACCTGGGACGCCAGCCA | |
| | ACTGTGATCCTGCTTTGTCCTTTGATGCTGTCAGCACTCTGAGGGGAGAAATCCTGAT | |
| | CTTTAAAGACAGGCACTTTTGGCGCAAATCCCTCAGGAAGCTTGAACCTGAATTGCAT | |
| | TTGATCTCTTCATTTTGGCCATCTCTTCCTTCAGGCGTGGATGCCGCATATGAAGTTA | |
| | CTAGCAAGGACCTCGTTTTCATTTTTAAAGGAAATCAATTCTGGGCCATCAGAGGAAA | |
| | TGAGGTACGAGCTGGATACCCAAGAGGCATCCACACCCTAGGTTTCCCTCCAACCGTG | |
| | AGGAAAATCGATGCAGCCATTTCTGATAAGGAAAAGAACAAAACATATTTCTTTGTAG | |
| | AGGACAAATACTGGAGATTTGATGAGAAGAGAAATTCCATGGAGCCAGGCTTTCCCAA | |
| | GCAAATAGCTGAAGACTTTCCAGGGATTGACTCAAAGATTGATGCTGTTTTTGAAGAA | |
| | TTTGGGTTCTTTTATTTCTTTACTGGATCTTCACAGTTGGAGTTTGACCCAAATGCAA | |
| | AGAAAGTGACACACACTTTGAAGAGTAACAGCTGGCTTAATTGTTGAAAGAGATATGT | |
| | AGAAGGCACAATATGGGCACTTTAAATGAAGCTAATAATTCTTCACCTAAGTCTCTGT | |
| | GAATTGAAATGTTCGTTTTCTCCTGCCTGTGCTGTGACTCGAGTCACACTCAAGGGAA | |
| | CTTGAGCGTGAATCTGTATCTTGCCGGTCATTTTTATGTTATTACAGGGCATTCAAAT | |
| | GGGCTGCTGCTTAGCTTGCACCTTGTCACATAGAGTGATCTTTCCCAAGAGAAGGGGA | |
| | AGCACTCGTGTGCAACAGACAAGTGACTGTATCTGTGTAGACTATTTGCTTATTTAAT | |
| | AAAGACGATTTGTCAGTTGTTTT | |
| | ORF Start: ATG at 64 | ORF Stop: TGA at 1495 |
| | SEQ ID NO:44 | 477 aa MW at 53976.7 kD |
| NOV14a, | MKSLPILLLLCVAVCSAYPLDGAARGEDTSMNLVQKYLENYYDLKKDVKQFVRRKDSG | |
| CG91708-01 Protein | PVVKKIREMQKFLGLEVTGKLDSDTLEVMRKPRCGVPDVGHFRTFPGIPKWRKTHLTY | |
| Sequence | RIVNYTPDLPKDAVDSAVEKALKVWEEVTPLTFSRLYEGEADIMISFAVREHGDFYPF | |
| | DGPGNVLAHAYAPGPGINGDAHFDDDEQWTKDTTGTNLFLVAAHEIGHSLGLFHSANT | |
| | EALMYPLYHSLTDLTRFRLSQDDINGIQSLYGPPPDSPETPLVPTEPVPPEPGTPANC | |
| | DPALSFDAVSTLRGEILIFKDRHFWRKSLRKLEPELHLISSFWPSLPSGVDAAYEVTS | |
| | KDLVFIFKGNQFWAIRGNEVRAGYPRGIHTLGFPPTVRKIDAAISDKEKNKTYFFVED | |
| | KYWRFDEKRNSMEPGFPKQIAEDFPGIDSKIDAVFEEFGFFYFFTGSSQLEFDPNAKK | |
| | VTHTLKSNSWLNC | |
| | SEQ ID NO:45 | 1580 bp |
| NOV14b, | CAAGACAGCAAGGCATAGAGACAACATAGAGCTAAGTAAAGCCAGTGGAAATGAAGAG | |
| CG91708-02 DNA | TCTTCCAATCCTACTGTTGCTGTGCGTGGCAGTTTGCTCAGCCTATCCATTGGATGGA | |
| Sequence | GCTGCAAGGGGTGAGGACACCAGCATGAACCTTGTTCAGAAATATCTAGAAAACTACT | |
| | ACGACCTCGAAAAAGATGTGAAACAGTTTGTTAGGAGAAAGGACAGTGGTCCTGTTGT | |
| | TAAAAAAATCCGAGAAATGCAGAAGTTCCTTGGATTGGAGGTGACGGGGAAGCTGGAC | |
| | TCCGACACTCTGGAGGTGATGCGCAAGCCCATGTGTGGAGTTCCTGACGTTGGTCACT | |
| | TCAGAACCTTTCCTGGCATCCCGAAGTGGAGGAAAACCCACCTTACATACAGGATTGT | |
| | GAATTATACACCAGATTTGCCAAAAGATGCTGTTGATTCTGCTGTTGAGAAAGCTCTG | |
| | AAAGTCTGGGAAGAGGTGACTCCACTCACATTCTCCAGGCTGTATGAAGGAGAGACTG | |
| | ATATAATGATCTCTTTTGCAGTTAGAGAACATGGAGACTTTTACCCTTTTGATGGACC | |
| | TGGAAATGTTTTGGCCCATGCCTATGCCCCTGGGCCAGGGATTAATGGAGATGCCCAC | |
| | TTTGATGATGATGAACAATGGACAAAGGATACAACAGGGACCAATTTATTTCTCGTTG | |
| | CTGCTCATGAAATTGGCCACTCCCTGGGTCTCTTTCACTCAGCCAACACTGAAGCTTT | |
| | GATGTACCCACTCTATCACTCACTCACAGACCTGACTCGGTTCCGCCTGTCTCAAGAT | |
| | GATATAAATGGCATTCAGTCCCTCTATGGACCTCCCCCTGACTCCCCTGAGACCCCCC | |
| | TGGTACCCACGGAACCTGTCCCTCCAGAACCTGGGACGCCAGCCAACTGTGATCCTGC | |
| | TTTGTCCTTTGATGCTGTCAGCACTCTGAGGGGAGAAATCCTGATCTTTAAAGACAGG | |
| | CACTTTTGGCGCAAATCCCTCAGGAAGCTTGAACCTGAATTGCATTTGATCTCTTCAT | |
| | TTTGGCCATCTCTTCCTTCAGGCGTGGATGCCGCATATGAAGTTACTAGCAAGGACCT | |
| | CGTTTTCATTTTTAAAGGAAATCAATTCTGGGCCATCAGAGGAAATGAGGTACGAGCT | |
| | GGATACCCAAGAGGCATCCACACCCTAGGTTTCCCTCCAACCGTGAGGAAAATCGATG | |
| | CAGCCATTTCTGATAAGGAAAAGAACAAAACATATTTCTTTGTAGAGGACAAATACTG | |
| | GAGATTTGATGAGAAGAGAAATTCCATGGAGCCAGGCTTTCCCAAGCAAATAGCTGAA | |
| | GACTTTCCAGGGATTGACTCAAAGATTGATGCTGTTTTTGAAGAATTTGGGTTCTTTT | |
| | ATTTCTTTACTGGATCTTCACAGTTGGAGTTTGACCCAATGCAAAGAAAAGTGACACA | |
| | CACTTTGAAGAGTAACAGCTGGCTTAATTGTTGAAAGAGATATGTAGAAGGCACAATA | |
| | TGGGCACTTTAAATGAAGCTAATAATTCTTCACCTAAGTCTCTGTGAATTGAAATGTT | |
| | CGTTTTCTCCTGCT | |
| | ORF Start: ATG at 51 | ORF Stop: TGA at 1482 |
| | SEQ ID NO:46 | 477 aa MW at 53982.7 kD |
| NOV14b, | MKSLPILLLLCVAVCSAYPLDGAARGEDTSMNLVQKYLENYYDLEKDVKQFVRRKDSG | |
| CG91708-02 Protein | PVVKKIREMQKFLGLEVTGKLDSDTLEVMRKPMCGVPDVGHFRTFPGIPKWRKTHLTY | |
| Sequence | RIVNYTPDLPKDAVDSAVEKALKVWEEVTPLTFSRLYEGETDIMISFAVREHGDFYPF | |
| | DGPGNVLAHAYAPGPGINGDAHFDDDEQWTKDTTGTNLFLVAAHEIGHSLGLFHSANT | |
| | EALMYPLYHSLTDLTRFRLSQDDINGIQSLYGPPPDSPETPLVPTEPVPPEPGTPANC | |
| | DPALSFDAVSTLRGEILIFKDRHFWRKSLRKLEPELHLISSFWPSLPSGVDAAYEVTS | |
| | KDLVFIFKGNQFWAIRGNEVRAGYPRGIHTLGFPPTVRKIDAAISDKEKNKTYFFVED | |
| | KYWRFDEKRNSMEPGFPKQIAEDFPGIDSKIDAVFEEFGFFYFFTGSSQLEFDPNAKK | |
| | VTHTLKSNSWLNC | |
| | SEQ ID NO:47 | 519 bp |
| NOV14c, | GGATCCACCTATCTAGAAAACTACTACGACCTCGAAAAAGATGTGAAACAGTTTGTTA | |
| 240317953 DNA | GGAGAAAGGACAGTGGTCCTGTTGTTAAAAAAATCCGAGAAATGCAGAAGTTCCTTGG | |
| Sequence | ATTGGAGGTGACGGGGAAGCAGGACTCCGACACTCTGGAGGTGATGCGCAAGCCCAGG | |
| | TGTGGAGTTCCTGACGTTGGTCACTTCAGAACCTTTCCTGGCATCCCGAAGTGGAGGA | |
| | AAACCCACCTTACATACAGGATTGTGAATTATACACCAGATTTGCCAAAAGATGCTGT | |
| | TGATTCTGCTGTTGAGAAAGCTCTGAAAGTCTGGGAAGAGGTGACTCCACTCACATTC | |
| | TCCAGGCTGTATGAAGGAGAGGCTGATATAATGATCTCTTTTGCAGTTAGAGAACATG | |
| | GAGACTTTTACCCTTTTGATGGACCTGGAAATGTTTTGGCCCATGCCTATGCCCCTGG | |
| | GCCAGGGATTAATGGAGATGCCCACTTTGATGATGATGAACAATGGACACTCGAG | |
| | ORF Start: at 1 | ORF Stop: end of sequence |
| | SEQ ID NO:48 | 173 aa MW at 19767.1 kD |
| NOV14c, | GSTYLENYYDLEKDVKQFVRRKDSGPVVKKIREMQKFLGLEVTGKQDSDTLEVMRKPR | |
| 240317953 Protein | CGVPDVGHFRTFPGIPKWRKTHLTYRIVNYTPDLPKDAVDSAVEKALKVWEEVTPLTF | |
| Sequence | SRLYEGEADIMISFAVREHGDFYPFDGPGNVLAHAYAPGPGINGDAHFDDDEQWTLE | |
| | SEQ ID NO:49 | 483 bp |
| NOV14d, | GGATCCACCACCCACCTTACATACAGGATTGTGAATTATACACCAGATTTGCCAAAAG | |
| 240317980 DNA | ATGCTGTTGATTCTGCTGTTGAGAAAGCTCTGAAAGTCTGGGAAGAGGTGACTCCACT | |
| Sequence | CACATTCTCCAGGCTGTATGAAGGAGAGGCTGATATAATGATCTCTTTTGCAGTTAGA | |
| | GAACATGGAGACTTTTACCCTTTTGATGGACCTGGAAATGTTTTGGCCCATGCCTATG | |
| | CCCCTGGGCCAGGGATTAATGGAGATGCCCACTTTGATGATGATGAACAATGGACAAA | |
| | GGATACAACAGGGACCAATTTATTTCTCGTTGCTGCTCATGAAATTGGCCACTCCCTG | |
| | GGTCTCTTTCACTCAGCCAACACTGAAGCTTTGATGTACCCACTCTATCACTCACTCA | |
| | CAGACCTGACTCGGTTCCGCCTGTCTCAAGATGATATAAATGGCATTCAGTCCCTCTA | |
| | TGGACCTCCCCCTCTCGAG | |
| | ORF Start: at 1 | ORF Stop: end of sequence |
| | SEQ ID NO:50 | 161 aa MW at 17838.5 kD |

| | |
|---|---|
| NOV14d, | GSTTHLTYRIVNYTPDLPKDAVDSAVEKALKVWEEVTPLTFSRLYEGEADIMISFAVR |
| 240317980 Protein | EHGDFYPFDGPGNVLAHAYAPGPGINGDAHFDDDEQWTKDTTGTNLFLVAAHEIGHSL |
| Sequence | GLFHSANTEALMYPLYHSLTDLTRFRLSQDDINGIQSLYGPPPLE |

Sequence comparison of the above protein sequences yields the following sequence relationships shown in Table 14B.

TABLE 14B
Comparison of NOV14a against NOV14b through NOV14d.

| | NOV14a Residues/ | Identities/Similarities |
|---|---|---|
| Protein Sequence | Match Residues | for the Matched Region |
| NOV14b | 1 . . . 477 | 446/477 (93%) |
| | 1 . . . 477 | 447/477 (93%) |
| NOV14c | 37 . . . 204 | 166/168 (98%) |
| | 4 . . . 171 | 167/168 (98%) |
| NOV14d | 112 . . . 267 | 156/156 (100%) |
| | 4 . . . 159 | 156/156 (100%) |

Further analysis of the NOV14a protein yielded the properties shown in Table 14C.

TABLE 14C
Protein Sequence Properties NOV14a

| | |
|---|---|
| PSort | 0.8200 probability located in outside; |
| analysis: | 0.3106 probability located in microbody (peroxisome); |
| | 0.1900 probability located in lysosome (lumen); |
| | 0.1000 probability located in endoplasmic |
| | reticulum (membrane) |
| SignalP | Cleavage site between residues 18 and 19 |
| analysis: | |

A search of the NOV14a protein against the Geneseq database, a proprietary database that contains sequences published in patents and patent publication, yielded several homologous proteins shown in Table 14D.

TABLE 14D
Geneseq Results for NOV14a

| Geneseq | Protein/Organism/Length | NOV14a Residues/ | Identities/Similarities | |
|---|---|---|---|---|
| Identifier | [Patent #, Date] | Match Residues | for the Matched Region | Expect Value |
| AAE10420 | Human matrix metalloprotinase-3 (MMP-3) | 1 . . . 477 | 477/477 (100%) | 0.0 |
| | protein - Homo sapiens, 477 aa. | 1 . . . 477 | 477/477 (100%) | |
| | [WO200166766-A2, 13-SEP-2001] | | | |
| AAY21993 | Human matrix metalloprotease-3 (MMP-3) - | 1 . . . 477 | 477/477 (100%) | 0.0 |
| | Homo sapiens, 477 aa. [JP11169176-A, | 1 . . . 477 | 477/477 (100%) | |
| | 29-JUN-1999] | | | |
| AAB84608 | Amino acid sequence of matrix | 1 . . . 477 | 476/477 (99%) | 0.0 |
| | metalloproteinase-3 stromelysin 1 - Homo | 1 . . . 477 | 477/477 (99%) | |
| | sapiens, 477 aa. [WO200149309-A2, | | | |
| | 12-JUL-2001] | | | |
| AAY21994 | Human matrix metalloprotease-3 (MMP-3) - | 1 . . . 477 | 472/477 (98%) | 0.0 |
| | Homo sapiens, 477 aa. [JP11169176-A, | 1 . . . 477 | 472/477 (98%) | |
| | 29-JUN-1999] | | | |
| AAP80257 | Sequence of human stromelysin - Homo | 1 . . . 477 | 469/477 (98%) | 0.0 |
| | sapiens, 477 aa. [WO8707907-A, | 1 . . . 477 | 472/477 (98%) | |
| | 30-DEC-1987] | | | |

In a BLAST search of public sequence databases, the NOV14a protein was found to have homology to the proteins shown in the BLASTP data in Table 14E.

TABLE 14E
Public BLASTP Results for NOV14a

| | | NOV14a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| P08254 | Stromelysin-1 precursor (EC 3.4.24.17) | 1 . . . 477 | 477/477 | (100%) | 0.0 |
| | (Matrix metalloproteinase-3) (MMP-3) | 1 . . . 477 | 477/477 | (100%) | |
| | (Transin-1) (SL-1) - Homo sapiens | | | | |
| | (Human), 477 aa. | | | | |
| P28863 | Stromelysin-1 precursor (EC 3.4.24.17) | 1 . . . 477 | 402/478 | (84%) | 0.0 |
| | (Matrix metalloproteinase-3) (MMP-3) | 1 . . . 478 | 435/478 | (90%) | |
| | (Transin-1) (SL-1) - Oryctolagus | | | | |
| | cuniculus (Rabbit), 478 aa. | | | | |
| Q28397 | Stromelysin-1 precursor (EC 3.4.24.17) | 1 . . . 477 | 388/477 | (81%) | 0.0 |
| | (Matrix metalloproteinase-3) (MMP-3) - | 1 . . . 477 | 429/477 | (89%) | |
| | Equus caballus (Horse), 477 aa. | | | | |
| P09238 | Stromelysin-2 precursor (EC 3.4.24.22) | 1 . . . 477 | 373/477 | (78%) | 0.0 |
| | (Matrix metalloproteinase-10) (MMP-10) | 1 . . . 476 | 420/477 | (87%) | |
| | (Transin-2) (SL-2) - Homo sapiens | | | | |
| | (Human), 476 aa. | | | | |
| Q922W6 | MATRIX METALLOPROTEINASE 3 - | 1 . . . 477 | 368/477 | (77%) | 0.0 |
| | Mus musculus (Mouse), 479 aa. | 3 . . . 479 | 415/477 | (86%) | |

PFam analysis predicts that the NOV14a protein contains the domains shown in the Table 14F.

TABLE 14F
Domain Analysis of NOV14a

| | | Identities/ | | |
|---|---|---|---|---|
| | | Similarities for | | Expect |
| Pfam Domain | NOV14a Match Region | the Matched Region | | Value |
| Peptidase_M10: domain 1 of 1 | 37 . . . 204 | 118/171 | (69%) | 4.4e−126 |
| | | 166/171 | (97%) | |
| Astacin: domain 1 of 1 | 112 . . . 267 | 36/226 | (16%) | 0.41 |
| | | 102/226 | (45%) | |
| hemopexin: domain 1 of 4 | 296 . . . 338 | 16/50 | (32%) | 5.1e−12 |
| | | 37/50 | (74%) | |
| hemopexin: domain 2 of 4 | 340 . . . 383 | 16/50 | (32%) | 5.6e−13 |
| | | 39/50 | (78%) | |
| hemopexin: domain 3 of 4 | 388 . . . 435 | 125/50 | (50%) | 6.6e−19 |
| | | 141/50 | (82%) | |
| hemopexin: domain 4 of 4 | 437 . . . 477 | 17/50 | (34%) | 1.5e−09 |
| | | 33/50 | (66%) | |

### Example 15

The NOV15 clone was analyzed, and the nucleotide and encoded polypeptide sequences are shown in Table 15A.

TABLE 15A
NOV15 Sequence Analysis

| | SEQ ID NO:51 | 2722 bp |
|---|---|---|
| NOV15a, | CAACAGTCCCCAGGCATCACCATTCAAGATGCATCCAGGGGTCCTGGCTGCCTTCCTC | |
| GG91729-01 DNA | TTCTTGAGCTGGACTCATTGTCGGGCCCTGCCCCTTCCCAGTGGTGGTGATGAAGATG | |
| Sequence | ATTTGTCTGAGGAAGACCTCCAGTTTGCAGAGCGCTACCTGAGATCATACTACCATCC | |
| | TACAAATCTCGCGGGAATCCTGAAGGAGAATGCAGCAAGCTCCATGACTGAGAGGCTC | |
| | CGAGAAATGCAGTCTTTCTTCGGCTTAGAGGTGACTGGCAAACTTGACGATAACACCT | |
| | TAGATGTCATGAAAAAGCCAAGATGCGGGGTTCCTGATGTGGGTGAATACAATGTTTT | |
| | CCCTCGAACTCTTAAATGGTCCAAAATGAATTTAACCTACAGAATTGTGAATTACACC | |
| | CCTGATATGACTCATTCTGAAGTCGAAAAGGCATTCAAAAAAGCCTTCAAAGTTTGGT | |
| | CCGATGTAACTCCTCTGAATTTTACCAGACTTCACGATGGCATTGCTGACATCATGAT | |
| | CTCTTTTGGAATTAAGGAGCATGGCGACTTCTACCCATTTGATGGGCCCTCTGGCCTG | |
| | CTGGCTCATGCTTTTCCTCCTGGGCCAAATTATGGAGGAGATGCCCATTTTGATGATG | |
| | ATGAAACCTGGACAAGTAGTTCCAAAGGCTACAACTTGTTTCTTGTTGCTGCGCATGA | |
| | GTTCGGCCACTCCTTAGGTCTTGACCACTCCAAGGACCCTGGAGCACTCATGTTTCCT | |
| | ATCTACACCTACACCGGCAAAAGCCACTTTATGCTTCCTGATGACGATGTACAAGGGA | |
| | TCCAGTCTCTCTATGGTCCAGGAGATGAAGACCCCAACCCTAAACATCCAAAAACGCC | |
| | AGACAAATGTGACCCTTCCTTATCCCTTGATGCCATTACCAGTCTCCCAGGAGAAACA | |
| | ATGATCTTTAAAGACAGATTCTTCTGGCGCCTGCATCCTCAGCAGGTTGATGCGGAGC | |
| | TGTTTTTAACGAAATCATTTTGGCCAGAACTTCCCAACCGTATTGATGCTGCATATGA | |
| | GCACCCTTCTCATGACCTCATCTTCATCTTCAGAGGTAGAAAATTTTGGGCTCTTAAT | |
| | GGTTATGACATTCTGGAAGGTTATCCCAAAAAAATATCTGAACTGGGTCTTCCAAAAG | |
| | AAGTTAAGAAGATAAGTGCAGCTGTTCACTTTGAGGATACAGGCAAGACTCTCCTGTT | |
| | CTCAGGAAACCAGGTCTGGAGATATGATGATACTAACCATATTATGGATAAAGACTAT | |
| | CCGAGACTAATAGAAGAAGACTTCCCAGGAATTGGTGATAAAGTAGATGCTGTCTATG | |
| | AGAAAAATGGTTATATCTATTTTTTCAACGGACCCATACAGTTTGAATACAGCATCTG | |
| | GAGTAACCGTATTGTTCGCGTCATGCCAGCAAATTCCATTTTGTGGTGTTAAGTGTCT | |
| | TTTTAAAAATTGTTATTTAAATCCTGAAGAGCATTTGGGGTAATACTTCCAGAAGTGC | |
| | GGGGTAGGGGAAGAAGAGCTATCAGGAGAAAGCTTGGTTCTGTGAACAAGCTTCAGTA | |
| | AGTTATCTTTGAATATGTAGTATCTATATGACTATGCGTGGCTGGAACCACATTGAAG | |
| | AATGTTAGAGTAATGAAATGGAGGATCTCTAAAGAGCATCTGATTCTTGTTGCTGTAC | |
| | AAAAGCAATGGTTGATGATACTTCCCACACCACAAATGGGACACATGGTCTGTCAATG | |
| | AGAGCATAATTTAAAAATATATTTATAAGGAAATTTTACAAGGGCATAAAGTAAATAC | |
| | ATGCATATAATGAATAAATCATTCTTACTAAAAAGTATAAAATAGTATGAAAATGGAA | |
| | ATTTGGGAGAGCCATACATAAAAGAAATAAACCAAAGGAAAATGTCTGTAATAATAGA | |
| | CTGTAACTTCCAAATAAATAATTTTCATTTTGCACTGAGGATATTCAGATGTATGTGC | |
| | CCTTCTTCACACAGACACTAACGAAATATCAAAGTCATTAAAGACAGGAGACAAAAGA | |
| | GCAGTGGTAAGAATAGTAGATGTGGCCTTTGAATTCTGTTTAATTTTCACTTTTGGCA | |
| | ATGACTCAAAGTCTGCTCTCATATAAGACAAATATTCCTTTGCATATTATAAAGGATA | |
| | AAGAAGGATGATGTCTTTTTATTAAAATATTTCAGGTTCTTCAGAAGTCACACATTAC | |
| | AAAGTTAAAATTGTTATCAAAATAGTCTAAGGCCATGGCATCCCTTTTTCATAAATTA | |
| | TTTGATTATTTAAGACTAAAAGTTGCATTTTAACCCTATTTTACCTAGCTAATTATTT | |
| | AATTGTCCGGTTTGTCTTGGATATATAGGCTATTTTCTAAAGACTTGTATAGCATGAA | |
| | ATAAAATATATCTTATAAAGTGGAAGTATGTATATTAAAAAAGAGACATCCAAATTTT | |
| | TTTTTAAAGCAGTCTACTAGATTGTGATCCCTTGAGATATGGAAGGATGCCTTTTTTT | |
| | CTCTGCATTTAAAAAAATCCCCCAGCACTTCCCACAGTGCCTATTGATACTTGGGGAG | |
| | GGTGCTTGGCACTTATTGAATATATGATCGGCCATCAAGGGAAGAACTATTGTGCTCA | |
| | GAGACACTGTTGATAAAAACTCAGGCAAAGAAAATGAAATGCATATTTGCAAAGTGTA | |
| | TTAGGAAGTGTTTATGTTGTTTATAATAAAAATATATTTTCAACAGAAAAAAAA | |
| | ORF Start: ATG at 29 | ORF Stop: TAA at 1442 |
| | SEQ ID NO:52 | 471 aa MW at 53819.2 kD |
| NOV15a, | MHPGVLAAFLFLSWTHCRALPLPSGGDEDDLSEEDLQFAERYLRSYYHPTNLAGILKE | |
| CG91729-01 Protein | NAASSMTERLREMQSFFGLEVTGKLDDNTLDVMKKPRCGVPDVGEYNVFPRTLKWSKM | |
| Sequence | NLTYRIVNYTPDMTHSEVEKAFKKAFKVWSDVTPLNFTRLHDGIADIMISFGIKEHGD | |
| | FYPFDGPSGLLAHAFPPGPNYGGDAHFDDDETWTSSSKGYNLFLVAAHEFGHSLGLDH | |
| | SKDPGALMFPIYTYTGKSHFMLPDDDVQGIQSLYGPGDEDPNPKHPKTPDKCDPSLSL | |
| | DAITSLRGETMIFKDRFFWRLHPQQVDAELFLTKSFWPELPNRIDAAYEHPSHDLIFI | |
| | FRGRKFWALNGYDILEGYPKKISELGLPKEVKKISAAVHFEDTGKTLLFSGNQVWRYD | |
| | DTNHIMDKDYPRLIEEDFPGIGDKVDAVYEKNGYIYFFNGPIQFEYSIWSNRIVRVMP | |
| | ANSILWC | |
| | SEQ ID NO:53 | 1426 bp |
| NOV15b, | CCATTCAAGATGCATCCAGGGGTCCTGGCTGCCTTCCTCTTCTTGAGCTGGACTCATT | |
| CG91729-02 DNA | GTCGGGCCCTGCCCCTTCCCAGTGGTGGTGATGAAGATGATTTGTCTGAGGAAGACCT | |
| Sequence | CCAGTTTGCAGAGCGCTACCTGAGATCATACTACCATCCTACAAATCTCGCGGGAATC | |
| | CTGAAGGAGAATGCAGCAAGCTCCATGACTGAGAGGCTCCGAGAAATGCAGTCTTTCT | |
| | TCGGCTTAGAGGTGACTGGCAAACTTGACGATAACACCTTAGATGTCATGAAAAAGCC | |
| | AAGATGCGGGGTTCCTGATGTGGGTGAATACAATGTTTTCCCTCGAACTCTTAAATGG | |
| | TCCAAAATGAATTTAACCTACAGAATTGTGAATTACACCCCTGATATGACTCATTCTG | |
| | AAGTCGAAAAGGCATTCAAAAAAGCCTTCAAAGTTTGGTCCGATGTAACTCCTCTGAA | |
| | TTTTACCAGACTTCACGATGGCATTGCTGACATCATGATCTCTTTTGGAATTAAGGAG | |
| | CATGGCGACTTCTACCCATTTGATGGGCCCTCTGGCCTGCTGGCTCATGCTTTTCCTC | |
| | CTGGGCCAAATTATGGAGGAGATGCCCATTTTGATGATGATGAAACCTGGACAAGTAG | |
| | TTCCAAAGGCTACAACTTGTTTCTTGTTGCTGCGCATGAGTTCGGCCACTCCTTAGGT | |
| | CTTGACCACTCCAAGGACCCTGGAGCACTCATGTTTCCTATCTACACCTACACCGGCA | |
| | AAAGCCACTTTATGCTTCCTGATGACGATGTACAAGGGATCCAGTCTCTCTATGGTCC | |
| | AGGAGATGAAGACCCCAACCCTAAACATCCAAAAACGCCAGACAAATGTGACCCCTCC | |
| | TTATCCCTTGATGCCATTACCAGTCTCCGAGGAGAAACAATGATCTTTAAAGACAGAT | |
| | TCTTCTGGCGCCTGCATCCTCAGCAGGTTGATGCGGAGCTGTTTTTAACGAAATCATT | |
| | TTGGCCAGAACTTCCCAACCGTATTGATGCTGCATATGAGCACCCTTCTCATGACCTC | |
| | ATCTTCATCTTCAGAGGTAGAAAATTTTGGGCTCTTAATGGTTATGACATTCTGGAAG | |
| | GTTATCCCAAAAAAATATCTGAACTGGGTCTTCCAAAAGAAGTTAAGAAGATAAGTGC | |
| | AGCTGTTCACTTTGAGGATACAGGCAAGACTCTCCTGTTCTCAGGAAACCAGGTCTGG | |
| | AGATATGATGATACTAACCATATTATGGATAAAGACTATCCGAGACTAATAGAAGAAG | |
| | ACTTCCCAGGAATTGGTGATAAAGTAGATGCTGTCTATGAGAAAAATGGTTATATCTA | |
| | TTTTTTCAACGGACCCATACAGTTTGAATACAGCATCTGGAGTAACCGTATTGTTCGC | |
| | GTCATGCCAGCAAATTCCATTTTGTGGTGTTAAG | |
| | ORF Start: ATG at 10 | ORF Stop: TAA at 1423 |
| | SEQ ID NO:54 | 471 aa MW at 53819.2 kD |

| | |
|---|---|
| NOV15b, | MHPGVLAAFLFLSWTHCRALPLPSGGDEDDLSEEDLQFAERYLRSYYHPTNLAGILKE |
| CG91729-02 Protein | NAASSMTERLREMQSFFGLEVTGKLDDNTLDVMKKPRCGVPDVGEYNVFPRTLKWSKM |
| Sequence | NLTYRIVNYTPDMTHSEVEKAFKKAFKVWSDVTPLNFTRLHDGIADIMISFGIKEHGD |
| | FYPFDGPSGLLAHAFPPGPNYGGDAHFDDDETWTSSSKGYNLFLVAAHEFGHSLGLDH |
| | SKDPGALMFPIYTYTGKSHFMLPDDDVQGIQSLYGPGDEDPNPKHPKTPDKCDPSLSL |
| | DAITSLRGETMIFKDRFFWRLHPQQVDAELFLTKSFWPELPNRIDAAYEHPSHDLIFI |
| | FRGRKFWALNGYDILEGYPKKISELGLPKEVKKISAAVHFEDTGKTLLFSGNQVWRYD |
| | DTNHIMDKDYPRLIEEDFPGIGDKVDAVYEKNGYIYFFNGPIQFEYSIWSNRIVRVMP |
| | ANSILWC |

Sequence comparison of the above protein sequences yields the following sequence relationships shown in Table 15B.

TABLE 15B
Comparison of NOV15a against NOV15b.

| | | Identities/ | |
|---|---|---|---|
| | NOV15a Residues/ | Similarities for | |
| Protein Sequence | Match Residues | the Matched Region | |
| NOV15b | 1 . . . 471 | 458/471 | (97%) |
| | 1 . . . 471 | 458/471 | (97%) |

Further analysis of the NOV15a protein yielded the following properties shown in Table 15C.

TABLE 15C
Protein Sequence Properties NOV15a

| | |
|---|---|
| PSort | 0.3700 probability located in outside; 0.2550 probability |
| analysis: | located in microbody (peroxisome); 0.1900 probability located |
| | in lysosome (lumen); 0.1000 probability located in |
| | endoplasmic reticulum (membrane) |
| SignalP | Cleavage site between residues 20 and 21 |
| analysis: | |

A search of the NOV15a protein against the Geneseq database, a proprietary database that contains sequences published in patents and patent publication, yielded several homologous proteins shown in Table 15D.

TABLE 15D
Geneseq Results for NOV15a

| | | NOV15a | | | |
|---|---|---|---|---|---|
| | | Residues/ | Identities/ | | |
| Geneseq | Protein/Organism/Length | Match | Similarities for | | Expect |
| Identifier | [Patent #,Date] | Residues | the Matched Region | | Value |
| AAB84615 | Amino acid sequence of matrix | 1 . . . 471 | 471/471 | (100%) | 0.0 |
| | metalloproteinase-13 - Homo sapiens, 471 | 1 . . . 471 | 471/471 | (100%) | |
| | aa. [WO200149309-A2, Jul. 12, 2001] | | | | |
| AAE10428 | Human matrix metalloprotinase-20P | 1 . . . 471 | 471/471 | (100%) | 0.0 |
| | (MMP-20P) protein - Homo sapiens, 471 | 1 . . . 471 | 471/471 | (100%) | |
| | aa. [WO200166766-A2, Sep. 13, 2001] | | | | |
| AAE10417 | Human matrix metalloprotinase-13 | 1 . . . 471 | 471/471 | (100%) | 0.0 |
| | (MMP-13) protein - Homo sapiens, 471 aa. | 1 . . . 471 | 471/471 | (100%) | |
| | [WO200166766-A2, Sep. 13, 2001] | | | | |
| AAY29419 | Human matrix metalloproteinase 13 - | 1 . . . 471 | 470/471 | (99%) | 0.0 |
| | Homo sapiens, 471 aa. [WO9931969-A2, | 1 . . . 471 | 470/471 | (99%) | |
| | Jul. 01, 1999] | | | | |
| AAB84608 | Amino acid sequence of matrix | 6 . . . 471 | 236/477 | (49%) | e−139 |
| | metalloproteinase-3 stromelysin 1 - Homo | 4 . . . 477 | 314/477 | (65%) | |
| | sapiens, 477 aa. [WO200149309-A2, | | | | |
| | Jul. 12, 2001] | | | | |

In a BLAST search of public sequence databases, the NOV15a protein was found to have homology to the proteins shown in the BLASTP data in Table 15E.

TABLE 15E
Public BLASTP Results for NOV15a

| | | NOV15a | | | |
|---|---|---|---|---|---|
| Protein | | Residues/ | Identities/ | | |
| Accession | | Match | Similarities for | | Expect |
| Number | Protein/Organism/Length | Residues | the Matched Portion | | Value |
| P45452 | Collagenase 3 precursor (EC 3.4.24.-) | 1 . . . 471 | 471/471 | (100%) | 0.0 |
| | (Matrix metalloproteinase-13) (MMP-13) - | 1 . . . 471 | 471/471 | (100%) | |
| | Homo sapiens (Human), 471 aa. | | | | |
| O18927 | Collagenase 3 precursor (EC 3.4.24.-) | 1 . . . 471 | 430/472 | (91%) | 0.0 |
| | (Matrix metalloproteinase-13) (MMP-13) - | 1 . . . 472 | 451/472 | (95%) | |
| | Equus caballus (Horse), 472 aa. | | | | |
| O62806 | Collagenase 3 precursor (EC 3.4.24.-) | 1 . . . 471 | 425/471 | (90%) | 0.0 |
| | (Matrix metalloproteinase-13) (MMP-13) - | 1 . . . 471 | 445/471 | (94%) | |
| | Oryctolagus cuniculus (Rabbit), 471 aa. | | | | |
| O77656 | Collagenase 3 precursor (EC 3.4.24.-) | 1 . . . 471 | 423/471 | (89%) | 0.0 |
| | (Matrix metalloproteinase-13) (MMP-13) - | 1 . . . 471 | 444/471 | (93%) | |
| | Bos taurus (Bovine), 471 aa. | | | | |
| Q9TT82 | MATRIX METALLOPROTEINASE-13 - | 8 . . . 457 | 419/450 | (93%) | 0.0 |
| | Canis familiaris (Dog), 452 aa (fragment). | 1 . . . 449 | 432/450 | (95%) | |

PFam analysis predicts that the NOV15a protein contains the domains shown in the Table 15F.

TABLE 15F
Domain Analysis of NOV15a

| | | Identities/ | | |
|---|---|---|---|---|
| | NOV15a | Similarities for | | |
| Pfam Domain | Match Region | the Matched Region | | Expect Value |
| Peptidase_M10: domain 1 of 1 | 42 . . . 208 | 113/171 | (66%) | 2.2e−121 |
| | | 164/171 | (96%) | |
| hemopexin: domain 1 of 4 | 290 . . . 332 | 17/50 | (34%) | 2.8e−10 |
| | | 37/50 | (74%) | |
| hemopexin: domain 2 of 4 | 334 . . . 377 | 19/50 | (38%) | 2.7e−13 |
| | | 38/50 | (76%) | |
| hemopexin: domain 3 of 4 | 382 . . . 429 | 19/50 | (38%) | 6.5e−16 |
| | | 40/50 | (80%) | |
| hemopexin: domain 4 of 4 | 431 . . . 471 | 110/50 | (20%) | 2.9e−05 |
| | | 28/50 | (56%) | |

### Example 16

The NOV16 clone was analyzed, and the nucleotide and encoded polypeptide sequences are shown in Table 16A.

TABLE 16A
NOV16 Sequence Analysis

| | SEQ ID NO:55 | 1680 bp |
|---|---|---|
| NOV16a, | AGACGCAGAGACAGACAAACAAACAGATAGGAGAGGCTCTCCAGGAGGCCGGGGGGCC | |
| CG92489-01 DNA | CACTCCGCCTATCGCTCCCCTCGGCTACGCTGCCACTTCAATGCCCCGCAGGTCGCGA | |
| | GCTGCTGTTCTTTCGAAGGCGTCGGAGAACCAGGGGCGTCCCGCGCCACCTCTGACTC | |
| | GGAGCAGCGCCGAGCACTGACGCTCCCGCCCTTGGGCAAGGACGCCAGTGCGCCCGCG | |
| | CGCGTCCCTCTGCGCGGCAGCCCGTCGCGGGCCCTCAAGGGGAAGCCCAGGCCAGGAT | |
| | GGCCCCGGGTCGCGCGGTGGCCGGGCTCCTGTTGCTGGCGGCCGCCGGCCTCGGAGGA | |
| | GTGGCGGAGGGGCCAGGGCTAGCCTTCAGCGAGGATGTGCTGAGCGTGTTCGGCGCGA | |
| | ATCTGAGCCTGTCGGCGGCGCAGCTCCAGCACTTGCTGGAGCAGATGGGAGCCGCCTC | |
| | CCGCGTGGGCGTCCCGGAGCCTGGCCAGCTGCACTTCAACCAGTGTTTAACTGCTGAA | |
| | GAGATCTTTTCCCTTCATGGCTTTTCAAATGCTACCCAAATAACCAGCTCCAAATTCT | |
| | CTGTCATCTGTCCAGCAGTCTTACAGCAATTGAACTTTCACCCATGTGAGGATCGGCC | |
| | CAAGCACAAAACAAGACCAAGTCATTCAGAAGTTTGGGGATATGGATTCCTGTCAGTG | |
| | ACGATTATTAATCTGGCATCTCTCCTCGGATTGATTTTGACTCCACTGATAAAGAAAT | |
| | CTTATTTCCCAAAGATTTTGACCTTTTTTGTGGGGCTGGCTATTGGGACTCTTTTTTC | |
| | AAATGCAATTTTCCAACTTATTCCAGAGGCATTTGGATTTGATCCCAAAGTCGACAGT | |
| | TATGTTGAGAAGGCAGTTGCTGTGTTTGGTGGATTTTACCTACTTTTCTTTTTTGAAA | |
| | GAATGCTAAAGATGTTATTAAAGACATATGGTCAGAATGGTCATACCCACTTTGGAAA | |
| | TGATAACTTTGGTCCTCAAGAAAAAACTCATCAACCTAAAGCATTACCTGCCATCAAT | |
| | GGTGTGACATGCTATGCAAATCCTGCTGTCACAGAAGCTAATGGACATATCCATTTTG | |
| | ATAATGTCAGTGTGGTATCTCTACAGGATGGAAAAAAAGAGCCAAGTTCATGTACCTG | |
| | TTTGAAGGGGCCCAAACTGTCAGAAATAGGGACGATTGCCTGGATGATAACGCTCTGC | |
| | GATGCCCTCCACAATTTCATCGATGGCCTGGCGATTGGGGCTTCCTGCACCTTGTCTC | |
| | TCCTTCAGGGACTCAGTACTTCCATAGCAATCCTATGTGAGGAGTTTCCCCACGAGTT | |
| | AGGAGACTTTGTGATCCTACTCAATGCAGGGATGAGCACTCGACAAGCCTTGCTATTC | |
| | AACTTCCTTTCTGCATGTTCCTGCTATGTTGGGCTAGCTTTTGGCATTTTGGTGGGCA | |
| | ACAATTTCGCTCCAAATATTATATTTGCACTTGCTGGAGGCATGTTCCTCTATATTTC | |
| | TCTGGCAGATATGTTTCCAGAGATGAATGATATGCTGAGAGAAAAGGTAACTGGAAGA | |
| | AAAACCGATTTCACCTTCTTCATGATTCAGAATGCTGGAATGTTAACTGGATTCACAG | |
| | CCATTCTACTCATTACCTTGTATGCAGGAGAAATCGAATTGGAGTAATAGAAAATG | |
| | ORF Start: ATG at 289 | ORF Stop: TAA at 1669 |
| | SEQ ID NO:56 | 460 aa MW at 49630.0 kD |
| NOV16a, | MAPGRAVAGLLLLAAAGLGGVAEGPGLAFSEDVLSVFGANLSLSAAQLQHLLEQMGAA | |
| CG92489-01 Protein | SRVGVPEPGQLHFNQCLTAEEIFSLHGFSNATQITSSKFSVICPAVLQQLNFHPCEDR | |
| Sequence | PKHKTRPSHSEVWGYGFLSVTIINLASLLGLILTPLIKKSYFPKILTFFVGLAIGTLF | |
| | SNAIFQLIPEAFGFDPKVDSYVEKAVAVFGGFYLLFFFERMLKMLLKTYGQNGHTHFG | |
| | NDNFGPQEKTHQPKALPAINGVTCYANPAVTEANGHIHFDNVSVVSLQDGKKEPSSCT | |
| | CLKGPKLSEIGTIAWMITLCDALHNFIDGLAIGASCTLSLLQGLSTSIAILCEEFPHE | |
| | LGDFVILLNAGMSTRQALLFNFLSACSCYVGLAFGILVGNNFAPNIIFALAGGMFLYI | |
| | SLADMFPEMNDMLREKVTGRKTDFTFFMIQNAGMLTGFTAILLITLYAGEIELE | |
| | SEQ ID NO:57 | 1326 bp |
| NOV16b, | GGATCCGAGGGGCCAGGGCTAGCCTTCAGCGAGGATGTGCTGAGCGTGTTCGGCGCGA | |
| 228495688 DNA | ATCTGAGCCTGTCGGCGGCGCAGCTCCAGCACTTGCTGGAGCAGATGGGAGCCGCCTC | |
| Sequence | CCGCGTGGGCGTCCCGGAGCCTGGCCAGCTGCACTTCAACCAGTGTTTAACTGCTGAA | |
| | GAGATCTTTTCCCTTCATGGCTTTTCAAATGCTACCCAAATAACCAGCTCCAAATTCT | |
| | CTGTCATCTGTCCAGCAGTCTTACAGCAATTGAACTTTCACCCATGTGAGGATCGGCC | |
| | CAAGCACAAAACAAGACCAAGTCATTCAGAAGTTTGGGGATATGGATTCCTGTCAGTG | |
| | ACGATTATTAATCTGGCATCTCTCCTCGGATTGATTTTGACTCCACTGATAAAGAAAT | |
| | CTTATTTCCCAAAGATTTTGACCTTTTTTGTGGGGCTGGCTATTGGGACTCTTTTTTC | |
| | AAATGCAATTTTCCAACTTATTCCAGAGGCATTTGGATTTGATCCCAAAGTCGACAGT | |
| | TATGTTGAGAAGGCAGTTGCTGTGTTTGGTGGATTTTACCTACTTTTCTTTTTTGAAA | |
| | GAATGCTAAAGATGTTATTAAAGACATATGGTCAGAATGGTCATACCCACTTTGGAAA | |
| | TGATAACTTTGGTCCTCAAGAAAAAACTCATCAACCTAAAGCATTACCTGCCATCAAT | |
| | GGTGTGACATGCTATGCAAATCCTGCTGTCACAGAAGCTAATGGACATATCCATTTTG | |
| | ATAATGTCAGTGTGGTATCTCTACAGGATGGAAAAAAAGAGCCAAGTTCATGTACCTG | |
| | TTTGAAGGGGCCCAAACTGTCAGAAATAGGGACGATTGCCTGGATGATAACGCTCTGC | |
| | GATGCCCTCCACAATTTCATCGATGGCCTGGCGATTGGGGCTTCCTGCACCTTGTCTC | |
| | TCCTTCAGGGACTCAGTACTTCCATAGCAATCCTATGTGAGGAGTTTCCCCACGAGTT | |
| | AGGAGACTTTGTGATCCTACTCAATGCAGGGATGAGCACTCGACAAGCCTTGCTATTC | |
| | AACTTCCTTTCTGCATGTTCCTGCTATGTTGGGCTAGCTTTTGGCATTTTGGTGGGCA | |
| | ACAATTTCGCTCCAAATATTATATTTGCACTTACTGGAGGCATGTTCCTCTATATTTT | |
| | TCTGGCAGATATGTTTCCAGAGATGAATGATATGCTGAGAGAAAAGGTAACTGGAAGA | |
| | AAAACCGATTTCACCTTCTTCATGATTCAGAATGCTGGAATGTTAACTGGATTCACAG | |
| | CCATTCTACTCATTACCTTGTATGCAGGAGAAATCGAATTGGAGCTCGAG | |
| | ORF Start: at 1 | ORF Stop: end of sequence |
| | SEQ ID NO:58 | 442 aa MW at 48175.2 kD |
| NOV16b, | GSEGPGLAFSEDVLSVFGANLSLSAAQLQHLLEQMGAASRVGVPEPGQLHFNQCLTAE | |
| 228495688 Protein | EIFSLHGFSNATQITSSKFSVICPAVLQQLNFHPCEDRPKHKTRPSHSEVWGYGFLSV | |
| Sequence | TIINLASLLGLILTPLIKKSYFPKILTFFVGLAIGTLFSNAIFQLIPEAFGFDFKVDS | |
| | YVEKAVAVFGGFYLLFFFERNLKMLLKTYGQNGHTHFGNDNFGPQEKTHQPKALPAIN | |
| | GVTCYANPAVTEANGHIHFDNVSVVSLQDGKKEPSSCTCLKGPKLSEIGTIAWMITLC | |
| | DALHNFIDGLAIGASCTLSLLQGLSTSIAILCEEFPHELGDFVILLNAGMSTRQALLF | |
| | NFLSACSCYVGLAFGILVGNNFAPNIIFALTGGMFLYIFLADMFPEMNDMLREKVTGR | |
| | KTDFTFFMIQNAGMLTGFTAILLITLYAGEIELELE | |
| | SEQ ID NO:59 | 1326 bp |
| NOV16c, | GGATCCGAGGGGCCAGGGCTAGCCTTCAGCGAGGATGTGCTGAGCGTGTTCGGCGCGA | |
| 228495693 DNA | ATCTGAGCCTGTCGGCGGCGCAGCTCCAGCACTTGCTGGAGCAGATGGGAGCCGCCTC | |
| Sequene | CCGCGTGGGCGTCCCGGAGCCTGGCCAGCTGCACTTCAACCAGTGTTTAACTGCTGAA | |
| | GAGATCTTTTCCCTTCATGGCTTTTCAAATGCTACCCAAATAACCAGCTCCAAATTCT | |
| | CTGTCATCTGTCCAGCAGTCTTACAGCAATTGAACTTTCACCCATGTGAGGATCGGCC | |
| | CAAGCACAAAACAAGACCAAGTCATTCAGAAGTTTGGGGATATGGATTCCTGTCAGTG | |
| | ACGATTATTAATCTGGCATCTCTCCTCGGATTGATTTTGACTCCACTGATAAAGAAAT | |
| | CTTATTTCCCAAAGATTTTGACCTTTTTTGTGGGGCTGGCTATTGGGACTCTTTTTTC | |
| | AAATGCAATTTTCCAACTTATTCCAGAGGCATTTGGATTTGATCCCAAAGTCGACAGT | |
| | TATGTTGAGAAGGCAGTTGCTGTGTTTGGTGGATTTTACCTACTTTTCTTTTTTGAAA | |
| | GAATGCTAAAGATGTTATTAAAGACATATGGTCAGAATGGTCATACCCACTTTGGAAA | |
| | TGATAACTTTGGTCCTCAAGAAAAAACTCATCAACCTAAAGCATTACCTGCCATCAAT | |
| | GGTGTGACATGCTATGCAAATCCTGCTGTCACAGAAGCTAATGGACATATCCATTTTG | |
| | ATAATGTCAGTGTGGTATCTCTACAGGATGGAAAAAAAGAGCCAAGTTCATGTACCCG | |
| | TTTGAAGGGGCCCAAACTGTCAGAAATAGGGACGATTGCCTGGATGATAACGCTCTGC | |
| | GATGCCCTCCACAATTTCATCGATGGCCTGGCGATTGGGGCTTCCTGCACCTTGTCTC | |
| | TCCTTCAGGGACTCAGTACTTCCATAGCAATCCTATGTGAGGAGTTTCCCCACGAGTT | |
| | AGGAGACTTTGTGATCCTACTCAATGCAGGGATGAGCACTCGACAAGCCTTGCTATTC | |
| | AACTTCCTTTCTGCATGTTCCTGCTATGTTGGGCTAGCTTTTGGCATTTTGGTGGGCA | |
| | ACAATTTCGCTCCAAATATTATATTTGCACTTGCTGGAGGCATGTTCCTCTATATTTC | |
| | TCTGGCAGATATGTTTCCAGAGATGAATGATATGCTGAGAGAAAAGGTAACTGGAAGA | |
| | AAAACCGATTTCACCTTCTTCATGATTCAGAATGCTGGAATGTTAACTGGATTCACAG | |
| | CCATTCTACTCATTACCTTGTATGCAGGAGAAATCGAATTGGAGCTCGAG | |
| | ORF Start: at 1 | ORF Stop: end of sequence |
| | SEQ ID NO:60 | 442 aa MW at 48138.2 kD |
| NOV16c, | GSEGPGLAFSEDVLSVFGANLSLSAAQLQHLLEQMGAASRVGVPEPGQLHFNQCLTAE | |
| 228495693 Protein | EIFSLHGFSNATQITSSKFSVICPAVLQQLNFHPCEDRPKHKTRPSHSEVWGYGFLSV | |
| Sequence | TIINLASLLGLILTPLIKKSYFPKILTFFVGLAIGTLFSNAIFQLIPEAFGFDPKVDS | |
| | YVEKAVAVFGGFYLLFFFERMLKMLLKTYGQNGHTHFGNDNFGPQEKTHQPKALPAIN | |
| | GVTCYANPAVTEANGHIHFDNVSVVSLQDGKKEPSSCTRLKGPKLSEIGTIAWMITLC | |
| | DALHNFIDGLAIGASCTLSLLQGLSTSIAILCEEFPHELGDFVILLNAGMSTRQALLF | |
| | NFLSACSCYVGLAFGILVGNNFAPNIIFALAGGMFLYISLADMFPEMNDMLREKVTGR | |
| | KTDFTFFMIQNAGMLTGFTAILLITLYAGETELELE | |
| | SEQ ID NO:61 | 1326 bp |
| NOV16d, | GGATCCGAGGGGCCAGGGCTAGCCTTCAGCGAGGATGTGCTGAGCGTGTTCGGCGCGA | |
| 228495882 DNA | ATCTGAGCCTGTCGGCGGCGCAGCTCCAGCACTTGCTGGAGCAGATGGGAGCCGCCTC | |
| Sequence | CCGCGTGGGCGTCCCGGAGCCTGGCCAGCTGCACTTCAACCAGTGTTTAACTGCTGAA | |
| | GAGATCTTTTCCCTTCATGGCTTTTCAAATGCTACCCAAATAACCAGCTCCAAATTCT | |
| | CTGTCATCTGTCCAGCAGTCTTACAGCAATTGAACTTTCACCCATGTGAGGATCGGCC | |
| | CAAGCACAAAACAAGACCAAGTCATTCAGAAGTTTGGGGATATGGATTCCTGTCAGTG | |
| | ACGATTATTAATCTGGCATCTCTCCTCGGATTGATTTTGACTCCACTGATAAAGAAAT | |
| | CTTATTTCCCAAAGATTTTGACCTTTTTTGTGGGGCTGGCTATTGGGACTCTTTTTTC | |
| | AAATGCAATTTTCCAACTTATTCCAGAGGCATTTGGATTTGATCCCAAAGTCGACAGT | |
| | TATGTTGAGAAGGCAGTTGCTGTGTTTGGTGGATTTTACCTACTTTTCTTTTTTGAAA | |
| | GAATGCTAAAGATGTTATTAAAGACATATGGTCAGAATGGTCATACCCACTTTGGAAA | |
| | TGATAACTTTGGTCCTCAAGAAAAAACTCATCAACCTAAAGCATTACCTGCCATCAAT | |
| | GGTGTGACATGCTATGCAAATCCTGCTGTCACAGAAGCTAATGGACATATCCATTTTG | |
| | ATAATGTCAGTGTGGTATCTCTACAGGATGGAAAAAAAGAGCCAAGTTCATATACCTG | |
| | TTTGAAGGGGCCCAAACTGTCAGAAATAGGGACGATTGCCTGGATGATAACGCTCTGC | |
| | GATGCCCTCCACAATTTCATCGATGGCCTGGCGATTGGGGCTTCCTGCACCTTGTCTC | |
| | TCCTTCAGGGACTCAGTACTTCCATAGCAATCCTATGTGAGGAGTTTCCCCACGAGTT | |
| | AGGAGACTTTGTGATCCTACTCAATGCAGGGATGAGCACTCGACAAGCCTTGCTATTC | |
| | AACTTCCTTTCTGCATGTTCCTGCTATGTTGGGCTAGCTTTTGGCATTTTGGTGGGCA | |
| | ACAATTTCGCTCCAAATATTATATTTGCACTTGCTGGAGGCATGTTCCTCTATATTTC | |
| | TCTGGCAGATATGTTTCCAGAGATGAATGATATGCTGAGAGAAAAGGTAACTGGAAGA | |
| | AAAACCGATTTCGCCTTCTTCATGATTCAGAATGCTGGAATGTTAACTGCATTCACAG | |
| | CCATTCTACTCATTACCTTGTATGCAGGAGAAATCGAATTGGAGCTCGAG | |
| | ORF Start: at 1 | ORF Stop: end of sequence |
| | SEQ ID NO:62 | 442 aa MW at 48115.1 kD |

| | |
|---|---|
| NOV16d, | GSEGPGLAFSEDVLSVFGANLSLSAAQLQHLLEQMGAASRVGVPEPGQLHFNQCLTAE |
| 228495882 Protein | EIFSLHGFSNATQITSSKFSVICPAVLQQLNFHPCEDRPKHKTRPSHSEVWGYGFLSV |
| Sequence | TIINLASLLGLILTPLIKKSYFPKILTFFVGLAIGTLFSNAIFQLIPEAFGFDPKVDS |
| | YVEKAVAVFGGFYLLFFFERMLKMLLKTYGQNGHTHFGNDNFGPQEKTHQPKALPAIN |
| | GVTCYANPAVTEANGHIHFDNVSVVSLQDGKKEPSSYTCLKGPKLSEIGTIAWMITLC |
| | DALHNFIDGLAIGASCTLSLLQGLSTSIAILCEEFPHELGDFVILLNAGMSTRQALLF |
| | NFLSACSCYVGLAFGILVGNNFAPNIIFALAGGMFLYISLADMFPEMNDMLREKVTGR |
| | KTDFAFFMIQNAGMLTGFTAILLITLYAGEIELELE |

Sequence comparison of the above protein sequences yields the following sequence relationships shown in Table 16B.

TABLE 16B
Comparison of NOV16a against NOV16b through NOV16d.

| | | Identities/ | |
|---|---|---|---|
| | NOV16a Residues/ | Similarities for | |
| Protein Sequence | Match Residues | the Matched Region | |
| NOV16b | 22 . . . 460 | 424/439 | (96%) |
| | 2 . . . 440 | 425/439 | (96%) |
| NOV16c | 22 . . . 460 | 425/439 | (96%) |
| | 2 . . . 440 | 426/439 | (96%) |
| NOV16d | 22 . . . 460 | 424/439 | (96%) |
| | 2 . . . 440 | 425/439 | (96%) |

Further analysis of the NOV16a protein yielded the following properties shown in Table 16C.

TABLE 16C
Protein Sequence Properties NOV16a

| | |
|---|---|
| PSort | 0.6400 probability located in plasma membrane; 0.4600 pro- |
| analysis: | bability located in Golgi body; 0.3700 probability located in |
| | endoplasmic reticulum (membrane); 0.1000 probability |
| | located in endoplasmic reticulum (lumen) |
| SignalP | Cleavage site between residues 23 and 24 |
| analysis: | |

A search of the NOV16a protein against the Geneseq database, a proprietary database that contains sequences published in patents and patent publication, yielded several homologous proteins shown in Table 16D.

TABLE 16D
Geneseq Results for NOV16a

| | | NOV16a | | | |
|---|---|---|---|---|---|
| | | Residues/ | Identities/ | | |
| Geneseq | Protein/Organism/Length | Match | Similarities for | | Expect |
| Identifier | [Patent #, Date] | Residues | the Matched Region | | Value |
| AAG81272 | Human AFP protein sequence SEQ ID | 1 . . . 460 | 459/460 | (99%) | 0.0 |
| | NO:62 - Homo sapiens, 460 aa. | 1 . . . 460 | 459/460 | (99%) | |
| | [WO200129221-A2, Apr. 26, 2001] | | | | |
| AAB95761 | Human protein sequence SEQ ID NO:18686 | 73 . . . 460 | 387/388 | (99%) | 0.0 |
| | - Homo sapiens, 393 aa. | 6 . . . 393 | 388/388 | (99%) | |
| | [EP1074617-A2, Feb. 07, 2001] | | | | |
| AAB60496 | Human cell cycle and proliferation protein | 15 . . . 459 | 230/466 | (49%) | e−116 |
| | CCYPR-44, SEQ ID NO:44 - Homo sapiens, | 75 . . . 536 | 315/466 | (67%) | |
| | 537 aa. [WO200107471-A2, Feb. 01, 2001] | | | | |
| AAY05376 | Human HCMV inducible gene protein, SEQ | 15 . . . 459 | 230/466 | (49%) | e−116 |
| | ID NO 20 - Homo sapiens, 531 aa. | 69 . . . 530 | 315/466 | (67%) | |
| | [WO9913075-A2, Mar. 18, 1999] | | | | |
| AAU30977 | Novel human secreted protein #1468 - | 15 . . . 459 | 224/466 | (48%) | e−110 |
| | Homo sapiens, 540 aa. [WO200179449-A2, | 78 . . . 539 | 304/466 | (65%) | |
| | Oct. 25, 2001] | | | | |

In a BLAST search of public sequence databases, the NOV16a protein was found to have homology to the proteins shown in the BLASTP data in Table 16E.

TABLE 16E
Public BLASTP Results for NOV16a

| Protein | | | | |
|---|---|---|---|---|
| Accession | | NOV16a Residues/ | Identities/Similarities | |
| Number | Protein/Organism/Length | Match Residues | for the Matched Portion | Expect Value |
| Q9C0K1 | BCG INDUCED INTEGRAL | 1 . . . 460 | 460/460 (100%) | 0.0 |
| | MEMBRANE PROTEIN BIGMO-103 | 1 . . . 460 | 460/460 (100%) | |
| | (UP-REGULATED BY BCG-CWS) - | | | |
| | Homo sapiens (Human), 460 aa. | | | |
| CAC38522 | SEQUENCE 61 FROM PATENT | 1 . . . 460 | 459/460 (99%) | 0.0 |
| | WO0129221 - Homo sapiens (Human), 460 aa. | 1 . . . 460 | 459/460 (99%) | |
| Q91W10 | RIKEN CDNA 4933419D20 GENE - Mus | 1 . . . 460 | 411/462 (88%) | 0.0 |
| | musculus (Mouse), 462 aa. | 1 . . . 462 | 431/462 (92%) | |
| Q9D5V4 | 4933419D20RIK PROTEIN - Mus musculus | 1 . . . 460 | 410/462 (88%) | 0.0 |
| | (Mouse), 462 aa. | 1 . . . 462 | 431/462 (92%) | |
| Q9D426 | 4933419D20RIK PROTEIN - Mus musculus | 1 . . . 460 | 410/462 (88%) | 0.0 |
| | (Mouse), 462 aa. | 1 . . . 462 | 431/462 (92%) | |

PFam analysis predicts that the NOV16a protein contains the domains shown in the Table 16F.

TABLE 16F
Domain Analysis of NOV16a

| | NOV16a | Identities/Similarities | Expect |
|---|---|---|---|
| Pfam Domain | Match Region | for the Matched Region | Value |
| Zip: domain 1 of 1 | 299 . . . 451 | 45/180 (25%) | 3.5e−26 |
| | | 116/180 (64%) | |

### Example 17

The NOV17 clone was analyzed, and the nucleotide and encoded polypeptide sequences are shown in Table 17A.

TABLE 17A
NOV17 Sequence Analysis

| | SEQ ID NO:63 | 1037 bp |
|---|---|---|
| NOV17a, | AGCTCGTCGACCTTTCTCTGAAGAGAAAATTGCTGTTGGGATGAAGCTTTGCAGCCTT | |
| CG93008-01 DNA | GCAGTCCTTGTACCCATTGTTCTCTTCTGTGAGCAGCATGTCTTCGCGTTTCAGAGTG | |
| Sequence | GCCAAGTTCTAGCTGCTCTTCCTAGAACCTCTAGGCAAGTTCAAGTTCTACAGAATCT | |
| | TACTACAACATATGAGATTGTTCTCTGGCAGCCGGTAACAGCTGACCTTATTGTGAAG | |
| | AAAAAACAAGTCCATTTTTTTGTAAATGCATCTGATGTCGACAATGTGAAAGCCCATT | |
| | TAAATGTGAGCGGAATTCCATGCAGTGTCTTGCTGGCAGACGTGGAAGATCTTATTCA | |
| | ACAGCAGATTTCCAACGACACAGTCAGCCCCCGAGCCTCCGCATCGTACTATGAACAG | |
| | TATCACTCACTAAATGAAATCTATTCTTGGATAGAATTTATAACTGAGAGGCATCCTG | |
| | ATATGCTTACAAAAATCCACATCGGATCCTCATTTGAGAAGTACCCACTCTATGTTTT | |
| | AAAGGTTTCTGGAAAAGAACAAGCAGCCAAAAATGCCATATGGATTGACTGTGGACTT | |
| | TATCCTGAGTCAGAACCAGAAGTGAAGGCAGTGGCTAGTTTCTTGAGAAGAAATATCA | |
| | ACCAGATTAAAGCATACATCAGCATGCATTCATACTCCCAGCATATAGTGTTTCCATA | |
| | TTCCTATACACGAAGTAAAAGCAAAGACCATGAGGAACTGTCTCTAGTAGCCAGTGAA | |
| | GCAGTTCGTGCTATTGAGAAAATTAGTAAAAATACCAGGTATACACATGGCCATGGCT | |
| | CAGAAACCTTATACCTAGCTCCTGGAGGTGGGGACGATTGGATCTATGATTTGGGCAT | |
| | CAAATATTCGTTTACAATTGAACTTCGAGATACGGGCACATACGGATTCTTGCTGCCG | |
| | GAGCGTTACATCAAACCCACCTGTAGAGAAGCTTTTGCCGCTGTCTCTAAAATAGCTT | |
| | GGCATGTCATTAGGAATGTTTAATGCCCCTGATTTTATCATTCTGCTTCTC | |
| | ORF Start: ATG at 41 | ORF Stop: TAA at 1007 |
| | SEQ ID NO:64 | 322 aa MW at 36554.4 kD |
| NOV17a, | MKLCSLAVLVPIVLFCEQHVFAFQSGQVLAALPRTSRQVQVLQNLTTTYEIVLWQPVT | |
| CG93008-01 Protein | ADLIVKKKQVHFFVNASDVDNVKAHLNVSGIPCSVLLADVEDLIQQQISNDTVSPRAS | |
| Sequence | ASYYEQYHSLNEIYSWIEFITERHPDMLTKIHIGSSFEKYPLYVLKVSGKEQAAKNAI | |
| | WIDCGLYPESEPEVKAVASFLRRNINQIKAYISMHSYSQHIVFPYSYTRSKSKDHEEL | |
| | SLVASEAVRAIEKISKNTRYTHGHGSETLYLAPGGGDDWIYDLGIKYSFTIELRDTGT | |
| | YGFLLPERYIKPTCREAFAAVSKIAWHVIRNV | |
| | SEQ ID NO:65 | 1132 bp |
| NOV17b, | AGCTCGTCGACCTTTCTCTGAAGAGAAAATTGCTGTTGGGATGAAGCTTTGCAGCCTT | |
| CG93008-02 DNA | GCAGTCCTTGTACCCATTGTTCTCTTCTGTGAGCAGCATGTCTTCGCGTTTCAGAGTG | |
| Sequence | GCCAAGTTCTAGCTGCTCTTCCTAGAACCTCTAGGCAAGTTCAAGTTCTACAGAATCT | |
| | TACTACAACATATGAGATTGTTCTCTGGCAGCCGGTAACAGCTGACCTTATTGTGAAG | |
| | AAAAAACAAGTCCATTTTTTTGTAAATGCATCTGATGTCGACAATGTGAAAGCCCATT | |
| | TAAATGTGAGCGGAATTCCATGCAGTGTCTTGCTGGCAGACGTGGAAGATCTTATTCA | |
| | ACAGCAGATTTCCAACGACACAGTCAGCCCCCGAGCCTCCGCATCGTACTATGAACAG | |
| | TATCACTCACTAAATGAAATCTATTCTTGGATAGAATTTATAACTGAGAGGCATCCTG | |
| | ATATGCTTACAAAAATCCACATTGGATCCTCATTTGAGAAGTACCCACTCTATGTTTT | |
| | AAAGGGTTTCTTTGAGCAGGTTTCTGGAAAAGAACAAGCAGCCAAAAATGCCATATGG | |
| | ATTGACTGTGGAATCCATGCCAGAGAATGGATCTCTCCTGCTTTCTGCTTGTGGTTCA | |
| | TAGGCCATATAACTCAATTCTATGGGATAATAGGGCAATATACCAATCTCCTGAGGCT | |
| | TGTGGATTTCTATGTTATGCCGGTGGTTAATGTGGATGGTTATGACTACTCATGGAAA | |
| | AAGAATCGAATGTGGAGAAAGAACCGTTCTTTCTATGCGAACAATCATTGCATCGGAA | |
| | CAGACCTGAATAGGAACTTTGCTTCCAAACACTGGTGTGAGGAAGGTGCATCCAGTTC | |
| | CTCATGCTCGGAAACCTACTGTGGACTTTATCCTGAGTCAGAAACCTTATACCTAGCT | |
| | CCTGGAGGTGGGGACGATTGGATCTATGATTTGGGCATCAAATATTCGTTTACAATTG | |
| | AACTTCGAGATACGGGCACATACGGATTCTTGCTGCCGGAGCGTTACATCAAACCCAC | |
| | CTGTAGAGAAGCTTTTGCCGCTGTCTCTAAAATAGCTTGGCATGTCATTAGGAATGTT | |
| | TAATGCCCCTGATTTTATCATTCTGCTTCC | |
| | ORF Start: ATG at 41 | ORF Stop: TAA at 1103 |
| | SEQ ID NO:66 | 354 aa MW at 40556.9 kD |
| NOV17b, | MKLCSLAVLVPIVLFCEQHVFAFQSGQVLAALPRTSRQVQVLQNLTTTYEIVLWQPVT | |
| CG93008-02 Protein | ADLIVKKKQVHFFVNASDVDNVKAHLNVSGIPCSVLLADVEDLIQQQISNDTVSPRAS | |
| Sequence | ASYYEQYHSLNEIYSWIEFITERHPDMLTKIHIGSsFEKYPLYVLKGFFEQVSGKEQA | |
| | AKNAIWIDCGIHAREWISPAFCLWFIGHITQFYGIIGQYTNLLRLVDFYVMPVVNVDG | |
| | YDYSWKKNRMWRKNRSFYANNHCIGTDLNRNFASKHWCEEGASSSSCSETYCGLYPES | |
| | ETLYLAPGGGDDWIYDLGIKYSFTIELRDTGTYGFLLPERYIKPTCREAFAAVSKIAW | |
| | HVIRNV | |
| | SEQ ID NO:67 | 1743 bp |
| NOV17c, | AGAGAAAATTGCTGTTGGGATGAAGCTTTGCAGCCTTGCAGTCCTTGTACCCATTGTT | |
| CG93008-03 DNA | CTCTTCTGTGAGCAGCATGTCTTCGCGTTTCAGAGTGGCCAAGTTCTAGCTGCTCTTC | |
| Sequence | CTAGAACCTCTAGGCAAGTTCAAGTTCTACAGAATCTTACTACAACATATGAGATTGT | |
| | TCTCTGGCAGCCGGTAACAGCTGACCTTATTGTGAAGAAAAAACAAGTCCATTTTTTT | |
| | GTAAATGCATCTGATGTCGACAATGTGAAAGCCCATTTAAATGTGAGCGGAATTCCAT | |
| | GCAGTGTCTTGCTGGCAGACGTGGAAGATCTTATTCAACAGCAGATTTCCAACGACAC | |
| | AGTCAGCCCCCGAGCCTCCGCATCGTACTATGAACAGTATCACTCACTAAATGAAATC | |
| | TATTCTTGGATAGAATTTATAACTGAGAGGCATCCTGATATGCTTACAAAAATCCACA | |
| | TTGGATCCTCATTTGAGAAGTACCCACTCTATGTTTTAAAGGGTTTCTTTGAGCAGGT | |
| | TTCTGGAAAAGAACAAGCAGCCAAAAATGCCATATGGATTGACTGTGGAATCCATGCC | |
| | AGAGAATGGATCTCTCCTGCTTTCTGCTTGTGGTTCATAGGCCATATAACTCAATTCT | |
| | ATGGGATAATAGGGCAATATACCAATCTCCTGAGGCTTGTGGATTTCTATGTTATGCC | |
| | AGTGGTTAATGTGGATGGTTATGACTACTCATGGAAAAAGAATCGAATGTGGAGAAAG | |
| | AACCGTTCTTTCTATGCGAACAATCATTGCATCGGAACAGACCTGAATAGGAACTTTG | |
| | CTTCCAAACACTGGTGTGAGGAAGGTGCATCCAGTTCCTCATGCTCGGAAACCTACTG | |
| | TGGACTTTATCCTGAGTCAGAACCAGAAGTGAAGGCAGTGGCTAGTTTCTTGAGAAGA | |
| | AATATCAACCAGATTAAAGCATACATCAGCATGCATTCATACTCCCAGCATATAGTGT | |
| | TTCCATATTCCTATACACGAAGTAAAAGCAAAGACCATGAGGAACTGTCTCTAGTAGC | |
| | CAGTGAAGCAGTTCGTGCTATTGAGAAAATTAGTAAAAATACCAGGTATACACATGGC | |
| | CATGGCTCAGAAACCTTATACCTAGCTCCTGGAGGTGGGGACGATTGGATCTATGATT | |
| | TGGGCATCAAATATTCGTTTACAATTGAACTTCGAGATACGGGCACATACGGATTCTT | |
| | GCTGCCGGAGCGTTACATCAAACCCACCTGTAGAGAAGCTTTTGCCGCTGTCTCTAAA | |
| | ATAGCTTGGCATGTCATTAGGAATGTTTAATGCCCCTGATTTTATCATTCTGCTTCCG | |
| | TATTTTAATTTACTGATTCCAGCAAGACCAAATCATTGTATCAGATTATTTTTAAGTT | |
| | TTATCCGTAGTTTTGATAAAAGATTTTCCTATTCCTTGGTTCTGTCAGAGAACCTAAT | |
| | AAGTGCTACTTTGCCATTAAGGCAGACTAGGGTTCATGTCTTTTTACCCTTTAAAAAA | |
| | AAATTGTAAAAGTCTAGTTACCTACTTTTTCTTTGATTTTCGACGTTTGACTAGCCAT | |
| | CTCAAGCAACTTTCGACGTTTGACTAGCCATCTCAAGCAAGTTTAATCAAAGATCATC | |
| | TCACGCTGATCATTGGATCCTACTCAACAAAAGGAAGGGTGGTCAGAAGTACATTAAA | |
| | GATTTCTGCTCCAAATTTTCAATAAATTTCTTCTTCTCCTTTAAAAAAAAAAAAAAAA | |
| | AAA | |
| | ORF Start: ATG at 20 | ORF Stop: TAA at 1304 |
| | SEQ ID NO:68 | 428 aa MW at 49032.4 kD |
| NOV17c, | MKLCSLAVLVPIVLFCEQHVFAFQSGQVLAALPRTSRQVQVLQNLTTTYEIVLWQPVT | |
| CG93008-03 Protein | ADLIVKKKQVHFFVNASDVDNVKAHLNVSGIPCSVLLADVEDLIQQQISNDTVSPRAS | |
| Sequence | ASYYEQYHSLNEIYSWIEFITERHPDMLTKIHIGSSFEKYPLYVLKGFFEQVSGKEQA | |
| | AKNAIWIDCGIHAREWISPAFCLWFIGHITQFYGIIGQYTNLLRLVDFYVMPVVNVDG | |
| | YDYSWKKNRMWRKNRSFYANNHCIGTDLNRNFASKHWCEEGASSSSCSETYCGLYPES | |
| | EPEVKAVASFLRRNINQIKAYISMHSYSQHIVFPYSYTRSKSKDHEELSLVASEAVRA | |
| | IEKISKNTRYTHGHGSETLYLAPGGGDDWIYDLGIKYSFTIELRDTGTYGFLLPERYI | |
| | KPTCREAFAAVSKIAWHVIRNV | |
| | SEQ ID NO:69 | 1344 bp |
| NOV17d, | GCCCTTTCTGAAGAGAAAATTGCTGTTGGGATGAAGCTTTGCAGCCTTGCAGTCCTTG | |
| CG93008-04 DNA | TACCCATTGTTCTCTTCTGTGAGCAGCATGTCTTCGCGTTTCAGAGTGGCCAAGTTCT | |
| Sequence | AGCTGCTCTTCCTAGAACCTCTAGGCAAGTTCAAGTTCTACAGAATCTTACTACAACA | |
| | TATGAGATTGTTCTCTGGCAGCCGGTAACAGCTGACCTTATTGTGAAGAAAAAACAAG | |
| | TCCATTTTTTTGTAAATGCATCTGATGTCGACAATGTGAAAGCCCATTTAAATGTGAG | |
| | CGGAATTCCATGCAGTGTCTTGCTGGCAGACGTGGAAGATCTTATTCAACAGCAGATT | |
| | TCCAACGACACAGTCAGCCCCCGAGCCTCCGCATCGTACTATGAACAGTATCACTCAC | |
| | TAAATGAAATCTATTCTTGGATAGAATTTATAACTGAGAGGCATCCTGATATGCTTAC | |
| | AAAAATCCACATTGGATCCTCATTTGAGAAGTACCCACTCTATGTTTTAAAGGGTTTC | |
| | TTTGAGCAGGTTTCTGGAAAAGAACAAGCAGCCAAAAATGCCATATGGATTGACTGTG | |
| | GAATCCATGCCAGAGAATGGATCTCTCCTGCTTTCTGCTTGTGGTTCATAGGCCATAT | |
| | AACTCAATTCTATGGGATAATAGGGCAATATACCAATCTCCTGAGGCTTGTGGATTTC | |
| | TATGTTATGCCGGTGGTTAATGTGGATGGTTATGACTACTCATGGAAAAAGAATCGAA | |
| | TGTGGAGAAAGAACCGTTCTTTCTATGCGAACAATCATTGCATCGGAACAGACCTGAA | |
| | TAGGAACTTTGCTTCCAAACACTGGTGTGAGGAAGGTGCATCCAGTTCCTCATGCTCG | |
| | GAAACCTACTGTGGACTTTATCCTGAGTCAGAACCAGAAGTGAAGGCAGTGGCTAGTT | |
| | TCTTGAGAAGAAATATCAACCAGATTAAAGCATACATCAGCATGCATTCATACTCCCA | |
| | GCATATAGTGTTTCCATATTCCTATACACGAAGTAAAAGCAAAGACCATGAGGAACTG | |
| | TCTCTAGTAGCCAGTGAAGCAGTTCGTGCTATTGAGAAAATTAGTAAAAATACCAGGT | |
| | ATACACATGGCCATGGCTCAGAAACCTTATACCTAGCTCCTGGAGGTGGGGACGATTG | |
| | GATCTATGATTTGGGCATCAAATATTCGTTTACAATTGAACTTCGAGATACGGGCACA | |
| | TACGGATTCTTGCTGCCGGAGCGTTACATCAAACCCACCTGTAGAGAAGCTTTTGCCG | |
| | CTGTCTCTAAAATAGCTTGGCATGTCATTAGGAATGTTTAATGCCCCTGATTTTATCA | |
| | TTCTGCTTCT | |
| | ORF Start: ATG at 31 | ORF Stop: TAA at 1315 |
| | SEQ ID NO:70 | 428 aa MW at 49032.4 kD |

| | |
|---|---|
| NOV17d, | MKLCSLAVLVPIVLFCEQHVFAFQSGQVLAALPRTSRQVQVLQNLTTTYEIVLWQPVT |
| CG93008-04 Protein | ADLIVKKKQVHFFVNASDVDNVKAHLNVSGIPCSVLLADVEDLIQQQISNDTVSPRAS |
| | ASYYEQYHSLNEIYSWIEFITERHPDMLTKIHIGSSFEKYPLYVLKGFFEQVSGKEQA |
| | AKNAIWIDCGIHAREWISPAFCLWFIGHITQFYGIIGQYTNLLRLVDFYVMPVVNVDG |
| | YDYSWKKNRMWRKNRSFYANNHCIGTDLNRNFASKHWCEEGASSSSCSETYCGLYPES |
| | EPEVKAVASFLRRNINQIKAYISMHSYSQHIVFPYSYTRSKSKDHEELSLVASEAVPA |
| | IEKISKNTRYTHGHGSETLYLAPGGGDDWIYDLGIKYSFTIELRDTGTYGFLLPERYI |
| | KPTCREAFAAVSKIAWHVIRNV |

Sequence comparison of the above protein sequences yields the following sequence relationships shown in Table 17B.

TABLE 17B
Comparison of NOV17a against NOV17b through NOV17d.

| | NOV17a Residues/ | Identities/Similarities |
|---|---|---|
| Protein Sequence | Match Residues | for the Matched Region |
| NOV17b | 1 . . . 322 | 259/356 (72%) |
| | 1 . . . 354 | 274/356 (76%) |
| NOV17c | 1 . . . 181 | 179/186 (96%) |
| | 1 . . . 186 | 181/186 (97%) |
| NOV17d | 1 . . . 181 | 179/186 (96%) |
| | 1 . . . 186 | 181/186 (97%) |

Further analysis of the NOV17a protein yielded the following properties shown in Table 17C.

TABLE 17C
Protein Sequence Properties NOV17a

| | |
|---|---|
| PSort | 0.6424 probability located in outside; |
| analysis: | 0.1900 probability located in lysosome (lumen); |
| | 0.1882 probability located in microbody (peroxisome); |
| | 0.1000 probability located in |
| | endoplasmic reticulum (membrane) |
| SignalP | Cleavage site between residues 23 and 24 |
| analysis: | |

A search of the NOV17a protein against the Geneseq database, a proprietary database that contains sequences published in patents and patent publication, yielded several homologous proteins shown in Table 17D.

TABLE 17D
Geneseq Results for NOV17a

| Geneseq | Protein/Organism/Length | NOV17a Residues/ | Identities/Similarities | |
|---|---|---|---|---|
| Identifier | [Patent #, Date] | Match Residues | for the Matched Region | Expect Value |
| AAB11457 | Human brain carboxypeptidase B protein - | 1 . . . 181 | 178/181 (98%) | e−100 |
| | Homo sapiens, 360 aa. | 1 . . . 181 | 180/181 (99%) | |
| | [WO200066717-A1, 09-NOV-2000] | | | |
| AAW92270 | Human plasma carboxypeptidase B | 1 . . . 181 | 178/181 (98%) | e−100 |
| | (PCPB) thr147 - Homo sapiens, 423 aa. | 1 . . . 181 | 180/181 (99%) | |
| | [WO9855645-A1, 10-DEC-1998] | | | |
| AAW14733 | Human plasma carboxypeptidase B - | 1 . . . 181 | 178/181 (98%) | e−100 |
| | Homo sapiens, 423 aa. [US5593674-A, | 1 . . . 181 | 180/181 (99%) | |
| | 14-JAN-1997] | | | |
| AAR90293 | Human plasma carboxypeptidase B - | 1 . . . 181 | 178/181 (98%) | e−100 |
| | Homo sapiens, 423 aa. [US5474901-A, | 1 . . . 181 | 180/181 (99%) | |
| | 12-DEC-1995] | | | |
| AAR36273 | Human plasma carboxypeptidase B - | 1 . . . 181 | 178/181 (98%) | e−100 |
| | Homo sapiens, 423 aa. [US5206161-A, | 1 . . . 181 | 180/181 (99%) | |
| | 27-APR-1993] | | | |

In a BLAST search of public sequence databases, the NOV17a protein was found to have homology to the proteins shown in the BLASTP data in Table 17E.

TABLE 17E
Public BLASTP Results for NOV17a

| Protein | | | | |
|---|---|---|---|---|
| Accession | | NOV17a Residues/ | Identities/Similarities | |
| Number | Protein/Organism/Length | Match Residues | for the Matched Portion | Expect Value |
| Q961Y4 | CARBOXYPEPTIDASE B2 (PLASMA) - | 1 . . . 181 | 179/181 (98%) | e−100 |
| | Homo sapiens (Human), 423 aa. | 1 . . . 181 | 181/181 (99%) | |
| Q9NTI8 | BA139H14.2 (CARBOXYPEPTIDASE B2 | 1 . . . 181 | 179/181 (98%) | e−100 |
| | (PLASMA)) - Homo sapiens (Human), 198 aa | 1 . . . 181 | 181/181 (99%) | |
| | (fragment). | | | |
| Q9P2Y6 | CARBOXYPEPTIDASE B-LIKE PROTEIN - | 1 . . . 181 | 178/181 (98%) | 1e−99 |
| | Homo sapiens (Human), 360 aa. | 1 . . . 181 | 181/181 (99%) | |
| Q15114 | PCPB PROTEIN - Homo sapiens (Human), | 1 . . . 181 | 178/181 (98%) | 2e−99 |
| | 423 aa. | 1 . . . 181 | 180/181 (99%) | |
| Q9JHH6 | CARBOXYPEPTIDASE R | 1 . . . 181 | 147/181 (81%) | 8e−80 |
| | (THROMBIN-ACTIVATABLE | 1 . . . 180 | 164/181 (90%) | |
| | FIBRINOLYSIS INHIBITOR) | | | |
| | (1110032P04RIK PROTEIN) - Mus musculus | | | |
| | (Mouse), 422 aa. | | | |

PFam analysis predicts that the NOV17a protein contains the domains shown in the

TABLE 17F
Domain Analysis of NOV17a

| | NOV17a | Identities/Similarities | Expect |
|---|---|---|---|
| Pfam Domain | Match Region | for the Matched Region | Value |
| Propep_M14: | 27 . . . 106 | 30/82 (37%) | 9.1e−38 |
| domain 1 of 1 | | 79/82 (96%) | |
| Zn_carbOpept: | 123 . . . 179 | 20/59 (34%) | 9.1e−13 |
| domain 1 of 2 | | 46/59 (78%) | |
| Zn_carbOpept: | 182 . . . 306 | 66/139 (47%) | 8.2e−42 |
| domain 2 of 2 | | 99/139 (71%) | |

### Example 18

The NOV18 clone was analyzed, and the nucleotide and encoded polypeptide sequences are shown in Table 18A.

TABLE 18A
NOV18 Sequence Analysis

| | SEQ ID NO:71 | 1187 bp |
|---|---|---|
| NOV18a, | TCTACTATGGTGGCCAAAGTTTCTCAGGTAGCAGTAAGATGGCTTTTTACGATTGGTC | |
| CG93252-01 DNA | TAATCAGATCCTCATTTCTTTTCCCTTCCTAGGTTTTGAAACATGAATCCTTCACTCC | |
| Sequence | TCCTTGCTGTCTTTTGCCTGAGATTAGCCTCAGCTAGTCTAACACTTGATCACAGTTT | |
| | AGATCAGTGGAAGGCAAAGCACAAGAGATTATATGGCATGAATGAAGAAGGATGGAGG | |
| | AGAGCAGTGTGGCAGAACATGAAGATGATTGAGCAGCACAATCAGGAATACAGGGAAG | |
| | GGAAACACAGCTTCACAATGGCCATGAACGCCTTTGGAGAAATGACCAGTGAAGAATT | |
| | CAGGCAGGTGATGAATGGCTTTCAAAACCGTAAGCCCAGGAAGGGGAAAGTGTTCCAG | |
| | GAACCTCTGTTTTATGAGGCCCCCAGATCTGTGGATTGGAGAGAGAAAGGCTACGTGA | |
| | CTCCTGTGAAGAATCAGGGTCAGTGTGGTTCTTGTTGGGCTTTTAGTGCTACTGGTGC | |
| | TCTTGAAGGACAGATGTTCCGGAAAACTGGGAGGCTTATCTCACTGAGTGAGCAGAAT | |
| | CTGGTAGACTGCTCTGGGCCTCAAGGCAATGAAGGCTGCAATGGTGGCCTAATGGATT | |
| | ATGCTTTCCAGTATGTTCAGGATAATGGAGGCCTGGACTCTGAGGAATCCTATCCATA | |
| | TGAGGCAACAGAAAAAGCCTGTAGGTACAATCCCAAGTATTCTGCTACTAATGACACT | |
| | GGGTACATGCAAATACTCCCTGTGGAAGAGAAGGCCCTAATGAAGGCTGTGGCAACTG | |
| | TGGGGCGCATCTCTGCTGTTGTTTATGGACTTCTTGATTCCTTCTGGTCCTATAAAAA | |
| | AGGCATTTATTTTGAGCCAGACTGTAGCAGTGAAGACATGGATCATGGTGTGCTGGTG | |
| | GTTGGCTACGGATTTGAAAGCACAGAATCAGATAACAATAAATATTGGCTGGTGAAGA | |
| | ACAGCTGGGGTGAAGAATGGGGCATGGGTGGCTACGTAAAGATGGCCAAAGACCGGAG | |
| | AAACCATTGTGGAATTGCCTCAGCAGCCAGCTACCCCACTGTGTGAGCTGGTGGACGG | |
| | TGATGAGGAAGGACTTGACTGGGGATGGCGCATGCATGGGAGGAATTCATCTTCAGTC | |
| | TACCAGCCCCCGCTGTGTCGGATACAC | |
| | ORF Start: ATG at 101 | ORF Stop: TGA at 1088 |
| | SEQ ID NO:72 | 329 aa MW at 37307.8 kD |
| NOV18a, | MNPSLLLAVFCLRLASASLTLDHSLDQWKAKHKRLYGMNEEGWRRAVWQNMKMIEQHN | |
| CG93252-01 Protein | QEYREGKHSFTMANNAFGEMTSEEFRQVMNGFQNRKPRKGKVFQEPLFYEAPRSVDWR | |
| Sequence | EKGYVTPVKNQGQCGSCWAFSATGALEGQMFRKTGRLISLSEQNLVDCSGPQGNEGCN | |
| | GGLMDYAFQYVQDNGGLDSEESYPYEATEKACRYNPKYSATNDTGYMQILPVEEKALM | |
| | KAVATVGRISAVVYGLLDSFWSYKKGIYFEPDCSSEDMDHGVLVVGYGFESTESDNNK | |
| | YWLVKNSWGEEWGMGGYVKMAKDRRNHCGIASAASYPTV | |
| | SEQ ID NO:73 | 1157 bp |
| NOV18b, | TCTACTATGGTGGCCAAAGTTTCTCAGGTAGCAGTAAGATGGCTTTTTAGGATTGGTC | |
| CG93252-02 DNA | TAATCAGATCCTCATTTCTTTTCCCTTCCTAGGTTTTGAAACATGAATCCTTCACTCC | |
| Sequence | TCCTTGCTGTCTTTTGCCTGAGATTAGCCTCAGCTAGTCTAACACTTGATCACAGTTT | |
| | AGATCAGTGGAAGGCAAAGCACAAGAGATTATATGGCATGAATGAAGAAGGATGGAGG | |
| | AGAGCAGTGTGGCAGAACATGAAGATGATTGAGCAGCACAATCAGGAATACAGGGAAG | |
| | GGAAACACAGCTTCACAATGGCCATGAACGCCTTTGGAGAAATGACCAGTGAAGAATT | |
| | CAGGCAGGTGATGAATGGCTTTCAAAACCGTAAGCCCAGGAAGGGGAAAGTGTTCCAG | |
| | GAACCTCTGTTTTATGAGGCCCCCAGATCTGTGGATTGGAGAGAGAAAGGCTACGTGA | |
| | CTCCTGTGAAGAATCAGGGTCAGTGTGGTTCTTGTTGGGCTTTTAGTGCTACTGGTGC | |
| | TCTTGAAGGACAGATGTTCCGGAAAACTGGGAGGCTTATCTCACTGAGTGAGCAGAAT | |
| | CTGGTAGACTGCTCTGGGCCTCAAGGCAATGAAGGCTGCAATGGTGGCCTAATGGATT | |
| | ATGCTTTCCAGTATGTTCAGGATAATGGAGGCCTGGACTCTGAGGAATCCTATCCATA | |
| | TGAGGCAACAGAAAAAGCCTGTAGGTACAATCCCAAGTATTCTGCTACTAATGACACT | |
| | GGGTACATGCAAATACTCCCTGTGGAAGAGAAGGCCCTAATGAAGGCTGTGGCAACTG | |
| | TGGGGCGCATCTCTGCTGTTGTTTATGGACTTCTTGATTCCTTCTGGTCCTATAAAAA | |
| | AGGCATTTATTTTGAGCCAGACTGTAGCAGTGAAGACATGGATCATGGTGTGCTGGTG | |
| | GTTGGCTACGGATTTGAAAGCACAGAATCAGATAACAATAAATATTGGCTGGTGAAGA | |
| | ACGATTGGAGAGAGAAAGGCTACGTGACTCCTGTGAAGGATCAGGTAAGACAGTGTCA | |
| | GATTCAGACCTCCCATCTCCCCAGGAAAGCCAAGAGGTGATCGACCTCTTTGCTTTAG | |
| | TGGAGTGTAGAACAACTTGCAGTTCATAGTATTCAGAAAGATGAGCTGTTGTCAA | |
| | ORF Start: ATG at 101 | ORF Stop: TGA at 1082 |
| | SEQ ID NO:74 | 327 aa MW at 37444.0 kD |
| NOV18b, | MNPSLLLAVFCLRLASASLTLDHSLDQWKAKHKRLYGMNEEGWRRAVWQNMKMIEQHN | |
| CG93252-02 Protein | QEYREGKHSFTMA1VINAFGEMTSEEFRQVMNGFQNRKPRKGKVFQEPLFYEAPRSWR | |
| Sequence | EKGYVTPVKNQGQCGSCWAFSATGALEGQMFRKTGRLISLSEQNLVDCSGPQGNEGCN | |
| | GGLMDYAFQYVQDNGGLDSEESYPYEATEKACRYNPKYSATNDTGYMQILPVEEKALM | |
| | KAVATVGRISAVVYGLLDSFWSYKKGIYFEPDCSSEDMDHGVLVVGYGFESTESDNNK | |
| | YWLVKNDWREKGYVTPVKDQVRQCQIQTSHLPRKAKR | |
| | SEQ ID NO:75 | 1031 bp |
| NOV18c, | CCTAGGTTTTGAAACATGAATCCTTCACTCCTCCTTGCTGTCTTTTGCCTGAGATTAG | |
| CG93252-03 DNA | CCTCAGCTAGTCTAACACTTGATCACAGTTTAGATCAGTGGAAGGCAAAGCACAAGAG | |
| Sequence | ATTATATGGCATGAATGAAGAAGGATGGAGGAGAGCAGTGTGGCAGAACATGAAGATG | |
| | ATTGAGCAGCACAATCAGGAATACAGGGAAGGGAAACACAGCTTCACAATGGCCATGA | |
| | ACGCCTTTGGAGAAATGACCAGTGAAGAATTCAGGCAGGTGGTGAATGGCTTTCAAAA | |
| | CCAGAAGCACAGGAAGGGGAAAGTGCTCCAGGAACCTCTGCTTCATGACATCCGCAAA | |
| | TCTGTGGATTGGAGAGAGAAAGGCTACGTGACTCCTGTGAAGGATCAGGTAAGACAGT | |
| | GTGCATCTTCTTATGCTTTTAGTGCAGCTGGGGCTCTGGACCTGGTGGACTGCTCTAG | |
| | GCTTCAAGGCAATGTTGGCTGCATTTTTGGAGAACCATTATTTTGCTTCCAGTATGTT | |
| | GCCGACAATGGAGGCCTGGACTCTGAGGAATCCTTTTCATATGAAGAAAAGGAAAAAG | |
| | CCTGTAGGTACAATCCCAAGTATTCTGCTACTAATGACACTGGGTACATGCAAATACT | |
| | CCCTGTGGAAGAGAAGGCCCTAATGAAGGCTGTGGCAACTGTGGGGCGCATCTCTGCT | |
| | GTTGTTTATGGACTTCTTGATTCCTTCTGGTCCTATAAAAAAAGAAGGGACCTTTCCC | |
| | CTCTATAGCGAGGGGTATTGTTTTCTCACAGACTATGGATTTTAACAACAGGAATGCA | |
| | AAAAAAAAAAAAGAATTGGTGTTCAGCATTAGACCTCCCAAACAGAATTTCTGACTTA | |
| | ACAATGGTCCACTCTGGAGACTGGAAAGTCCAAGGTCACAGAGGTGCATCTGGTGAGA | |
| | GCCTTCTTGCTAGTGGGGAATCTCAGCAGAGTCCTGAGGTGGCACAGTCCTGTCTGCA | |
| | TTAAAAGATTCAGTGGAAAAATGAGAAGCCAATAGAAGCAACATC | |
| | ORF Start: ATG at 16 | ORF Stop: TAG at 760 |
| | SEQ ID NO:76 | 248 aa MW at 28420.1 kD |

| | |
|---|---|
| NOV18c, | MNPSLLLAVFCLRLASASLTLDHSLDQWKAKHKRLYGMNEEGWRRAVWQNMKMIEQHN |
| CG93252-03 Protein | QEYREGKHSFTMAMNAFGEMTSEEFRQVVNGFQNQKHRKGKVLQEPLLHDIRKSVDWR |
| Sequence | EKGYVTPVKDQVRQCASSYAFSAAGALDLVDCSRLQGNVGCIFGEPLFCFQYVADNGG |
| | LDSEESFSYEEKEKACRYNPKYSATNDTGYMQILPVEEKALMKAVATVGRISAVVYGL |
| | LDSFWSYKKRRDLSPL |

Sequence comparison of the above protein sequences yields the following sequence relationships shown in Table 18B.

TABLE 18B
Comparison of NOV18a against NOV18b and NOV18c.

| | NOV18a Residues/ | Identities/Similarities |
|---|---|---|
| Protein Sequence | Match Residues | for the Matched Region |
| NOV18b | 1 . . . 323 | 305/323 (94%) |
| | 1 . . . 319 | 309/323 (95%) |
| NOV18c | 1 . . . 257 | 200/258 (77%) |
| | 1 . . . 241 | 209/258 (80%) |

Further analysis of the NOV18a protein yielded the following properties shown in Table 18C.

TABLE 18C
Protein Sequence Properties NOV18a

| | |
|---|---|
| PSort | 0.7427 probability located in outside; |
| analysis: | 0.1430 probability located in microbody |
| | (peroxisome); |
| | 0.1000 probability located in endoplasmic |
| | reticulum (membrane); |
| | 0.1000 probability located in endoplasmic |
| | reticulum (lumen) |
| SignalP | Cleavage site between residues 18 and 19 |
| analysis: | |

A search of the NOV18a protein against the Geneseq database, a proprietary database that contains sequences published in patents and patent publication, yielded several homologous proteins shown in Table 18D.

TABLE 18D
Geneseq Results for NOV18a

| Geneseq | Protein/Organism/Length | NOV18a Residues/ | Identities/Similarities | |
|---|---|---|---|---|
| Identifier | [Patent #, Date] | Match Residues | for the Matched Region | Expect Value |
| AAW47031 | Human procathepsin L - Homo sapiens, | 1 . . . 329 | 292/334 (87%) | e-176 |
| | 333 aa. [US5710014-A, 20-JAN-1998] | 1 . . . 333 | 309/334 (92%) | |
| AAM93531 | Human polypeptide, SEQ ID NO:3271- | 1 . . . 329 | 291/334 (87%) | e−175 |
| | Homo sapiens, 333 aa. [EP1130094-A2, | 1 . . . 333 | 308/334 (92%) | |
| | 05-SEP-2001] | | | |
| AAR28829 | Human procathepsin L - Homo sapiens, | 1 . . . 329 | 293/334 (87%) | e−175 |
| | 333 aa. [WO9219756-A, 12-NOV-1992] | 1 . . . 333 | 309/334 (91%) | |
| AAP82094 | pHu-16 sequence encoded human | 1 . . . 329 | 286/334 (85%) | e−173 |
| | procathepsin L - Homo sapiens, 333 aa. | 1 . . . 333 | 308/334 (91%) | |
| | [USN7154692-N, 11-FEB-1988] | | | |
| AAU12177 | Human PRO305 polypeptide sequence - | 1 . . . 329 | 239/334 (71%) | e−143 |
| | Homo sapiens, 334 aa. | 1 . . . 334 | 275/334 (81%) | |
| | [WO200140466-A2, 07-JUN-2001] | | | |

In a BLAST search of public sequence databases, the NOV18a protein was found to have homology to the proteins shown in the BLASTP data in Table 18E.

TABLE 18E
Public BLASTP Results for NOV18a

| | | NOV18a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P07711 | Cathepsin L precursor (EC 3.4.22.15) | 1 . . . 329 | 292/334 (87%) | e−175 |
| | (Major excreted protein) (MEP) - Homo | 1 . . . 333 | 309/334 (92%) | |
| | sapiens (Human), 333 aa. | | | |
| Q96QJ0 | SIMILAR TO CATHEPSIN L - Homo | 1 . . . 329 | 291/334 (87%) | e−175 |
| | sapiens (Human), 333 aa. | 1 . . . 333 | 309/334 (92%) | |
| Q9GKL8 | CYSTElNE PROTEASE - Cercopithecus | 1 . . . 329 | 280/334 (83%) | e−170 |
| | aethiops (Green monkey) (Grivet), 333 aa. | 1 . . . 333 | 304/334 (90%) | |
| Q9GL24 | CATHEPSIN L (EC 3.4.22.15) - Canis | 1 . . . 329 | 249/335 (74%) | e−146 |
| | familiaris (Dog), 333 aa. | 1 . . . 333 | 281/335 (83%) | |
| P25975 | Cathepsin L precursor (EC 3.4.22.15) - | 1 . . . 329 | 242/335 (72%) | e−144 |
| | Bos taurus (Bovine), 334 aa. | 1 . . . 334 | 279/335 (83%) | |

PFam analysis predicts that the NOV18a protein contains the domains shown in the Table 18F.

TABLE 18F
Domain Analysis of NOV18a

| | | Identities/ | |
|---|---|---|---|
| | | Similarities | |
| | NOV18a | for the | |
| Pfam Domain | Match Region | Matched Region | Expect Value |
| Peptidase_C1: | 109 . . . 328 | 122/338 (36%) | 8.2e−117 |
| domain 1 of 1 | | 192/338 (57%) | |

### Example 19

The NOV19 clone was analyzed, and the nucleotide and encoded polypeptide sequences are shown in Table 19A.

TABLE 19A
NOV19 Sequence Analysis

| | |
|---|---|
| SEQ ID NO:77 | 1071 bp |

| | |
|---|---|
| NOV19, | GCACTGAGAAGGAAGACAAAGGCCAGCATGTCCAGGCTTTTTTTTTTTTTTTTTTTTT |
| CG93285-01 | |
| DNA Sequence | TGCTGCTGGTGCTGCTCTGGGGTGTGGGGTTGCACAGCTTCCCAGCGACTCCAGAAAC |
| | ACAAGAACAAGATGCAGAGATAGTCCAGAAATACCTAGAAAACTGCTACTACAACTTG |
| | AAGAGTGAAATCAATCAAATTGGAAGGCAGAGAGACAGTAGCCCAGTGCTTGAGAAGC |
| | TGAAGCAAATGCAGAATTTCTTTGGGCTGAAGGTAACTGGGAAGCCAGATTTGATGAA |
| | GCAGCCCAGATGTGGGGTGCCTGATGTGGCTTCCCTCATCCTCACTCAAGAGAGCCCT |
| | TGTTGGGAGCAAACAAATCTGACCCACAGGGATCAAAACTACATGCCAAATCTGCCTC |
| | AAGAGGATGTGGACCGTGCCACTGGGAAAGCCTTTGAACTCTGGAGTAAGGCCTCGGC |
| | CCTGACCTTCACCAGGGACTTTGAGAGTGAAGGGGACATAATATTATCCTTTGTGCTT |
| | GCAGATCTCCATGACAATTCTCCCTTTTATGGACATGATGGTTGTCTTGCTCATGCAT |
| | TCCCACCTGGACCAGGTATCGGAGGAGATGTTCATTTTGATAATGATGAAACAAGGAC |
| | CAAGGATTTCAGAAGTGAGTACTATTGGGTCGTTCAGGAGGATCAACTGCTGAGTGGC |
| | TACCCCAGGGACGTCTACAGCTCCTTTGTCTTCCCTGAAAGGGTGAAGAAAATTGATG |
| | CTGCCATTTATGAGAAGGACACTGGAAAGACACATTTCTTTGTTGCCAATGAGTATTG |
| | GAGGAGGTATGATGAAAATATGCAGTCCGTGGATGCAGGTTATCCCAAAATCATTGAT |
| | GACCTCCCCGGAATTAGTAAAAAAGGTTTTTTCTATTTCTTTTGTAGAAGAAGGCAGT |
| | ATGAATGTAATCCTAAAATGAAGCAAATTTTGACTCTCCTGAAAGCTAACATCTGGTT |
| | CAAGTGCAGAAATAACTGATGGTTGACTATCACCAAACAGAAAATAAAAAGTATTTTT |
| | AATGAGCCCAAAATATGTTCTTTTCTA |

| | | |
|---|---|---|
| ORF Start: ATG at 28 | | ORF Stop: TGA at 1003 |
| SEQ ID NO:78 | 325aa | MW at 37891.6kD |

| | |
|---|---|
| NOV19, | MSRLFFFFFFLLLVLLWGVGLHSFPATPETQEQDAEIVQKYLENCYYNLKSEINQIGR |
| CG93285-01 | |
| Protein Sequence | QRDSSPVLEKLKQMQNFFGLKVTGKPDLMKQPRCGVPDVASLILTQESPCWEQTNLTH |
| | RDQNYMPNLPQEDVDRATGKAFELWSKASALTFTRDFESEGDIILSFVLADLHDNSPF |
| | YGHDGCLAHAFPPGPGIGGDVHFDNDETRTKDFRSEYYWVVQEDQLLSGYPRDVYSSF |
| | VFPERVKKIDAAIYEKDTGKTHFFVANEYWRRYDENMQSVDAGYPKIIDDLPGISKKG |
| | FFYFFCRRRQYECNPKMKQILTLLKANIWFKCRNN |

Further analysis of the NOV19 protein yielded the following properties shown in Table 19B.

TABLE 19B
Protein Sequence Properties NOV19

| | |
|---|---|
| PSort | 0.8200 probability located in outside; 0.2294 probability |
| analysis: | located in microbody (peroxisome); 0.1000 probability |
| | located in endoplasmic reticulum (membrane); 0.1000 |
| | probability located in endoplasmic reticulum (lumen) |
| SignalP | Cleavage site between residues 24 and 25 |
| analysis: | |

A search of the NOV19 protein against the Geneseq database, a proprietary database that contains sequences published in patents and patent publication, yielded several homologous proteins shown in Table 19C.

TABLE 19C
Geneseq Results for NOV19

| | | NOV19 | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAG75509 | Human colon cancer antigen protein SEQ | 11 . . . 208 | 129/203 (63%) | 9e−70 |
| | ID NO:6273 - Homo sapiens, 496 aa. | 34 . . . 235 | 155/203 (75%) | |
| | [WO200122920-A2, 05-APR-2001] | | | |
| AAB84606 | Amino acid sequence of matrix | 11 . . . 208 | 129/203 (63%) | 9e−70 |
| | metalloproteinase collagenase 1 - Homo | 7 . . . 208 | 155/203 (75%) | |
| | sapiens, 469 aa. [WO200149309-A2, | | | |
| | 12-JUL-2001] | | | |
| AAE10415 | Human matrix metalloprotinase-1 (MMP-1) | 11 . . . 208 | 129/203 (63%) | 9e−70 |
| | protein - Homo sapiens, 469 aa. | 7 . . . 208 | 155/203 (75%) | |
| | [WO200166766-A2, 13-SEP-2001] | | | |
| AAP70611 | Sequence encoded by human skin | 11 . . . 208 | 128/203 (63%) | 4e−69 |
| | collagenase cDNA - Homo sapiens, 469 aa. | 7 . . . 208 | 154/203 (75%) | |
| | [GB2182665-A, 20-MAY-1987] | | | |
| AAP93628 | Sequence of human interstitial | 24 . . . 208 | 119/190 (62%) | 8e−64 |
| | procollagenase - Homo sapiens, 457 aa. | 8 . . . 196 | 144/190 (75%) | |
| | [GB2209526-A, 17-MAY-1989] | | | |

In a BLAST search of public sequence databases, the NOV19 protein was found to have homology to the proteins shown in the BLASTP data in Table 19D.

TABLE 19D
Public BLASTP Results for NOV19

| | | NOV19 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9XSZ5 | Interstitial collagenase precursor (EC | 11 . . . 209 | 132/205 (64%) | 1e−69 |
| | 3.4.24.7) (Matrix metalloproteinase-1) | 6 . . . 209 | 157/205 (76%) | |
| | (MMP-1) - Equus caballus (Horse), 469 aa. | | | |
| P03956 | Interstitial collagenase precursor (EC | 11 . . . 208 | 129/203 (63%) | 2e−69 |
| | 3.4.24.7) (Matrix metalloproteinase-1) | 7 . . . 208 | 155/203 (75%) | |
| | (MMP-1) (Fibroblast collagenase) - Homo | | | |
| | sapiens (Human), 469 aa. | | | |
| P13943 | Interstitial collagenase precursor (EC | 11 . . . 220 | 130/215 (60%) | 6e−68 |
| | 3.4.24.7) (Matrix metalloproteinase-1) | 6 . . . 219 | 157/215 (72%) | |
| | (MMP-1) - Oryctolagus cuniculus (Rabbit), | | | |
| | 468 aa. | | | |
| P21692 | Interstitial collagenase precursor (EC | 7 . . . 220 | 132/220 (60%) | 7e−66 |
| | 3.4.24.7) (Matrix metalloproteinase-1) | 2 . . . 220 | 156/220 (70%) | |
| | (MMP-1) - Sus scrofa (Pig), 469 aa. | | | |
| P28053 | Interstitial collagenase precursor (EC | 11 . . . 208 | 124/204 (60%) | 3e−64 |
| | 3.4.24.7) (Matrix metalloproteinase-1) | 6 . . . 208 | 147/204 (71%) | |
| | (MMP-1) (Fibroblast collagenase) - Bos | | | |
| | taurus (Bovine), 469 aa. | | | |

PFam analysis predicts that the NOV19 protein contains the domains shown in the Table 19E.

TABLE 19E
Domain Analysis of NOV19

| | | Identities/ | |
|---|---|---|---|
| | | Similarities | |
| Pfam | NOV19 | for the | Expect |
| Domain | Match Region | Matched Region | Value |
| Peptidase_M10: | 41 . . . 204 | 90/172 (52%) | 4.2e−67 |
| domain 1 of 1 | | 135/172 (78%) | |
| hemopexin: domain 1 of 1 | 241 . . . 288 | 26/51 (51%) | 2.2e−09 |
| | | 38/51 (75%) | |

### Example 20

The NOV20 clone was analyzed, and the nucleotide and encoded polypeptide sequences are shown in Table 20A.

TABLE 20A
NOV20 Sequence Analysis

| | |
|---|---|
| SEQ ID NO:79 | 4401 bp |

| | |
|---|---|
| NOV20a, | GGTGCCGAGCACTCCGGACTCTACGTGAACAACAACGGGATCATCTCCTTCCTGAAGG |
| GG93387-01 DNA | |
| Sequence | AGGTTTCTCAGTTCACCCCAGTGGCCTTCCCCATTGCCAAGGACCGCTGCGTGGTGGC |
| | AGCCTTCTGGGCAGATGTGGACAACCGGCGTGCAGGCGACGTGTACTACCGGGAGGCC |
| | ACCGACCCAGCCATGCTGCGCCGAGCCACGGAGGACGTCAGGCACTACTTCCCCGAGC |
| | TCCTGGACTTCAATGCCACCTGGGTTTTTGTTGCCACCTGGTACCGAGTGACCTTCTT |
| | TGGAGGCAGTTCCTCATCCCCTGTCAACACATTCCAGACTGTGCTCATCACAGACGGC |
| | AAGCTCTCCTTCACCATCTTCAACTATGAGTCCATCGTGTGGACCACAGGCACACACG |
| | CCAGCAGCGGGGGCAACGCCACTGGCCTCGGGGGCATCGCAGCCCAGGCTGGCTTCAA |
| | CGCAGGCGATGGGCAGCGTTACTTCAGTATCCCCGGCTCGCGCACAGCAGACATGGCC |
| | GAGGTGGAGACCACCACCAACGTGGGTGTGCCCGGGCGCTGGGCGTTCAGAATCGATG |
| | ATGCCCAGGTGCGCGTGGGGGGCTGCGGCCATACAACGTCCGTGTGCCTGGCCCTGCG |
| | CCCCTGCCTCAACGGCGGCAAGTGCATCGACGACTGCGTCACGGGCAACCCCTCCTAC |
| | ACCTGCTCCTGCCTCTCGGGCTTCACGGGGCGGAGGTGCCACCTGGACGTGAACGAAT |
| | GTGCCTCCCAGCCCTGTCAGAATGGTGGGACCTGTACTCACGGCATCAACAGTTTCCG |
| | CTGCCAGTGCCCGGCTGGCTTTGGGGGACCCACCTGTGAGACAGCCCAATCCCCCTGT |
| | GACACCAAAGAGTGTCAACATGGTGGCCAGTGCCAGGTGGAGAACGGCTCTGCGGTGT |
| | GTGTGTGCCAGGCCGGATACACCGGAGCAGCCTGCGAGATGGATGTGGACGACTGCAG |
| | CCCTGACCCCTGCCTGAATGGAGGCTCTTGTGTTGACCTAGTGGGGAATTACACCTGC |
| | TTGTGTGCCGAGCCCTTCAAGGGACTTCGCTGTGAGACAGGAGACCATCCAGTGCCAG |
| | ACGCCTGCCTCTCGGCCCCTTGCCACAATGGGGGCACCTGTGTGGATGCGGACCAGGG |
| | CTACGTGTGCGAGTGCCCCGAAGGCTTCATGGGCCTGGACTGCAGGGAGAGAGTCCCC |
| | GATGACTGTGAGTGCCGCAACGGAGGCAGATGCCTGGGCGCCAACACCACCCTCTGCC |
| | AGTGCCCCCTGGGATTCTTTGGGCTTCTCTGTGAATTTGAAATCACAGCCATGCCCTG |
| | CAACATGAACACACAGTGCCCAGATGGGGGCTACTGCATGGAGCACGGCGGGAGCTAC |
| | CTCTGCGTCTGCCACACCGACCACAATGCCAGCCACTCCCTGCCATCACCCTGCGACT |
| | CGGACCCCTGCTTCAACGGAGGCTCCTGCGATGCCCATGACGACTCCTACACCTGCGA |
| | GTGCCCGCGCGGGTTCCACGGCAAGCACTGCGAGAAAGCCCGGCCACACCTGTGCAGC |
| | TCAGGGCCCTGCCGGAACGGGGGCACGTGCAAGGAGGCGGGCGGCGAGTACCACTGCA |
| | GCTGCCCCTACCGCTTCACTGGGAGGCACTGTGAGATCGGGAAGCCAGACTCGTGTGC |
| | CTCTGGCCCCTGTCACAACGGCGGCACCTGCTTCCACTACATTGGCAAATACAAGTGT |
| | GACTGTCCCCCAGGCTTCTCCGGGCGGCACTGCGAGATAGCCCCCTCCCCCTGCTTCC |
| | GGAGCCCGTGTGTGAATGGGGGCACCTGCGAGGACCGGGACACGGATTTCTTCTGCCA |
| | CTGCCAAGCAGGGTACATGGGACGCCGGTGCCAGGCAGAGGTGGACTGCGGCCCCCCG |
| | GAGGAGGTGAAGCACGCCACACTGCGCTTCAACGGCACGCGGCTGGGCGCGGTGGCCC |
| | TGTATGCATGTGACCGTGGCTACAGCCTGAGCGCCCCCAGCCGCATCCGGGTCTGCCA |
| | GCCACACGGTGTCTGGAGTGAGCCTCCCCAGTGCCTTGAAATCGATGAGTGCCGGTCT |
| | CAGCCGTGCCTGCATGGGGGCTCTTGTCAGGACCGCGTTGCTGGGTACCTGTGCCTCT |
| | GCAGCACAGGCTATGAGGGCGCCCACTGTGAGCTGGAGAGGGATGAGTGCCGAGCTCA |
| | CCCGTGCAGAAATGGAGGGTCCTGCAGGAACCTCCCAGGGGCCTATGTCTGCCGGTGC |
| | CCTGCAGGCTTCGTTGGAGTCCACTGTGAGACAGAGGTGGACGCCTGCGACTCCAGCC |
| | CCTGCCAGCATGGAGGCCGGTGTGAGAGCGGCGGCGGGGCCTACCTGTGCGTCTGCCC |
| | AGAGAGCTTCTTCGGCTACCACTGCGAGACAGTGAGTGACCCCTGCTTCTCCAGCCCC |
| | TGTGGGGGCCGTGGCTATTGCCTGGCCAGCAACGGCTCCCACAGCTGCACCTGCAAAG |
| | TGGGCTACACGGGCGAGGACTGCGCCAAAGAGCTCTTCCCACCGACGGCCCTCAAGAT |
| | GGAGAGAGTGGAGGAGAGTGGGGTCTCTATCTCCTGGAACCCGCCCAATGGTCCAGCC |
| | GCCAGGCAGATGCTTGATGGCTACGCGGTCACCTACGTCTCCTCCGACGGCTCCTACC |
| | GCCGCACAGACTTTGTGGACAGGACCCGCTCCTCGCACCAGCTCCAGGCCCTGGCGGC |
| | CGGCAGGGCCTACAACATCTCCGTCTTCTCAGTGAAGCGAAACAGTAACAACAAGAAT |
| | GACATCAGCAGGCCTGCCGTGCTGCTGGCCCGCACGCGACCCCGCCCTGTGGAAGGCT |
| | TCGAGGTCACCAATGTGACGGCTAGCACCATCTCAGTGCAGTGGGCCCTGCACAGGAT |
| | CCGCCATGCCACCGTCAGTGGGGTCCGTGTGTCCATCCGCCACCCTGAGGCCCTCAGG |
| | GACCAGGCCACCGATGTGGACAGGAGTGTGGACAGGTTCACCTTTAGGGCCCTGCTGC |
| | CTGGGAAGAGGTACACCATCCAGCTGACCACCCTCAGTGGGCTCAGGGGAGAGGAGCA |
| | CCCCACAGAGAGCCTGGCCACCGCGCCGACGCACGTGTGGACCCGGCCCCTGCCTCCA |
| | GCAAACCTGACCGCCGCCCGAGTCACTGCCACCTCTGCCCACGTGGTCTGGGATGCCC |
| | CGACTCCAGGCAGCTTGCTGGAGGCTTATGTCATCAATGTGACCACCAGCCAGAGCAC |
| | CAAGAGCCGCTATGTCCCCAACGGGAAGCTGGCGTCCTACACGGTGCGCGACCTGCTG |
| | CCGGGACGGCGGTACCAGCCCTCTGTGATAGCAGTGCAGAGCACGGAGCTCGGGCCGC |
| | AGCACAGCGAGCCCGCCCACCTCTACATCATCACCTCCCCCAGGGATGGCGCTGACAG |
| | ACGCTGGCACCAGGGAGGACACCACCCTCGGGTGCTCAAGAACAGACCGCCCCCGGCG |
| | CGCCTGCCGGAGCTGCGCCTGCTCAATGACCACAGCGCCCCCGAGACCCCCACCCAGC |
| | CCCCCAGGTTCTCGGAGTTTGTGGACGGCAGAGGAAGAGTGAGCGCCAGGTTCGGTGG |
| | CTCACCCAGCAAAGCAGCCACCGTGAGATCACAACCCACAGCCTCGGCGCAGCTCGAG |
| | AACATGGAGGAAGCCCCCAAGCGGGTCAGCCCGGCCCTCCAGCTCCCTGAACACGGCA |
| | GCAAGGACATCGGAAACGTCCCTGGCAACTGTTCAGAAAACCCCTGTCAGAACGGAGG |
| | CACTTGTGTGCCGGGCGCAGACGCCCACAGCTGTGACTGCGGGCCAGGGTTCAAAGGC |
| | AGACGCTGCGAGCTCGCCTGTATAAAGGTGTCCCGCCCCTGCACAAGGCTGTTCTCCG |
| | AGACAAAGGCCTTTCCAGTCTGGGAGGGAGGCGTCTGTCACCACGTGTATAAAAGAGT |
| | CTACCGAGTTCACCAAGACATCTGCTTCAAAGAGAGCTGTGAAAGCACAAGCCTCAAG |
| | AAGACCCCAAACAGGAAACAAAGTAAGAGTCAGACACTGGAGAAATCTTAAGAAAGAA |
| | GGAACAGGCAATGTAGAGAAGCTGTCAAATGGTGGACTCCCAAACCGTTCCACCACTG |
| | CCTCAAAAAACATCTTGACCAGCAGAAGGTGGAGCTCAATGAAGGGTCAAGAGCTCAG |
| | CGAAGGGTAACTAGGTGGAACTGAGAGAAACCACGTTCACAAACTGCGTAATGCGGAC |
| | TTCCTGCCGCCCTGGAGACCCCTCAACTCTCTGTCCATGTAAGGCCCTTAAAGAGATT |
| | CATAGGAACTTTGAGCATCCTTNAGATGTGAATATTGTTGGGGGCAGGATTGGGGGAT |
| | AAATAGAAGGGAAGGCCACTCCACGAGTATCCCATGAACCTGGCCAGATCT |

| | | |
|---|---|---|
| ORF Start: ATG at 187 | | ORF Stop: TAA at 4051 |
| SEQ ID NO:80 | 1288 aa | MW at 138908.1 kD |

| | |
|---|---|
| NOV20a, | MLRRATEDVRHYFPELLDFNATWVFVATWYRVTFFGGSSSSPVNTFQTVLITDGKLSF |
| CG93387-01 Protein | |
| Sequence | TIFNYESIVWTTGTHASSGGNATGLGGIAAQAGFNAGDGQRYFSIPGSRTADMAEVET |
| | TTNVGVPGRWAFRIDDAQVRVGGCGHTTSVCLALRPCLNGGKCIDDCVTGNPSYTCSC |
| | LSGFTGRRCHLDVNECASQPCQNGGTCTHGINSFRCQCPAGFGGPTCETAQSPCDTKE |
| | CQHGGQCQVENGSAVCVCQAGYTGAACEMDVDDCSPDPCLNGGSCVDLVGNYTCLCAE |
| | PFKGLRCETGDHPVPDACLSAPCHNGGTCVDADQGYVCECPEGFMGLDCRERVPDDCE |
| | CRNGGRCLGANTTLCQCPLGFFGLLCEFEITAMPCNMNTQCPDGGYCMEHGGSYLCVC |
| | HTDHNASHSLPSPCDSDPCFNGGSCDAHDDSYTCECPRGFHGKHCEKARPHLCSSGPC |
| | RNGGTCKEAGGEYHCSCPYRFTGRHCEIGKPDSCASGPCHNGGTCFHYIGKYKCDCPP |
| | GFSGRHCEIAPSPCFRSPCVNGGTCEDRDTDFFCHCQAGYMGRRCQAEVDCGPPEEVK |
| | HATLRFNGTRLGAVALYACDRGYSLSAPSRIRVCQPHGVWSEPPQCLEIDECRSQPCL |
| | HGGSCQDRVAGYLCLCSTGYEGAHCELERDECRAHPCRNGGSCRNLPGAYVCRCPAGF |
| | VGVHCETEVDACDSSPCQHGGRCESGGGAYLCVCPESFFGYHCETVSDPCFSSPCGGR |
| | GYCLASNGSHSCTCKVGYTGEDCAKELFPPTALKMERVEESGVSISWNPPNGPAARQM |
| | LDGYAVTYVSSDGSYRRTDFVDRTRSSHQLQALAAGRAYNISVFSVKRNSNNKNDISR |
| | PAVLLARTRPRPVEGFEVTNVTASTISVQWALHRIRHATVSGVRVSIRHPEALRDQAT |
| | DVDRSVDRFTFRALLPGKRYTIQLTTLSGLRGEEHPTESLATAPTHVWTRPLPPANLT |
| | AARVTATSAHVVWDAPTPGSLLEAYVINVTTSQSTKSRYVPNGKLASYTVRDLLPGRR |
| | YQPSVIAVQSTELGPQHSEPAHLYIITSPRDGADRRWHQGGHHPRVLKNRPPPARLPE |
| | LRLLNDHSAPETPTQPPRFSEFVDGRGRVSARFGGSPSKAATVRSQPTASAQLENMEE |
| | APKRVSPALQLPEHGSKDIGNVPGNCSENPCQNGGTCVPGADAHSCDCGPGFKGRRCE |
| | LACIKVSRPCTRLFSETKAFPVWEGGVCHHVYKRVYRVHQDICFKESCESTSLKKTPN |
| | RKQSKSQTLEKS |

| | |
|---|---|
| SEQ ID NO:81 | 4413 bp |

| | |
|---|---|
| NOV20b, | GAGCACTCCGGACTCTACGTGAACAACAACGGGATCATCTCCTTCCTGAAGGAGGTTT |
| CG93387-02 DNA | |
| Sequence | CTCAGTTCACCCCAGTGGCCTTCCCCATTGCCAAGGACCGCTGCGTGGTGGCAGCCTT |
| | CTGGGCAGATGTGGACAACCGGCGTGCAGGCGACGTGTACTACCGGGAGGCCACCGAC |
| | CCAGCCATGCTGCGCCGAGCCACGGAGGACGTCAGGCACTACTTCCCCGAGCTCCTGG |
| | ACTTCAATGCCACCTGGGTTTTTGTTGCCACCTGGTACCGAGTGACCTTCTTTGGAGG |
| | CAGTTCCTCATCCCCTGTCAACACATTCCAGACTGTGCTCATCACAGACGGCAAGCTC |
| | TCCTTCACCATCTTCAACTATGAGTCCATCGTGTGGACCACAGGCACACACGCCAGCA |
| | GCGGGGGCAACGCCACTGGCCTCGGGGGCATCGCAGCCCAGGCTGGCTTCAACGCAGG |
| | CGATGGGCAGCGTTACTTCAGTATCCCCGGCTCGCGCACAGCAGACATGGCCGAGGTG |
| | GAGACCACCACCATCGTGGTTGTGCCCGGGCGCTGGGCGTTCATAATCGATGATGCCC |
| | AGGTGCGCGTGGGGGGCTGCGGCCATACAACGTCCGTGTGCCTGGCCCTGCGCCCCTG |
| | CCTCAACGGCGGCAAGTGCATCGACGACTGCGTCACGGGCAACCCCTCCTACACCTGC |
| | TCCTGCCTCTCGGGCTTCACGGGGCGGAGGTGCCACCTGGACGTGAACGAATGTGCCT |
| | CCCAGCCCTGTCAGAATGGTGGGACCTGTACTCACGGCATCAACAGTTTCCGCTGCCA |
| | GTGCCCGGCTGGCTTTGGGGGACCCACCTGTGAGACAGCCCAATCCCCCTGTGACACC |
| | AAAGAGTGTCAACATGGTGGCCAGTGCCAGGTGGAGAATGGCTCTGCGGTGTGTGTGT |
| | GCCAGGCCGGATACACCGGAGCAGCCTGCGAGATGGATGTGGACGACTGCAGCCCTGA |
| | CCCCTGCCTGAATGGAGGCTCTTGTGTTGACCTAGTGGGGAATTACACCTGCTTGTGT |
| | GCCGAGCCCTTCAAGGGACTTCGCTGTGAGACAGGAGACCATCNNCAGTGCCAGACGC |
| | CTGCCTCTCGGCCCCTTGCCACAATGGGGGCACCTGTGTGGATGCGGACCAGGGCTAC |
| | GTGTGCGAGTGCCCCGAAGGCTTCATGGGCCTGGACTGCAGGGAGAGAGTCCCCGATG |
| | ACTGTGAGTGCCGCAACGGAGGCAGATGCCTGGGCGCCAACACCACCCTCTGCCCAGT |
| | GCCCCCTGGGATTCTTTGGGCTTCTCTGTGAATTTGAAATCACAGCCATGCCCTGCAA |
| | CATGAACACACAGTGCCCAGATGGGGGCTACTGCATGGAGCACGGCGGGAGCTACCTC |
| | TGCGTCTGCCACACCGACCACAATGCCAGCCACTCCCTGCCATCACCCTGCGACTCGG |
| | ACCCCTGCTTCAACGGAGGCTCCTGCGATGCCCATGACGACTCCTACACCTGCGAGTG |
| | CCCGCGCGGGTTCCACGGCAAGCACTGCGAGAAAGCCCGGCCACACCTGTGCAGCTCA |
| | GGGCCCTGCCGGAACGGGGGCACGTGCAAGGAGGCGGGCGGCGAGTACCACTGCAGCT |
| | GCCCCTACCGCTTCACTGGGAGGCACTGTGAGATCGGGAAGCCAGACTCGTGTGCCTC |
| | TGGCCCCTGTCACAACGGCGGCACCTGCTTCCACTACATTGGCAAATACAAGTGTGAC |
| | TGTCCCCCAGGCTTCTCCGGGCGGCACTGCGAGATAGCCCCCTCCCCCTGCTTCCGGA |
| | GCCCGTGTGTGAATGGGGGCACCTGCGAGGACCGGGACACGGATTTCTTCTGCCACTG |
| | CCAAGCAGGGTACATGGGACGCCGGTGCCAGGCAGAGGTGGACTGCGGCCCCCCGGAG |
| | GAGGTGAAGCACGCCACACTGCGCTTCAACGGCACCCGGCTGGGCGCGGTGGCCCTGT |
| | ATGCATGTGACCGTGGCTACAGCCTGAGCGCCCCCAGCCGCATCCGGGTCTGCCAGCC |
| | ACACGGTGTCTGGAAAATCGATGAGTGCCGGTCTCAGCCGTGCCTGCATGGGGGCTCT |
| | TGTCAGGACCGCGTTGCTGGGTACCTGTGCCTCTGCAGCACAGGCTATGAGGGCGCCC |
| | ACTGTGAGCTGGAGAGGGATGAGTGCCGAGCTCACCCGTGCAGAAATGGAGGGTCCTG |
| | CAGGAACCTCCCAGGGGCCTATGTCTGCCGGTGCCCTGCAGGCTTCGTTGGAGTCCAC |
| | TGTGAGACAGAGGTGGACGCCTGCGACTCCAGCCCCTGCCAGCATGGAGGCCGGTGTG |
| | AGAGCGGCGGCGGGGCCTACCTGTGCGTCTGCCCAGAGAGCTTCTTCGGCTACCACTG |
| | CGAGACAGTGAGTGACCCCTGCTTCTCCAGCCCCTGTGGGGGCCGTGGCTATTGCCTG |
| | GCCAGCAACGGCTCCCACAGCTGCACCTGCAAAGTGGGCTACACGGGCGAGGACTGCG |
| | CCAAAGAGCTCTTCCCACCGACGGCCCTCAAGATGGAGAGAGTGGAGGAGAGTGGGGT |
| | CTCTATCTCCTGGAACCCGCCCAATGGTCCAGCCGCCAGGCAGATGCTTGATGGCTAC |
| | GCGGTCACCTACGTCTCCTCCGACGGCTCCTACCGCCGCACAGACTTTGTGGACAGGA |
| | CCCGCTCCTCGCACCAGCTCCAGGCCCTGGCGGCCGGCAGGGCCTACAACATCTCCGT |
| | CTTCTCAGTGAAGCGAAACAGTAACAACAAGAATGACATCAGCAGGCCTGCCGTGCTG |
| | CTGGCCCGCACGCGACCCCGCCCTGTGGAAGGCTTCGAGGTCACCAATGTGACGGCTA |
| | GCACCATCTCAGTGCAGTGGGCCCTGCACAGGATCCGCCATGCCACCGTCAGTGGGGT |
| | CCGTGTGTCCATCCGCCACCCTGAGGCCCTCAGGGACCAGGCCACCGATGTGGACAGG |
| | AGTGTGGACAGGTTCACCTTTAGGGCCCTGCTGCCTGGGAAGAGGTACACCATCCAGC |
| | TGACCACCCTCAGTGGGCTCAGGGGAGAGGAGCACCCCACAGAGAGCCTGGCCACCGC |
| | GCCGACGCACGTGTGGACCCGGCCCCTGCCTCCAGCAAACCTGACCGCCGCCCGAGTC |
| | ACTGCCACCTCTGCCCACGTGGTCTGGGATGCCCCGACTCCAGGCAGCTTGCTGGAGG |
| | CTTATGTCATCAATGTGACCACCAGCCAGAGCACCAAGAGCCGCTATGTCCCCAACGG |
| | GAAGCTGGCGTCCTACACGGTGCGCGACCTGCTGCCGGGACGGCGGTACCAGCTCTCT |
| | GTGATAGCAGTGCAGAGCACGGAGCTCGGGCCGCAGCACAGCGAGCCCGCCCACCTCT |
| | ACATCATCACCTCCCCCAGGGATGGCGCTGACAGACGCTGGCACCAGGGAGGACACCA |
| | CCCTCGGGTGCTCAAGAACAGACCGCCCCCGGCGCGCCTGCCGGAGCTGCGCCTGCTC |
| | AATGACCACAGCGCCCCCGAGACCCCCACCCAGCCCCCCAGGTTCTCGGAGCTTGTGG |
| | ACGGCAGAGGAAGAGTGAGCGCCAGGTTCGGTGGCTCACCCAGCAAAGCAGCCACCGT |
| | GAGATCACGTCCTGTCCCCTACATGATGAGCCCACCCCCACCGCCAGCGCAGTCTCCA |
| | GCCAGTGACCCCCACCCCGACTGTGCACAAGGCGCGGGGCTCGTGGGCCGCCGGCAGC |
| | ATGCACCTCCATGGCAGGAGGGGCAGCTCGGACATCCGTGCTCCCTGAGATATAGAAG |
| | CACTCAAAAGGGTGGCCCCAGGACCATCCCGGGTGCAAAGCAGCTGCGCCGTGTGGTC |
| | ACCGCCTGGCTTCTCCTAGAACCCACAGCCTCGGCGCAGCTCGAGAACATGGAGGAAG |
| | CCCCCAAGCGGGTCAGCCTGGCCCTCCAGCTCCCTGAACACGGCAGCAAGGACATCGG |
| | AAGTTATGCAGGACCTGAACTGTCTCCTAGTCCGGGGCTCTGCCTCGTGAGGATCGAG |
| | GCCAGCACGTCCCTGCAGGGCACCAAGCATCTGCTGAGCACCTGCAGCACACAAGCAA |
| | AGGAGCAGGGTGGAGCCTTCACGCTGCCGTGCCTGTGTGGACCAGTCCAGGGTGACCA |
| | CGGGGTAGGTGAGGGAAAGCCTGTCTTCACAGACCACTCTCCAGCTGACGTCCCTGGC |
| | AACTGTTCAGAAAACCCCTGTCAGAACGGAGGCACTTGTGTGCCGGGCGCAGACGCCC |
| | ACAGCTGTGACTGCGGGCCAGGGTTCAAAGGCAGACGCTGCGAGCTCGGTATAAAAGA |
| | GTCTACCGAGTTCACCAAGACATCTGCTTCAAAGAGAGCTGTGAAAGCACAAGCCTCA |
| | AGAAGACCCCAAACAGGTGCCTCTGGGGAGCAGGCCCATGCCGTGTCCTGCATGTAGN |
| | NNNNN |

| | | |
|---|---|---|
| ORF Start: at 1090 | | ORF Stop: end of sequence |
| SEQ ID NO:82 | 1408 aa | MW at 150587.4 kD |

| | |
|---|---|
| NOV20b, | MLRRATEDVRHYFPELLDFNATWVFVATWYRVTFFGGSSSSPVNTFQTVLITDGKLSF |
| CG93387-02 Protein | |
| Sequence | TIFNYESIVWTTGTHASSGGNATGLGGIAAQAGFNAGDGQRYFSIPGSRTADMAEVET |
| | TTIVVVPGRWAFIIDDAQVRVGGCGHTTSVCLALRPCLNGGKCIDDCVTGNPSYTCSC |
| | LSGFTGRRCHLDVNECASQPCQNGGTCTHGINSFRCQCPAGFGGPTCETAQSPCDTKE |
| | CQHGGQCQVENGSAVCVCQAGYTGAACEMDVDDCSPDPCLNGGSCVDLVGNYTCLCAE |
| | PFKGLRCETGDHXQCQTPASRPLATMGAPVWMRTRATCASAPKASWAWTAGRESPMTV |
| | SAATEADAWAPTPPSAQCPLGFFGLLCEFEITAMPCNMNTQCPDGGYCMEHGGSYLCV |
| | CHTDHNASHSLPSPCDSDPCFNGGSCDAHDDSYTCECPRGFHGKHCEKARPHLCSSGP |
| | CRNGGTCKEAGGEYHCSCPYRFTGRHCEIGKPDSCASGPCHNGGTCFHYIGKYKCDCP |
| | PGFSGRHCEIAPSPCFRSPCVNGGTCEDRDTDFFCHCQAGYMGRRCQAEVDCGPPEEV |
| | KHATLRFNGTRLGAVALYACDRGYSLSAPSRIRVCQPHGVWKIDECRSQPCLHGGSCQ |
| | DRVAGYLCLCSTGYEGAHCELERDECRAHPCRNGGSCRNLPGAYVCRCPAGFVGVHCE |
| | TEVDACDSSPCQHGGRCESGGGAYLCVCPESFFGYHCETVSDPCFSSPCGGRGYCLAS |
| | NGSHSCTCKVGYTGEDCAKELFPPTAlKMERVEESGVSISWNPPNGPAARQMLDGYAV |
| | TYVSSDGSYRRTDFVDRTRSSHQLQALAAGRAYNISVFSVKRNSNNKNDISRPAVLLA |
| | RTRPRPVEGFEVTNVTASTISVQWALHRIRHATVSGVRVSIRHPEALRDQATDVDRSV |
| | DRFTFRALLPGKRYTIQLTTLSGLRGEEHPTESLATAPTHVWTRPLPPANLTAARVTA |
| | TSAHVVWDAPTPGSLLEAYVINVTTSQSTKSRYVPNGKLASYTVRDLLPGRRYQLSVI |
| | AVQSTELGPQHSEPAHLYIITSPRDGADRRWHQGGHHPRVLKNRPPPARLPELRLLND |
| | HSAPETPTQPPRFSELVDGRGRVSARFGGSPSKAATVRSRPVPYMMSPPPPPAQSPAS |
| | DPHPDCAQGAGLVGRRQHAPPWQEGQLGHPCSLRYRSTQKGGPRTIPGAKQLRRVVTA |
| | WLLLEPTASAQLENMEEAPKRVSLALQLPEHGSKDIGSYAGPELSPSPGLCLVRIEAS |
| | TSLQGTKHLLSTCSTQAKEQGGAFTLPCLCGPVQGDHGVGEGKPVFTDHSPADVPGNC |
| | SENPCQNGGTCVPGADAHSCDCGPGFKGRRCELGIKESTEFTKTSASKRAVKAQASRR |
| | PQTGASGEQAHAVSCM |

Sequence comparison of the above protein sequences yields the following sequence relationships shown in Table 20B.

TABLE 20B
Comparison of NOV20a against NOV20b.

| | | Identities/ |
|---|---|---|
| | NOV20a Residues/ | Similarities for |
| Protein Sequence | Match Residues | the Matched Region |
| NOV20b | 1 . . . 1146 | 1066/1147 (92%) |
| | 1 . . . 1140 | 1068/1147 (92%) |

Further analysis of the NOV20a protein yielded the following properties shown in Table 20C.

TABLE 20C
Protein Sequence Properties NOV20a

| | |
|---|---|
| PSort | 0.4500 probability located in cytoplasm; 0.3000 probability |
| analysis: | located in microbody (peroxisome); 0.1000 probability located |
| | in mitochondrial matrix space; 0.1000 probability located |
| | in lysosome (lumen) |
| SignalP | Cleavage site between residues 41 and 42 |
| analysis: | |

A search of the NOV20a protein against the Geneseq database, a proprietary database that contains sequences published in patents and patent publication, yielded several homologous proteins shown in Table 20D.

TABLE 20D
Geneseq Results for NOV20a

| | | NOV20a | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAB82249 | Human insulin-responsive sequence DNA | 261 . . . 1288 | 1025/1028 (99%) | 0.0 |
| | binding protein-1 - Homo sapiens, 1028 aa. | 1 . . . 1028 | 1025/1028 (99%) | |
| | [WO200132873-A1, 10-MAY-2001] | | | |
| AAB82247 | Rat insulin-responsive sequence DNA | 271 . . . 1273 | 817/1003 (81%) | 0.0 |
| | binding protein-1 - Rattus sp, 1008 aa. | 1 . . . 1002 | 895/1003 (88%) | |
| | [WO200132873-A1, 10-MAY-2001] | | | |
| AAB42900 | Human ORFX ORF2664 polypeptide | 1 . . . 627 | 592/629 (94%) | 0.0 |
| | sequence SEQ ID NO:5328 - Homo | 61 . . . 689 | 593/629 (94%) | |
| | sapiens, 694 aa. [WO200058473-A2, | | | |
| | 05-OCT-2000] | | | |
| AAB82251 | Rat insulin-responsive sequence DNA | 780 . . . 1273 | 388/494 (78%) | 0.0 |
| | binding protein-1 (truncated) - Rattus sp, | 1 . . . 493 | 433/494 (87%) | |
| | 499 aa. [WO200132873-A1, | | | |
| | 10-MAY-2001] | | | |
| AAB82250 | Human insulin-responsive sequence DNA | 813 . . . 1181 | 365/369 (98%) | 0.0 |
| | binding protein-1 (variant) - Homo sapiens, | 1 . . . 369 | 366/369 (98%) | |
| | 387 aa. [WO200132873-A1, 10-MAY-2001] | | | |

In a BLAST search of public sequence databases, the NOV20a protein was found to have homology to the proteins shown in the BLASTP data in Table 20E. It

TABLE 20E
Public BLASTP Results for NOV20a

| | | NOV20a | | |
|---|---|---|---|---|
| Protein | | Residues/ | Identities/ | |
| Accession | | Match | Similarities for the | Expect |
| Number | Protein/Organism/Length | Residues | Matched Portion | Value |
| BAB84888 | FLJ00133 PROTEIN - Homo sapiens | 7 . . . 1288 | 1279/1282 (99%) | 0.0 |
| | (Human), 1282 aa (fragment). | 1 . . . 1282 | 1279/1282 (99%) | |
| BAB84901 | FLJ00146 PROTEIN - Homo sapiens | 706 . . . 1288 | 519/583 (89%) | 0.0 |
| | (Human), 522 aa (fragment). | 1 . . . 522 | 520/583 (89%) | |
| P10079 | Fibropellin I precursor (Epidermal growth | 140 . . . 777 | 261/679 (38%) | e−146 |
| | factor-related protein 1) (UEGF-1) - | 249 . . . 895 | 339/679 (49%) | |
| | Strongylocentrotus purpuratus (Purple sea | | | |
| | urchin), 1064 aa. | | | |
| O16004 | NOTCH HOMOLOG - Lytechinus | 151 . . . 781 | 251/651 (38%) | e−137 |
| | variegatus (Sea urchin), 2531 aa. | 672 . . . 1290 | 330/651 (50%) | |
| A24420 | notch protein - fruit fly (Drosophila | 152 . . . 777 | 239/665 (35%) | e−136 |
| | melanogaster), 2703 aa. | 685 . . . 1334 | 343/665 (50%) | |

PFam analysis predicts that the NOV20a protein contains the domains shown in the Table 20F.

TABLE 20F
Domain Analysis of NOV20a

| | | Identities/ | |
|---|---|---|---|
| | NOV20a | Similarities for | Expect |
| Pfam Domain | Match Region | the Matched Region | Value |
| EGF: domain 1 of 16 | 147 . . . 183 | 16/47 (34%) | 3e−05 |
| | | 28/47 (60%) | |
| EGF: domain 2 of 16 | 190 . . . 221 | 15/47 (32%) | 6.9e−08 |
| | | 28/47 (60%) | |
| EGF: domain 3 of 16 | 228 . . . 259 | 13/47 (28%) | 1.4e−05 |
| | | 21/47 (45%) | |
| EGF: domain 4 of 16 | 266 . . . 297 | 17/47 (36%) | 1.5e−09 |
| | | 26/47 (55%) | |
| EGF: domain 5 of 16 | 308 . . . 339 | 18/47 (38%) | 2.8e−09 |
| | | 25/47 (53%) | |
| EGF: domain 6 of 16 | 343 . . . 374 | 12/47 (26%) | 2.2 |
| | | 19/47 (40%) | |
| EGF: domain 7 of 16 | 383 . . . 419 | 11/47 (23%) | 4.2 |
| | | 23/47 (49%) | |
| EGF: domain 8 of 16 | 420 . . . 451 | 17/47 (36%) | 4.2e−07 |
| | | 25/47 (53%) | |
| laminin_EGF: domain 1 | 404 . . . 464 | 15/68 (22%) | 5.8 |
| of 1 | | 40/68 (59%) | |
| EGF: domain 9 of 16 | 459 . . . 490 | 16/47 (34%) | 1.4e−05 |
| | | 26/47 (55%) | |
| EGF: domain 10 of 16 | 498 . . . 529 | 18/47 (38%) | 4.9e−09 |
| | | 29/47 (62%) | |
| EGF: domain 11 of 16 | 536 . . . 567 | 15/47 (32%) | 4.6e−06 |
| | | 22/47 (47%) | |
| sushi: domain 1 of 1 | 573 . . . 626 | 16/64 (25%) | 3.8e−05 |
| | | 36/64 (56%) | |
| EGF: domain 12 of 16 | 632 . . . 663 | 14/47 (30%) | 7.6e−07 |
| | | 21/47 (45%) | |
| EGF: domain 13 of 16 | 670 . . . 701 | 17/47 (36%) | 3.3e−07 |
| | | 23/47 (49%) | |
| EGF: domain 14 of 16 | 708 . . . 739 | 13/47 (28%) | 1.4e−05 |
| | | 25/47 (53%) | |
| EGF: domain 15 of 16 | 746 . . . 777 | 13/47 (28%) | 3.5e−05 |
| | | 26/47 (55%) | |
| fn3: domain 1 of 3 | 781 . . . 862 | 24/88 (27%) | 3.9e−08 |
| | | 60/88 (68%) | |
| fn3: domain 2 of 3 | 880 . . . 963 | 18/87 (21%) | 2e−09 |
| | | 62/87 (71%) | |
| fn3: domain 3 of 3 | 979 . . . 1061 | 27/86 (31%) | 3e−08 |
| | | 58/86 (67%) | |
| EGF: domain 16 of 16 | 1186 . . . 1217 | 17/47 (36%) | 4.1e−08 |
| | | 28/47 (60%) | |

### Example 21

The NOV21 clone was analyzed, and the nucleotide and encoded polypeptide sequences are shown in Table 21A.

TABLE 21A
NOV21 Sequence Analysis

| | |
|---|---|
| SEQ ID NO:83 | 1713 bp |

| | |
|---|---|
| NOV21, | AAACCCTGGGCAGTGGTGCCCAGCATCTTTCACAGGACACCGCGTGAGTGCAGATGGA |
| CG93702-01 DNA | |
| Sequence | GATCCACTGAGCACTCTGCTAGGGAGCAATTCATGGGGAGCACCCCTCCAGAGAGGGA |
| | TGGCTCGCACAGGCCCTCAGCCCAGCCCCTTGCAGGCTGGACCTTGGAGAGTGAGGCC |
| | CTGAGACGAGACATGGGCACCTGGCTTCTGGCCTGCACCTGCGTCTGCACCTGTGTCT |
| | GCTCGGGAGTCTCTGTCTCAGGGGATGGACGAGGTGGGCCAAGGGCTGGAACCTCCAC |
| | CTGCCTCACCAACAACATTCTCAGGATTGATTGCCACTGGTCTGCCCCAGAGCTGGGT |
| | CAGGGCTCCAGCCCCGGGCTCCCCTTCACAAGCAACCAGGCTGCTGGTGGCACACAGA |
| | AGTGCATCTGGCAGGGCAGTGAGTGCACTGTAGTGTTGCCGCCCAAGGCAGCACTCCT |
| | GCCATCTGACAATTTCATCATCACTTTCTACCACTGCATGTCCGGGAGGGATCAGGTC |
| | AGCCTGGTGGACCTGGAGTACCTGCCCTGGAGACACGGTGAACAGCAGCTATCTGACT |
| | TGCAGAGCACGTCAGCTCGCCACTGCATCCTGACCTGGAGCCTCAGTCCTGCCTTGGA |
| | GTCAATGACCACACTTCTCAGCTATGAGCTGGACTTCAAGAGGCAGGAAGAGGCCTGG |
| | GAGGTAACAGCCCAGCACAGGGATCACATTGTCGGGGTGACCTGGCTCATACTTGAAG |
| | CCTTTGAGCTGGACCCTGGCTTTATCCTTGAGGCCAGGCTGCGTGTCCAGACGGCCAT |
| | GCTGGGGGATGACGGGGCACAGGAGGAGCGAGGGAGGAGCGAGGGGAGCCAGCCCGTG |
| | TGCTTCCAGGCTCCCCAGAGACAAGGTCCTCTGATCCCACCCTGGGGGTGGCCAGGCA |
| | ACACCTTTGTTGCTGTGTCCATCTTTCTCCTGCTGACTGGCCCGACCTACCTCCTGTT |
| | CAAGCTGTCGCCCAGGGTGAAGAGAACCTTCTACCAGAATGTGCCCTCTCTAGCGGTG |
| | TTCTCCCAGCCCCTCTACGGTGTGCACAATGGGAACTTCCAGACTCGGATGGGGGCCC |
| | ACAGGGCTGGTGTGCTGCTGAGCCAGGACTGTGCTGGCACCCGACGAGGAGCCTTGGA |
| | GCCCTGCGTCCAGGAGGCCACTGCACTGTTCACCTGTGGCCCAGCGGGTCCTTGGAAA |
| | TCTGTGGGCCTGGAGGAGGAGCAGGAAGGGCCTGGAGCAGGAAGGCACTGGGACCTGA |
| | GCTCAGAGCATGTGCTGCCAGCAGGGTGTACGGAGTGGAGGGCACAGCCCCTTGCCTA |
| | TCTGCCACAGGAGGACTTGGCCCCCACGTCCACCAGGGCATGTTACTCCCTTCCGTCC |
| | TTAGCAAGGCTTGGTCCTAATCCCAGCACTTTGGGATGCCGAGGCGGGTGGCTTCTCC |
| | CACGGATCTTTGCAACCTGCAGATCAGGAGGTCCCCTGGTGAGCTCAGCCATGGCCTT |
| | GGGTCTGAAGCACAGAGCTGTGTGGAGTCTGGGCGGAATGCTCGCTGGCTCACTGGGG |
| | CCCCACGTCCACCAGGGCATGTTACTCCCTTCCGTCCTTAGCAAGGCTTGGTCCTGGA |
| | TGTCCTGAGTCCCTGACTTGCCAGATGAATCATGTCCATTTTGGGAAAGTGGACTTAA |
| | GTCTCCGGAGCCCTTGTCTGGGACTGAACCT |

| | | |
|---|---|---|
| ORF Start: ATG at 91 | | ORF Stop: TGA at 1630 |
| SEQ ID NO:84 | 513 aa | MW at 55570.7 kD |

| | |
|---|---|
| NOV21, | MGSTPPERDGSHRPSAQPLAGWTLESEALRRDMGTWLLACTCVCTCVCSGVSVSGDGR |
| CG93702-01 Protein | |
| Sequence | GGPRAGTSTCLTNNILRIDCHWSAPELGQGSSPGLPFTSNQAAGGTQKCIWQGSECTV |
| | VLPPKAALLPSDNFIITFYHCMSGRDQVSLVDLEYLPWRHGEQQLSDLQSTSARHCIL |
| | TWSLSPALESMTTLLSYELDFKRQEEAWEVTAQHRDHIVGVTWLILEAFELDPGFILE |
| | ARLRVQTAMLGDDGAQEERGRSEGSQPVCFQAPQRQGPLIPPWGWPGNTFVAVSIFLL |
| | LTGPTYLLFKLSPRVKRTFYQNVPSLAVFSQPLYGVHNGNFQTRMGAHRAGVLLSQDC |
| | AGTRRGALEPCVQEATALFTCGPAGPWKSVGLEEEQEGPGAGRHWDLSSEHVLPAGCT |
| | EWRAQPLAYLPQEDLAPTSTRACYSLPSLARLGPNPSTLGCRGGWLLPRIFATCRSGG |
| | PLVSSAMALGLKHRAVWSLGGMLAGSLGPHVHQGMLLPSVLSKAWSWMS |

Further analysis of the NOV21 protein yielded the following properties shown in Table 21B.

TABLE 21B
Protein Sequence Properties NOV21

| | |
|---|---|
| PSort | 0.6000 probability located in plasma membrane; 0.4000 |
| analysis: | probability located in Golgi body; 0.3000 probability located |
| | in endoplasmic reticulum (membrane); 0.3000 |
| | probability located in microbody (peroxisome) |
| SignalP | Cleavage site between residues 49 and 50 |
| analysis: | |

A search of the NOV21 protein against the Geneseq database, a proprietary database that contains sequences published in patents and patent publication, yielded several homologous proteins shown in Table 21C.

TABLE 21C
Geneseq Results for NOV21

| | | NOV21 | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAW64055 | Human IL-9 receptor protein - Homo | 33 . . . 511 | 361/501 (72%) | 0.0 |
| | sapiens, 501 aa. [WO9824904-A2, | 1 . . . 499 | 389/501 (77%) | |
| | 11-JUN-1998] | | | |
| AAW64057 | Human IL-9 receptor protein variant #2 - | 33 . . . 511 | 361/501 (72%) | 0.0 |
| | Homo sapiens, 500 aa. [WO9824904-A2, | 1 . . . 498 | 389/501 (77%) | |
| | 11-JUN-1998] | | | |
| AAW64056 | Human IL-9 receptor protein variant #1 - | 33 . . . 511 | 361/501 (72%) | 0.0 |
| | Homo sapiens, 501 aa. [WO9824904-A2, | 1 . . . 499 | 389/501 (77%) | |
| | 11-JUN-1998] | | | |
| AAW64058 | Human IL-9 receptor protein variant #3 - | 33 . . . 305 | 223/278 (80%) | e−124 |
| | Homo sapiens, 286 aa. [WO9824904-A2, | 1 . . . 276 | 239/278 (85%) | |
| | 11-JUN-1998] | | | |
| AAW64061 | Human IL-9 receptor protein variant | 33 . . . 188 | 107/156 (68%) | 1e−56 |
| | fragment #3 - Homo sapiens, 150 aa. | 1 . . . 141 | 119/156 (75%) | |
| | [WO9824904-A2, 11-JUN-1998] | | | |

In a BLAST search of public sequence databases, the NOV21 protein was found to have homology to the proteins shown in the BLASTP data in Table 21D.

TABLE 21D
Public BLASTP Results for NOV21

| | | NOV21 | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q01113 | Interleukin-9 receptor precursor | 21 . . . 511 | 373/513 (72%) | 0.0 |
| | (IL-9R) - Homo sapiens (Human), 522 | 10 . . . 520 | 401/513 (77%) | |
| | aa. | | | |
| Q96TF0 | INTERLEUKIN 9 RECEPTOR - | 21 . . . 511 | 372/512 (72%) | 0.0 |
| | Homo sapiens (Human), 521 aa. | 10 . . . 519 | 400/512 (77%) | |
| AAL55435 | INTERLEUKIN 9 RECEPTOR - | 21 . . . 511 | 372/513 (72%) | 0.0 |
| | Homo sapiens (Human), 522 aa. | 10 . . . 520 | 400/513 (77%) | |
| Q01114 | Interleukin-9 receptor precursor | 21 . . . 423 | 218/410 (53%) | e−106 |
| | (IL-9R) - Mus musculus (Mouse), 468 | 10 . . . 413 | 261/410 (63%) | |
| | aa. | | | |
| Q63216 | GFI-2 PROTEIN - Rattus norvegicus | 21 . . . 423 | 214/411 (52%) | 2e−98 |
| | (Rat), 467 aa. | 10 . . . 412 | 258/411 (62%) | |

PFam analysis predicts that the NOV21 protein contains the domains shown in the Table 21E.

TABLE 21E
Domain Analysis of NOV21

| | | | |
|---|---|---|---|
| | | Identities/ | |
| | NOV21 | Similarities for | |
| Pfam Domain | Match Region | the Matched Region | Expect Value |

No Significant Known Matches Found

### Example 22

The NOV22 clone was analyzed, and the nucleotide and encoded polypeptide sequences are shown in Table 22A.

TABLE 22A
NOV22 Sequence Analysis

| | |
|---|---|
| SEQ ID NO:85 | 2264 bp |

| | |
|---|---|
| NOV22, | CTGGGTAGGCCGGGACAAAAACACCGTACGTTCTCACTGCAGTCCATGGAAGAGGTAG |
| CG93792-01 DNA | |
| Sequence | CCCAGCCCCCAGGCTTCAGGTTGTCCTTAGCTTGAAGGTGGGGCTTCACCGGGGACCC |
| | ATCCCTTTTTGCCCATCTGCTCCCTGCCACCATTAACCTGCCATCTACCATGTCCATG |
| | GCACCCAGGCAGTTCGCGTCATGGGGCACCTTTCAGGCCTGTGCCAAGCTGCTCCCGG |
| | AATGGACTCTCTGGGAAGAATGCACAAGGAGCTGTGGACGCGGCAACCAAACCAGGAC |
| | CAGGACTTGCAATAATCCATCAGTTCAGCATGGTGGGCGGCCATGTGAAGGGAATGCT |
| | GTGGAAATAATTATGTGCAACATTAGGCCTTGCCCAGTTCATGGAGCATGGAGCGCTT |
| | GGCAGCCTTGGGGAACATGCAGCGAAAGTTGTGGGAAAGGTACTCAGACAAGAGCAAG |
| | ACTTTGTAATAACCCACCACCAGCGTTTGGTGGGTCCTACTGTGATGGAGCAGAAACA |
| | CAGATACAAGTTTGCAATGAAAGAAATTGTCCAATTCATGGCAAGTGGGCGACTTGGG |
| | CCAGTTGGAGTGCCTGTTCTGTGTCATGTGGAGGAGGTGCCAGACAGAGAACAAGGGG |
| | CTGCTCCGACCCTGTGCCCCAGTATGGAGGAAGGAAATGCGAAGGGAGTGATGTCCAG |
| | AGTGATTTTTGCAACAGTGACCCTTGCCCAACCCATGGTAACTGGAGTCCTTGGAGTG |
| | GCTGGGGAACATGCAGCCGGACGTGTAACGGAGGGCAGATGCGGCGGTACCGCACATG |
| | TGATAACCCTCCTCCCTCCAATGGGGGAAGAGCTTGTGGGGGACCAGACTCCCAGATC |
| | CAGAGGTGCAACACTGACATGTGTCCTGTGGATGGAAGTTGGGGAAGCTGGCATAGTT |
| | GGAGCCAGTGCTCTGCCTCCTGTGGAGGAGGTGAAAAGACTCGGAAGCGGCTGTGCGA |
| | CCATCCTGTGCCAGTTAAAGGTGGCCGTCCCTGTCCCGGAGACACTACTCAGGTGACC |
| | AGGTGCAATGTACAAGCATGTCCAGGTGGGCCCCAGCGAGCCAGAGGAAGTGTTATTG |
| | GAAATATTAATGATGTTGAATTTGGAATTGCTTTCCTTAATGCCACAATAACTGATAG |
| | CCCTAACTCTGATACTAGAATAATACGTGCCAAAATTACCAATGTACCTCGTAGTCTT |
| | GGTTCAGCAATGAGAAAGATAGTTTCTATTCTAAATCCCATTTATTGGACAACAGCAA |
| | AGGAAATAGGAGAAGCAGTCAATGGCTTTACCCTCACCAATGCAGTCTTCAAAAGAGA |
| | AACTCAAGTGGAATTTGCAACTGGAGAAATCTTGCAGATGAGTCATATTGCCCGGGGC |
| | TTGGATTCCGATGGTTCTTTGCTGCTAGATATCGTTGTGAGTGGCTATGTCCTACAGC |
| | TTCAGTCACCTGCTGAAGTCACTGTAAAGGATTACACAGAGGACTACATTCAAACAGG |
| | TCCTGGGCAGCTGTACGCCTACTCAACCCGGCTGTTCACCATTGATGGCATCAGCATC |
| | CCATACACATGGAACCACACCGTTTTCTATGATCAGGCACAGGGAAGAATGCCTTTCT |
| | TGGTTGAAACACTTCATGCATCCTCTGTGGAATCTGACTATAACCAGATAGAAGAGAC |
| | ACTGGGTTTTAAAATTCATGCTTCAATATCCAAAGGAGATCGCAGTAATCAGTGCCCC |
| | CCCGGGTTTACCTTAGACTCAGTTGGACCTTTTTGTGCTGATGAGGATGAATGTGCAG |
| | CAGGGAATCCCTGCTCCCATAGCTGCCACAATGCCATGGGGACTTACTACTGCTCCTG |
| | CCCTAAAGGCCTCACCATAGCTGCAGATGGAAGAACTTGTCAAGATATTGATGAGTGT |
| | GCTTTGGGTAGGCATACCTGCCACGCTGGTCAGGACTGTGACAATACGATTGGATCTT |
| | ATCGCTGTGTGGTCCGTTGTGGAAGTGGCTTTCGAAGAACCTCTGATGGGCTGAGTCG |
| | TCAAGGTATAAAAATGGAGGCCTTTTCTTTATGTTCATGACAGTAAGAATTAGACCCA |
| | CCTTTTGACTCCTCAAAAGTTAACTGTCTCAGAAACTCCACGAGGAAGGGACCACATA |
| | AAAGGGAGAGAATGAGGAGATATCCAGCAAGAGGGACTCCTGTCTCTCCGGAGGACTT |
| | AAACTTCATTTTATATGTTTTATAAGTTGAGCTTCTTCATAAGCTTTTATTCAGATAT |
| | AT |

| | | |
|---|---|---|
| ORF Start: ATG at 166 | | ORF Stop: TGA at 2068 |
| SEQ ID NO:86 | 634 aa | MW at 68742.1 kD |

| | |
|---|---|
| NOV22, | MSMAPRQFASWGTFQACAKLLPEWTLWEECTRSCGRGNQTRTRTCNNPSVQHGGRPCE |
| CG93792-01 Protein | |
| Sequence | GNAVEIIMCNIRPCPVHGAWSAWQPWGTCSESCGKGTQTRARLCNNFPPAFGGSYCDG |
| | AETQIQVCNERNCPIHGKWATWASWSACSVSCGGGARQRTRGCSDPVPQYGGRKCEGS |
| | DVQSDFCNSDPCPTHGNWSPWSGWGTCSRTCNGGQMRRYRTCDNPPPSNGGRACGGPD |
| | SQIQRCNTDMCPVDGSWGSWHSWSQCSASCGGGEKTRKRLCDHPVPVKGGRPCPGDTT |
| | QVTRCNVQACPGGPQRARGSVIGNINDVEFGIAFLNATITDSPNSDTRIIRAKITNVP |
| | RSLGSAMRKIVSILNPIYWTTAKEIGEAVNGFTLTNAVFKRETQVEFATGEILQMSHI |
| | ARGLDSDGSLLLDIVVSGYVLQLQSPAEVTVKDYTEDYIQTGPGQLYAYSTRLFTIDG |
| | ISIPYTWNHTVFYDQAQGRMPFLVETLHASSVESDYNQIEETLGFKIHASISKGDRSN |
| | QCPPGFTLDSVGPFCADEDECAAGNPCSHSCHNANGTYYCSCPKGLTIAADGRTCQDI |
| | DECALGRHTCHAGQDCDNTIGSYRCVVRCGSGFRRTSDGLSRQGIKMEAFSLCS |

Further analysis of the NOV22 protein yielded the following properties shown in Table 22B.

TABLE 22B
Protein Sequence Properties NOV22

| | |
|---|---|
| PSort | 0.4993 probability located in mitochondrial matrix space; |
| analysis: | 0.3000 probability located in microbody (peroxisome); 0.2177 |
| | probability located in mitochondrial inner membrane; 0.2177 |
| | probability located in mitochondrial intermembrane space |
| SignalP | Cleavage site between residues 19 and 20 |
| analysis: | |

A search of the NOV22 protein against the Geneseq database, a proprietary database that contains sequences published in patents and patent publication, yielded several homologous proteins shown in Table 22C.

TABLE 22C
Geneseq Results for NOV22

| | | NOV22 | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAB47771 | Human thrombospondin protein, | 23 . . . 625 | 598/603 (99%) | 0.0 |
| | BTL.012 - Homo sapiens, 1336 aa. | 718 . . . 1320 | 600/603 (99%) | |
| | [WO200174852-A2, 11-OCT-2001] | | | |
| AAG67244 | Amino acid sequence of murine | 23 . . . 625 | 525/603 (87%) | 0.0 |
| | thrombospondin 1-like protein - Mus | 141 . . . 743 | 569/603 (94%) | |
| | musculus, 1068 aa. [WO200109321-A1, | | | |
| | 08-FEB-2001] | | | |
| AAU16959 | Human novel secreted protein, SEQ ID | 76 . . . 625 | 546/550 (99%) | 0.0 |
| | 200 - Homo sapiens, 877 aa. | 3 . . . 552 | 547/550 (99%) | |
| | [WO200155441-A2, 02-AUG-2001] | | | |
| AAU17031 | Human novel secreted protein, SEQ ID | 76 . . . 625 | 544/550 (98%) | 0.0 |
| | 272 - Homo sapiens, 800 aa. | 12 . . . 561 | 545/550 (98%) | |
| | [WO200155441-A2, 02-AUG-2001] | | | |
| AAU18148 | Novel human uterine motility-association | 76 . . . 625 | 544/550 (98%) | 0.0 |
| | polypeptide #55 - Homo sapiens, 800 aa. | 12 . . . 561 | 545/550 (98%) | |
| | [WO200155201-A1, 02-AUG-2001] | | | |

In a BLAST search of public sequence databases, the NOV22 protein was found to have homology to the proteins shown in the BLASTP data in Table 22D.

TABLE 22D
Public BLASTP Results for NOV22

| | | NOV22 | Identities | |
|---|---|---|---|---|
| Protein | | Residues/ | Similarities for | |
| Accession | | Match | the Matched | Expect |
| Number | Protein/Organism/Length | Residues | Portion | Value |
| Q96RW7 | HEMICENTIN - Homo sapiens (Human), | 23 . . . 625 | 598/603 (99%) | 0.0 |
| | 5636 aa. | 4592 . . . 5194 | 600/603 (99%) | |
| Q96SC3 | FIBUL1N-6 - Homo sapiens (Human), 2673 | 23 . . . 625 | 597/603 (99%) | 0.0 |
| | aa (fragment). | 1629 . . . 2231 | 600/603 (99%) | |
| Q96K89 | CDNA FLJ14438 FIS, CLONE | 210 . . . 625 | 413/416 (99%) | 0.0 |
| | HEMBB1000317, WEAKLY SIMILAR TO | 1 . . . 416 | 413/416 (99%) | |
| | FIBULIN-1, ISOFORM D PRECURSOR - | | | |
| | Homo sapiens (Human), 741 aa. | | | |
| Q60519 | Semaphorin 5B precursor (Semaphorin G) | 24 . . . 303 | 122/305 (40%) | 7e−62 |
| | (Sema G) - Mus musculus (Mouse), 1093 aa. | 612 . . . 909 | 155/305 (50%) | |
| Q62217 | Semaphorin 5A precursor (Semaphorin F) | 24 . . . 301 | 117/302 (38%) | 2e−60 |
| | (Sema F) - Mus musculus (Mouse), 1077 aa. | 601 . . . 896 | 145/302 (47%) | |

PFam analysis predicts that the NOV22 protein contains the domains shown in the Table 22E.

TABLE 22E
Domain Analysis of NOV22

| | | Identities/ | |
|---|---|---|---|
| | NOV22 | Similarities for | Expect |
| Pfam Domain | Match Region | the Matched Region | Value |
| tsp_1: domain 1 of 5 | 22 . . . 72 | 23/54 (43%) | 3.6e−12 |
| | | 40/54 (74%) | |
| tsp_1: domain 2 of 5 | 79 . . . 129 | 22/54 (41%) | 6.8e−13 |
| | | 36/54 (67%) | |
| tsp_1: domain 3 of 5 | 136 . . . 186 | 23/54 (43%) | 1.9e−14 |
| | | 37/54 (69%) | |
| tsp_1: domain 4 of 5 | 193 . . . 243 | 23/54 (43%) | 9.8e−09 |
| | | 36/54 (67%) | |
| tsp_1: domain 5 of 5 | 250 . . . 300 | 23/54 (43%) | 6.7e−13 |
| | | 39/54 (72%) | |
| EGF: domain 1 of 2 | 543 . . . 577 | 16/47 (34%) | 8.4e−06 |
| | | 25/47 (53%) | |
| granulin: domain 1 of 1 | 564 . . . 579 | 7/16 (44%) | 4.2 |
| | | 11/16 (69%) | |
| TIL: domain 1 of 1 | 524 . . . 583 | 18/74 (24%) | 7.1 |
| | | 33/74 (45%) | |
| EGF: domain 2 of 2 | 583 . . . 622 | 13/48 (27%) | 23 |
| | | 24/48 (50%) | |

### Example 23

The NOV23 clone was analyzed, and the nucleotide and encoded polypeptide sequences are shown in Table 23A.

TABLE 23A
NOV23 Sequence Analysis

| | |
|---|---|
| SEQ ID NO:87 | 5935 bp |

| | |
|---|---|
| NOV23, | ATGGGTATGACCAAAAAGATAAGAGATAACAAGAGTCGGCAAGGATGTGGAGAAAAGG |
| CG94013-01 DNA | |
| Sequence | GGACACTTGCACACTGTTGGTGGGATTCCCCTAAAATGACCTGGATGAAAGATGGCCG |
| | GCCCCTTCCACAGACGGATCAAGTGCAAACTCTAGGAGGAGGAGAGGTTCTTCGAATT |
| | TCTACTGCTCAGGTGGAGGATACAGGAAGATATACATGTCTGGCATCCAGTCCTGCAG |
| | GAGATGATGATAAGGAATATCTAGTGAGAGTGCATGTACCTCCTAATATTGCTGGAAC |
| | TGATGAGCCCCGGGATATCACTGTGTTACGGAACAGACAAGTGACATTGGAATGCAAG |
| | TCAGATGCAGTGCCCCCACCTGTAATTACTTGGCTCAGAAATGGAGAACGGTTACAGG |
| | CAACACCTCGAGTGCGAATCCTATCTGGAGGGAGATACTTGCAAATCAACAATGCTGA |
| | CCTAGGTGATACAGCCAATTATACCTGTGTTGCCAGCAACATTGCAGGAAAGACTACA |
| | AGAGAATTTATTCTCACTGTAAATGTTCCTCCAAACATAAAGGGGGGCCCCCAGAGCC |
| | TTGTAATTCTTTTAAATAAGTCAACTGTATTGGAATGCATCGCTGAAGGTGTGCCAAC |
| | TCCAAGGATAACATGGAGAAAGGATGGAGCTGTTCTAGCTGGGAATCATGCAAGATAT |
| | TCCATCTTGGAAAATGGATTCCTTCATATTCAATCAGCACATGTCACTGACACTGGAC |
| | GGTATTTGTGTATGGCCACCAATGCTGCTGGAACAGATCGCAGGCGAATAGATTTACA |
| | GGTCCATGGTTCACTAGTAATTATTTCCCCTTCTGTGGATGACACTGCAACCTATGCC |
| | TGTACTGTGACAAACGGTGCTGGAGATGATAAAAGAACTGTGGATCTCACTGTCCAAG |
| | TTCCACCTTCCATAGCTGATGAGCCTACAGATTTCCTAGTAACCAAACATGCCCCAGC |
| | AGTAATTACCTGCACTGCTTCGGGAGTTCCATTTCCCTCAATTCACTGGACCAAAAAT |
| | GGTATAAGACTGCTTCCCAGGGGAGATGGCTATAGAATTCTGTCCTCAGGAGCAATTG |
| | AAATACTTGCCACCCAATTAAACCATGCTGGAAGATACACTTGTGTCGCTAGGAATGC |
| | GGCTGGCTCTGCACATCGACACGTGACCCTTCATGTTCATGAGCCTCCAGTCATTCAG |
| | CCCCAACCAAGTGAACTACACGTCATTCTGAACAATCCTATTTTATTACCATGTGAAG |
| | CAACAGGGACACCCAGTCCTTTCATTACTTGGCAAAAAGAAGGCATCAATGTTAACAC |
| | TTCAGGCAGAAACCATGCAGTTCTTCCTAGTGGCGGCTTACAGATCTCCAGAGCTGTC |
| | CGAGAGGATGCTGGCACTTACATGTGTGTGGCCCAGAACCCGGCTGGTACAGCCTTGG |
| | GCAAAATCAAGTTAAATGTCCAAGTTCCTCCAGTCATTAGCCCTCATCTAAAGGAATA |
| | TGTTATTGCTGTGGACAAGCCCATCACGTTATCCTGTGAAGCAGATGGCCTCCCTCCG |
| | CCTGACATTACATGGCATAAAGATGGGCGTGCAATTGTGGAATCTATCCGCCAGCGCG |
| | TCCTCAGCTCTGGCTCTCTGCAAATAGCATTTGTCCAGCCTGGTGATGCTGGCCATTA |
| | CACGTGCATGGCAGCCAATGTAGCAGGATCAAGCAGCACAAGCACCAAGCTCACCGTC |
| | CATGTACCACCCAGGATCAGAAGTACAGAAGGACACTACACGGTCAATGAGAATTCAC |
| | AAGCCATTCTTCCATGCGTAGCTGATGGAATCCCCACACCAGCAATTAACTGGAAAAA |
| | AGACAATGTTCTTTTAGCTAACTTGTTAGGAAAATACACTGCTGAACCATATGGAGAA |
| | CTCATTTTAGAAAATGTTGTGCTGGAGGATTCTGGCTTCTATACCTGTGTTGCTAACA |
| | ATGCTGCAGGTGAAGATACACACACTGTCAGCCTGACTGTGCATGTTCTCCCCACTTT |
| | TACTGAACTTCCTGGAGACGTGTCATTAAATAAAGGAGAACAGCTACGATTAAGCTGT |
| | AAAGCTACTGGTATTCCATTGCCCAAATTAACATGGACCTTCAATAACAATATTATTC |
| | CAGCCCACTTTGACAGTGTGAATGGACACAGTGAACTTGTTATTGAAAGAGTGTCAAA |
| | AGAGGATTCAGGTACTTATGTGTGCACCGCAGAGAACAGCGTTGGCTTTGTGAAGGCA |
| | ATTGGATTTGTTTATGTGAAAGAACCTCCAGTCTTCAAAGGTGATTATCCTTCTAACT |
| | GGATTGAACCACTTGGTGGGAATGCAATCCTGAATTGTGAGGTGAAAGGAGACCCCAC |
| | CCCAACCATCCAGTGGAACAGAAAGGGAGTGGATATTGAAATTAGCCACAGAATCCGG |
| | CAACTGGGCAATGGCTCCCTGGCCATCTATGGCACTGTTAATGAAGATGCCGGTGACT |
| | ATACATGTGTAGCTACCAATGAAGCTGGGGTGGTGGAGCGCAGCATGAGTCTGACTCT |
| | GCAAAGTCCTCCTATTATCACTCTTGAGCCAGTGGAAACTGTTATTAATGCTGGTGGC |
| | AAAATCATATTGAATTGTCAGGCAACTGGAGAGCCTCAACCAACCATTACATGGTCCC |
| | GTCAAGGGCACTCTATTTCCTGGGATGACCGGGTTAACGTGTTGTCCAACAACTCATT |
| | ATATATTGCTGATGCTCAGAAAGAAGATACCTCTGAATTTGAATGCGTTGCTCGAAAC |
| | TTAATGGGTTCTGTCCTTGTCAGAGTGCCAGTCATAGTCCAGGTTCATGGTGGATTTT |
| | CCCAGTGGTCTGCATGGAGAGCCTGCAGTGTCACCTGTGGAAAAGGCATCCAAAAGAG |
| | GATTTGGAAATGCGAAACTGTCAAAATAAGCCTTGTCCAGTGGATGGTCAGCTGGTCG |
| | CTGAATGGAGTCTTTGGGAAGAATGCATCATTTGTTATGTTTCATTTGGTTCAGTTTC |
| | AATTCTCTTAGACTTGGACCAGGACTTGCAATTATGCATCAGTTCAGCAGGAGTGGTC |
| | GTTTATGTTATAGGTGAATGCTTTGGTTTTAAACATACACGGTTCTGTGACTTGCAAC |
| | TGTCTTTTGGGGTGTTTGCCCAGTTCATGGAGCATGGAGCGCTTGGCAGCCTTGGGGA |
| | ACATGCAGCGAAAGTTGTGGGAAAGGTACTCAGACAAGAGCAAGACTTTGTAATAACC |
| | CACCACCAGCGTTTGGTGGGTCCTACTGTGATGGAGCAGAAACACAGATGCAAGTTTG |
| | CAATGAAAGAAATTGTCCAATTCATGGCAAGTGGGCGACTTGGGCCAGTTGGAGTGCC |
| | TGTTCTGTGTCATGTGGAGGAGGTGCCAGACAGAGAACAAGGGGCTGCTCCGACCCTG |
| | TGCCCCAGTATGGAGGAAGGAAATGCGAAGGGAGTGATGTCCAGAGTGATTTTTGCAA |
| | CAGTGACCCTTGCCCAAGTGAGTGTTGGAAATACCCATGGTAACTGGAGTCCTTGGAG |
| | TGGCTGGGGAACATGCAGCCGGACGTGTAACGGAGGGCAGATGCGGCGGTACCGCACA |
| | TGTGATAACCCTCCTCCCTCCAATGGGGGAAGAGCTTGTGGGGGACCAGACTCCCAGA |
| | TCCAGAGGTGCAACACTGACATGTGTCCTGTGGATGGAAGTTGGGGAAGCTGGCATAG |
| | TTGGAGCCAGTGCTCTGCCTCCTGTGGAGGAGGTGAAAAGACTCGGAAGCGGCTGTGC |
| | GACCATCCTGTGCCAGTTAAAGGTGGCCGTCCCTGTCCCGGAGACACTACTCAGGTGA |
| | CCAGGTGCAATGTACAAGCATGTCCAGGTGGGCCCCAGCGAGCCAGAGGAAGTGTTAT |
| | TGGAAATATTAATGATGTTGAATTTGGAATTGCTTTCCTTAATGCCACAATAACTGAT |
| | AGCCCTAACTCTGATACTAGAATAATACGTGCCAAAATTACCAATGTACCTCGTAGTC |
| | TTGGTTCAGCAATGAGAAAGATAGTTTCTATTCTAAATCCCATTTATTGGACAACAGC |
| | AAAGGAAATAGGAGAAGCAGTCAATGGCTTTACCCTCACCAATGCAGTCTTCAAAAGA |
| | GAAACTCAAGTGGAATTTGCAACTGGAGAAATCTTGCAGATGAGTCATATTGCCCGGG |
| | GCTTGGATTCCGATGGTTCTTTGCTGCTAGATATCGTTGTGAGTGGCTATGTCCTACA |
| | GCTTCAGTCACCTGCTGAAGTCACTGTAAAGGATTACACAGAGGACTACATTCAAACA |
| | GGTCCTGGGCAGCTGTACGCCTACTCAACCCGGCTGTTCACCATTGATGGCATCAGCA |
| | TCCCATACACATGGAACCACACCGTTTTCTATGATCAGGCACAGGGAAGAATGCCTTT |
| | CTTGGTTGAAACACTTCATGCATCCTCTGTGGAATCTGACTATAACCAGATAGAAGAG |
| | ACACTGGGTTTTAAAATTCATGCTTCAATATCCAAAGGAGATCGCAGTAATCAGTGCC |
| | CCTCCGGGTTTACCTTAGACTCAGTTGGACCTTTTTGTGCTGATGAGGATGAATGTGC |
| | AGCAGGGAATCCCTGCTCCCATAGCTGCCACAATGCCATGGGGACTTACTACTGCTCC |
| | TGCCCTAAAGGCCTCACCATAGCTGCAGATGGAAGAACTTGTCAAGATATTGATGAGT |
| | GTGCTTTGGGTAGGCATACCTGCCACGCTGGTCAGGACTGTGACAATACGATTGGATC |
| | TTATCGCTGTGTGGTCCGTTGTGGAAGTGGCTTTCGAAGAACCTCTGATGGGCTGAGT |
| | TGTCAAGATATTAATGAATGTCAAGAATCCAGCCCCTGTCACCAGCGCTGTTTCAATG |
| | CCATAGGAAGTTTCCATTGTGGATGTGAACCTGGGTATCAGCTCAAAGGCAGAAAATG |
| | CATGGATGTGAACGAGTGTAGACAAAATGTATGCAGACCAGATCAGCACTGTAAGAAC |
| | ACCCGTGGTGGCTATAAGTGCATTGATCTTTGTCCAAATGGAATGACCAAGGCAGAAA |
| | ATGGAACCTGTATTGATATTGATGAATGTAAAGATGGGACCCATCAGTGCAGATATAA |
| | CCAGATATGTGAGAATACAAGAGGCAGCTATCGTTGTGTATGCCCAAGAGGTTATCGG |
| | TCTCAAGGAGTTGGAAGACCCTGCATGGATATTGATGAATGTGAAAATACAGATGCCT |
| | GCCAGCATGAGTGTAAGAATACCTTTGGAAGTTATCAGTGCATCTGCCCACCTGGCTA |
| | TCAACTCACACACAATGGAAAGACATGCCAAGATATCGATGAATGTCTGGAGCAGAAT |
| | GTGCACTGTGGACCCAATCGCATGTGCTTCAACATGAGAGGAAGCTACCAGTGCATCG |
| | ATACACCCTGTCCACCCAACTACCAACGGGATCCTGTTTCAGGGTTCTGCCTCAAGAA |
| | CTGTCCACCCAATGATTTGGAATGTGCCTTGAGCCCATATGCCTTGGAATACAAACTC |
| | GTCTCCCTCCCATTTGGAATAGCCACCAATCAAGATTTAATCCGGCTGGTTGCATACA |
| | CACAGGATGGAGTGATGCATCCCAGGACAACTTTCCTCATGGTAGATGAGGAACAGAC |
| | TGTTCCTTTTGCCTTGAGGGATGaAAACCTGAAAGGAGTGGTGTATACAACACGACCA |
| | CTACGAGAAGCAGAGACCTACCGCATGAGGGTCCGAGCCTCATCCTACAGTGCCAATG |
| | GGACCATTGAATATCAGACCACATTCATAGTTTATATAGCTGTGTCCGCCTATCCATA |
| | CTAAGGAACTCTCCAAAGCCTATTCCACATATTTAAACCGCATTAATCATGGCAATCA |
| | AGCCCCCTTCCAGATTACT |

| | | |
|---|---|---|
| ORF Start: ATG at 1 | | ORF Stop: TAA at 5860 |
| SEQ ID NO:88 | 1953 aa | MW at 213066.1kD |

| | |
|---|---|
| NOV23, | MGMTKKIRDNKSRQGCGEKGTLAHCWWDSPKMTWMKDGRPLPQTDQVQTLGGGEVLRI |
| CG94013-01 Protein | |
| Sequence | STAQVEDTGRYTCLASSPAGDDDKEYLVRVHVPPNIAGTDEPRDITVLRNRQVTLECK |
| | SDAVPPPVITWLRNGERLQATPRVRILSGGRYLQINNADLGDTANYTCVASNIAGKTT |
| | REFILTVNVPPNIKGGPQSLVILLNKSTVLECIAEGVPTPRITWRKDGAVLAGNHARY |
| | SILENGFLHIQSAHVTDTGRYLCMATNAAGTDRRRIDLQVHGSLVIISPSVDDTATYE |
| | CTVTNGAGDDKRTVDLTVQVPPSIADEPTDFLVTKHAPAVITCTASGVPFPSIHWTKN |
| | GIRLLPRGDGYRILSSGAIEILATQLNHAGRYTCVARNAAGSAHRHVTLHVHEPPVIQ |
| | PQPSELHVILNNPILLPCEATGTPSPFITWQKEGINVNTSGRNHAVLPSGGLQISRAV |
| | REDAGTYMCVAQNPAGTALGKIKLNVQVPPVISPHLKEYVIAVDKPITLSCEADGLPP |
| | PDITWHKDGRAIVESIRQRVLSSGSLQIAFVQPGDAGHYTCMAANVAGSSSTSTKLTV |
| | HVPPRIRSTEGHYTVNENSQAILPCVADGIPTPAINWKKDNVLLANLLGKYTAEPYGE |
| | LILENVVLEDSGFYTCVANNAAGEDTHTVSLTVHVLPTFTELPGDVSLNKGEQLRLSC |
| | KATGIPLPKLTWTFNNNIIPAHFDSVNGHSELVIERVSKEDSGTYVCTAENSVGFVKA |
| | IGFVYVKEPPVFKGDYPSNWIEPLGGNAILNCEVKGDPTPTIQWNRKGVDIEISHRIR |
| | QLGNGSLAIYGTVNEDAGDYTCVATNEAGVVERSMSLTLQSPPIITLEPVETVINAGG |
| | KIILNCQATGEPQPTITWSRQGHSISWDDRVNVLSNNSLYIADAQKEDTSEFECVARN |
| | LMGSVLVRVPVIVQVHGGFSQWSAWRACSVTCGKGIQKRSRLCNQPLPANGGKPCQGS |
| | DLEMRNCQNKPCPVDGQLVAEWSLWEECIICYVSFGSVSILLDLDQDLQLCISSAGVV |
| | VYVIGECFGFKHTRFCDLQLSFGVFAQFMEHGALGSLGEHAAKVVGKVLRQEQDFVIT |
| | HHQRLVGPTVMEQKHRCKFAMKEIVQFMASGRLGPVGVPVLCHVEEVPDREQGAAPTL |
| | CPSMEEGNAKGVMSRVIFATVTLAQVSVGNTHGNWSPWSGWGTCSRTCNGGQMRRYRT |
| | CDNPPPSNGGRACGGPDSQIQRCNTDMCPVDGSWGSWHSWSQCSASCGGGEKTRKRLC |
| | DHPVPVKGGRPCPGDTTQVTRCNVQACPGGPQRARGSVIGNINDVEFGIAFLNATITD |
| | SPNSDTRIIRAKITNVPRSLGSAMRKIVSILNPIYWTTAKEIGEAVNGFTLTNAVFKR |
| | ETQVEFATGEILQMSHIARGLDSDGSLLLDIVVSGYVLQLQSPAEVTVKDYTEDYIQT |
| | GPGQLYAYSTRLFTIDGISIPYTWNHTVFYDQAQGRMPFLVETLHASSVESDYNQIEE |
| | TLGFKIHASISKGDRSNQCPSGFTLDSVGPFCADEDECAAGNPCSHSCHNAMGTYYCS |
| | CPKGLTIAADGRTCQDIDECALGRHTCHAGQDCDNTIGSYRCVVRCGSGFRRTSDGLS |
| | CQDINECQESSPCHQRCFNAIGSFHCGCEPGYQLKGRKCMDVNECRQNVCRPDQHCKN |
| | TRGGYKCIDLCPNGMTKAENGTCIDIDECKDGTHQCRYNQICENTRGSYRCVCPRGYR |
| | SQGVGRPCMDIDECENTDACQHECKNTFGSYQCICPPGYQLTHNGKTCQDIDECLEQN |
| | VHCGPNRMCFNMRGSYQCIDTPCPPNYQRDPVSGFCLKNCPPNDLECALSPYALEYKL |
| | VSLPFGIATNQDLIRLVAYTQDGVMHPRTTFLMVDEEQTVPFALRDENLKGVVYTTRP |
| | LREAETYRMRVRASSYSANGTIEYQTTFIVYIAVSAYPY |

Further analysis of the NOV23 protein yielded the following properties shown in Table 23B.

TABLE 23B
Protein Sequence Properties NOV23

| | |
|---|---|
| PSort | 0.6000 probability located in plasma membrane; 0.4000 |
| analysis: | probability located in Golgi body; 0.3000 probability located |
| | in endoplasmic reticulum (membrane); 0.3000 |
| | probability located in microbody (peroxisome) |
| SignalP | No Known Signal Sequence Predicted |
| analysis: | |

A search of the NOV23 protein against the Geneseq database, a proprietary database that contains sequences published in patents and patent publication, yielded several homologous proteins shown in Table 23C.

TABLE 23C
Geneseq Results for NOV23

| | | NOV23 | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAU16959 | Human novel secreted protein, SEQ ID | 1191 . . . 1953 | 763/763 (100%) | 0.0 |
| | 200 - Homo sapiens, 877 aa. | 115 . . . 877 | 763/763 (100%) | |
| | [WO200155441-A2, 02-AUG-2001] | | | |
| AAG67241 | Amino acid sequence of human | 1191 . . . 1953 | 762/763 (99%) | 0.0 |
| | thrombospondin 1-like protein - Homo | 18 . . . 780 | 762/763 (99%) | |
| | sapiens, 780 aa. [WO200109321-A1, | | | |
| | 08-FEB-2001] | | | |
| AAB95002 | Human protein sequence SEQ ID | 1213 . . . 1953 | 741/741 (100%) | 0.0 |
| | NO: 16644 - Homo sapiens, 741 aa. | 1 . . . 741 | 741/741 (100%) | |
| | [EP1074617-A2, 07-FEB-2001] | | | |
| AAG67244 | Amino acid sequence of murine | 1191 . . . 1953 | 695/763 (91%) | 0.0 |
| | thrombospondin 1-like protein - Mus | 306 . . . 1068 | 729/763 (95%) | |
| | musculus, 1068 aa. [WO200109321-A1, | | | |
| | 08-FEB-2001] | | | |
| AAG67243 | Amino acid sequence of murine | 1210 . . . 1953 | 676/744 (90%) | 0.0 |
| | thrombospondin 1-like protein - Mus | 1 . . . 744 | 710/744 (94%) | |
| | musculus, 744 aa. [WO200109321-A1, | | | |
| | 08-FEB-2001] | | | |

In a BLAST search of public sequence databases, the NOV23 protein was found to have homology to the proteins shown in the BLASTP data in Table 23D.

TABLE 23D
Public BLASTP Results for NOV23

| | | NOV23 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96RW7 | HEMICENTIN - Homo sapiens (Human), | 29 . . . 1014 | 967/1043 (92%) | 0.0 |
| | 5636 aa. | 3558 . . . 4599 | 972/1043 (92%) | |
| Q96SC3 | FIBULIN-6 - Homo sapiens (Human), 2673 aa | 29 . . . 1014 | 966/1043 (92%) | 0.0 |
| | (fragment). | 595 . . . 1636 | 972/1043 (92%) | |
| Q96K89 | CDNA FLJ14438 FIS, CLONE | 1213 . . . 1953 | 741/741 (100%) | 0.0 |
| | HEMBB1000317, WEAKLY SIMILAR TO | 1 . . . 741 | 741/741 (100%) | |
| | FIBULIN-1, ISOFORM D PRECURSOR - | | | |
| | Homo sapiens (Human), 741 aa. | | | |
| Q96DN3 | CDNA FLJ31995 FIS, CLONE | 5 . . . 931 | 295/951 (31%) | e−130 |
| | NT2RP7009236, WEAKLY SIMILAR TO | 348 . . . 1252 | 460/951 (48%) | |
| | BASEMENT MEMBRANE-SPECIFIC | | | |
| | HEPARAN SULFATE PROTEOGLYCAN | | | |
| | CORE PROTEIN PRECURSOR - Homo | | | |
| | sapiens (Human), 1252 aa (fragment). | | | |
| T20992 | hypothetical protein F15G9.4a - | 10 . . . 982 | 297/1059 (28%) | e−106 |
| | Caenorhabditis elegans, 5175 aa. | 2494 . . . 3521 | 458/1059 (43%) | |

PFam analysis predicts that the NOV23 protein contains the domains shown in the Table 23E.

TABLE 23E
Domain Analysis of NOV23

| | | Identities/ | |
|---|---|---|---|
| | | Similarities | |
| Pfam Domain | NOV23 Match Region | for the Matched Region | Expect Value |
| ig: domain 1 of 12 | 28 . . . 73 | 12/47 (26%) | 2e−05 |
| | | 38/47 (81%) | |
| ig: domain 2 of 12 | 108 . . . 166 | 19/62 (31%) | 1.2e−08 |
| | | 43/62 (69%) | |
| ig: domain 3 of 12 | 199 . . . 257 | 16/62 (26%) | 8.4e−08 |
| | | 37/62 (60%) | |
| ig: domain 4 of 12 | 275 . . . 293 | 9/20 (45%) | 0.033 |
| | | 15/20 (75%) | |
| ig: domain 5 of 12 | 326 . . . 384 | 15/62 (24%) | 1.5e−08 |
| | | 43/62 (69%) | |
| ig: domain 6 of 12 | 417 . . . 475 | 17/62 (27%) | 1.6e−09 |
| | | 47/62 (76%) | |
| FmdA_AmdA: domain 1 of 1 | 264 . . . 494 | 60/422 (14%) | 6.5 |
| | | 145/422 (34%) | |
| ig: domain 7 of 12 | 508 . . . 565 | 19/61 (31%) | 1.1e−10 |
| | | 43/61 (70%) | |
| ig: domain 8 of 12 | 598 . . . 656 | 16/62 (26%) | 1e−08 |
| | | 39/62 (63%) | |
| ig: domain 9 of 12 | 689 . . . 745 | 20/60 (33%) | 9.5e−12 |
| | | 43/60 (72%) | |
| ig: domain 10 of 12 | 779 . . . 836 | 20/61 (33%) | 2.7e−10 |
| | | 42/61 (69%) | |
| Marek_A: domain 1 of 1 | 846 . . . 869 | 7/25 (28%) | 8 |
| | | 16/25 (64%) | |
| ig: domain 11 of 12 | 869 . . . 926 | 17/61 (28%) | 1.6e−09 |
| | | 42/61 (69%) | |
| tsp_1: domain 1 of 3 | 948 . . . 998 | 28/54 (52%) | 1.1e−16 |
| | | 37/54 (69%) | |
| tsp_1: domain 2 of 3 | 1196 . . . 1246 | 23/54 (43%) | 9.8e−09 |
| | | 36/54 (67%) | |
| tsp_1: domain 3 of 3 | 1253 . . . 1303 | 23/54 (43%) | 6.7e−13 |
| | | 39/54 (72%) | |
| EGF: domain 1 of 7 | 1546 . . . 1580 | 16/47 (34%) | 8.4e−06 |
| | | 25/47 (53%) | |
| granulin: domain 1 of 1 | 1567 . . . 1582 | 7/16 (44%) | 4.2 |
| | | 11/16 (69%) | |
| ig: domain 12 of 12 | 1604 . . . 1610 | 5/7 (71%) | 54 |
| | | 6/7 (86%) | |
| EGF: domain 2 of 7 | 1586 . . . 1625 | 14/48 (29%) | 2 |
| | | 25/48 (52%) | |
| EGF: domain 3 of 7 | 1631 . . . 1663 | 12/47 (26%) | 0.0045 |
| | | 24/47 (51%) | |
| EGF: domain 4 of 7 | 1669 . . . 1705 | 14/47 (30%) | 13 |
| | | 24/47 (51%) | |
| TILa: domain 1 of 1 | 1679 . . . 1734 | 20/62 (32%) | 7.7 |
| | | 32/62 (52%) | |
| Keratin_B2: domain 1 of 1 | 1595 . . . 1737 | 34/191 (18%) | 8.7 |
| | | 70/191 (37%) | |
| EGF: domain 5 of 7 | 1711 . . . 1748 | 14/47 (30%) | 0.0013 |
| | | 28/47 (60%) | |
| EGF: domain 6 of 7 | 1754 . . . 1788 | 17/47 (36%) | 1.3e−07 |
| | | 28/47 (60%) | |
| fn2: domain 1 of 1 | 1823 . . . 1834 | 7/12 (58%) | 7.8 |
| | | 8/12 (67%) | |
| EGF: domain 7 of 7 | 1794 . . . 1834 | 13/49 (27%) | 17 |
| | | 26/49 (53%) | |
| cadherin: domain 1 of 1 | 1855 . . . 1947 | 15/107 (14%) | 5.2 |
| | | 54/107 (50%) | |

### Example 24

The NOV24 clone was analyzed, and the nucleotide and encoded polypeptide sequences are shown in Table 24A.

TABLE 24A
NOV24 Sequence Analysis

| | |
|---|---|
| SEQ ID NO:89 | 1767 bp |

| | |
|---|---|
| NOV24, | ATGTGGCTCCCTGCTCTTGTCCTGGCCACTCTCGCTGCTTCCGCGGCTTGGGGTCATC |
| CG94442-01 DNA | |
| Sequence | GGTCCTCGCCACTTTTGGTGAACACCTTGCATGGCAAAGTGTTGGGCAAGTTCGTCAG |
| | CTTAGAAGGATTTGCACAGCCTGTGGCCGTTTTCTTGGGAATCCCTTTTGCCAAGCCG |
| | CCTCTTGGACCCCTGAGGTTTACTCTACCACAGCCTGCAGAGCCATGGAACTTTGTGA |
| | AGAATGCCACCTCGTACCCTCCTATGTGCACCCAAGATCCCAAGGTAGGGCAGTTTCT |
| | CTCAGAACTATTGACCAACCGAAAGGAGAACATTCCTTTCAAGCTTTCTGAAGACTGT |
| | CTTTACCTCAATATTTACACTCCTGCTGACTTGACCAAGAAAAACAGGCTGCTGGTAA |
| | TGGTGTGGATCCACGGAGGGGGGCTGATGGTGGGTGCGGCATCAACCTACGATGGGCT |
| | GGCCCTTGCTGCCCATGAAAACGTGGTGGTGGTGACCATTCAATATCGCCTGGGCATC |
| | TGGGGATTCTTCTCCCTCGCTGACAGTCACTCTAGAGGATCCTGGGGGCCAATGGGGC |
| | TTACGTATTTAATCTCAGAAAGGACGGCATCGTTTAGTGGATCAACAGGAAGCGTTTC |
| | GCCATTCGGCTCCGGCGGGAAACGGGTGTGTACTGTGGTGTGCTTACCACTGGCCAGA |
| | TCTTCATCGATGATCTCACGGATTTCTGAGAGTGATGTGGCCCTCACTCCTGCTCTGG |
| | TGGAGAAGGGTGACGTCAAGCCCCTGGCTGAGCAAATTGCTAACACTGTTGGGTGTGA |
| | AACCACCAACTCAGCTGTCATGGCTCACTGTCTGCGGCAGAAGATGGAAGAGGAGCTC |
| | TTGGAGACGACATTGAAAATGAAATTCTTATCTCTGGACTTACAGGGAGACCTCAAAG |
| | AGAGTCACCACTATTTGGCCACCGTGATTGATGGGGTGGTGCTGCTGAAAACACCTGA |
| | AAGAGCTTCAAGCTGAAGGAAGTTCCACACTGTCCCCTACATGGTCGGAATTAACAAG |
| | CAGGAGTTTGGCTGGATGCTTCCAATGCAGTTGATGAGCTATCTACTCTCCGAAGGGA |
| | AACTGGACCAGAAGACAGCCATGTCACTCTTCTGGAAGTCCTATCCCTTTGTTGTAAT |
| | TCCTAAGGAATTGATTCCAGAAGCCATTGAGAAGTACTTAGGAGGAACAGATGACCCT |
| | GTCAAGAAGAAAGACCTGTTCCTGGACTTAATGGGGGACGTACTGTTCGGTGTCCCAT |
| | CTGTGACTGTGGCCCGGAACCACAGAGATGCTGGAGCACCCACCTACATGTATGAGTT |
| | TCAGTACCGTCCAAGCTTCTCATCAGACATGAAACCCAAGACGGTGATAGGAGACCAC |
| | GGGGATGAGCTCTTCTCCGTCCTTGGGGCCCCATCTTTAAAAGAGGGTGCCTCAGAAG |
| | AGGAGATCAGACTTAGCAAGATGGTGATGAAATTCTGGGCCAACTTTGCTCGCAATGG |
| | GAACCCCAATGGAGAAGGGCTGCCGCACTGGCCAGAGTACAACCAGGAGGAAGGGTAC |
| | CTGCAGATTGGTGCTAACACCCAGGCAGCCCAGAAGCTGAAGGACAAGGAAGTAGCTT |
| | TCTGGACCAAACTCTTCGCCAAGAAGGCAGTGGAGAAGCCACCCCAGATAGAACTAAG |
| | CCATGGAGCTGACTGCCTTCGCGCTTATCCCTATGTACATCAAGAAAACTGAGGCCAA |
| | AAGGGTTTAGGTACTAATTTAGGTCCC |

| | | |
|---|---|---|
| ORF Start: ATG at 1 | | ORF Stop: TGA at 1732 |
| SEQ ID NO:90 | 577 aa | MW at 63826.1 kD |

| | |
|---|---|
| NOV24, | MWLPALVLATLAASAAWGHRSSPLLVNTLHGKVLGKFVSLEGFAQPVAVFLGIPFAKP |
| CG94442-01 Protein | |
| Sequence | PLGPLRFTLPQPAEPWNFVKNATSYPPMCTQDPKVGQFLSELLTNRKENIPFKLSEDC |
| | LYLNIYTPADLTKKNRLLVMVWIHGGGLMVGAASTYDGLALAAHENVVVVTIQYRLGI |
| | WGFFSLADSHSRGSWGPMGLTYLISERTASFSGSTGSVSPFGSGGKRVCTVVCLPLAR |
| | SSSMISRISESDVALTPALVEKGDVKPLAEQIANTVGCETTNSAVMAHCLRQKMEEEL |
| | LETTLKMKFLSLDLQGDLKESHHYLATVIDGVVLLKTPEELQAERKFHTVPYMVGINK |
| | QEFGWMLPMQLMSYLLSEGKLDQKTAMSLFWKSYPFVVIPKELIPEAIEKYLGGTDDP |
| | VKKKDLFLDLMGDVLFGVPSVTVARNHRDAGAPTYMYEFQYRPSFSSDMKPKTVIGDH |
| | GDELFSVLGAPSLKEGASEEEIRLSKMVMKFWANFARNGNPNGEGLPHWPEYNQEEGY |
| | LQIGANTQAAQKLKDKEVAFWTKLFAKKAVEKPPQIELSHGADCLRAYPYVHQEN |

Further analysis of the NOV24 protein yielded the following properties shown in Table 24B.

TABLE 24B
Protein Sequence Properties NOV24

| | |
|---|---|
| PSort | 0.5278 probability located in outside; 0.1022 probability |
| analysis: | located in microbody (peroxisome); 0.1000 probability located |
| | in endoplasmic reticulum (membrane); 0.1000 |
| | probability located in endoplasmic reticulum (lumen) |
| SignalP | Cleavage site between residues 19 and 20 |
| analysis: | |

A search of the NOV24 protein against the Geneseq database, a proprietary database that contains sequences published in patents and patent publication, yielded several homologous proteins shown in Table 24C.

TABLE 24C
Geneseq Results for NOV24

| | | NOV24 | Identities/ | |
|---|---|---|---|---|
| | | Residues/ | Similarities for | |
| Geneseq | | Match | the Matched | Expect |
| Identifier | Protein/Organism/Length [Patent #, Date] | Residues | Region | Value |
| AAB43732 | Human cancer associated protein sequence | 1 . . . 559 | 467/565 (82%) | 0.0 |
| | SEQ ID NO: 1177 - Homo sapiens, 583 | 16 . . . 579 | 496/565 (87%) | |
| | aa. [WO200055350-A1, 21-SEP-2000] | | | |
| AAB73263 | Human triacylglycerol hydrolase, TGH - | 1 . . . 559 | 464/564 (82%) | 0.0 |
| | Homo sapiens, 566 aa. | 1 . . . 562 | 493/564 (87%) | |
| | [WO200116358-A2, 08-MAR-2001] | | | |
| AAY33145 | Rabbit liver carboxylesterase protein - | 1 . . . 559 | 400/564 (70%) | 0.0 |
| | Oryctolagus cuniculus, 565 aa. | 1 . . . 561 | 461/564 (80%) | |
| | [WO9942593-A1, 26-AUG-1999] | | | |
| AAB08202 | Amino acid sequence of a rabbit liver | 6 . . . 559 | 394/559 (70%) | 0.0 |
| | esterase 3 designated RLE-3 - Oryctolagus | 7 . . . 562 | 454/559 (80%) | |
| | cuniculus, 566 aa. [US6107549-A, | | | |
| | 22-AUG-2000] | | | |
| AAY33146 | Rabbit liver carboxylesterase protein | 1 . . . 540 | 390/545 (71%) | 0.0 |
| | fragment - Oryctolagus cuniculus, 543 aa. | 1 . . . 543 | 446/545 (81%) | |
| | [WO9942593-A1, 26-AUG-1999] | | | |

In a BLAST search of public sequence databases, the NOV24 protein was found to have homology to the proteins shown in the BLASTP data in Table 24D.

TABLE 24D
Public BLASTP Results for NOV24

| | | NOV24 | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96EE8 | UNKNOWN (PROTEIN FOR MGC: 9220) - | 1 . . . 559 | 470/564 (83%) | 0.0 |
| | Homo sapiens (Human), 566 aa. | 1 . . . 562 | 497/564 (87%) | |
| P23141 | Liver carboxylesterase precursor (EC 3.1.1.1) | 1 . . . 559 | 467/564 (82%) | 0.0 |
| | (Acyl coenzyme A: cholesterol acyltransferase) | 1 . . . 563 | 496/564 (87%) | |
| | (ACAT) (Monocyte/macrophage serine | | | |
| | esterase) (HMSE) (Serine esterase 1) - Homo | | | |
| | sapiens (Human), 567 aa. | | | |
| Q9UK77 | EGASYN - Homo sapiens (Human), 567 aa. | 1 . . . 559 | 466/564 (82%) | 0.0 |
| | | 1 . . . 563 | 495/564 (87%) | |
| O46421 | CARBOXYLESTERASE PRECURSOR (EC | 1 . . . 559 | 455/564 (80%) | 0.0 |
| | 3.1.1.1) - Macaca fascicularis (Crab eating | 1 . . . 562 | 484/564 (85%) | |
| | macaque) (Cynomolgus monkey), 566 aa. | | | |
| O77540 | LIVER CARBOXYLESTERASE (EC 3.1.1.1) - | 1 . . . 559 | 400/564 (70%) | 0.0 |
| | Oryctolagus cuniculus (Rabbit), 565 aa. | 1 . . . 561 | 461/564 (80%) | |

PFam analysis predicts that the NOV24 protein contains the domains shown in the Table 24E.

TABLE 24E
Domain Analysis of NOV24

| | | Identities/ | |
|---|---|---|---|
| | NOV24 | Similarities for | |
| Pfam Domain | Match Region | the Matched Region | Expect Value |
| COesterase: | 1 . . . 184 | 89/205 (43%) | 2.7e−80 |
| domain 1 of 2 | | 162/205 (79%) | |
| G6PD_C: | 187 . . . 208 | 6/22 (27%) | 4.3 |
| domain 1 of 1 | | 15/22 (68%) | |
| COesterase: | 240 . . . 543 | 112/347 (32%) | 6e−90 |
| domain 2 of 2 | | 257/347 (74%) | |

### Example 25

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology:

The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E.coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example 26

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraToolsm program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOV1 SNP Data:

NOV1 has two SNP variants, whose variant positions for their nucleotide and amino acid sequences are numbered according to SEQ ID NOs:1 and 2, respectively. The nucleotide sequences of the NOV1 variants differ as shown in Table 26A.

TABLE 26A
SNP data for NOV1

| | Nucleotides | | | Amino Acids | | |
|---|---|---|---|---|---|---|
| Variant | Position | Initial | Modified | Position | Initial | Modified |
| 13374666 | 221 | C | T | 74 | Pro | Leu |
| 13374665 | 353 | T | C | 118 | Val | Ala |

NOV2a SNP Data:

NOV2a has four SNP variants, whose variant positions for their nucleotide and amino acid sequences are numbered according to SEQ ID NOs:3 and 4, respectively. The nucleotide sequences of the NOV2a variants differ as shown in Table 26B.

TABLE 26B
SNP data for NOV2a

| | Nucleotides | | | Amino Acids | | |
|---|---|---|---|---|---|---|
| Variant | Position | Initial | Modified | Position | Initial | Modified |
| 13374586 | 228 | T | C | 43 | Leu | Pro |
| 13374587 | 470 | A | T | 124 | Thr | Ser |
| 13374588 | 480 | C | A | 127 | Ser | Tyr |
| 13374590 | 798 | G | C | 233 | Arg | Thr |

NOV4 SNP data:

NOV4 has one SNP variant, whose variant positions for its nucleotide and amino acid sequences is numbered according to SEQ ID NOs:9 and 10, respectively. The nucleotide sequence of the NOV4 variant differs as shown in Table 26C.

TABLE 26C
SNP data for NOV4

| | Nucleotides | | | Amino Acids | | |
|---|---|---|---|---|---|---|
| Variant | Position | Initial | Modified | Position | Initial | Modified |
| 13377694 | 1929 | C | T | 616 | Thr | Ile |

NOV5 SNP Data:

NOV5 has six SNP variants, whose variant positions for their nucleotide and amino acid sequences are numbered according to SEQ ID NOs: 11 and 12, respectively. The nucleotide sequences of the NOV5 variants differ as shown in Table 26D.

TABLE 26D
SNP data for NOV5

| | Nucleotides | | | Amino Acids | | |
|---|---|---|---|---|---|---|
| Variant | Position | Initial | Modified | Position | Initial | Modified |
| 13377696 | 88 | G | A | 30 | Glu | Lys |
| 13377697 | 117 | G | A | 39 | Gln | Gln |
| 13377700 | 265 | C | A | 89 | Leu | Ile |
| 13377701 | 290 | A | G | 97 | Asp | Gly |
| 13377702 | 407 | T | C | 136 | Ile | Thr |
| 13377703 | 500 | G | C | 167 | Trp | Ser |

NOV6 SNP Data:

NOV6 has three SNP variants, whose variant positions for their nucleotide and amino acid sequences are numbered according to SEQ ID NOs: 13 and 14, respectively. The nucleotide sequences of the NOV6 variants differ as shown in Table 26E.

TABLE 26E
SNP data for NOV6

| | Nucleotides | | | Amino Acids | | |
|---|---|---|---|---|---|---|
| Variant | Position | Initial | Modified | Position | Initial | Modified |
| 13377705 | 169 | T | C | 53 | Ile | Ile |
| 13377706 | 338 | T | C | 110 | Ser | Pro |
| 13377707 | 466 | T | C | 152 | Phe | Phe |

NOV8 SNP Data:

NOV8 has one SNP variant, whose variant positions for its nucleotide and amino acid sequences is numbered according to SEQ ID NOs: 17 and 18, respectively. The nucleotide sequence of the NOV8 variant differs as shown in Table 26F.

TABLE 26F
SNP data for NOV8

| | Nucleotides | | | Amino Acids | | |
|---|---|---|---|---|---|---|
| Variant | Position | Initial | Modified | Position | Initial | Modified |
| 13377708 | 212 | C | T | 62 | Pro | Leu |

NOV9a SNP Data:

NOV9a has one SNP variant, whose variant positions for its nucleotide and amino acid sequences is numbered according to SEQ ID NOs: 19 and 20, respectively. The nucleotide sequence of the NOV9a variant differs as shown in Table 26G.

TABLE 26G
SNP data for NOV9a

| | Nucleotides | | | Amino Acids | | |
|---|---|---|---|---|---|---|
| Variant | Position | Initial | Modified | Position | Initial | Modified |
| 13374583 | 138 | A | G | 19 | Thr | Ala |

NOV11a SNP Data:

NOV11a has two SNP variants, whose variant positions for their nucleotide and amino acid sequences are numbered according to SEQ ID NOs:25 and 26, respectively. The nucleotide sequences of the NOV11a variants differ as shown in Table 26H.

TABLE 26H
SNP data for NOV11a

| | Nucleotides | | | Amino Acids | | |
|---|---|---|---|---|---|---|
| Variant | Position | Initial | Modified | Position | Initial | Modified |
| 13377709 | 1255 | T | C | 399 | Tyr | His |
| 13377710 | 1415 | C | T | 452 | Ala | Val |

NOV12a SNP Data:

NOV12a has two SNP variants, whose variant positions for their nucleotide and amino acid sequences are numbered according to SEQ ID NOs:29 and 30, respectively. The nucleotide sequences of the NOV12a variants differ as shown in Table 26I.

TABLE 26I
SNP data for NOV12a

| | Nucleotides | | | Amino Acids | | |
|---|---|---|---|---|---|---|
| Variant | Position | Initial | Modified | Position | Initial | Modified |
| 13377676 | 1544 | C | T | 0 | | |
| 13377675 | 1750 | C | T | 0 | | |

NOV13 SNP Data:

NOV13 has one SNP variant, whose variant positions for its nucleotide and amino acid sequences is numbered according to SEQ ID NOs:41 and 42, respectively. The nucleotide sequence of the NOV13 variant differs as shown in Table 26J.

TABLE 26J
SNP data for NOV13

| | Nucleotides | | | Amino Acids | | |
|---|---|---|---|---|---|---|
| Variant | Position | Initial | Modified | Position | Initial | Modified |
| 13377711 | 1383 | C | T | 461 | Asn | Asn |

NOV14a SNP Data:

NOV14a has four SNP variants, whose variant positions for their nucleotide and amino acid sequences are numbered according to SEQ ID NOs:43 and 44, respectively. The nucleotide sequences of the NOV14a variants differ as shown in Table 26K.

TABLE 26K
SNP data for NOV14a

| | Nucleotides | | | Amino Acids | | |
|---|---|---|---|---|---|---|
| Variant | Position | Initial | Modified | Position | Initial | Modified |
| 13377674 | 299 | T | A | 79 | Leu | Gln |
| 13377673 | 335 | G | T | 91 | Arg | Met |
| 13377672 | 532 | G | A | 157 | Ala | Thr |
| 13377671 | 1149 | C | T | 362 | Ala | Ala |

NOV15a SNP Data:

NOV15a has three SNP variants, whose variant positions for their nucleotide and amino acid sequences are numbered according to SEQ ID NOs:51 and 52, respectively. The nucleotide sequences of the NOV15a variants differ as shown in Table 26L.

TABLE 26L
SNP data for NOV15a

| | Nucleotides | | | Amino Acids | | |
|---|---|---|---|---|---|---|
| Variant | Position | Initial | Modified | Position | Initial | Modified |
| 13377670 | 206 | G | A | 60 | Ala | Thr |
| 13377669 | 886 | T | C | 286 | Pro | Pro |
| 13377668 | 1059 | A | G | 344 | Asp | Gly |

NOV20a SNP Data:

NOV20a has three SNP variants, whose variant positions for their nucleotide and amino acid sequences are numbered according to SEQ ID NOs:79 and 80, respectively. The nucleotide sequences of the NOV20a variants differ as shown in Table 26M.

TABLE 26M
SNP data for NOV20a

| | Nucleotides | | | Amino Acids | | |
|---|---|---|---|---|---|---|
| Variant | Position | Initial | Modified | Position | Initial | Modified |
| 13377712 | 300 | T | C | 38 | Ser | Ser |
| 13377713 | 366 | C | T | 60 | Ile | Ile |
| 13377714 | 396 | A | G | 70 | Thr | Thr |

### Example 27

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tₘ=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca. carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel v1.4 and General_screening_panel_v1.5

The plates for Panels 1.4 and 1.5 include 2 control wells (genomic DNA control and chemistry control) and 94 wells containing cDNA from various samples. The samples in Panels 1.4 and 1.5 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panels 1.4 and 1.5 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples.

Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-21 g/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturers instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 M non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶cells/ml in DMEM 5% FCS (Hyclone), 1001M non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (11 g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μ/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NC1-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 351 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive panel_v1.0

The plates for AI comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets l from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the SD and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supemuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra.

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. NOV2a (CG59783-01): CGI-67 Secretory Protein

Expression of gene CG59783-01 was assessed using the primer-probe set Ag3566, described in Table AA. Results of the RTQ-PCR runs are shown in Tables AB, AC and AD.

TABLE AA
Probe Name Ag3566

| | | | Start | SEQ ID |
|---|---|---|---|---|
| Primers | Sequences | Length | Position | No |
| Forward | 5′-gccttccctaacatcgagaa-3′ | 20 | 737 | 91 |
| Probe | TET- | 26 | 764 | 92 |
| | 5′-aagatcacgtctcccgtgctcatcat-3′- | | | |
| | TAMRA | | | |
| Reverse | 5′-agaagtcgatcacctcgtcc-3′ | 20 | 802 | 93 |

TABLE AB
CNS_neurodegeneration_v1.0

| | Rel. Exp. (%) | | Rel. Exp. (%) |
|---|---|---|---|
| | Ag3566, Run | | Ag3566, Run |
| Tissue Name | 210641093 | Tissue Name | 210641093 |
| AD 1 Hippo | 23.2 | Control (Path) 3 Temporal Ctx | 8.8 |
| AD 2 Hippo | 33.0 | Control (Path) 4 Temporal Ctx | 18.2 |
| AD 3 Hippo | 7.6 | AD 1 Occipital Ctx | 14.4 |
| AD 4 Hippo | 5.1 | AD 2 Occipital Ctx (Missing) | 0.0 |
| AD 5 Hippo | 100.0 | AD 3 Occipital Ctx | 6.6 |
| AD 6 Hippo | 62.0 | AD 4 Occipital Ctx | 11.1 |
| Control 2 Hippo | 29.7 | AD 5 Occipital Ctx | 47.6 |
| Control 4 Hippo | 15.6 | AD 6 Occipital Ctx | 17.0 |
| Control (Path) 3 Hippo | 7.4 | Control 1 Occipital Ctx | 7.1 |
| AD 1 Temporal Ctx | 11.5 | Control 2 Occipital Ctx | 88.9 |
| AD 2 Temporal Ctx | 25.5 | Control 3 Occipital Ctx | 16.6 |
| AD 3 Temporal Ctx | 4.9 | Control 4 Occipital Ctx | 8.6 |
| AD 4 Temporal Ctx | 12.1 | Control (Path) 1 Occipital Ctx | 77.9 |
| AD 5 Inf Temporal Ctx | 73.2 | Control (Path) 2 Occipital Ctx | 10.3 |
| AD 5 Sup Temporal Ctx | 51.4 | Control (Path) 3 Occipital Ctx | 7.0 |
| AD 6 Inf Temporal Ctx | 42.9 | Control (Path) 4 Occipital Ctx | 18.3 |
| AD 6 Sup Temporal Ctx | 62.0 | Control 1 Parietal Ctx | 14.0 |
| Control 1 Temporal Ctx | 7.6 | Control 2 Parietal Ctx | 43.2 |
| Control 2 Temporal Ctx | 39.2 | Control 3 Parietal Ctx | 30.4 |
| Control 3 Temporal Ctx | 13.4 | Control (Path) 1 Parietal Ctx | 62.9 |
| Control 3 Temporal Ctx | 9.7 | Control (Path) 2 Parietal Ctx | 13.0 |
| Control (Path) 1 Temporal | 42.0 | Control (Path) 3 Parietal Ctx | 6.2 |
| Ctx | | | |
| Control (Path) 2 Temporal | 28.5 | Control (Path) 4 Parietal Ctx | 44.4 |
| Ctx | | | |

TABLE AC
General_screening_panel_v1.4

| | Rel. Exp. (%) | | Rel. Exp. (%) |
|---|---|---|---|
| | Ag3566, Run | | Ag3566, Run |
| Tissue Name | 217311327 | Tissue Name | 217311327 |
| Adipose | 2.9 | Renal ca. TK-10 | 9.7 |
| Melanoma* Hs688(A).T | 15.5 | Bladder | 12.9 |
| Melanoma* Hs688(B).T | 13.6 | Gastric ca. (liver met.) | 8.1 |
| | | NCI-N87 | |
| Melanoma* M14 | 13.6 | Gastric ca. KATO III | 17.0 |
| Melanoma* LOXIMVI | 9.2 | Colon ca. SW-948 | 10.4 |
| Melanoma* SK-MEL-5 | 8.1 | Colon ca. SW480 | 26.6 |
| Squamous cell carcinoma | 10.3 | Colon ca.* (SW480 met) | 16.6 |
| SCC-4 | | SW620 | |
| Testis Pool | 3.4 | Colon ca. HT29 | 8.5 |
| Prostate ca.* (bone met) PC-3 | 11.7 | Colon ca. HCT-116 | 36.3 |
| Prostate Pool | 2.8 | Colon ca. CaCo-2 | 10.2 |
| Placenta | 11.5 | Colon cancer tissue | 16.6 |
| Uterus Pool | 0.8 | Colon ca. SW1116 | 11.7 |
| Ovarian ca. OVCAR-3 | 20.3 | Colon ca. Colo-205 | 4.3 |
| Ovarian ca. SK-OV-3 | 26.1 | Colon ca. SW-48 | 6.0 |
| Ovarian ca. OVCAR-4 | 7.7 | Colon Pool | 6.7 |
| Ovarian ca. OVCAR-5 | 23.5 | Small Intestine Pool | 6.0 |
| Ovarian ca. IGROV-1 | 31.0 | Stomach Pool | 3.2 |
| Ovarian ca. OVCAR-8 | 19.9 | Bone Marrow Pool | 2.1 |
| Ovary | 6.1 | Fetal Heart | 6.6 |
| Breast ca. MCF-7 | 18.8 | Heart Pool | 4.1 |
| Breast ca. MDA-MB-231 | 36.6 | Lymph Node Pool | 6.5 |
| Breast ca. BT 549 | 42.9 | Fetal Skeletal Muscle | 4.6 |
| Breast ca. T47D | 100.0 | Skeletal Muscle Pool | 7.4 |
| Breast ca. MDA-N | 31.4 | Spleen Pool | 6.8 |
| Breast Pool | 5.7 | Thymus pool | 8.4 |
| Trachea | 8.9 | CNS cancer (glio/astro) | 26.6 |
| | | U87-MG | |
| Lung | 1.5 | CNS cancer (glio/astro) | 36.9 |
| | | U-118-MG | |
| Fetal Lung | 14.9 | CNS cancer (neuro; met) | 29.1 |
| | | SK-N-AS | |
| Lung ca. NCI-N417 | 9.3 | CNS cancer (astro) SF-539 | 9.3 |
| Lung ca. LX-1 | 15.9 | CNS cancer (astro) SNB-75 | 37.1 |
| Lung ca. NCI-H146 | 9.7 | CNS cancer (glio) SNB-19 | 27.4 |
| Lung ca. SHP-77 | 21.3 | CNS cancer (glio) SF-295 | 25.5 |
| Lung ca. A549 | 10.2 | Brain (Amygdala) Pool | 21.5 |
| Lung ca. NCI-H526 | 8.3 | Brain (cerebellum) | 22.4 |
| Lung ca. NCI-H23 | 12.4 | Brain (fetal) | 12.3 |
| Lung ca. NCI-H460 | 4.8 | Brain (Hippocampus) Pool | 17.8 |
| Lung ca. HOP-62 | 6.5 | Cerebral Cortex Pool | 16.8 |
| Lung ca. NCI-H522 | 9.3 | Brain (Substantia nigra) Pool | 25.9 |
| Liver | 1.3 | Brain (Thalamus) Pool | 23.5 |
| Fetal Liver | 7.4 | Brain (whole) | 15.1 |
| Liver ca. HepG2 | 8.4 | Spinal Cord Pool | 20.4 |
| Kidney Pool | 12.2 | Adrenal Gland | 5.9 |
| Fetal Kidney | 8.7 | Pituitary gland Pool | 1.7 |
| Renal ca. 786-0 | 11.7 | Salivary Gland | 6.2 |
| Renal ca. A498 | 5.2 | Thyroid (female) | 8.8 |
| Renal ca. ACHN | 5.2 | Pancreatic ca. CAPAN2 | 6.7 |
| Renal ca. UO-31 | 9.7 | Pancreas Pool | 12.2 |

TABLE AD
Panel 4.1D

| | Rel. Exp. (%) | | Rel. Exp. (%) |
|---|---|---|---|
| | Ag3566, Run | | Ag3566, Run |
| Tissue Name | 169851074 | Tissue Name | 169851074 |
| Secondary Th1 act | 56.6 | HUVEC IL-1beta | 40.3 |
| Secondary Th2 act | 80.7 | HUVEC IFN gamma | 39.5 |
| Secondary Tr1 act | 68.3 | HUVEC TNF alpha + IFN | 39.0 |
| | | gamma | |
| Secondary Th1 rest | 82.9 | HUVEC TNF alpha + IL4 | 31.6 |
| Secondary Th2 rest | 90.1 | HUVEC IL-11 | 23.2 |
| Secondary Tr1 rest | 82.9 | Lung Microvascular EC | 69.7 |
| | | none | |
| Primary Th1 act | 46.3 | Lung Microvascular EC | 66.0 |
| | | TNF alpha + IL-1beta | |
| Primary Th2 act | 72.7 | Microvascular Dermal EC | 46.7 |
| | | none | |
| Primary Tr1 act | 46.3 | Microsvasular Dermal EC | 41.2 |
| | | TNF alpha + IL-1beta | |
| Primary Th1 rest | 77.4 | Bronchial epithelium | 19.3 |
| | | TNF alpha + IL1beta | |
| Primary Th2 rest | 63.3 | Small airway epithelium | 10.7 |
| | | none | |
| Primary Tr1 rest | 73.2 | Small airway epithelium | 33.2 |
| | | TNF alpha + IL-1beta | |
| CD45RA CD4 lymphocyte act | 42.6 | Coronery artery SMC rest | 26.1 |
| CD45RO CD4 lymphocyte act | 70.7 | Coronery artery SMC | 24.1 |
| | | TNF alpha + IL-1beta | |
| CD8 lymphocyte act | 84.1 | Astrocytes rest | 23.8 |
| Secondary CD8 lymphocyte | 48.0 | Astrocytes TNF alpha + | 22.7 |
| rest | | IL-1beta | |
| Secondary CD8 lymphocyte | 48.3 | KU-812 (Basophil) rest | 37.9 |
| act | | | |
| CD4 lymphocyte none | 32.1 | KU-812 (Basophil) | 48.6 |
| | | PMA/ionomycin | |
| 2ry Th1/Th2/Tr1_anti-CD95 | 79.6 | CCD1106 (Keratinocytes) | 48.3 |
| CH11 | | none | |
| LAK cells rest | 37.6 | CCD1106 (Keratinocytes) | 49.7 |
| | | TNF alpha + IL-1beta | |
| LAK cells IL-2 | 51.4 | Liver cirrhosis | 4.7 |
| LAK cells IL-2 + IL-12 | 39.2 | NCI-H292 none | 25.7 |
| LAK cells IL-2 + IFN gamma | 45.1 | NCI-H292 IL-4 | 34.4 |
| LAK cells IL-2 + IL-18 | 37.1 | NCI-H292 IL-9 | 36.1 |
| LAK cells PMA/ionomycin | 20.6 | NCI-H292 IL-13 | 46.3 |
| NK Cells IL-2 rest | 100.0 | NCI-H292 IFN gamma | 30.4 |
| Two Way MLR 3 day | 47.3 | HPAEC none | 34.4 |
| Two Way MLR 5 day | 49.7 | HPAEC TNF alpha + | 44.8 |
| | | IL-1beta | |
| Two Way MLR 7 day | 46.3 | Lung fibroblast none | 36.6 |
| PBMC rest | 39.2 | Lung fibroblast TNF alpha + | 21.6 |
| | | IL-1beta | |
| PBMC PWM | 42.6 | Lung fibroblast IL-4 | 42.9 |
| PBMC PHA-L | 53.6 | Lung fibroblast IL-9 | 44.8 |
| Ramos (B cell) none | 24.5 | Lung fibroblast IL-13 | 35.8 |
| Ramos (B cell) ionomycin | 21.3 | Lung fibroblast IFN gamma | 48.0 |
| B lymphocytes PWM | 18.9 | Dermal fibroblast CCD1070 | 30.8 |
| | | rest | |
| B lymphocytes CD40L and | 33.7 | Dermal fibroblast CCD1070 | 94.0 |
| IL-4 | | TNF alpha | |
| EOL-1 dbcAMP | 45.1 | Dermal fibroblast CCD1070 | 35.1 |
| | | IL-1beta | |
| EOL-1 dbcAMP | 46.0 | Dermal fibroblast IFN | 31.2 |
| PMA/ionomycin | | gamma | |
| Dendritic cells none | 36.9 | Dermal fibroblast IL-4 | 39.2 |
| Dendritic cells LPS | 21.9 | Dermal Fibroblasts rest | 24.3 |
| Dendritic cells anti-CD40 | 40.3 | Neutrophils TNFa + LPS | 3.6 |
| Monocytes rest | 48.6 | Neutrophils rest | 9.4 |
| Monocytes LPS | 21.3 | Colon | 19.2 |
| Macrophages rest | 47.6 | Lung | 30.6 |
| Macrophages LPS | 31.4 | Thymus | 23.5 |
| HUVEC none | 25.0 | Kidney | 14.3 |
| HUVEC starved | 37.9 | | |

CNS_neurodegeneration_v1.0 Summary: Ag3566 This panel does not show differential expression of the CG9783-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3566 The CG9783-01 gene is ubiquitously expressed in this panel, with highest expression in a breast cancer cell line (CT=26.1). Significant levels of expression are also seen in a cluster of samples derived from breast cancer cell lines. Thus, expression of this gene could be used to differentiate between these samples and other samples on this panel and as a marker to detect the presence of breast cancer. Furthermore, therapeutic modulation of the expression or function of this gene may be effective in the treatment of breast cancer.

This molecule is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at much higher levels in fetal lung (CT=28.8) when compared to expression in the adult counterpart (CT=32). Thus, expression of this gene may be used to differentiate between the fetal and adult source of this tissue.

Panel 4.1D Summary: Ag3566 The CG9783-01 gene is ubiquitously expressed in this panel, with highest expression in IL-2 treated NK cells (CT=28). In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v0.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

B. NOV3 (CG59873-01): Cystatin—Isoform 1

Expression of gene CG59873-01 was assessed using the primer-probe set Ag3624, described in Table BA. Results of the RTQ-PCR runs are shown in Tables BB.

TABLE BA

Probe Name Ag3624

| | | | Start | SEQ ID |
|---|---|---|---|---|
| Primers | Sequences | Length | Position | No |
| Forward | 5′-ggaaggagcagggttatgataa-3′ | 22 | 250 | 94 |
| Probe | TET-5′-acattctccatgaatctgcaactggg-3′-TAMRA | 26 | 276 | 95 |
| Reverse | 5′-atcttcaaatttcccacacatg-3′ | 22 | 308 | 96 |

TABLE BB
Panel 4.1D

| | Rel. | | Rel. |
|---|---|---|---|
| | Exp. (%) | | Exp. (%) |
| | Ag3624, | | Ag3624, |
| | Run | | Run |
| Tissue Name | 169945972 | Tissue Name | 169945972 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 0.0 | Bronchial epithelium TNF | 0.0 |
| | | alpha + IL-1beta | |
| Primary Th2 rest | 0.0 | Small airway epithelium none | 0.0 |
| Primary Tr1 rest | 0.0 | Small airway epithelium | 0.0 |
| | | TNF alpha + IL-1beta | |
| CD45RA CD4 lymphocyte act | 0.0 | Coronery artery SMC rest | 5.8 |
| CD45RO CD4 lymphocyte act | 0.0 | Coronery artery SMC | 0.0 |
| | | TNF alpha + IL-1beta | |
| CD8 lymphocyte act | 0.0 | Astrocytes rest | 0.0 |
| Secondary CD8 lymphocyte rest | 0.0 | Astrocytes TNF alpha + | 9.3 |
| | | IL-1beta | |
| Secondary CD8 lymphocyte act | 3.0 | KU-812 (Basophil) rest | 0.0 |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) none | 2.9 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 12.7 |
| LAK cells IL-2 + IFN gamma | 0.0 | NCI-H292 IL-4 | 0.0 |
| LAK cells IL-2 + IL-18 | 0.0 | NCI-H292 IL-9 | 4.7 |
| LAK cells PMA/ionomycin | 0.0 | NCI-H292 IL-13 | 3.2 |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1beta | 0.0 |
| Two Way MLR 7 day | 0.0 | Lung fibroblast none | 0.0 |
| PBMC rest | 0.0 | Lung fibroblast TNF alpha + | 3.2 |
| | | IL-1beta | |
| PBMC PWM | 0.0 | Lung fibroblast IL-4 | 4.2 |
| PBMC PHA-L | 3.3 | Lung fibroblast IL-9 | 4.5 |
| Ramos (B cell) none | 0.0 | Lung fibroblast IL-13 | 47.0 |
| Ramos (B cell) ionomycin | 0.0 | Lung fibroblast IFN gamma | 14.9 |
| B lymphocytes PWM | 0.0 | Dermal fibroblast CCD1070 | 39.0 |
| | | rest | |
| B lymphocytes CD40L and IL-4 | 0.0 | Dermal fibroblast CCD1070 | 100.0 |
| | | TNF alpha | |
| EOL-1 dbcAMP | 0.0 | Dermal fibroblast CCD1070 | 15.0 |
| | | IL-1beta | |
| EOL-1 dbcAMP PMA/ | 0.0 | Dermal fibroblast IFN gamma | 0.0 |
| ionomycin | | | |
| Dendritic cells none | 0.0 | Dermal fibroblast IL-4 | 0.0 |
| Dendritic cells LPS | 0.0 | Dermal Fibroblasts rest | 28.1 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.0 |
| Macrophages rest | 0.0 | Lung | 7.7 |
| Macrophages LPS | 0.0 | Thymus | 2.8 |
| HUVEC none | 0.0 | Kidney | 5.8 |
| HUVEC starved | 0.0 | | |

CNS_neurodegeneration_v1.0 Summary: Ag3624 Expression of the CG59873-01 gene is low/undetectable in all samples on this panel (CTs>35).

General_screening_panel_v1.4 Summary: Ag3624 Expression of the CG59873-01 gene is low/undetectable in all samples on this panel (CTs>35).

Panel 4.1D Summary: Ag3624 Expression of the CG59873-01 gene is restricted to TNF-alpha treated dermal fibroblasts. Thus, expression of this gene could be used as a marker of this cell type. Furthermore, therapeutic modulation of the activity or function of this gene may be useful in the treatment of skin disorders such as psoriasis.

C. NOV4 (CG89060-01): Collagen Alpha 1(XIV) Chain Precursor (Undulin)

Expression of gene CG89060-01 was assessed using the primer-probe set Ag3686, described in Table CA. Results of the RTQ-PCR runs are shown in Tables CB, CC and CD.

TABLE CA

Probe Name Ag3686

| | | | Start | SEQ ID |
|---|---|---|---|---|
| Primers | Sequences | Length | Position | No |
| Forward | 5′-tgttactttcgaaggacctgaa-3′ | 22 | 4105 | 97 |
| Probe | TET-5′-tggaagctttcacaagctacacattg-3′-TAMRA | 26 | 4144 | 98 |
| Reverse | 5′-gaccaaagcctcactgacaa-3′ | 20 | 4170 | 99 |

TABLE CB
CNS_neurodegeneration_v1.0

| | Rel. Exp. (%) | | Rel. Exp. (%) |
|---|---|---|---|
| | Ag3686, Run | | Ag3686, Run |
| Tissue Name | 211144674 | Tissue Name | 211144674 |
| AD 1 Hippo | 3.6 | Control (Path) 3 Temporal | 9.3 |
| | | Ctx | |
| AD 2 Hippo | 5.8 | Control (Path) 4 Temporal | 7.8 |
| | | Ctx | |
| AD 3 Hippo | 2.9 | AD 1 Occipital Ctx | 3.6 |
| AD 4 Hippo | 1.9 | AD 2 Occipital Ctx | 0.0 |
| | | (Missing) | |
| AD 5 Hippo | 15.2 | AD 3 Occipital Ctx | 3.0 |
| AD 6 Hippo | 11.3 | AD 4 Occipital Ctx | 6.2 |
| Control 2 Hippo | 3.2 | AD 5 Occipital Ctx | 12.5 |
| Control 4 Hippo | 9.0 | AD 6 Occipital Ctx | 7.2 |
| Control (Path) 3 Hippo | 9.0 | Control 1 Occipital Ctx | 5.8 |
| AD 1 Temporal Ctx | 9.6 | Control 2 Occipital Ctx | 11.8 |
| AD 2 Temporal Ctx | 9.0 | Control 3 Occipital Ctx | 6.2 |
| AD 3 Temporal Ctx | 1.5 | Control 4 Occipital Ctx | 2.9 |
| AD 4 Temporal Ctx | 11.0 | Control (Path) 1 Occipital | 7.2 |
| | | Ctx | |
| AD 5 Inf Temporal Ctx | 9.2 | Control (Path) 2 Occipital | 4.0 |
| | | Ctx | |
| AD 5 Sup Temporal Ctx | 10.7 | Control (Path) 3 Occipital | 2.3 |
| | | Ctx | |
| AD 6 Inf Temporal Ctx | 7.1 | Control (Path) 4 Occipital | 11.6 |
| | | Ctx | |
| AD 6 Sup Temporal Ctx | 100.0 | Control 1 Parietal Ctx | 6.7 |
| Control 1 Temporal Ctx | 3.6 | Control 2 Parietal Ctx | 11.0 |
| Control 2 Temporal Ctx | 4.1 | Control 3 Parietal Ctx | 3.2 |
| Control 3 Temporal Ctx | 6.9 | Control (Path) 1 Parietal Ctx | 4.5 |
| Control 3 Temporal Ctx | 7.8 | Control (Path) 2 Parietal Ctx | 10.2 |
| Control (Path) 1 Temporal | 17.7 | Control (Path) 3 Parietal Ctx | 6.5 |
| Ctx | | | |
| Control (Path) 2 Temporal | 7.3 | Control (Path) 4 Parietal Ctx | 9.9 |
| Ctx | | | |

TABLE CC
General_screening_panel_v1.4

| | Rel. Exp. (%) | | Rel. Exp. (%) |
|---|---|---|---|
| | Ag3686, Run | | Ag3686, Run |
| Tissue Name | 218941312 | Tissue Name | 218941312 |
| Adipose | 10.2 | Renal ca. TK-10 | 9.2 |
| Melanoma* Hs688(A).T | 1.2 | Bladder | 11.2 |
| Melanoma* Hs688(B).T | 1.4 | Gastric ca. (liver met.) NCI-N87 | 0.0 |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell carcinoma | 0.0 | Colon ca.* (SW480 met) | 0.0 |
| SCC-4 | | SW620 | |
| Testis Pool | 5.4 | Colon ca. HT29 | 0.0 |
| Prostate ca.* (bone met) PC-3 | 0.0 | Colon ca. HCT-116 | 0.0 |
| Prostate Pool | 9.9 | Colon ca. CaCo-2 | 0.1 |
| Placenta | 4.0 | Colon cancer tissue | 26.6 |
| Uterus Pool | 7.1 | Colon ca. SW1116 | 0.0 |
| Ovarian ca. OVCAR-3 | 0.0 | Colon ca. Colo-205 | 0.0 |
| Ovarian ca. SK-OV-3 | 0.2 | Colon ca. SW-48 | 0.0 |
| Ovarian ca. OVCAR-4 | 0.0 | Colon Pool | 30.4 |
| Ovarian ca. OVCAR-5 | 3.4 | Small Intestine Pool | 11.2 |
| Ovarian ca. IGROV-1 | 11.8 | Stomach Pool | 3.9 |
| Ovarian ca. OVCAR-8 | 12.6 | Bone Marrow Pool | 15.0 |
| Ovary | 24.8 | Fetal Heart | 4.3 |
| Breast ca. MCF-7 | 0.0 | Heart Pool | 13.8 |
| Breast ca. MDA-MB-231 | 0.0 | Lymph Node Pool | 33.9 |
| Breast ca. BT 549 | 0.2 | Fetal Skeletal Muscle | 6.4 |
| Breast ca. T47D | 5.6 | Skeletal Muscle Pool | 2.0 |
| Breast ca. MDA-N | 0.0 | Spleen Pool | 6.5 |
| Breast Pool | 33.7 | Thymus Pool | 15.4 |
| Trachea | 12.3 | CNS cancer (glio/astro) | 0.1 |
| | | U87-MG | |
| Lung | 5.6 | CNS cancer (glio/astro) | 100.0 |
| | | U-118-MG | |
| Fetal Lung | 31.2 | CNS cancer (neuro; met) | 0.0 |
| | | SK-N-AS | |
| Lung ca. NCI-N417 | 0.0 | CNS cancer (astro) SF-539 | 1.5 |
| Lung ca. LX-1 | 0.0 | CNS cancer (astro) SNB-75 | 54.0 |
| Lung ca. NCI-H146 | 0.0 | CNS cancer (glio) SNB-19 | 11.6 |
| Lung ca. SHP-77 | 0.4 | CNS cancer (glio) SF-295 | 5.0 |
| Lung ca. A549 | 0.0 | Brain (Amygdala) Pool | 0.3 |
| Lung ca. NCI-H526 | 0.7 | Brain (cerebellum) | 0.1 |
| Lung ca. NCI-H23 | 4.4 | Brain (fetal) | 0.4 |
| Lung ca. NCI-H460 | 0.0 | Brain (Hippocampus) Pool | 1.5 |
| Lung ca. HOP-62 | 0.7 | Cerebral Cortex Pool | 0.5 |
| Lung ca. NCI-H522 | 65.1 | Brain (Substantia nigra) Pool | 0.2 |
| Liver | 0.4 | Brain (Thalamus) Pool | 0.6 |
| Fetal Liver | 6.8 | Brain (whole) | 0.5 |
| Liver ca. HepG2 | 0.0 | Spinal Cord Pool | 1.9 |
| Kidney Pool | 40.6 | Adrenal Gland | 2.8 |
| Fetal Kidney | 4.4 | Pituitary gland Pool | 0.2 |
| Renal ca. 786-0 | 3.8 | Salivary Gland | 3.8 |
| Renal ca. A498 | 0.1 | Thyroid (female) | 5.0 |
| Renal ca. ACHN | 1.9 | Pancreatic ca. CAPAN2 | 0.0 |
| Renal ca. UO-31 | 0.0 | Pancreas Pool | 11.2 |

TABLE CD
Panel 4.1D

| | Rel. Exp. (%) | | Rel. Exp. (%) |
|---|---|---|---|
| | Ag3686, Run | | Ag3686, Run |
| Tissue Name | 169988044 | Tissue Name | 169988044 |
| Secondary Th1 act | 0.0 | HUVEC IL-1beta | 0.0 |
| Secondary Th2 act | 0.0 | HUVEC IFN gamma | 0.1 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.1 |
| Secondary Th2 rest | 0.0 | HUVEC IL-11 | 0.1 |
| Secondary Tr1 rest | 0.0 | Lung Microvascular EC none | 0.1 |
| Primary Th1 act | 0.0 | Lung Microvascular EC | 0.1 |
| | | TNF alpha + IL-1beta | |
| Primary Th2 act | 0.0 | Microvascular Dermal EC none | 0.0 |
| Primary Tr1 act | 0.0 | Microvascular Dermal EC | 0.0 |
| | | TNF alpha + IL-1beta | |
| Primary Th1 rest | 0.0 | Bronchial epithelium TNF | 0.0 |
| | | alpha + IL1beta | |
| Primary Th2 rest | 0.0 | Small airway epithelium none | 0.1 |
| Primary Tr1 rest | 0.0 | Small airway epithelium | 0.1 |
| | | TNF alpha + IL-1beta | |
| CD45RA CD4 lymphocyte | 0.0 | Coronery artery SMC rest | 0.2 |
| act | | | |
| CD45RO CD4 lymphocyte | 0.0 | Coronery artery SMC TNF | 0.1 |
| act | | alpha + IL-1beta | |
| CD8 lymphocyte act | 0.0 | Astrocytes rest | 4.7 |
| Secondary CD8 lymphocyte | 0.0 | Astrocytes TNF alpha + IL-1beta | 1.9 |
| rest | | | |
| Secondary CD8 lymphocyte | 0.0 | KU-812 (Basophil) rest | 1.2 |
| act | | | |
| CD4 lymphocyte none | 0.0 | KU-812 (Basophil) | 0.5 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 5.0 |
| LAK cells IL-2 + IL-12 | 0.0 | NCI-H292 none | 0.0 |
| LAK cells IL-2 + IFN gamma | 0.0 | NCI-H292 IL-4 | 0.0 |
| LAK cells IL-2 + IL-18 | 0.0 | NCI-H292 IL-9 | 0.0 |
| LAK cells PMA/ionomycin | 0.0 | NCI-H292 IL-13 | 0.0 |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1beta | 0.0 |
| Two Way MLR 7 day | 0.0 | Lung fibroblast none | 5.6 |
| PBMC rest | 0.1 | Lung fibroblast TNF alpha + | 1.1 |
| | | IL-1beta | |
| PBMC PWM | 0.0 | Lung fibroblast IL-4 | 7.7 |
| PBMC PHA-L | 0.0 | Lung fibroblast IL-9 | 5.6 |
| Ramos (B cell) none | 0.0 | Lung fibroblast IL-13 | 9.1 |
| Ramos (B cell) ionomycin | 0.1 | Lung fibroblast IFN gamma | 10.2 |
| B lymphocytes PWM | 0.0 | Dermal fibroblast CCD1070 rest | 0.2 |
| B lymphocytes CD40L and | 0.0 | Dermal fibroblast CCD1070 | 0.2 |
| IL-4 | | TNF alpha | |
| EOL-1 dbcAMP | 0.0 | Dermal fibroblast CCD1070 | 0.1 |
| | | IL-1beta | |
| EOL-1 dbcAMP | 0.0 | Dermal fibroblast IFN gamma | 20.9 |
| PMA/ionomycin | | | |
| Dendritic cells none | 0.0 | Dermal fibroblast IL-4 | 100.0 |
| Dendritic cells LPS | 0.0 | Dermal Fibroblasts rest | 8.8 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 5.3 |
| Macrophages rest | 0.0 | Lung | 24.8 |
| Macrophages LPS | 0.0 | Thymus | 2.7 |
| HUVEC none | 0.0 | Kidney | 4.0 |
| HUVEC starved | 0.0 | | |

CNS_neurodegeneration_v1.0 Summary: Ag3686 This panel does not show differential expression of the CG89060-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3686 Expression of the CG89060-01 gene is highest in a brain cancer cell line (CT=27). Significant expression is also seen in a lung cancer cell line and a second brain cancer cell line. Thus, expression of this gene could be used to differentiate between these samples and other samples on this panel and as a marker of lung and brain cancers. Expression of undulin, of which this gene product is a homolog, has been shown to be associated with certain brain cancer cell lines. See, Paulus W. et al. Am J Pathol July 1993;143(1):154-63 (PMID: 8317546). Therefore, therapeutic modulation of the expression or function of this gene may be effective in the treatment of these cancers.

Among tissues with metabolic function, this gene is expressed at moderate to low levels in pituitary, adipose, adrenal gland, pancreas, thyroid, fetal liver and adult and fetal skeletal muscle and heart. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at much higher levels in fetal liver tissue (CT=30) when compared to expression in the adult counterpart (CT=35). Thus, expression of this gene may be used to differentiate between the fetal and adult source of this tissue.

This gene is also expressed at low but significant levels in the hippocampus, thalamus and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag3686 Expression of the CG89060-01 gene is limited to a few samples in this panel, with highest expression in IL-4 treated dermal fibroblasts. Moderate levels of expression are also seen in IFN-gamma stimulated dermal fibroblasts, the lung, and a cluster of treated and untreated lung fibroblast samples. Thus, expression of this gene could be used to differentiate activated dermal fibroblasts from other samples on this panel and as a marker for fibroblasts. Furthermore, therapeutic modulation of the expression or function of this gene product may be useful in treating lung or skin disorders including psoriasis, asthma, emphysema, and allergy.

D. NOV8 (CG90155-01): Secreted Protein

Expression of gene CG90155-01 was assessed using the primer-probe set Ag3792, described in Table DA. Results of the RTQ-PCR runs are shown in Tables DB and DC.

TABLE DA

Probe Name Ag3792

| | | | | |
|---|---|---|---|---|
| Primers | Sequences | Length | Start Position | SEQ ID No |

| | | | | |
|---|---|---|---|---|
| Forward | 5′-cacctaaccgagggtgactc-3′ | 20 | 316 | 100 |
| Probe | TET-5′-accaccagctggagagccctagct-3′-TAMRA | 24 | 355 | 101 |
| Reverse | 5′-atgttgatccaaagctgctg-3′ | 20 | 380 | 102 |

TABLE DB
General_screening_panel_v1.4

| | Rel. Exp. (%) | | Rel. Exp. (%) |
|---|---|---|---|
| | Ag3792, Run | | Ag3792, Run |
| Tissue Name | 218905932 | Tissue Name | 218905932 |
| Adipose | 0.0 | Renal ca. TK-10 | 1.2 |
| Melanoma* Hs688(A).T | 0.0 | Bladder | 3.4 |
| Melanoma* Hs688(B).T | 0.0 | Gastric ca. (liver met.) NCI-N87 | 0.0 |
| Melanoma* M14 | 3.1 | Gastric ca. KATO III | 38.2 |
| Melanoma* LOXIMVI | 0.0 | Colon ca. SW-948 | 5.8 |
| Melanoma* SK-MEL-5 | 8.4 | Colon ca. SW480 | 17.8 |
| Squamous cell carcinoma | 26.2 | Colon ca.* (SW480 met) | 26.8 |
| SCC-4 | | SW620 | |
| Testis Pool | 15.7 | Colon ca. HT29 | 7.2 |
| Prostate ca.* (bone met) PC-3 | 0.0 | Colon ca. HCT-116 | 0.0 |
| Prostate Pool | 0.0 | Colon ca. CaCo-2 | 0.0 |
| Placenta | 100.0 | Colon cancer tissue | 0.0 |
| Uterus Pool | 1.0 | Colon ca. SW1116 | 11.1 |
| Ovarian ca. OVCAR-3 | 18.9 | Colon ca. Colo-205 | 36.1 |
| Ovarian ca. SK-OV-3 | 0.0 | Colon ca. SW-48 | 11.6 |
| Ovarian ca. OVCAR-4 | 1.2 | Colon Pool | 0.0 |
| Ovarian ca. OVCAR-5 | 0.0 | Small Intestine Pool | 24.3 |
| Ovarian ca. IGROV-1 | 0.0 | Stomach Pool | 18.8 |
| Ovarian ca. OVCAR-8 | 37.4 | Bone Marrow Pool | 0.0 |
| Ovary | 10.0 | Fetal Heart | 3.9 |
| Breast ca. MCF-7 | 7.5 | Heart Pool | 0.0 |
| Breast ca. MDA-MB-231 | 0.0 | Lymph Node Pool | 3.0 |
| Breast ca. BT 549 | 21.0 | Fetal Skeletal Muscle | 0.0 |
| Breast ca. T47D | 0.0 | Skeletal Muscle Pool | 73.2 |
| Breast ca. MDA-N | 0.0 | Spleen Pool | 3.0 |
| Breast Pool | 0.0 | Thymus Pool | 0.0 |
| Trachea | 6.9 | CNS cancer (glio/astro) | 49.3 |
| | | U87-MG | |
| Lung | 2.2 | CNS cancer (glio/astro) | 15.9 |
| | | U-118-MG | |
| Fetal Lung | 6.1 | CNS cancer (neuro; met) | 0.0 |
| | | SK-N-AS | |
| Lung ca. NCI-N417 | 0.0 | CNS cancer (astro) SF-539 | 38.2 |
| Lung ca. LX-1 | 55.5 | CNS cancer (astro) SNB-75 | 3.7 |
| Lung ca. NCI-H146 | 3.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 29.3 |
| Lung ca. NCI-H23 | 5.9 | Brain (fetal) | 0.0 |
| Lung ca. NCI-H460 | 47.6 | Brain (Hippocampus) Pool | 0.0 |
| Lung ca. HOP-62 | 44.4 | Cerebral Cortex Pool | 5.5 |
| Lung ca. NCI-H522 | 17.6 | Brain (Substantia nigra) Pool | 0.0 |
| Liver | 0.0 | Brain (Thalamus) Pool | 22.1 |
| Fetal Liver | 0.0 | Brain (whole) | 0.0 |
| Liver ca. HepG2 | 22.4 | Spinal Cord Pool | 0.0 |
| Kidney Pool | 43.5 | Adrenal Gland | 35.1 |
| Fetal Kidney | 25.9 | Pituitary gland Pool | 18.2 |
| Renal ca. 786-0 | 13.0 | Salivary Gland | 3.7 |
| Renal ca. A498 | 56.6 | Thyroid (female) | 33.7 |
| Renal ca. ACHN | 0.0 | Pancreatic ca. CAPAN2 | 54.0 |
| Renal ca. UO-31 | 22.5 | Pancreas Pool | 2.9 |

TABLE DC
Panel 4.1D

| | Rel. Exp. (%) | | Rel. Exp. (%) |
|---|---|---|---|
| | Ag3792, Run | | Ag3792, Run |
| Tissue Name | 169997316 | Tissue Name | 169997316 |
| Secondary Th1 act | 24.0 | HUVEC IL-1beta | 5.3 |
| Secondary Th2 act | 9.9 | HUVEC IFN gamma | 0.0 |
| Secondary Tr1 act | 20.4 | HUVEC TNF alpha + IFN | 23.8 |
| | | gamma | |
| Secondary Th1 rest | 22.2 | HUVEC TNF alpha + IL4 | 0.0 |
| Secondary Th2 rest | 17.7 | HUVEC IL-11 | 74.2 |
| Secondary Tr1 rest | 0.0 | Lung Microvascular EC none | 0.0 |
| Primary Th1 act | 17.4 | Lung Microvascular EC | 25.7 |
| | | TNF alpha + IL-1beta | |
| Primary Th2 act | 20.7 | Microvascular Dermal EC none | 12.0 |
| Primary Tr1 act | 46.0 | Microsvasular Dermal EC | 29.3 |
| | | TNF alpha + IL-1beta | |
| Primary Th1 rest | 26.6 | Bronchial epithelium TNF | 0.0 |
| | | alpha + IL1beta | |
| Primary Th2 rest | 34.2 | Small airway epithelium none | 18.2 |
| Primary Tr1 rest | 34.4 | Small airway epithelium | 29.3 |
| | | TNF alpha + IL-1beta | |
| CD45RA CD4 lymphocyte | 70.2 | Coronery artery SMC rest | 55.5 |
| act | | | |
| CD45RO CD4 lymphocyte | 0.0 | Coronery artery SMC | 15.3 |
| act | | TNF alpha + IL-1beta | |
| CD8 lymphocyte act | 0.0 | Astrocytes rest | 21.0 |
| Secondary CD8 lymphocyte | 29.3 | Astrocytes TNF alpha + | 40.6 |
| rest | | IL-1beta | |
| Secondary CD8 lymphocyte | 0.0 | KU-812 (Basophil) rest | 0.0 |
| act | | | |
| CD4 lymphocyte none | 0.0 | KU-812 (Basophil) | 16.3 |
| | | PMA/ionomycin | |
| 2ry Th1/Th2/Tr1_anti-CD95 | 16.7 | CCD1106 (Keratinocytes) none | 0.0 |
| CH11 | | | |
| LAK cells rest | 0.0 | CCD1106 (Keratinocytes) | 65.1 |
| | | TNF alpha + IL-1beta | |
| LAK cells IL-2 | 0.0 | Liver cirrhosis | 0.0 |
| LAK cells IL-2 + IL-12 | 67.8 | NCI-H292 none | 33.9 |
| LAK cells IL-2 + IFN gamma | 19.9 | NCI-H292 IL-4 | 61.1 |
| LAK cells IL-2 + IL-18 | 9.5 | NCI-H292 IL-9 | 0.0 |
| LAK cells PMA/ionomycin | 32.3 | NCI-H292 IL-13 | 40.1 |
| NK Cells IL-2 rest | 26.1 | NCI-H292 IFN gamma | 42.9 |
| Two Way MLR 3 day | 0.0 | HPAEC none | 12.4 |
| Two Way MLR 5 day | 33.4 | HPAEC TNF alpha + IL-1beta | 0.0 |
| Two Way MLR 7 day | 43.5 | Lung fibroblast none | 2.9 |
| PBMC rest | 0.0 | Lung fibroblast TNF alpha + | 0.0 |
| | | IL-1beta | |
| PBMC PWM | 10.5 | Lung fibroblast IL-4 | 11.1 |
| PBMC PHA-L | 20.2 | Lung fibroblast IL-9 | 0.0 |
| Ramos (B cell) none | 0.0 | Lung fibroblast IL-13 | 36.3 |
| Ramos (B cell) ionomycin | 10.8 | Lung fibroblast IFN gamma | 0.0 |
| B lymphocytes PWM | 26.6 | Dermal fibroblast CCD1070 | 0.0 |
| | | rest | |
| B lymphocytes CD40L and | 0.0 | Dermal fibroblast CCD1070 | 43.8 |
| IL-4 | | TNF alpha | |
| EOL-1 dbcAMP | 5.1 | Dermal fibroblast CCD1070 | 6.1 |
| | | IL-1beta | |
| EOL-1 dbcAMP | 34.4 | Dermal fibroblast IFN gamma | 9.9 |
| PMA/ionomycin | | | |
| Dendritic cells none | 40.3 | Dermal fibroblast IL-4 | 0.0 |
| Dendritic cells LPS | 0.0 | Dermal Fibroblast rest | 18.6 |
| Dendritic cells anti-CD40 | 16.5 | Neutrophils TNFa + LPS | 8.3 |
| Monocytes rest | 100.0 | Neutrophils rest | 20.6 |
| Monocytes LPS | 0.0 | Colon | 0.0 |
| Macrophages rest | 0.0 | Lung | 0.0 |
| Macrophages LPS | 64.6 | Thymus | 1.9 |
| HUVEC none | 0.0 | Kidney | 79.6 |
| HUVEC starved | 44.4 | | |

CNS_neurodegeneration_v1.0 Summary: Ag3792 Expression of the CG90155-01 gene is low/undetectable in all samples on this panel (CTs>35).

General_screening_panel_v1.4 Summary: Ag3792 Highest expression of the CG90155-01 gene is seen in the placenta (CT=33). Thus, expression of this gene could be used to differentiate between this sample and other samples on this panel.

Low but significant levels of expression are also seen in cell lines from pancreatic cancer, brain cancer and renal cancer. Thus, expression of this gene could be used to differentiate between these cell lines and other samples on this panel and as a marker for these cancers. Furthermore, therapeutic modulation of the expression or function of this gene may be useful in the treatment of pancreatic, brain and renal cancers.

Among metabolic tissues, low but significant levels of expression are seen in thyroid, adrenal, and skeletal muscle. Thus, this gene product may be involved in the diagnosis and/or treatment of metabolic disorders, such as obesity and diabetes.

Panel 4.1D Summary: Ag3792 Highest expression of the CG90155-01 gene is seen in resting monocytes (CT=33.8). The expression of this gene in resting cells of these lineages suggests that the protein encoded by this transcript may be involved in normal immunological processes.

E. NOV9a (CG90750-01): HGT Keratin

Expression of gene CG90750-01 was assessed using the primer-probe set Ag3714, described in Table EA. Results of the RTQ-PCR runs are shown in Table EB.

TABLE EA

Probe Name Ag3714

| | | | | |
|---|---|---|---|---|
| | | | Start | SEQ ID |
| Primers | Sequences | Length | Position | No |

| | | | | |
|---|---|---|---|---|
| Forward | 5′-ctgtacgggaagagaccttcat-3′ | 22 | 3 | 103 |
| Probe | TET-5′-ttgggtaacttacccttcacaatcca-3′-TAMRA | 26 | 31 | 104 |
| Reverse | 5′-gcagcaattgagaaggatttag-3′ | 22 | 58 | 105 |

TABLE EB
General_screening_panel_v1.4

| | Rel. Exp. (%) | | Rel. Exp. (%) |
|---|---|---|---|
| | Ag3714, Run | | Ag3714, Run |
| Tissue Name | 218267284 | Tissue Name | 218267284 |
| Adipose | 0.0 | Renal ca. TK-10 | 0.0 |
| Melanoma* Hs688(A).T | 0.0 | Bladder | 9.7 |
| Melanoma* Hs688(B).T | 0.0 | Gastric ca. (liver met.) | 0.0 |
| | | NCI-N87 | |
| Melanoma* M14 | 41.5 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 9.8 |
| Squamous cell carcinoma | 0.0 | Colon ca.* (SW480 met) | 0.0 |
| SCC-4 | | SW620 | |
| Testis Pool | 66.9 | Colon ca. HT29 | 0.0 |
| Prostate ca.* (bone met) | 0.0 | Colon ca. HCT-116 | 0.0 |
| PC-3 | | | |
| Prostate Pool | 10.7 | Colon ca. CaCo-2 | 0.0 |
| Placenta | 0.0 | Colon cancer tissue | 0.0 |
| Uterus Pool | 4.1 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 50.3 |
| Ovarian ca. OVCAR-5 | 0.0 | Small Intestine Pool | 0.0 |
| Ovarian ca. IGROV-1 | 10.5 | Stomach Pool | 0.0 |
| Ovarian ca. OVCAR-8 | 0.0 | Bone Marrow Pool | 62.4 |
| Ovary | 0.0 | Fetal Heart | 10.2 |
| Breast ca. MCF-7 | 0.0 | Heart Pool | 13.6 |
| Breast ca. MDA-MB-231 | 9.0 | Lymph Node Pool | 0.0 |
| Breast ca. BT 549 | 0.0 | Fetal Skeletal Muscle | 0.0 |
| Breast ca. T47D | 0.0 | Skeletal Muscle Pool | 9.7 |
| Breast ca. MDA-N | 20.0 | Spleen Pool | 0.0 |
| Breast Pool | 7.7 | Thymus Pool | 8.5 |
| Trachea | 0.0 | CNS cancer (glio/astro) | 0.0 |
| | | U87-MG | |
| Lung | 0.0 | CNS cancer (glio/astro) | 0.0 |
| | | U-118-MG | |
| Fetal Lung | 8.9 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 7.9 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 4.6 |
| Lung ca. NCI-H23 | 0.0 | Brain (fetal) | 13.9 |
| Lung ca. NCI-H460 | 0.0 | Brain (Hippocampus) Pool | 0.0 |
| Lung ca. HOP-62 | 0.0 | Cerebral Cortex Pool | 21.2 |
| Lung ca. NCI-H522 | 0.0 | Brain (Substantia nigra) | 0.0 |
| | | Pool | |
| Liver | 0.0 | Brain (Thalamus) Pool | 12.9 |
| Fetal Liver | 19.1 | Brain (whole) | 0.0 |
| Liver ca. HepG2 | 0.0 | Spinal Cord Pool | 12.2 |
| Kidney Pool | 18.3 | Adrenal Gland | 8.1 |
| Fetal Kidney | 100.0 | Pituitary gland Pool | 12.8 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 73.2 |

CNS_neurodegeneration_v1.0 Summary: Ag3714 Expression of the CG90750-01 gene is low/undetectable in all samples on this panel (CTs>35).

General_screening panel_v1.4 Summary: Ag3714 Expression of the CG90750-01 gene is restricted to the fetal kidney (CT=34.8). Thus, expression of this gene could be used to differentiate between this sample and other samples and as a marker of fetal kidney tissue.

Panel 4.1D Summary: Ag3714 Expression of the CG90750-01 gene is low/undetectable in all samples on this panel (CTs>35).

F. NOV10 (CG91235-01): Interleukin 8.

Expression of gene CG91235-01 was assessed using the primer-probe sets Ag3838 and Ag3723, described in Tables FA and FB. Results of the RTQ-PCR runs are shown in Tables FC and FD.

TABLE FA

Probe Name Ag3838

| | | | | |
|---|---|---|---|---|
| Primers | Sequences | Length | Start Position | SEQ ID No |

| | | | | |
|---|---|---|---|---|
| Forward | 5′-catagtcagactgaaagatgg-3′ | 21 | 228 | 106 |
| Probe | TET-5′-ttagtcatcacccatgtagcctca-3′-TAMRA | 24 | 270 | 107 |
| Reverse | 5′-acctgtccataatctctttgat-3′ | 22 | 299 | 108 |

TABLE FB

Probe Name Ag3723

| | | | | |
|---|---|---|---|---|
| | | | Start | SEQ ID |
| Primers | Sequences | Length | Position | No |

| | | | | |
|---|---|---|---|---|
| Forward | 5′-gctgttgctctactgctttctt-3′ | 22 | 43 | 109 |
| Probe | TET-5′-atgttcactgcttccattgtgccaag-3′-TAMRA | 26 | 85 | 110 |
| Reverse | 5′-cactggcattgtggtactgtac-3′ | 22 | 116 | 111 |

TABLE FC
General_screening_panel_v1.4

| | Rel. Exp. (%) | | Rel. Exp. (%) |
|---|---|---|---|
| | Ag3838, Run | | Ag3838, Run |
| Tissue Name | 213604098 | Tissue Name | 213604098 |
| Adipose | 2.2 | Renal ca. TK-10 | 7.4 |
| Melanoma* Hs688(A).T | 0.0 | Bladder | 14.8 |
| Melanoma* Hs688(B).T | 0.0 | Gastric ca. (liver met.) NCI-N87 | 11.4 |
| Melanoma* M14 | 0.0 | Gastric ca. KATO III | 100.0 |
| Melanoma* LOXIMVI | 0.0 | Colon ca. SW-948 | 5.3 |
| Melanoma* SK-MEL-5 | 4.3 | Colon ca. SW480 | 0.0 |
| Squamous cell carcinoma | 0.0 | Colon ca.* (SW480 met) | 17.0 |
| SCC-4 | | SW620 | |
| Testis Pool | 1.1 | Colon ca. HT29 | 3.8 |
| Prostate ca.* (bone met) | 11.1 | Colon ca. HCT-116 | 2.6 |
| PC-3 | | | |
| Prostate Pool | 0.0 | Colon ca. CaCo-2 | 1.1 |
| Placenta | 0.0 | Colon cancer tissue | 7.8 |
| Uterus Pool | 0.0 | Colon ca. SW1116 | 0.0 |
| Ovarian ca. OVCAR-3 | 2.5 | Colon ca. Colo-205 | 0.0 |
| Ovarian ca. SK-OV-3 | 2.1 | Colon ca. SW-48 | 0.0 |
| Ovarian ca. OVCAR-4 | 0.0 | Colon Pool | 0.0 |
| Ovarian ca. OVCAR-5 | 3.1 | Small Intestine Pool | 0.0 |
| Ovarian ca. IGROV-1 | 6.5 | Stomach Pool | 0.0 |
| Ovarian ca. OVCAR-8 | 4.0 | Bone Marrow Pool | 0.0 |
| Ovary | 1.0 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 3.9 | Fetal Skeletal Muscle | 2.2 |
| Breast ca. T47D | 6.7 | Skeletal Muscle Pool | 0.0 |
| Breast ca. MDA-N | 0.0 | Spleen Pool | 1.9 |
| Breast Pool | 0.0 | Thymus Pool | 3.5 |
| Trachea | 0.0 | CNS cancer (glio/astro) | 12.9 |
| | | U87-MG | |
| Lung | 0.0 | CNS cancer (glio/astro) | 5.1 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 12.4 | CNS cancer (astro) SNB-75 | 0.0 |
| Lung ca. NCI-H146 | 8.2 | CNS cancer (glio) SNB-19 | 0.0 |
| Lung ca. SHP-77 | 16.4 | CNS cancer (glio) SF-295 | 10.3 |
| Lung ca. A549 | 12.1 | Brain (Amygdala) Pool | 1.3 |
| Lung ca. NCI-H526 | 0.0 | Brain (cerebellum) | 0.0 |
| Lung ca. NCI-H23 | 25.7 | Brain (fetal) | 0.0 |
| Lung ca. NCI-H460 | 35.8 | Brain (Hippocampus) Pool | 6.5 |
| Lung ca. HOP-62 | 1.5 | Cerebral Cortex Pool | 12.1 |
| Lung ca. NCI-H522 | 0.0 | Brain (Substantia nigra) Pool | 4.4 |
| Liver | 0.0 | Brain (Thalamus) Pool | 3.1 |
| Fetal Liver | 5.7 | Brain (whole) | 1.7 |
| Liver ca. HepG2 | 0.0 | Spinal Cord Pool | 8.2 |
| Kidney Pool | 1.1 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 1.7 | Pancreatic ca. CAPAN2 | 1.6 |
| Renal ca. UO-31 | 7.8 | Pancreas Pool | 0.0 |

TABLE FD
Panel 4.1D

| | Rel. Exp. (%) | | Rel. Exp. (%) |
|---|---|---|---|
| | Ag3838, Run | | Ag3838, Run |
| Tissue Name | 170127333 | Tissue Name | 170127333 |
| Secondary Th1 act | 8.2 | HUVEC IL-1beta | 0.0 |
| Secondary Th2 act | 0.0 | HUVEC IFN gamma | 8.4 |
| Secondary Tr1 act | 4.7 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 5.8 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium none | 0.0 |
| Primary Tr1 rest | 0.0 | Small airway epithelium | 10.0 |
| | | TNF alpha + IL-1beta | |
| CD45RA CD4 lymphocyte act | 0.0 | Coronery artery SMC rest | 9.4 |
| CD45RO CD4 lymphocyte act | 0.0 | Coronery artery SMC | 0.0 |
| | | TNF alpha + IL-1beta | |
| CD8 lymphocyte act | 0.0 | Astrocytes rest | 0.0 |
| Secondary CD8 lymphocyte rest | 0.0 | Astrocytes TNF alpha + | 0.0 |
| | | IL-1beta | |
| Secondary CD8 lymphocyte act | 0.0 | KU-812 (Basophil) rest | 6.0 |
| CD4 lymphocyte none | 0.0 | KU-812 (Basophil) | 3.4 |
| | | PMA/ionomycin | |
| 2ry Th1/Th2/Tr1_anti-CD95 | 0.0 | CCD1106 (Keratinocytes) | 14.6 |
| CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 16.2 |
| LAK cells IL-2 + IL-12 | 0.0 | NCI-H292 none | 0.0 |
| LAK cells IL-2 + IFN gamma | 0.0 | NCI-H292 IL-4 | 0.0 |
| LAK cells IL-2 + IL-18 | 0.0 | NCI-H292 IL-9 | 3.0 |
| LAK cells PMA/ionomycin | 40.9 | NCI-H292 IL-13 | 0.0 |
| NK Cells IL-2 rest | 0.0 | NCI-H292 IFN gamma | 10.1 |
| Two Way MLR 3 day | 0.0 | HPAEC none | 0.0 |
| Two Way MLR 5 day | 0.0 | HPAEC TNF alpha + IL-1 | 27.0 |
| | | beta | |
| Two Way MLR 7 day | 0.0 | Lung fibroblast none | 0.0 |
| PBMC rest | 0.0 | Lung fibroblast TNF alpha + | 16.2 |
| | | IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 9.4 |
| B lymphocytes PWM | 0.0 | Dermal fibroblast CCD1070 | 7.6 |
| | | rest | |
| B lymphocytes CD40L and IL-4 | 0.0 | Dermal fibroblast CCD1070 | 8.4 |
| | | TNF alpha | |
| EOL-1 dbcAMP | 0.0 | Dermal fibroblast CCD1070 | 0.0 |
| | | IL-1beta | |
| EOL-1 dbcAMP | 5.4 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 10.5 | Neutrophils TNFa + LPS | 19.6 |
| Monocytes rest | 0.0 | Neutrophils rest | 15.0 |
| Monocytes LPS | 100.0 | Colon | 8.3 |
| Macrophages rest | 0.0 | Lung | 0.0 |
| Macrophages LPS | 0.0 | Thymus | 92.0 |
| HUVEC none | 0.0 | Kidney | 0.0 |
| HUVEC starved | 0.0 | | |

CNS_neurodegeneration v1.0 Summary: Ag3838 Expression of the CG91235-01 gene is low/undetectable in all samples on this panel (CTs>35).

General_screening_panel_v1.4 Summary: Ag3838 Significant expression of the CG91235-01 gene in this panel is restricted to samples derived from gastric and lung cancer cell lines (CTs=32.5-34). Thus, expression of this gene could be used to differentiate between these samples and other samples on this panel and as a marker to detect the presence of gastric and lung cancers. Furthermore, therapeutic modulation of the expression or function of this gene may be effective in the treatment of gastric and lung cancers. A second experiment with the probe and primer set Ag3723 shows low/undetectable levels of expression (CTs>35).

Panel 2.2 Summary: Ag3838 Expression of the CG91235-01 gene is low/undetectable in all samples on this panel (CTs>35).

Panel 4.1D Summary: Ag3838 Significant expression of the CG91235-01 gene in this panel is restricted to LPS stimulated monocytes and the thymus (CTs=34.5). Upon activation with pathogens such as LPS, monocytes contribute to the innate and specific immunity by migrating to the site of tissue injury and releasing inflammatory cytokines. This release contributes to the inflammation process. Therefore, modulation of the expression of the putative IL-8 protein encoded by this transcript may prevent the recruitment of monocytes and the initiation of the inflammatory process, and reduce the symptoms of patients suffering from autoimmune and inflammatory diseases such as asthma, allergies, inflammatory bowel disease, lupus erythematosus, or rheumatoid arthritis.

G. NOV11a and NOV11b (CG91657-01 and CG91657-02): Brush Border Protein Precursor

Expression of gene CG91657-01 was assessed using the primer-probe set Ag3735, described in Table GA. Results of the RTQ-PCR runs are shown in Table GB. Please note that CG91657-02 represents a full-length physical clone of the CG91657-01 gene, validating the prediction of the gene sequence.

TABLE GA

Probe Name Ag3735

| | | | Start | SEQ ID |
|---|---|---|---|---|
| Primers | Sequences | Length | Position | No |
| Forward | 5′-cctctttgaaaggtcaaatgtg-3′ | 22 | 882 | 112 |
| Probe | TET-5′-tcaatacaattagtgtctccaaatgcaa-3′-TAMRA | 28 | 926 | 113 |
| Reverse | 5′-tttcattgcaactgtttctttg-3′ | 22 | 954 | 114 |

TABLE GB
General_screening_panel_v1.4

| | Rel. Exp. (%) | | Rel. Exp. (%) |
|---|---|---|---|
| | Ag3735, Run | | Ag3735, Run |
| Tissue Name | 218275229 | Tissue Name | 218275229 |
| Adipose | 1.5 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI-N87 | 0.0 |
| Melanoma* M14 | 0.0 | Gastric ca. KATO III | 0.0 |
| Melanoma* LOXIMVI | 0.0 | Colon ca. SW-948 | 1.5 |
| Melanoma* SK-MEL-5 | 0.0 | Colon ca. SW480 | 0.0 |
| Squamous cell carcinoma | 0.0 | Colon ca.* (SW480 met) | 0.0 |
| SCC-4 | | SW620 | |
| Testis Pool | 0.0 | Colon ca. HT29 | 0.0 |
| Prostate ca.* (bone met) PC-3 | 0.0 | Colon ca. HCT-116 | 0.0 |
| Prostate Pool | 0.8 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 5.1 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 2.6 | CNS cancer (glio/astro) | 0.0 |
| | | U87-MG | |
| Lung | 0.0 | CNS cancer (glio/astro) | 0.0 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.6 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.0 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 100.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

CNS_neurodegeneration_v1.0 Summary: Ag3735 Expression of the CG91657-01 gene is low/undetectable in all samples on this panel (CTs>35).

General_screening_panel_v1.4 Summary: Ag3735 Expression of the CG91657-01 gene is exclusive to the salivary gland (CT=32.5). Thus, expression of this gene could be used to differentiate this sample from other samples on this panel and as a marker to identify this glandular tissue.

Panel 4.1D Summary: Ag3735 Expression of the CG91657-01 gene is low/undetectable in all samples on this panel (CTs>35).

H. NOV12a and NOV12f (CG91678-01 and CG91678-03): MMP1

Expression of gene CG91678-01 and full length physical clone CG91678-03 was assessed using the primer-probe set Ag3394, described in Table HA. Results of the RTQ-PCR runs are shown in Tables HB, HC, HD, HE, HF, HG and HH.

TABLE HA

Probe Name Ag3394

| | | | Start | SEQ ID |
|---|---|---|---|---|
| Primers | Sequences | Length | Position | No |
| Forward | 5′-tggaccaacaatttcagagagt-3′ | 22 | 678 | 115 |
| Probe | TET-5′-acaacttacatcgtgttgcggctcat-3′-TAMRA | 26 | 700 | 116 |
| Reverse | 5′-agaatgggagagtccaagagaa-3′ | 22 | 737 | 117 |

TABLE HB
AI_comprehensive panel_v1.0

| | Rel. Exp. (%) | | Rel. Exp. (%) |
|---|---|---|---|
| | Ag3394, Run | | Ag3394, Run |
| Tissue Name | 217700461 | Tissue Name | 217700461 |
| 110967 COPD-F | 0.0 | 112427 Match Control | 0.0 |
| | | Psoriasis-F | |
| 110980 COPD-F | 0.0 | 112418 Psoriasis-M | 0.0 |
| 110968 COPD-M | 0.0 | 112723 Match Control | 0.5 |
| | | Psoriasis-M | |
| 110977 COPD-M | 0.0 | 112419 Psoriasis-M | 0.4 |
| 110989 Emphysema-F | 0.0 | 112424 Match Control | 0.0 |
| | | Psoriasis-M | |
| 110992 Emphysema-F | 0.1 | 112420 Psoriasis-M | 0.0 |
| 110993 Emphysema-F | 0.0 | 112425 Match Control | 0.0 |
| | | Psoriasis-M | |
| 110994 Emphysema-F | 0.1 | 104689 (MF) OA Bone-Backus | 31.6 |
| 110995 Emphysema-F | 0.0 | 104690 (MF) Adj “Normal” | 0.9 |
| | | Bone-Backus | |
| 110996 Emphysema-F | 0.0 | 104691 (MF) OA | 3.3 |
| | | Synovium-Backus | |
| 110997 Asthma-M | 0.1 | 104692 (BA) OA | 2.2 |
| | | Cartilage-Backus | |
| 111001 Asthma-F | 0.0 | 104694 (BA) OA Bone-Backus | 6.4 |
| 111002 Asthma-F | 0.0 | 104695 (BA) Adj “Normal” | 1.1 |
| | | Bone-Backus | |
| 111003 Atopic Asthma-F | 0.0 | 104696 (BA) OA | 100.0 |
| | | Synovium-Backus | |
| 111004 Atopic Asthma-F | 0.0 | 104700 (SS) OA Bone-Backus | 1.9 |
| 111005 Atopic Asthma-F | 0.0 | 104701 (SS) Adj “Normal” | 42.0 |
| | | Bone-Backus | |
| 111006 Atopic Asthma-F | 0.0 | 104702 (SS) OA | 0.8 |
| | | Synovium-Backus | |
| 111417 Allergy-M | 0.0 | 117093 OA Cartilage Rep7 | 4.7 |
| 112347 Allergy-M | 0.0 | 112672 OA Bone5 | 7.3 |
| 112349 Normal Lung-F | 0.0 | 112673 OA Synovium5 | 2.0 |
| 112357 Normal Lung-F | 2.0 | 112674 OA Synovial Fluid | 3.4 |
| | | cells5 | |
| 112354 Normal Lung-M | 0.0 | 117100 OA Cartilage Rep14 | 0.0 |
| 112374 Crohns-F | 0.5 | 112756 OA Bone9 | 1.3 |
| 112389 Match Control | 0.3 | 112757 OA Synovium9 | 0.0 |
| Crohns-F | | | |
| 112375 Crohns-F | 0.6 | 112758 OA Synovial Fluid | 0.0 |
| | | Cells9 | |
| 112732 Match Control | 0.0 | 117125 RA Cartilage Rep2 | 0.0 |
| Crohns-F | | | |
| 112725 Crohns-M | 0.0 | 113492 Bone2 RA | 1.4 |
| 112387 Match Control | 0.4 | 113493 Synovium2 RA | 0.3 |
| Crohns-M | | | |
| 112378 Crohns-M | 0.0 | 113494 Syn Fluid Cells RA | 1.2 |
| 112390 Match Control | 0.0 | 113499 Cartilage4 RA | 0.5 |
| Crohns-M | | | |
| 112726 Crohns-M | 0.1 | 113500 Bone4 RA | 0.6 |
| 112731 Match Control | 0.0 | 113501 Synovium4 RA | 0.0 |
| Crohns-M | | | |
| 112380 Ulcer Col-F | 0.0 | 113502 Syn Fluid Cells4 RA | 0.3 |
| 112734 Match Control Ulcer | 1.9 | 113495 Cartilage3 RA | 0.0 |
| Col-F | | | |
| 112384 Ulcer Col-F | 0.0 | 113496 Bone3 RA | 0.0 |
| 112737 Match Control Ulcer | 0.0 | 113497 Synovium3 RA | 0.0 |
| Col-F | | | |
| 112386 Ulcer Col-F | 0.0 | 113498 Syn Fluid Cells3 RA | 0.2 |
| 112738 Match Control Ulcer | 34.9 | 117106 Normal Cartilage Rep20 | 0.0 |
| Col-F | | | |
| 112381 Ulcer Col-M | 0.0 | 113663 Bone3 Normal | 0.0 |
| 112735 Match Control Ulcer | 0.0 | 113664 Synovium3 Normal | 0.0 |
| Col-M | | | |
| 112382 Ulcer Col-M | 0.0 | 113665 Syn Fluid Cells3 Normal | 0.0 |
| 112394 Match Control Ulcer | 0.0 | 117107 Normal Cartilage Rep22 | 0.0 |
| Col-M | | | |
| 112383 Ulcer Col-M | 0.1 | 113667 Bone4 Normal | 0.0 |
| 112736 Match Control Ulcer | 0.4 | 113668 Synovium4 Normal | 0.0 |
| Col-M | | | |
| 112423 Psoriasis-F | 0.0 | 113669 Syn Fluid Cells4 Normal | 0.0 |

TABLE HC
General_screening_panel_v1.4

| | Rel. | Rel. | | Rel. | Rel. |
|---|---|---|---|---|---|
| | Exp. (%) | Exp. (%) | | Exp. (%) | Exp. (%) |
| | Ag3394, | Ag3394, | | Ag3394, | Ag3394, |
| | Run | Run | | Run | Run |
| Tissue Name | 208033837 | 212142252 | Tissue Name | 208033837 | 212142252 |
| Adipose | 0.1 | 0.1 | Renal ca. TK-10 | 0.0 | 0.0 |
| Melanoma* | 0.2 | 0.2 | Bladder | 0.2 | 0.3 |
| Hs688(A).T | | | | | |
| Melanoma* | 6.3 | 5.2 | Gastric ca. (liver met.) | 0.0 | 0.0 |
| Hs688(B).T | | | NCI-N87 | | |
| Melanoma* M14 | 0.7 | 0.5 | Gastric ca. KATO III | 0.5 | 0.5 |
| Melanoma* | 0.9 | 0.9 | Colon ca. SW-948 | 0.0 | 0.0 |
| LOXIMVI | | | | | |
| Melanoma* | 0.1 | 0.1 | Colon ca. SW480 | 0.0 | 0.0 |
| SK-MEL-5 | | | | | |
| Squamous cell | 0.9 | 0.6 | Colon ca.* (SW480 met) | 0.0 | 0.0 |
| carcinoma SCC-4 | | | SW620 | | |
| Testis Pool | 0.0 | 0.0 | Colon ca. HT29 | 0.0 | 0.0 |
| Prostate ca.* (bone | 1.4 | 0.7 | Colon ca. HCT-116 | 0.0 | 0.0 |
| met) PC-3 | | | | | |
| Prostate Pool | 0.0 | 0.0 | Colon ca. CaCo-2 | 1.0 | 0.8 |
| Placenta | 0.3 | 0.2 | Colon cancer tissue | 12.2 | 10.7 |
| Uterus Pool | 0.0 | 0.0 | Colon ca. SW1116 | 0.0 | 0.0 |
| Ovarian ca. | 0.0 | 0.0 | Colon ca. Colo-205 | 0.0 | 0.0 |
| OVCAR-3 | | | | | |
| Ovarian ca. | 3.0 | 2.5 | Colon ca. SW-48 | 0.0 | 0.0 |
| SK-OV-3 | | | | | |
| Ovarian ca. | 0.0 | 0.0 | Colon Pool | 0.0 | 0.0 |
| OVCAR-4 | | | | | |
| Ovarian ca. | 0.0 | 0.0 | Small Intestine Pool | 0.0 | 0.0 |
| OVCAR-5 | | | | | |
| Ovarian ca. | 0.7 | 0.7 | Stomach Pool | 2.3 | 1.7 |
| IGROV-1 | | | | | |
| Ovarian ca. | 0.0 | 0.0 | Bone Marrow Pool | 0.0 | 0.0 |
| OVCAR-8 | | | | | |
| Ovary | 0.0 | 0.0 | Fetal Heart | 0.0 | 0.0 |
| Breast ca. MCF-7 | 0.0 | 0.0 | Heart Pool | 0.0 | 0.0 |
| Breast ca. | 0.4 | 0.6 | Lymph Node Pool | 0.0 | 0.0 |
| MDA-MB-231 | | | | | |
| Breast ca. BT 549 | 1.2 | 1.8 | Fetal Skeletal Muscle | 0.0 | 0.0 |
| Breast ca. T47D | 0.0 | 0.0 | Skeletal Muscle Pool | 0.0 | 0.0 |
| Breast ca. MDA-N | 0.1 | 0.1 | Spleen Pool | 0.0 | 0.0 |
| Breast Pool | 0.0 | 0.0 | Thymus Pool | 0.0 | 0.0 |
| Trachea | 0.1 | 0.0 | CNS cancer (glio/astro) | 1.6 | 1.3 |
| | | | U87-MG | | |
| Lung | 0.0 | 0.0 | CNS cancer (glio/astro) | 24.3 | 20.3 |
| | | | U-118-MG | | |
| Fetal Lung | 0.0 | 0.0 | CNS cancer (neuro; met) | 0.1 | 0.1 |
| | | | SK-N-AS | | |
| Lung ca. NCI-N417 | 0.0 | 0.0 | CNS cancer (astro) SF-539 | 0.0 | 0.0 |
| Lung ca. LX-1 | 0.0 | 0.0 | CNS cancer (astro) | | |
| | | | SNB-75 | | |
| Lung ca. NCI-H146 | 0.0 | 0.0 | CNS cancer (glio) SNB-19 | 0.4 | 0.6 |
| Lung ca. SHP-77 | 0.0 | 0.0 | CNS cancer (glio) SF-295 | 100.0 | 100.0 |
| Lung ca. A549 | 0.0 | 0.0 | Brain (Amygdala) Pool | 0.0 | 0.0 |
| Lung ca. NCI-H526 | 0.0 | 0.0 | Brain (cerebellum) | 0.0 | 0.0 |
| Lung ca. NCI-H23 | 0.2 | 0.1 | Brain (fetal) | 0.0 | 0.0 |
| Lung ca. NCI-H460 | 0.1 | 0.0 | Brain (Hippocampus) Pool | 0.0 | 0.0 |
| Lung ca. HOP-62 | 0.0 | 0.0 | Cerebral Cortex Pool | 0.0 | 0.0 |
| Lung ca. NCI-H522 | 0.1 | 0.1 | Brain (Substantia nigra) | 0.0 | 0.0 |
| | | | Pool | | |
| Liver | 0.0 | 0.0 | Brain (Thalamus) Pool | 0.0 | 0.0 |
| Fetal Liver | 0.0 | 0.0 | Brain (whole) | 0.0 | 0.0 |
| Liver ca. HepG2 | 0.0 | 0.0 | Spinal Cord Pool | 0.0 | 0.0 |
| Kidney Pool | 0.0 | 0.0 | Adrenal Gland | 0.0 | 0.0 |
| Fetal Kidney | 0.0 | 0.0 | Pituitary gland Pool | 0.0 | 0.0 |
| Renal ca. 786-0 | 0.0 | 0.0 | Salivary Gland | 0.0 | 0.0 |
| Renal ca. A498 | 0.0 | 0.0 | Thyroid (female) | 0.0 | 0.0 |
| Renal ca. ACHN | 0.0 | 1.9 | Pancreatic ca. CAPAN2 | 0.0 | 0.0 |
| Renal ca. UO-31 | 1.1 | 0.8 | Pancreas Pool | 0.1 | 0.0 |

TABLE HD
Panel 1.3D

| | Rel. Exp. (%) | Rel. Exp. (%) | | Rel. Exp. (%) | Rel. Exp. (%) |
|---|---|---|---|---|---|
| | Ag3394, Run | Ag3394, Run | | Ag3394, Run | Ag3394, Run |
| Tissue Name | 165524929 | 167595301 | Tissue Name | 165524929 | 167595301 |
| Liver | 0.0 | 0.0 | Kidney (fetal) | 0.0 | 0.2 |
| adenocarcinoma | | | | | |
| Pancreas | 0.0 | 0.0 | Renal ca. 786-0 | 0.1 | 0.0 |
| Pancreatic ca. | 0.0 | 0.0 | Renal ca. A498 | 0.0 | 0.0 |
| CAPAN 2 | | | | | |
| Adrenal gland | 0.0 | 0.0 | Renal ca. RXF | 0.1 | 0.1 |
| | | | 393 | | |
| Thyroid | 0.1 | 0.0 | Renal ca. ACHN | 0.0 | 0.0 |
| Salivary gland | 0.1 | 0.0 | Renal ca. UO-31 | 3.8 | 0.7 |
| Pituitary gland | 0.1 | 0.0 | Renal ca. TK-10 | 0.0 | 0.0 |
| Brain (fetal) | 0.0 | 0.0 | Liver | 0.0 | 0.0 |
| Brain (whole) | 0.0 | 0.0 | Liver (fetal) | 0.1 | 0.1 |
| Brain | 0.0 | 0.0 | Liver ca. | 0.0 | 0.0 |
| (amygdala) | | | (hepatoblast) | | |
| | | | HepG2 | | |
| Brain | 0.0 | 0.0 | Lung | 0.1 | 0.0 |
| (cerebellum) | | | | | |
| Brain | 0.0 | 0.0 | Lung (fetal) | 1.2 | 0.5 |
| (hippocampus) | | | | | |
| Brain (substantia | 0.0 | 0.0 | Lung ca. (small | 0.0 | 0.0 |
| nigra) | | | cell) LX-1 | | |
| Brain (thalamus) | 0.0 | 0.0 | Lung ca. (small | 0.0 | 0.0 |
| | | | cell) NCI-H69 | | |
| Cerebral Cortex | 0.0 | 0.0 | Lung ca. (s.cell | 0.0 | 0.0 |
| | | | var.) SHP-77 | | |
| Spinal cord | 0.0 | 0.0 | Lung ca. (large | 0.4 | 0.0 |
| | | | cell)NCI-H460 | | |
| glio/astro | 2.1 | 1.1 | Lung ca. | 0.0 | 0.0 |
| U87-MG | | | (non-sm. cell) | | |
| | | | A549 | | |
| glio/astro | 66.0 | 14.2 | Lung ca. | 0.0 | 0.0 |
| U-118-MG | | | (non-s.cell) | | |
| | | | NCI-H23 | | |
| astrocytoma | 40.3 | 18.6 | Lung ca. | 0.0 | 0.0 |
| SW1783 | | | (non-s.cell) | | |
| | | | HOP-62 | | |
| neuro*; met | 0.4 | 0.0 | Lung ca. | 0.3 | 0.0 |
| SK-N-AS | | | (non-s.cl) | | |
| | | | NCI-H522 | | |
| astrocytoma | 0.1 | 0.0 | Lung ca. | 3.1 | 1.5 |
| SF-539 | | | (squam.) SW | | |
| | | | 900 | | |
| astrocytoma | 1.2 | 0.6 | Lung ca. | 0.0 | 0.0 |
| SNB-75 | | | (squam.) | | |
| | | | NCI-H596 | | |
| glioma SNB-19 | 0.0 | 0.0 | Mammary gland | 0.1 | 0.0 |
| glioma U251 | 0.2 | 0.0 | Breast ca.* | 0.0 | 0.0 |
| | | | (pl.ef) MCF-7 | | |
| glioma SF-295 | 100.0 | 100.0 | Breast ca.* | 2.4 | 0.3 |
| | | | (pl.ef) | | |
| | | | MDA-MB-231 | | |
| Heart (fetal) | 0.0 | 0.0 | Breast ca.* | 0.0 | 0.0 |
| | | | (pl.ef) T47D | | |
| Heart | 0.0 | 0.0 | Breast ca. | 13.1 | 1.1 |
| | | | BT-549 | | |
| Skeletal muscle | 0.0 | 0.0 | Breast ca. | 0.2 | 0.1 |
| (fetal) | | | MDA-N | | |
| Skeletal muscle | 0.0 | 0.0 | Ovary | 0.0 | 0.0 |
| Bone marrow | 0.1 | 0.0 | Ovarian ca. | 0.1 | 0.0 |
| | | | OVCAR-3 | | |
| Thymus | 0.0 | 0.0 | Ovarian ca. | 0.0 | 0.0 |
| | | | OVCAR-4 | | |
| Spleen | 0.0 | 0.0 | Ovarian ca. | 0.0 | 0.1 |
| | | | OVCAR-5 | | |
| Lymph node | 0.1 | 0.0 | Ovarian ca. | 0.1 | 0.0 |
| | | | OVCAR-8 | | |
| Colorectal | 0.1 | 0.0 | Ovarian ca. | 1.4 | 0.6 |
| | | | IGROV-1 | | |
| Stomach | 2.7 | 0.4 | Ovarian ca.* | 2.7 | 6.5 |
| | | | (ascites) | | |
| | | | SK-OV-3 | | |
| Small intestine | 0.8 | 0.1 | Uterus | 3.6 | 0.3 |
| Colon ca. SW480 | 0.4 | 0.0 | Placenta | 0.3 | 0.0 |
| Colon ca.* | 0.0 | 0.2 | Prostate | 0.0 | 0.0 |
| SW620(SW480 | | | | | |
| met) | | | | | |
| Colon ca. HT29 | 0.0 | 0.0 | Prostate ca.* | 0.8 | 0.6 |
| | | | (bone met)PC-3 | | |
| Colon ca. | 0.0 | 0.0 | Testis | 0.0 | 0.0 |
| HCT-116 | | | | | |
| Colon ca. CaCo-2 | 1.7 | 0.8 | Melanoma | 0.5 | 0.3 |
| | | | Hs688(A).T | | |
| Colon ca. | 31.4 | 8.9 | Melanoma* (met) | 7.1 | 2.3 |
| tissue(ODO3866) | | | Hs688(B).T | | |
| Colon ca. | 0.0 | 0.0 | Melanoma | 0.0 | 0.0 |
| HCC-2998 | | | UACC-62 | | |
| Gastric ca.* (liver | 1.7 | 0.0 | Melanoma M14 | 0.2 | 0.0 |
| met) NCI-N87 | | | | | |
| Bladder | 0.4 | 0.3 | Melanoma LOX | 0.5 | 0.6 |
| | | | IMVI | | |
| Trachea | 0.4 | 0.0 | Melanoma* (met) | 0.1 | 0.0 |
| | | | SK-MEL-5 | | |
| Kidney | 0.0 | 0.0 | Adipose | 0.2 | 0.2 |

TABLE HE
Panel 2D

| | Rel. Exp. (%) | | Rel. Exp. (%) |
|---|---|---|---|
| | Ag3394, Run | | Ag3394, Run |
| Tissue Name | 165471510 | Tissue Name | 165471510 |
| Normal Colon | 2.0 | Kidney Margin 8120608 | 0.0 |
| CC Well to Mod Diff | 88.3 | Kidney Cancer 8120613 | 0.7 |
| (ODO3866) | | | |
| CC Margin (ODO3866) | 1.4 | Kidney Margin 8120614 | 0.0 |
| CC Gr.2 rectosigmoid | 13.3 | Kidney Cancer 9010320 | 3.2 |
| (ODO3868) | | | |
| CC Margin (ODO3868) | 0.0 | Kidney Margin 9010321 | 0.0 |
| CC Mod Diff (ODO3920) | 2.1 | Normal Uterus | 0.0 |
| CC Margin (ODO3920) | 0.1 | Uterus Cancer 064011 | 0.1 |
| CC Gr.2 ascend colon | 33.7 | Normal Thyroid | 0.2 |
| (ODO3921) | | | |
| CC Margin (ODO3921) | 0.9 | Thyroid Cancer 064010 | 0.2 |
| CC from Partial Hepatectomy | 20.2 | Thyroid Cancer A302152 | 2.8 |
| (ODO4309) Mets | | | |
| Liver Margin (ODO4309) | 0.2 | Thyroid Margin A302153 | 0.1 |
| Colon mets to lung | 1.2 | Normal Breast | 0.0 |
| (ODO4451-01) | | | |
| Lung Margin (OD04451-02) | 0.1 | Breast Cancer (OD04566) | 0.3 |
| Normal Prostate 6546-1 | 0.2 | Breast Cancer | 0.0 |
| | | (OD04590-01) | |
| Prostate Cancer (OD04410) | 0.2 | Breast Cancer Mets | 0.0 |
| | | (OD04590-03) | |
| Prostate Margin (OD04410) | 0.5 | Breast Cancer Metastasis | 0.0 |
| | | (OD04655-05) | |
| Prostate Cancer (OD04720-01) | 0.0 | Breast Cancer 064006 | 3.6 |
| Prostate Margin (OD04720-02) | 1.0 | Breast Cancer 1024 | 0.0 |
| Normal Lung 061010 | 1.0 | Breast Cancer 9100266 | 3.7 |
| Lung Met to Muscle | 2.9 | Breast Margin 9100265 | 1.6 |
| (ODO4286) | | | |
| Muscle Margin (ODO4286) | 0.2 | Breast Cancer A209073 | 1.1 |
| Lung Malignant Cancer | 31.4 | Breast Margin A209073 | 0.3 |
| (OD03126) | | | |
| Lung Margin (OD03126) | 1.0 | Normal Liver | 0.0 |
| Lung Cancer (OD04404) | 77.4 | Liver Cancer 064003 | 0.0 |
| Lung Margin (OD04404) | 4.0 | Liver Cancer 1025 | 0.0 |
| Lung Cancer (OD04565) | 91.4 | Liver Cancer 1026 | 0.3 |
| Lung Margin (OD04565) | 0.1 | Liver Cancer 6004-T | 0.0 |
| Lung Cancer (OD04237-01) | 33.9 | Liver Tissue 6004-N | 0.7 |
| Lung Margin (OD04237-02) | 2.7 | Liver Cancer 6005-T | 0.1 |
| Ocular Mel Met to Liver | 0.0 | Liver Tissue 6005-N | 0.0 |
| (ODO4310) | | | |
| Liver Margin (ODO4310) | 0.0 | Normal Bladder | 2.9 |
| Melanoma Mets to Lung | 3.3 | Bladder Cancer 1023 | 2.5 |
| (OD04321) | | | |
| Lung Margin (OD04321) | 0.1 | Bladder Cancer A302173 | 2.7 |
| Normal Kidney | 0.0 | Bladder Cancer | 59.9 |
| | | (OD04718-01) | |
| Kidney Ca, Nuclear grade 2 | 0.2 | Bladder Normal Adjacent | 0.6 |
| (OD04338) | | (OD04718-03) | |
| Kidney Margin (OD04338) | 0.2 | Normal Ovary | 0.0 |
| Kidney Ca Nuclear grade 1/2 | 1.4 | Ovarian Cancer 064008 | 0.8 |
| (OD04339) | | | |
| Kidney Margin (OD04339) | 0.0 | Ovarian Cancer | 1.0 |
| | | (OD04768-07) | |
| Kidney Ca, Clear cell type | 3.1 | Ovary Margin | 0.2 |
| (OD04340) | | (OD04768-08) | |
| Kidney Margin (OD04340) | 0.0 | Normal Stomach | 1.8 |
| Kidney Ca, Nuclear grade 3 | 2.3 | Gastric Cancer 9060358 | 5.9 |
| (OD04348) | | | |
| Kidney Margin (OD04348) | 0.0 | Stomach Margin 9060359 | 1.8 |
| Kidney Cancer (OD04622-01) | 5.7 | Gastric Cancer 9060395 | 100.0 |
| Kidney Margin (OD04622-03) | 0.3 | Stomach Margin 9060394 | 10.2 |
| Kidney Cancer (OD04450-01) | 0.1 | Gastric Cancer 9060397 | 13.0 |
| Kidney Margin (OD04450-03) | 0.0 | Stomach Margin 9060396 | 1.6 |
| Kidney Cancer 8120607 | 0.2 | Gastric Cancer 064005 | 27.2 |

TABLE HF
Panel 3D

| | Rel. Exp. (%) | | Rel. Exp. (%) |
|---|---|---|---|
| | Ag3394, Run | | Ag3394, Run |
| Tissue Name | 165902490 | Tissue Name | 165902490 |
| Daoy-Medulloblastoma | 0.0 | Ca Ski-Cervical epidermoid | 0.0 |
| | | carcinoma (metastasis) | |
| TE671-Medulloblastoma | 0.0 | ES-2-Ovarian clear cell | 100.0 |
| | | carcinoma | |
| D283 Med- | 0.0 | Ramos-Stimulated with | 0.0 |
| Medulloblastoma | | PMA/ionomycin 6 h | |
| PFSK-1-Primitive | 0.0 | Ramos-Stimulated with | 0.0 |
| Neuroectodermal | | PMA/ionomycin 14 h | |
| XF-498-CNS | 0.0 | MEG-01-Chronic myelogenous | 0.0 |
| | | leukemia (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.0 |
| T98G-Glioblastoma | 0.3 | U266-B-cell plasmacytoma | 0.0 |
| SK-N-SH-Neuroblastoma | 0.3 | CA46-Burkitt's lymphoma | 0.0 |
| (metastasis) | | | |
| SF-295-Glioblastoma | 31.2 | RL-non-Hodgkin's B-cell | 0.0 |
| | | lymphoma | |
| Cerebellum | 0.0 | JM1-pre-B-cell lymphoma | 0.0 |
| Cerebellum | 0.0 | Jurkat-T cell leukemia | 0.0 |
| NCI-H292- | 0.1 | TF-1-Erythroleukemia | 0.0 |
| Mucoepidermoid lung | | | |
| carcinoma | | | |
| DMS-114-Small cell lung | 0.0 | HUT 78-T-cell lymphoma | 0.0 |
| cancer | | | |
| DMS-79-Small cell lung | 0.0 | U937-Histiocytic lymphoma | 0.0 |
| cancer | | | |
| NCI-H146-Small cell lung | 0.0 | KU-812-Myelogenous leukemia | 0.0 |
| cancer | | | |
| NCI-H526-Small cell lung | 0.0 | 769-P-Clear cell renal | 0.1 |
| cancer | | carcinoma | |
| NCI-N417-Small cell lung | 0.0 | Caki-2-Clear cell renal | 0.0 |
| cancer | | carcinoma | |
| NCI-H82-Small cell lung | 0.0 | SW 839-Clear cell renal | 0.0 |
| cancer | | carcinoma | |
| NCI-H157-Squamous cell | 0.0 | G401-Wilms' tumor | 0.0 |
| lung cancer (metastasis) | | | |
| NCI-H1155-Large cell lung | 0.0 | Hs766T-Pancreatic carcinoma | 0.1 |
| cancer | | (LN metastasis) | |
| NCI-H1299-Large cell lung | 0.1 | CAPAN-1-Pancreatic | 0.0 |
| cancer | | adenocarcinoma (liver | |
| | | metastasis) | |
| NCI-H727-Lung carcinoid | 0.2 | SU86.86-Pancreatic carcinoma | 1.0 |
| | | (liver metastasis) | |
| NCI-UMC-11-Lung | 0.0 | BxPC-3-Pancreatic | 0.8 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 0.0 | MIA PaCa-2-Pancreatic | 0.0 |
| | | carcinoma | |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic epithelioid | 0.0 |
| | | ductal carcinoma | |
| NCI-H716-Colon cancer | 0.0 | T24-Bladder carcinma | 0.0 |
| | | (transitional cell) | |
| SW-48-Colon | 0.0 | 5637-Bladder carcinoma | 11.3 |
| adenocarcinoma | | | |
| SW1116-Colon | 0.0 | HT-1197-Bladder carcinoma | 0.1 |
| adenocarcinoma | | | |
| LS 174T-Colon | 0.1 | UM-UC-3-Bladder carcinma | 0.4 |
| adenocarcinoma | | (transitional cell) | |
| SW-948-Colon | 0.0 | A204-Rhabdomyosarcoma | 0.5 |
| adenocarcinoma | | | |
| SW-480-Colon | 0.0 | HT-1080-Fibrosarcoma | 0.0 |
| adenocarcinoma | | | |
| NCI-SNU-5-Gastric | 0.0 | MG-63-Osteosarcoma | 0.0 |
| carcinoma | | | |
| KATO III-Gastric | 0.2 | SK-LMS-1-Leiomyosarcoma | 10.2 |
| carcinoma | | (vulva) | |
| NCI-SNU-16-Gastric | 2.3 | SJRH30-Rhabdomyosarcoma | 0.0 |
| carcinoma | | (met to bone marrow) | |
| NCI-SNU-1-Gastric | 0.3 | A431-Epidermoid carcinoma | 0.0 |
| carcinoma | | | |
| RF-1-Gastric | 0.0 | WM266-4-Melanoma | 0.4 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 0.0 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine carcinoma | 0.0 | SCC-15-Squamous cell | 0.0 |
| | | carcinoma of tongue | |
| HelaS3-Cervical | 0.0 | CAL 27-Squamous cell | 0.0 |
| adenocarcinoma | | carcinoma of tongue | |

TABLE HG
Panel 4.1D

| | Rel. Exp. (%) | | Rel. Exp. (%) |
|---|---|---|---|
| | Ag3394, Run | | Ag3394, Run |
| Tissue Name | 169838992 | Tissue Name | 169838992 |
| Secondary Th1 act | 0.0 | HUVEC IL-1beta | 13.2 |
| Secondary Th2 act | 0.0 | HUVEC IFN gamma | 14.9 |
| Secondary Tr1 act | 0.0 | HUVEC TNF alpha + IFN | 2.8 |
| | | gamma | |
| Secondary Th1 rest | 0.0 | HUVEC TNF alpha + IL4 | 7.7 |
| Secondary Th2 rest | 0.0 | HUVEC IL-11 | 11.3 |
| Secondary Tr1 rest | 0.0 | Lung Microvascular EC none | 0.1 |
| Primary Th1 act | 0.0 | Lung Microvascular EC | 0.9 |
| | | TNF alpha + IL-1beta | |
| Primary Th2 act | 0.0 | Microvascular Dermal EC | 0.7 |
| | | none | |
| Primary Tr1 act | 0.0 | Microsvasular Dermal EC | 2.0 |
| | | TNF alpha + IL-1beta | |
| Primary Th1 rest | 0.0 | Bronchial epithelium | 1.6 |
| | | TNF alpha + IL-1beta | |
| Primary Th2 rest | 0.0 | Small airway epithelium none | 2.9 |
| Primary Tr1 rest | 0.0 | Small airway epithelium | 1.9 |
| | | TNF alpha + IL-1beta | |
| CD45RA CD4 lymphocyte act | 13.6 | Coroney artery SMC rest | 84.1 |
| CD45RO CD4 lymphocyte act | 0.0 | Coronery artery SMC | 90.8 |
| | | TNF alpha + IL-1beta | |
| CD8 lymphocyte act | 0.0 | Astrocytes rest | 0.0 |
| Secondary CD8 lymphocyte rest | 0.0 | Astrocytes TNF alpha + | 0.2 |
| | | IL-1beta | |
| Secondary CD8 lymphocyte act | 0.0 | KU-812 (Basophil) rest | 0.0 |
| CD4 lymphocyte none | 0.0 | KU-812 (Basophil) | 0.5 |
| | | PMA/ionomycin | |
| 2ry Th1/Th2/Tr1_anti-CD95 | 0.0 | CCD1106 (Keratinocytes) | 0.9 |
| CH11 | | none | |
| LAK cells rest | 0.2 | CCD1106 (Keratinocytes) | 1.9 |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN gamma | 0.0 | NCI-H292 IL-4 | 0.0 |
| LAK cells IL-2 + IL-18 | 0.0 | NCI-H292 IL-9 | 0.0 |
| LAK cells PMA/ionomycin | 0.0 | NCI-H292 IL-13 | 0.0 |
| NK Cells IL-2 rest | 0.0 | NCI-H292 IFN gamma | 0.0 |
| Two Way MLR 3 day | 0.0 | HPAEC none | 15.1 |
| Two Way MLR 5 day | 0.0 | HPAEC TNF alpha + IL-1 | 100.0 |
| | | beta | |
| Two Way MLR 7 day | 0.0 | Lung fibroblast none | 5.6 |
| PBMC rest | 0.0 | Lung fibroblast TNF alpha + | 86.5 |
| | | IL-1beta | |
| PBMC PWM | 0.0 | Lung fibroblast IL-4 | 6.6 |
| PBMC PHA-L | 0.2 | Lung fibroblast IL-9 | 19.3 |
| Ramos (B cell) none | 0.0 | Lung fibroblast IL-13 | 4.6 |
| Ramos (B cell) ionomycin | 0.0 | Lung fibroblast IFN gamma | 7.2 |
| B lymphocytes PWM | 0.1 | Dermal fibroblast CCD1070 | 6.5 |
| | | rest | |
| B lymphocytes CD40L and IL-4 | 0.2 | Dermal fibroblast CCD1070 | 19.6 |
| | | TNF alpha | |
| EOL-1 dbcAMP | 0.0 | Dermal fibroblast CCD1070 | 23.8 |
| | | IL-1beta | |
| EOL-1 dbcAMP | 0.0 | Dermal fibroblast IFN gamma | 15.5 |
| PMA/ionomycin | | | |
| Dendritic cells none | 0.1 | Dermal fibroblast IL-4 | 27.7 |
| Dendritic cells LPS | 0.1 | Dermal Fibroblasts rest | 13.9 |
| Dendritic cells anti-CD40 | 0.0 | Neutrophils TNFa + LPS | 0.0 |
| Monocytes rest | 0.0 | Neutrophils rest | 0.0 |
| Monocytes LPS | 15.8 | Colon | 0.0 |
| Macrophages rest | 0.0 | Lung | 0.7 |
| Macrophages LPS | 0.3 | Thymus | 0.0 |
| HUVEC none | 6.7 | Kidney | 0.0 |
| HUVEC starved | 4.9 | | |

TABLE HH
Panel 4D

| | Rel. Exp. (%) | | Rel. Exp. (%) |
|---|---|---|---|
| | Ag3394, Run | | Ag3394, Run |
| Tissue Name | 165222526 | Tissue Name | 165222526 |
| Secondary Th1 act | 0.0 | HUVEC IL-1beta | 5.7 |
| Secondary Th2 act | 0.0 | HUVEC IFN gamma | 12.9 |
| Secondary Tr1 act | 0.0 | HUVEC TNF alpha + IFN | 4.0 |
| | | gamma | |
| Secondary Th1 rest | 0.0 | HUVEC TNF alpha + IL4 | 9.5 |
| Secondary Th2 rest | 0.0 | HUVEC IL-11 | 10.6 |
| Secondary Tr1 rest | 0.0 | Lung Microvascular EC none | 0.0 |
| Primary Th1 act | 0.0 | Lung Microvascular EC | 1.2 |
| | | TNF alpha + IL-1beta | |
| Primary Th2 act | 0.0 | Microvascular Dermal EC | 0.7 |
| | | none | |
| Primary Tr1 act | 0.0 | Microvasular Dermal EC | 2.0 |
| | | TNF alpha + IL-1beta | |
| Primary Th1 rest | 0.0 | Bronchial epithelium | 2.1 |
| | | TNF alpha + IL1beta | |
| Primary Th2 rest | 0.0 | Small airway epithelium none | 3.5 |
| Primary Tr1 rest | 0.0 | Small airway epithelium | 6.4 |
| | | TNF alpha + IL-1beta | |
| CD45RA CD4 lymphocyte act | 8.5 | Coronery artery SMC rest | 100.0 |
| CD45RO CD4 lymphocyte act | 0.0 | Coronery artery SMC | 72.2 |
| | | TNF alpha + IL-1beta | |
| CD8 lymphocyte act | 0.0 | Astrocytes rest | 0.0 |
| Secondary CD8 lymphocyte act | 0.0 | Astrocytes TNF alpha + | 0.3 |
| | | IL-1beta | |
| Secondary CD8 lymphocyte act | 0.0 | KU-812 (Basophil) rest | 0.0 |
| CD4 lymphocyte none | 0.0 | KU-812 (Basophil) | 0.6 |
| | | PMA/ionomycin | |
| 2ry Th1/Th2/Tr1_anti-CD95 | 0.0 | CCD1106 (keratinocytes) | 1.1 |
| CH11 | | none | |
| LAK cells rest | 0.2 | CCD1106 (Keratinocytes) | 1.1 |
| | | TNF alpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 12.6 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 18.3 |
| Two Way MLR 7 day | 0.0 | HPAEC TNF alpha + | 72.7 |
| | | IL-1beta | |
| PBMC rest | 0.0 | Lung fibroblast none | 3.8 |
| PBMC PWM | 0.1 | Lung fibroblast TNF alpha + | 76.3 |
| | | IL-1beta | |
| PBMC PHA-L | 0.3 | Lung fibroblast IL-4 | 9.5 |
| Ramos (B cell) none | 0.0 | Lung fibroblast IL-9 | 19.1 |
| Ramos (B cell) ionomycin | 0.0 | Lung fibroblast IL-13 | 4.2 |
| B lymphocytes PWM | 0.1 | Lung fibroblast IFN gamma | 10.8 |
| B lymphocytes CD40L and IL-4 | 0.2 | Dermal fibroblast CCD1070 | 11.0 |
| | | rest | |
| EOL-1 dbcAMP | 0.0 | Dermal fibroblast CCD1070 | 32.1 |
| | | TNF alpha | |
| EOL-1 dbcAMP | 0.3 | Dermal fibroblast CCD1070 | 26.4 |
| PMA/ionomycin | | IL-1beta | |
| Dendritic cells none | 0.0 | Dermal fibroblast IFN gamma | 17.2 |
| Dendritic cells LPS | 0.0 | Dermal fibroblast IL-4 | 23.5 |
| Dendritic cells anti-CD40 | 0.0 | IBD Colitis 2 | 0.0 |
| Monocytes rest | 0.0 | IBD Crohn's | 0.0 |
| Monocytes LPS | 9.1 | Colon | 0.0 |
| Macrophages rest | 0.0 | Lung | 0.5 |
| Macrophages LPS | 0.4 | Thymus | 0.0 |
| HUVEC none | 11.0 | Kidney | 0.0 |
| HUVEC starved | 9.3 | | |

AI_comprehensive panel_v1.0 Summary: Ag3394 The CG91678-01 transcript is expressed in OA tissue but not in control tissue (CTs=28-30). The transcript encodes a molecule homologous to MMP1 which has been shown to be present in OA joint tissue and may contribute to the pathology of this disease. Although the transcript is not expressed at significant levels in the lung tissue on this panel, it is expressed in lung derived cell types and may be involved in lung remodeling associated with asthma, allergy, and emphysema (see panel 4 for references).

CNS_neurodegeneration_v1.0 Summary: Ag3394 Expression of the CG91678-01 gene is low/undetectable in all samples on this panel (CTs>35).

General_screening_panel_v1.4 Summary: Ag3394 Two experiments with the same probe and primer set produce results that are in excellent agreement, with highest expression of the CG91678-01 gene in a brain cancer cell line (CTs=20-22). Significant levels of expression are also seen in a cluster of cell lines derived from brain, colon, breast, ovarian and melanoma cancers. Thus, expression of this gene could be used to differentiate between the brain cancer cell lines and other samples on this panel and as a marker for brain cancer. Furthermore, therapeutic modulation of the expression or function of this gene may be effective in the treatment of brain, colon, breast, ovarian and melanoma cancers.

Among tissues with metabolic function, this gene is expressed at low but significant levels in pancreas, thyroid, adipose and fetal heart, and liver. This pattern of expression among these tissues suggests that this gene product may play a role in normal neuroendocrine and metabolic and that disregulated expression of this gene may contribute to neuroendocrine disorders or metabolic diseases, such as obesity and diabetes.

Panel 1.3D Summary: Ag3394 Two experiments with the same probe and primer set produce results that are in excellent agreement, with highest expression of the CG91678-01 gene in a brain cancer cell line (CTs=23.7-25.2). This expression in in concordance with the profile seen in Panel 1.4. Overall, expression is higher in cancer cell lines than in normal tissue samples, with significant levels of expression also seen in ovarian, breast, colon and lung cancer cell lines. Thus, expression of this gene could be used to differentiate between the brain cancer cell lines and other samples on this panel and as a marker for brain cancer. Furthermore, therapeutic modulation of the expression or function of this gene may be effective in the treatment of brain, ovarian, breast, colon and lung cancers.

Low but significant levels of expression are also seen in adipose. Thus, this gene product may be involved in the diagnosis and/or treatment of obesity.

Panel 2D Summary: Ag3394 Highest expression of the CG91678-01 gene is seen in a gastric cancer (CT=27). In addition, higher levels of expression are seen in gastric, lung, colon and bladder cancers when compared to the expression in the corresponding normal adjacent tissue. Thus, therapeutic targeting with a small molecule drugs, protein therapeutics or human monoclonal antibody is anticipated to limit or block the extent of tumor cell migration, invasion, growth and metastasis, preferably in gastric, bladder, lung and colon tumors.

Panel 3D Summary: Ag3394 The expression of this gene appears to be highest in a sample derived from a ovarian cancer cell line (ES-2). In addition, there appears to be substantial expression in other samples derived from bladder cancer cell lines, gastric cancer cell lines and brain cancer cell lines. Thus, the expression of this gene could be used to distinguish ES-2 cells from other samples in the panel. Moreover, therapeutic modulation of this gene, through the use of small molecule drugs, protein therapeutics or antibodies could be of benefit in the treatment of ovarian, bladder, gastric or brain cancer.

Panels 4D and 4.1D Summary: Ag3394 The CG91678-01 transcript is induced in lung fibroblasts and in human pullmonary aortic endothelial cellsHPAEC) after stimulation with IL-1beta and TNF alpha (CTs=22). Thus, this gene product may be involved in the destruction of joint tissue, lung tissue, and the remodeling of these tissues. Since this gene encodes a protein homologous to MMP1, therapeutic targeting with a human monoclonal antibody may inhibit or block inflammation, tissue destruction and recruitment of inflammatory cells into the lung due to asthma/allergy, emphysema or to the joint as a result of arthritis. See, Ohnishi K, et al. Lab Invest September 1998;78(9):1077-87.

I. NOV13 (CG91698-01): HPSE: heparanase

Expression of gene CG91698-01 was assessed using the primer-probe set Ag3069, described in Table IA. Results of the RTQ-PCR runs are shown in Tables IB, IC, ID, IE, IF and IG.

TABLE IA

Probe Name Ag3069

| | | | Start | SEQ ID |
|---|---|---|---|---|
| Primers | Sequences | Length | Position | No |
| Forward | 5′-tttgggacctcatggattactt-3′ | 22 | 1452 | 118 |
| Probe | TET-5′-tccaaatctgtccaactcaatggtct-3′-TAMRA | 26 | 1474 | 119 |
| Reverse | 5′-aggtttgatcatccaccatctt-3′ | 22 | 1507 | 120 |

TABLE IB
General_screening_panel_v1.4

| | Rel. Exp. (%) | | Rel. Exp. (%) |
|---|---|---|---|
| | Ag3069, Run | | Ag3069, Run |
| Tissue Name | 208023808 | Tissue Name | 208023808 |
| Adipose | 3.1 | Renal ca. TK-10 | 9.9 |
| Melanoma* Hs688(A).T | 0.6 | Bladder | 5.6 |
| Melanoma* Hs688(B).T | 0.2 | Gastric ca. (liver met.) | 9.7 |
| | | NCI-N87 | |
| Melanoma* M14 | 10.2 | Gastric ca. KATO III | 25.0 |
| Melanoma* LOXIMVI | 1.0 | Colon ca. SW-948 | 3.8 |
| Melanoma* SK-MEL-5 | 5.3 | Colon ca. SW480 | 1.2 |
| Squamous cell carcinoma | 4.7 | Colon ca.* (SW480 met) | 0.0 |
| SCC-4 | | SW620 | |
| Testis Pool | 1.5 | Colon ca. HT29 | 0.9 |
| Prostate ca.* (bone met) | 16.6 | Colon ca. HCT-116 | 15.6 |
| PC-3 | | | |
| Prostate Pool | 0.2 | Colon ca. CaCo-2 | 0.6 |
| Placenta | 4.7 | Colon cancer tissue | 10.0 |
| Uterus Pool | 1.4 | Colon ca. SW1116 | 6.6 |
| Ovarian ca. OVCAR-3 | 6.7 | Colon ca. Colo-205 | 0.6 |
| Ovarian ca. SK-OV-3 | 12.5 | Colon ca. SW-48 | 2.2 |
| Ovarian ca. OVCAR-4 | 2.3 | Colon Pool | 1.1 |
| Ovarian ca. OVCAR-5 | 8.1 | Small Intestine Pool | 2.4 |
| Ovarian ca. IGROV-1 | 1.6 | Stomach Pool | 0.7 |
| Ovarian ca. OVCAR-8 | 5.1 | Bone Marrow Pool | 1.1 |
| Ovary | 2.3 | Fetal Heart | 0.4 |
| Breast ca. MCF-7 | 1.3 | Heart Pool | 0.8 |
| Breast ca. MDA-MB-231 | 6.6 | Lymph Node Pool | 2.9 |
| Breast ca. BT 549 | 100.0 | Fetal Skeletal Muscle | 0.2 |
| Breast ca. T47D | 7.1 | Skeletal Muscle Pool | 0.3 |
| Breast ca. MDA-N | 1.3 | Spleen Pool | 2.6 |
| Breast Pool | 3.3 | Thymus Pool | 2.3 |
| Trachea | 4.1 | CNS cancer (glio/astro) | 17.7 |
| | | U87-MG | |
| Lung | 2.4 | CNS cancer (glio/astro) | 0.3 |
| | | U-118-MG | |
| Fetal Lung | 3.7 | CNS cancer (neuro; met) | 5.3 |
| | | SK-N-AS | |
| Lung ca. NCI-N417 | 0.2 | CNS cancer (astro) SF-539 | 5.0 |
| Lung ca. LX-1 | 0.7 | CNS cancer (astro) SNB-75 | 3.4 |
| Lung ca. NCI-H146 | 2.5 | CNS cancer (glio) SNB-19 | 1.6 |
| Lung ca. SHP-77 | 26.1 | CNS cancer (glio) SF-295 | 2.8 |
| Lung ca. A549 | 0.9 | Brain (Amygdala) Pool | 0.5 |
| Lung ca. NCI-H526 | 0.1 | Brain (cerebellum) | 1.4 |
| Lung ca. NCI-H23 | 2.8 | Brain (fetal) | 0.5 |
| Lung ca. NCI-H460 | 1.0 | Brain (Hippocampus) Pool | 0.8 |
| Lung ca. HOP-62 | 0.8 | Cerebral Cortex Pool | 0.7 |
| Lung ca. NCI-H522 | 0.4 | Brain (Substantia nigra) Pool | 0.8 |
| Liver | 0.7 | Brain (Thalamus) Pool | 1.2 |
| Fetal Liver | 3.1 | Brain (whole) | 0.9 |
| Liver ca. HepG2 | 0.0 | Spinal Cord Pool | 2.1 |
| Kidney Pool | 2.6 | Adrenal Gland | 2.6 |
| Fetal Kidney | 0.8 | Pituitary gland Pool | 0.8 |
| Renal ca. 786-0 | 3.7 | Salivary Gland | 2.2 |
| Renal ca. A498 | 0.3 | Thyroid (female) | 1.0 |
| Renal ca. ACHN | 2.2 | Pancreatic ca. CAPAN2 | 19.6 |
| Renal ca. UO-31 | 0.8 | Pancreas Pool | 5.4 |

TABLE IC
Panel 1.3D

| | Rel. | Rel. | | Rel. | Rel. |
|---|---|---|---|---|---|
| | Exp. (%) | Exp. (%) | | Exp. (%) | Exp. (%) |
| | Ag3069, | Ag3069, | | Ag3069, | Ag3069, |
| | Run | Run | | Run | Run |
| Tissue Name | 165527061 | 167595279 | Tissue Name | 165527061 | 167595279 |
| Liver | 4.3 | 5.6 | Kidney (fetal) | 5.3 | 9.0 |
| adenocarcinoma | | | | | |
| Pancreas | 0.4 | 0.0 | Renal ca. 786-0 | 3.7 | 4.0 |
| Pancreatic ca. | 12.9 | 8.4 | Renal ca. A498 | 7.2 | 4.9 |
| CAPAN 2 | | | | | |
| Adrenal gland | 4.9 | 0.6 | Renal ca. RXF 393 | 0.0 | 0.4 |
| Thyroid | 2.9 | 0.3 | Renal ca. ACHN | 3.0 | 2.0 |
| Salivary gland | 5.9 | 1.0 | Renal ca. UO-31 | 3.9 | 2.0 |
| Pituitary gland | 7.9 | 1.2 | Renal ca. TK-10 | 8.3 | 11.7 |
| Brain (fetal) | 0.3 | 0.8 | Liver | 1.6 | 0.4 |
| Brain (whole) | 12.5 | 3.3 | Liver (fetal) | 10.0 | 4.7 |
| Brain | 4.1 | 2.7 | Liver ca. (hepatoblast) | 0.0 | 0.0 |
| (amygdala) | | | HepG2 | | |
| Brain | 3.8 | 1.6 | Lung | 3.3 | 0.7 |
| (cerebellum) | | | | | |
| Brain | 3.7 | 2.0 | Lung (fetal) | 1.7 | 4.9 |
| (hippocampus) | | | | | |
| Brain | 4.7 | 2.0 | Lung ca. (small cell) LX-1 | 0.5 | 0.6 |
| (substantia | | | | | |
| nigra) | | | | | |
| Brain | 3.8 | 1.1 | Lung ca. (small cell) | 4.6 | 9.5 |
| (thalamus) | | | NCI-H69 | | |
| Cerebral Cortex | 1.1 | 1.4 | Lung ca. (s.cell var.) | 28.5 | 100.0 |
| | | | SHP-77 | | |
| Spinal cord | 8.5 | 6.3 | Lung ca. (large | 2.5 | 0.0 |
| | | | cell)NCI-H460 | | |
| glio/astro | 13.4 | 11.4 | Lung ca. (non-sm. cell) | 0.0 | 1.5 |
| | | | A549 | | |
| glio/astro | 0.0 | 0.2 | Lung ca. (non-s.cell) | 6.2 | 4.6 |
| U-118-MG | | | NCI-H23 | | |
| astrocytoma | 6.9 | 6.0 | Lung ca. (non-s.cell) | 2.2 | 1.3 |
| SW1783 | | | HOP-62 | | |
| neuro*; met | 8.7 | 5.3 | Lung ca. (non-s.cl) | 0.4 | 0.1 |
| SK-N-AS | | | NCI-H522 | | |
| astrocytoma | 4.3 | 1.9 | Lung ca. (squam.) SW 900 | 2.8 | 3.5 |
| SF-539 | | | | | |
| astrocytoma | 5.1 | 5.3 | Lung ca. (squam.) | 12.9 | 13.8 |
| SNB-75 | | | NCI-H596 | | |
| glioma SNB-19 | 4.0 | 5.0 | Mammary gland | 2.2 | 0.5 |
| glioma U251 | 34.6 | 23.8 | Breast ca.* (pl.ef) MCF-7 | 0.7 | 1.2 |
| glioma SF-295 | 1.9 | 3.7 | Breast ca.* (pl.ef) | 14.5 | 5.5 |
| | | | MDA-MB-231 | | |
| Heart (fetal) | 0.4 | 1.4 | Breast ca.* (pl.ef) T47D | 0.0 | 0.0 |
| Heart | 3.2 | 0.3 | Breast ca. BT-549 | 100.0 | 30.8 |
| Skeletal muscle | 0.0 | 0.3 | Breast ca. MDA-N | 0.0 | 1.0 |
| (fetal) | | | | | |
| Skeletal muscle | 1.4 | 0.2 | Ovary | 4.2 | 3.4 |
| Bone marrow | 8.2 | 4.1 | Ovarian ca. OVCAR-3 | 3.3 | 5.1 |
| Thymus | 0.9 | 2.1 | Ovarian ca. OVCAR-4 | 2.9 | 2.9 |
| Spleen | 11.1 | 4.1 | Ovarian ca. OVCAR-5 | 7.5 | 18.3 |
| Lymph node | 21.3 | 5.6 | Ovarian ca. OVCAR-8 | 5.4 | 2.8 |
| Colorectal | 10.9 | 8.4 | Ovarian ca. IGROV-1 | 0.4 | 1.0 |
| Stomach | 3.8 | 1.3 | Ovarian ca.* (ascites) | 6.4 | 28.3 |
| | | | SK-OV-3 | | |
| Small intestine | 5.1 | 1.2 | Uterus | 10.6 | 4.1 |
| Colon ca. | 2.1 | 1.5 | Placenta | 41.8 | 2.5 |
| SW480 | | | | | |
| Colon ca.* | 0.0 | 0.6 | Prostate | 2.9 | 0.5 |
| SW620(SW480 | | | | | |
| met) | | | | | |
| Colon ca. HT29 | 0.4 | 0.3 | Prostate ca.* (bone | 6.5 | 10.5 |
| | | | met)PC-3 | | |
| Colon ca. | 7.9 | 11.1 | Testis | 8.8 | 0.4 |
| HCT-116 | | | | | |
| Colon ca. | 1.9 | 1.4 | Melanoma Hs688(A).T | 0.8 | 0.2 |
| CaCo-2 | | | | | |
| Colon ca. | 5.6 | 4.6 | Melanoma* (met) | 0.0 | 0.0 |
| tissue(ODO386 | | | Hs688(B).T | | |
| 6) | | | | | |
| Colon ca. | 6.9 | 9.5 | Melanoma UACC-62 | 0.4 | 1.1 |
| HCC-2998 | | | | | |
| Gastric ca.* | 7.8 | 3.5 | Melanoma M14 | 8.1 | 0.4 |
| (liver met) | | | | | |
| NCI-N87 | | | | | |
| Bladder | 5.6 | 5.7 | Melanoma LOX IMVI | 0.9 | 0.2 |
| Trachea | 7.2 | 2.1 | Melanoma* (met) | 0.7 | 4.5 |
| | | | SK-MEL-5 | | |
| Kidney | 3.2 | 1.7 | Adipose | 6.5 | 9.0 |

TABLE ID
Panel 2.2

| | Rel. Exp. (%) | Rel. Exp. (%) | | Rel. Exp. (%) | Rel. Exp. (%) |
|---|---|---|---|---|---|
| | Ag3069, Run | Ag3069, Run | | Ag3069, Run | Ag3069, Run |
| Tissue Name | 173800589 | 184372174 | Tissue Name | 173800589 | 184372174 |
| Normal Colon | 39.8 | 57.8 | Kidney Margin | 100.0 | 52.1 |
| | | | (OD04348) | | |
| Colon cancer | 69.3 | 100.0 | Kidney | 8.6 | 2.5 |
| (OD06064) | | | malignant cancer | | |
| | | | (OD06204B) | | |
| Colon Margin | 29.1 | 0.0 | Kidney normal | 8.2 | 16.4 |
| (OD06064) | | | adjacent tissue | | |
| | | | (OD06204E) | | |
| Colon cancer | 4.2 | 7.6 | Kidney Cancer | 5.8 | 3.2 |
| (OD06159) | | | (OD04450-01) | | |
| Colon Margin | 45.7 | 28.5 | Kidney Margin | 12.8 | 38.7 |
| (OD06159) | | | (OD04450-03) | | |
| Colon cancer | 8.2 | 21.0 | Kidney Cancer | 0.0 | 0.8 |
| (OD06297-04) | | | 8120613 | | |
| Colon Margin | 41.2 | 0.0 | Kidney Margin | 0.0 | 6.1 |
| (OD06297-05) | | | 8120614 | | |
| CC Gr.2 ascend | 18.3 | 30.1 | Kidney Cancer | 5.6 | 28.5 |
| colon | | | 9010320 | | |
| (ODO3921) | | | | | |
| CC Margin | 12.2 | 32.3 | Kidney Margin | 2.0 | 5.4 |
| (ODO3921) | | | 9010321 | | |
| Colon cancer | 14.9 | 25.7 | Kidney Cancer | 7.5 | 10.9 |
| metastasis | | | 8120607 | | |
| (OD06104) | | | | | |
| Lung Margin | 47.6 | 96.6 | Kidney Margin | 0.0 | 6.5 |
| (OD06104) | | | 8120608 | | |
| Colon mets to | 3.0 | 15.7 | Normal Uterus | 17.6 | 12.0 |
| lung | | | | | |
| (OD04451-01) | | | | | |
| Lung Margin | 27.5 | 26.2 | Uterine Cancer | 3.4 | 21.0 |
| (OD04451-02) | | | 064011 | | |
| Normal Prostate | 9.2 | 20.9 | Normal Thyroid | 0.0 | 12.3 |
| Prostate Cancer | 0.0 | 4.8 | Thyroid Cancer | 28.7 | 87.7 |
| (OD04410) | | | 064010 | | |
| Prostate Margin | 2.8 | 3.6 | Thyroid Cancer | 9.1 | 38.2 |
| (OD04410) | | | A302152 | | |
| Normal Ovary | 16.0 | 32.8 | Thyroid Margin | 0.0 | 8.4 |
| | | | A302153 | | |
| Ovarian cancer | 24.3 | 47.6 | Normal Breast | 17.2 | 11.0 |
| (OD06283-03) | | | | | |
| Ovarian Margin | 7.0 | 10.2 | Breast Cancer | 6.9 | 30.6 |
| (OD06283-07) | | | (OD04566) | | |
| Ovarian Cancer | 5.4 | 14.5 | Breast Cancer | 5.9 | 5.0 |
| 064008 | | | 1024 | | |
| Ovarian cancer | 37.9 | 94.0 | Breast Cancer | 4.1 | 0.0 |
| (OD06145) | | | (OD04590-01) | | |
| Ovarian Margin | 54.3 | 82.4 | Breast Cancer | 9.8 | 21.0 |
| (OD06145) | | | Mets | | |
| | | | (OD04590-03) | | |
| Ovarian cancer | 4.2 | 6.3 | Breast Cancer | 17.0 | 21.5 |
| (OD06455-03) | | | Metastasis | | |
| | | | (OD04655-05) | | |
| Ovarian Margin | 15.8 | 0.0 | Breast Cancer | 16.4 | 23.3 |
| (OD06455-07) | | | 064006 | | |
| Normal Lung | 12.9 | 22.5 | Breast Cancer | 3.6 | 7.0 |
| | | | 9100266 | | |
| Invasive poor | 15.9 | 35.8 | Breast Margin | 2.1 | 7.2 |
| diff. lung adeno | | | 9100265 | | |
| (ODO4945-01) | | | | | |
| Lung Margin | 9.9 | 7.9 | Breast Cancer | 6.8 | 9.5 |
| (ODO4945-03) | | | A209073 | | |
| Lung Malignant | 11.6 | 31.6 | Breast Margin | 1.8 | 6.3 |
| Cancer | | | A2090734 | | |
| (OD03126) | | | | | |
| Lung Margin | 2.1 | 41.5 | Breast cancer | 23.3 | 12.6 |
| (OD03126) | | | (OD06083) | | |
| Lung Cancer | 6.6 | 94.0 | Breast cancer | 11.8 | 41.8 |
| (OD05014A) | | | node metastasis | | |
| | | | (OD06083) | | |
| Lung Margin | 55.1 | 55.9 | Normal Liver | 4.5 | 10.7 |
| (OD05014B) | | | | | |
| Lung cancer | 39.8 | 61.1 | Liver Cancer | 6.8 | 7.9 |
| (OD06081) | | | 1026 | | |
| Lung Margin | 15.7 | 7.2 | Liver Cancer | 7.3 | 39.5 |
| (OD06081) | | | 1025 | | |
| Lung Cancer | 4.9 | 12.6 | Liver Cancer | 15.3 | 24.8 |
| (OD04237-01) | | | 6004-T | | |
| Lung Margin | 22.5 | 28.3 | Liver Tissue | 5.6 | 5.1 |
| (OD04237-02) | | | 6004-N | | |
| Ocular Melanoma | 0.0 | 2.9 | Liver Cancer | 22.1 | 12.5 |
| Metastasis | | | 6005-T | | |
| Ocular Melanoma | 12.9 | 48.3 | Liver Tissue | 28.9 | 24.8 |
| Margin (Liver) | | | 6005-N | | |
| Melanoma | 20.7 | 19.9 | Liver Cancer | 3.2 | 6.4 |
| Metastasis | | | 064003 | | |
| Melanoma | 27.9 | 27.4 | Normal Bladder | 25.0 | 47.0 |
| Margin (Lung) | | | | | |
| Normal Kidney | 4.2 | 12.9 | Bladder Cancer | 0.0 | 15.8 |
| | | | 1023 | | |
| Kidney Ca, | 25.5 | 46.0 | Bladder Cancer | 20.9 | 43.5 |
| Nuclear grade 2 | | | A302173 | | |
| (OD04338) | | | | | |
| Kidney Margin | 8.9 | 48.0 | Normal Stomach | 23.0 | 27.0 |
| (OD04338) | | | | | |
| Kidney Ca | 8.3 | 26.1 | Gastric Cancer | 21.2 | 72.7 |
| Nuclear grade 1/2 | | | 9060397 | | |
| (OD04339) | | | | | |
| Kidney Margin | 11.6 | 9.4 | Stomach Margin | 12.5 | 13.4 |
| (OD04339) | | | 9060396 | | |
| Kidney Ca, Clear | 16.4 | 42.6 | Gastric Cancer | 17.2 | 39.0 |
| cell type | | | 9060395 | | |
| (OD04340) | | | | | |
| Kidney Margin | 19.9 | 16.0 | Stomach Margin | 45.1 | 50.3 |
| (OD04340) | | | 9060394 | | |
| Kidney Ca, | 47.3 | 74.2 | Gastric Cancer | 22.7 | 49.7 |
| Nuclear grade 3 | | | 064005 | | |
| (OD04348) | | | | | |

TABLE IE
Panel 2D

| | Rel. Exp. (%) | | Rel. Exp. (%) |
|---|---|---|---|
| | Ag3069, Run | | Ag3069, Run |
| Tissue Name | 165301664 | Tissue Name | 165301664 |
| Normal Colon | 49.7 | Kidney Margin 8120608 | 2.2 |
| CC Well to Mod Diff | 17.7 | Kidney Cancer 8120613 | 0.6 |
| (ODO3866) | | | |
| CC Margin (ODO3866) | 10.6 | Kidney Margin 8120614 | 1.4 |
| CC Gr.2 rectosigmoid | 5.7 | Kidney Cancer 9010320 | 25.7 |
| (ODO3868) | | | |
| CC Margin (ODO3868) | 0.6 | Kidney Margin 9010321 | 6.7 |
| CC Mod Diff (ODO3920) | 3.0 | Normal Uterus | 0.8 |
| CC Margin (ODO3920) | 9.4 | Uterus Cancer 064011 | 5.8 |
| CC Gr.2 ascend colon | 42.6 | Normal Thyroid | 3.0 |
| (ODO3921) | | | |
| CC Margin (ODO3921) | 13.8 | Thyroid Cancer 064010 | 42.6 |
| CC from Partial Hepatectomy | 22.8 | Thyroid Cancer A302152 | 7.0 |
| (ODO4309) Mets | | | |
| Liver Margin (ODO4309) | 10.8 | Thyroid Margin A302153 | 2.9 |
| Colon mets to lung | 3.1 | Normal Breast | 3.3 |
| (OD04451-01) | | | |
| Lung Margin (OD04451-02) | 5.8 | Breast Cancer (OD04566) | 8.8 |
| Normal Prostate 6546-1 | 5.4 | Breast Cancer (OD04590-01) | 10.1 |
| Prostate Cancer (OD04410) | 7.3 | Breast Cancer Mets | 12.3 |
| | | (OD04590-03) | |
| Prostate Margin (OD04410) | 2.3 | Breast Cancer Metastasis | 7.6 |
| | | (OD04655-05) | |
| Prostate Cancer (OD04720-01) | 6.1 | Breast Cancer 064006 | 9.2 |
| Prostate Margin (OD04720-02) | 12.3 | Breast Cancer 1024 | 6.9 |
| Normal Lung 061010 | 25.7 | Breast Cancer 9100266 | 4.3 |
| Lung Met to Muscle | 49.7 | Breast Margin 9100265 | 3.0 |
| (ODO4286) | | | |
| Muscle Margin (ODO4286) | 6.1 | Breast Cancer A209073 | 12.4 |
| Lung Malignant Cancer | 25.7 | Breast Margin A209073 | 0.7 |
| (OD03126) | | | |
| Lung Margin (OD03126) | 11.3 | Normal Liver | 2.0 |
| Lung Cancer (OD04404) | 37.6 | Liver Cancer 064003 | 3.1 |
| Lung Margin (OD04404) | 12.7 | Liver Cancer 1025 | 3.8 |
| Lung Cancer (OD04565) | 7.1 | Liver Cancer 1026 | 2.5 |
| Lung Margin (OD04565) | 9.7 | Liver Cancer 6004-T | 6.3 |
| Lung Cancer (OD04237-01) | 19.9 | Liver Tissue 6004-N | 2.7 |
| Lung Margin (OD04237-02) | 14.5 | Liver Cancer 6005-T | 3.1 |
| Ocular Mel Met to Liver | 1.1 | Liver Tissue 6005-N | 2.4 |
| (ODO4310) | | | |
| Liver Margin (ODO4310) | 10.0 | Normal Bladder | 21.6 |
| Melanoma Mets to Lung | 13.0 | Bladder Cancer 1023 | 4.5 |
| (OD04321) | | | |
| Lung Margin (OD04321) | 19.9 | Bladder Cancer A302173 | 14.9 |
| Normal Kidney | 12.9 | Bladder Cancer | 76.3 |
| | | (OD04718-01) | |
| Kidney Ca, Nuclear grade 2 | 12.1 | Bladder Normal Adjacent | 10.5 |
| (OD04338) | | (OD04718-03) | |
| Kidney Margin (OD04338) | 14.5 | Normal Ovary | 5.9 |
| Kidney Ca Nuclear grade 1/2 | 7.3 | Ovarian Cancer 064008 | 14.9 |
| (OD04339) | | | |
| Kidney Margin (OD04339) | 7.6 | Ovarian Cancer | 100.0 |
| | | (OD04768-07) | |
| Kidney Ca, Clear cell type | 35.6 | Ovary Margin (OD04768-08) | 4.0 |
| (OD04340) | | | |
| Kidney Margin (OD04340) | 25.9 | Normal Stomach | 4.5 |
| Kidney Ca, Nuclear grade 3 | 20.4 | Gastric Cancer 9060358 | 3.1 |
| (OD04348) | | | |
| Kidney Margin (OD04348) | 15.6 | Stomach Margin 9060359 | 9.8 |
| Kidney Cancer (OD04622-01) | 19.5 | Gastric Cancer 9060395 | 25.9 |
| Kidney Margin (OD04622-03) | 1.9 | Stomach Margin 9060394 | 11.9 |
| Kidney Cancer (OD04450-01) | 0.6 | Gastric Cancer 9060397 | 55.1 |
| Kidney Margin (OD04450-03) | 5.5 | Stomach Margin 9060396 | 3.1 |
| Kidney Cancer 8120607 | 3.2 | Gastric Cancer 064005 | 36.6 |

TABLE IF
Panel 4.1D

| | Rel. Exp. (%) | | Rel. Exp. (%) |
|---|---|---|---|
| | Ag3069, Run | | Ag3069, Run |
| Tissue Name | 169838255 | Tissue Name | 169838255 |
| Secondary Th1 act | 2.3 | HUVEC IL-1beta | 11.5 |
| Secondary Th2 act | 11.6 | HUVEC IFN gamma | 8.4 |
| Secondary Tr1 act | 6.1 | HUVEC TNF alpha + IFN | 4.6 |
| | | gamma | |
| Secondary Th1 rest | 2.0 | HUVEC TNF alpha + IL4 | 3.5 |
| Secondary Th2 rest | 4.5 | HUVEC IL-11 | 4.9 |
| Secondary Tr1 rest | 2.9 | Lung Microvascular EC none | 33.2 |
| Primary Th1 act | 4.4 | Lung Microvascular EC | 25.5 |
| | | TNF alpha + IL-1beta | |
| Primary Th2 act | 7.7 | Microvascular Dermal EC | 13.4 |
| | | none | |
| Primary Tr1 act | 10.7 | Microsvasular Dermal EC | 12.5 |
| | | TNF alpha + IL-1beta | |
| Primary Th1 rest | 6.4 | Bronchial epithelium | 2.0 |
| | | TNF alpha + IL1beta | |
| Primary Th2 rest | 3.0 | Small airway epithelium none | 0.3 |
| Primary Tr1 rest | 4.8 | Small airway epithelium | 4.8 |
| | | TNF alpha + IL-1beta | |
| CD45RA CD4 lymphocyte act | 7.4 | Coronery artery SMC rest | 2.4 |
| CD45RO CD4 lymphocyte act | 16.5 | Coronery artery SMC | 2.7 |
| | | TNF alpha + IL-1beta | |
| CD8 lymphocyte act | 8.8 | Astrocytes rest | 0.0 |
| Secondary CD8 lymphocyte | 14.6 | Astrocytes TNF alpha + | 0.0 |
| rest | | IL-1beta | |
| Secondary CD8 lymphocyte act | 5.8 | KU-812 (Basophil) rest | 0.6 |
| CD4 lymphocyte none | 2.9 | KU-812 (Basophil) | 2.9 |
| | | PMA/ionomycin | |
| 2ry Th1/Th2/Tr1_anti-CD95 | 1.8 | CCD1106 (Keratinocytes) | 7.2 |
| CH11 | | none | |
| LAK cells rest | 6.0 | CCD1106 (Keratinocytes) | 10.4 |
| | | TNF alpha + IL-1beta | |
| LAK cells IL-2 | 8.3 | Liver cirrhosis | 3.6 |
| LAK cells IL-2 + IL-12 | 8.3 | NCI-H292 none | 5.7 |
| LAK cells IL-2 + IFN gamma | 14.2 | NCI-H292 IL-4 | 2.5 |
| LAK cells IL-2 + IL-18 | 10.8 | NCI-H292 IL-9 | 7.1 |
| LAK cells PMA/ionomycin | 5.6 | NCI-H292 IL-13 | 2.6 |
| NK Cells IL-2 rest | 11.4 | NCI-H292 IFN gamma | 5.0 |
| Two Way MLR 3 day | 9.7 | HPAEC none | 13.6 |
| Two Way MLR 5 day | 8.2 | HPAEC TNF alpha + | 29.3 |
| | | IL-1beta | |
| Two Way MLR 7 day | 7.2 | Lung fibroblast none | 0.0 |
| PBMC rest | 8.8 | Lung fibroblast TNF alpha + | 0.6 |
| | | IL-1beta | |
| PBMC PWM | 11.6 | Lung fibroblast IL-4 | 0.2 |
| PBMC PHA-L | 11.3 | Lung fibroblast IL-9 | 0.3 |
| Ramos (B cell) none | 11.2 | Lung fibroblast IL-13 | 0.0 |
| Ramos (B cell) ionomycin | 12.3 | Lung fibroblast IFN gamma | 0.1 |
| B lymphocytes PWM | 2.1 | Dermal fibroblast CCD1070 | 1.2 |
| | | rest | |
| B lymphocytes CD40L and IL-4 | 2.0 | Dermal fibroblast CCD1070 | 8.2 |
| | | TNF alpha | |
| EOL-1 dbcAMP | 0.7 | Dermal fibroblast CCD1070 | 0.7 |
| | | IL-1beta | |
| EOL-1 dbcAMP | 1.1 | Dermal fibroblast IFN gamma | 0.4 |
| PMA/ionomycin | | | |
| Dendritic cells none | 1.1 | Dermal fibroblast IL-4 | 0.9 |
| Dendritic cells LPS | 0.0 | Dermal Fibroblasts rest | 1.0 |
| Dendritic cells anti-CD40 | 0.0 | Neutrophils TNFa + LPS | 16.7 |
| Monocytes rest | 100.0 | Neutrophils rest | 36.9 |
| Monocytes LPS | 80.1 | Colon | 2.8 |
| Macrophages rest | 3.7 | Lung | 4.1 |
| Macrophages LPS | 7.5 | Thymus | 2.0 |
| HUVEC none | 4.3 | Kidney | 1.3 |
| HUVEC starved | 4.2 | | |

TABLE IG
Panel 4D

| | Rel. Exp. (%) | | Rel. Exp. (%) |
|---|---|---|---|
| | Ag3069, Run | | Ag3069, Run |
| Tissue Name | 164525656 | Tissue Name | 164525656 |
| Secondary Th1 act | 4.1 | HUVEC IL-1beta | 4.8 |
| Secondary Th2 act | 6.9 | HUVEC IFN gamma | 10.9 |
| Secondary Tr1 act | 5.9 | HUVEC TNF alpha + IFN | 10.7 |
| | | gamma | |
| Secondary Th1 rest | 1.7 | HUVEC TNF alpha + IL4 | 5.9 |
| Secondary Th2 rest | 3.3 | HUVEC IL-11 | 5.8 |
| Secondary Tr1 rest | 2.4 | Lung Microvascular EC none | 32.3 |
| Primary Th1 act | 3.7 | Lung Microvascular EC | 24.0 |
| | | TNF alpha + IL-1beta | |
| Primary Th2 act | 7.1 | Microvascular Dermal EC | 23.3 |
| | | none | |
| Primary Tr1 act | 10.2 | Microsvasular Dermal EC | 13.0 |
| | | TNF alpha + IL-1beta | |
| Primary Th1 rest | 19.1 | Bronchial epithelium | 4.8 |
| | | TNF alpha + IL1beta | |
| Primary Th2 rest | 6.4 | Small airway epithelium none | 2.0 |
| Primary Tr1 rest | 7.0 | Small airway epithelium | 25.2 |
| | | TNF alpha + IL-1beta | |
| CD45RA CD4 lymphocyte act | 7.6 | Coronery artery SMC rest | 3.2 |
| CD45RO CD4 lymphocyte act | 14.0 | Coronery artery SMC | 1.9 |
| | | TNF alpha + IL-1beta | |
| CD8 lymphocyte act | 9.1 | Astrocytes rest | 0.3 |
| Secondary CD8 lymphocyte rest | 17.4 | Astrocytes TNF alpha + | 0.3 |
| | | IL-1beta | |
| Secondary CD8 lymphocyte act | 6.6 | KU-812 (Basophil) rest | 0.3 |
| CD4 lymphocyte none | 3.5 | KU-812 (Basophil) | 7.9 |
| | | PMA/ionomycin | |
| 2ry Th1/Th2/Tr1_anti-CD95 | 3.6 | CCD1106 (Keratinocytes) | 8.5 |
| CH11 | | none | |
| LAK cells rest | 11.3 | CCD1106 (Keratinocytes) | 5.5 |
| | | TNF alpha + IL-1beta | |
| LAK cells IL-2 | 12.8 | Liver cirrhosis | 1.0 |
| LAK cells IL-2 + IL-12 | 11.7 | Lupus kidney | 1.4 |
| LAK cells IL-2 + IFN gamma | 20.4 | NCI-H292 none | 11.6 |
| LAK cells IL-2 + IL-18 | 20.4 | NCI-H292 IL-4 | 9.9 |
| LAK cells PMA/ionomycin | 5.2 | NCI-H292 IL-9 | 13.3 |
| NK Cells IL-2 rest | 9.6 | NCI-H292 IL-13 | 5.3 |
| Two Way MLR 3 day | 11.6 | NCI-H292 IFN gamma | 7.2 |
| Two Way MLR 5 day | 3.9 | HPAEC none | 23.2 |
| Two Way MLR 7 day | 8.0 | HPAEC TNF alpha + | 28.7 |
| | | IL-1beta | |
| PBMC rest | 13.7 | Lung fibroblast none | 0.5 |
| PBMC PWM | 47.6 | Lung fibroblast TNF alpha + | 1.6 |
| | | IL-1beta | |
| PBMC PHA-L | 21.3 | Lung fibroblast IL-4 | 0.3 |
| Ramos (B cell) none | 9.9 | Lung fibroblast IL-9 | 0.1 |
| Ramos (B cell) ionomycin | 48.3 | Lung fibroblast IL-13 | 0.1 |
| B lymphocytes PWM | 23.7 | Lung fibroblast IFN gamma | 1.3 |
| B lymphocytes CD40L and IL-4 | 3.5 | Dermal fibroblast CCD1070 | 5.8 |
| | | rest | |
| EOL-1 dbcAMP | 1.6 | Dermal fibroblast CCD1070 | 21.9 |
| | | TNF alpha | |
| EOL-1 dbcAMP | 1.3 | Dermal fibroblast CCD1070 | 0.8 |
| PMA/ionomycin | | IL-1beta | |
| Dendritic cells none | 0.3 | Dermal fibroblast IFN gamma | 1.6 |
| Dendritic cells LPS | 0.8 | Dermal fibroblast IL-4 | 1.1 |
| Dendritic cells anti-CD40 | 0.0 | IBD Colitis 2 | 0.3 |
| Monocytes rest | 100.0 | IBD Crohn's | 0.9 |
| Monocytes LPS | 44.4 | Colon | 8.0 |
| Macrophages rest | 3.8 | Lung | 5.9 |
| Macrophages LPS | 8.8 | Thymus | 3.8 |
| HUVEC none | 10.9 | Kidney | 4.3 |
| HUVEC starved | 12.8 | | |

General_screening_panel v1.4 Summary: Ag3069 Highest expression of the CG91698-01 gene is detected in Breast cancer cell line BT 549 (CT=25.9). In addition, high expression of this gene is also seen in cluster of cancer cell lines (Pancreatic, CNS, colon, gastric, lung, breast, ovarian, prostate and melanoma) used in this panel. This gene codes for heparanase protein, an endoglucuronidase capable of specifically degrading heparan sulfate, and its activity is associated with the metastatic potential of tumor cells. Expression of heparanase correlates with the metastatic potential of tumor cells, and treatment with heparanase inhibitors markedly reduces the incidence of metastasis in experimental animals. See, Zcharia E., et al J Mammary Gland Biol Neoplasia 6(3):311-22 (PMID: 11547900); Uno F, et al. (2001) Cancer Res 61(21):7855-60 (PMID: 11691803). Therefore, therapeutic modulation of the activity of this gene or its protein product, through the use of small molecule drugs, or antibodies, might be beneficial in the treatment of these cancers and its metastasis.

Among tissues with metabolic or endocrine function, this gene is expressed at low to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity diabetes and atherogenesis.

In addition, this gene is expressed at low levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 1.3D Summary: Ag3069 Highest expression of the CG91698-01 gene is detected in a breast cancer cell line BT 549 (CT=30.4) and lung cancer cell line SHP-77 (CT=29). In addition significant expression of this gene is also seen in many of the cancer cell lines used in this panel. Please see panel 1.4 for the utility of this gene.

Interestingly, this gene is expressed at much higher levels in fetal (CT 33) when compared to adult liver (CT=36-37). This observation suggests that expression of this gene can be used to distinguish fetal from adult liver.

Panel 2.2 Summary: Ag3069 Highest expression of the CG91698-01 gene is detected in kidney margin (OD04348) (CT=33) and colon cancer (OD06064) (CT=30). Two independent experiments with same primer and probe sets are in excellent agreement with significant expression of this gene in both normal and cancer tissues. Interestingly, expression of this gene is higher in liver margin (ODO4310) (CTs=31-35) as compared to the sample derived from ocular MeI metastasis to Liver (ODO4310)sample. Thus, expression of this gene can be used to distinguish these two samples. Please see panel 1.4 for utility of this gene.

Panel 2D Summary: Ag3069 Highest expression of the CG91698-01 gene is detected in ovarian cancer (OD04768-07) tissue sample (CT=30). In addition expression of this gene is lower in the control margin tissue (OD04768-08) (CT=34.7). Similar differential expression is also detected in bladder cancer (CT=30) and control (OD04718-01) tissue (CT=33). Therefore, expression of this gene can be used in distinguishing these tissues and also as marker in detection of bladder and ovarian cancer.

In addition, significant expression of this gene is also seen in many of the normal and cancer tissues used in this panel. Please see panel 1.4 for utility of this gene.

Panel 4.1D Summary: Ag3069 Highest expression of the CG91698-01 gene is detected in monocytes (Cts=28). In addition, this gene is expressed at low to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 4D Summary: Ag3069 Highest expression of the CG91698-01 gene is detected in monocytes (Cts=28-29), with expression in this panel in excellent agreement with expression in Panel 4.1D. In addition, this gene is expressed at low to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Interestingly, expression of this gene is decreased in colon samples from patients with IBD colitis and Crohn's disease (CTs=35-36) relative to normal colon (CT=32). Therefore, therapeutic modulation of the activity of the protein encoded by this gene may be useful in the treatment of inflammatory bowel disease.

J. NOV14a and NOV14b (CG91708-01 and CG91708-02): MMP3

Expression of gene CG91708-01 and full length physical clone CG91708-02 was assessed using the primer-probe set Ag3395, described in Table JA. Results of the RTQ-PCR runs are shown in Tables JB, JC, JD, JE, JF and JG.

TABLE JA

Probe Name Ag3395

| | | | Start | SEQ ID |
|---|---|---|---|---|
| Primers | Sequences | Length | Position | No |
| Forward | 5′-gtaaagccagtggaaatgaaga-3′ | 22 | 49 | 121 |
| Probe | TET-5′-tcttccaatcctactgttgctgtgcg-3′-TAMRA | 26 | 72 | 122 |
| Reverse | 5′-caatggataggctgagcaaac-3′ | 21 | 103 | 123 |

TABLE JB
AI_comprehensive panel_v1.0

| | Rel. Exp. (%) | | Rel. Exp. (%) |
|---|---|---|---|
| | Ag3395, Run | | Ag3395, Run |
| Tissue Name | 217700657 | Tissue Name | 217700657 |
| 110967 COPD-F | 0.0 | 112427 Match Control | 0.0 |
| | | Psoriasis-F | |
| 110980 COPD-F | 0.0 | 112418 Psoriasis-M | 0.0 |
| 110968 COPD-M | 0.0 | 112723 Match Control | 0.0 |
| | | Psoriasis-M | |
| 110977 COPD-M | 0.0 | 112419 Psoriasis-M | 0.0 |
| 110989 Emphysema-F | 0.0 | 112424 Match Control | 0.0 |
| | | Psoriasis-M | |
| 110992 Emphysema-F | 0.0 | 112420 Psoriasis-M | 0.0 |
| 110993 Emphysema-F | 0.0 | 112425 Match Control | 0.0 |
| | | Psoriasis-M | |
| 110994 Emphysema-F | 0.0 | 104689 (MF) OA Bone-Backus | 1.0 |
| 110995 Emphysema-F | 0.0 | 104690 (MF) Adj “Normal” | 2.3 |
| | | Bone-Backus | |
| 110996 Emphysema-F | 0.0 | 104691 (MF) OA | 4.9 |
| | | Synovium-Backus | |
| 110997 Asthma-M | 0.0 | 104692 (BA) OA | 27.9 |
| | | Cartilage-Backus | |
| 111001 Asthma-F | 0.0 | 104694 (BA) OA Bone-Backus | 2.6 |
| 111002 Asthma-F | 0.0 | 104695 (BA) Adj “Normal” | 90.1 |
| | | Bone-Backus | |
| 111003 Atopic Asthma-F | 0.0 | 104696 (BA) OA | 100.0 |
| | | Synovium-Backus | |
| 111004 Atopic Asthma-F | 0.0 | 104700 (SS) OA Bone-Backus | 0.7 |
| 111005 Atopic Asthma-F | 0.0 | 104701 (SS) Adj “Normal” | 14.1 |
| | | Bone-Backus | |
| 111006 Atopic Asthma-F | 0.0 | 104702 (SS) OA | 1.6 |
| | | Synovium-Backus | |
| 111417 Allergy-M | 0.0 | 117093 OA Cartilage Rep7 | 0.3 |
| 112347 Allergy-M | 0.0 | 112672 OA Bone5 | 0.6 |
| 112349 Normal Lung-F | 0.0 | 112673 OA Synovium5 | 0.3 |
| 112357 Normal Lung-F | 0.0 | 112674 OA Synovial Fluid | 0.3 |
| | | cells5 | |
| 112354 Normal Lung-M | 0.0 | 117100 OA Cartilage Rep14 | 0.0 |
| 112374 Crohns-F | 0.0 | 112756 OA Bone9 | 0.0 |
| 112389 Match Control | 0.1 | 112757 OA Synovium9 | 0.0 |
| Crohns-F | | | |
| 112375 Crohns-F | 0.0 | 112758 OA Synovial Fluid | 0.0 |
| | | Cells9 | |
| 112732 Match Control | 0.0 | 117125 RA Cartilage Rep2 | 0.0 |
| Crohns-F | | | |
| 112725 Crohns-M | 0.1 | 113492 Bone2 RA | 0.0 |
| 112387 Match Control | 0.2 | 113493 Synovium2 RA | 0.0 |
| Crohns-M | | | |
| 112378 Crohns-M | 0.0 | 113494 Syn Fluid Cells RA | 0.0 |
| 112390 Match Control | 0.0 | 113499 Cartilage4 RA | 0.0 |
| Crohns-M | | | |
| 112726 Crohns-M | 0.0 | 113500 Bone4 RA | 0.0 |
| 112731 Match Control | 0.0 | 113501 Synovium4 RA | 0.0 |
| Crohns-M | | | |
| 112380 Ulcer Col-F | 0.0 | 113502 Syn Fluid Cells4 RA | 0.0 |
| 112734 Match Control Ulcer | 0.3 | 113495 Cartilage3 RA | 0.0 |
| Col-F | | | |
| 112384 Ulcer Col-F | 0.0 | 113496 Bone3 RA | 0.0 |
| 112737 Match Control Ulcer | 0.0 | 113497 Synovium3 RA | 0.0 |
| Col-F | | | |
| 112386 Ulcer Col-F | 0.3 | 113498 Syn Fluid Cells3 RA | 0.1 |
| 112738 Match Control Ulcer | 3.0 | 117106 Normal Cartilage Rep20 | 0.0 |
| Col-F | | | |
| 112381 Ulcer Col-M | 0.0 | 113663 Bone3 Normal | 0.0 |
| 112735 Match Control Ulcer | 0.2 | 113664 Synovium3 Normal | 0.0 |
| Col-M | | | |
| 112382 Ulcer Col-M | 0.0 | 113665 Syn Fluid Cells3 Normal | 0.0 |
| 112394 Match Control Ulcer | 0.1 | 117107 Normal Cartilage Rep22 | 0.0 |
| Col-M | | | |
| 112383 Ulcer Col-M | 0.0 | 113667 Bone4 Normal | 0.0 |
| 112736 Match Control Ulcer | 0.0 | 113668 Synovium4 Normal | 0.0 |
| Col-M | | | |
| 112423 Psoriasis-F | 0.0 | 113669 Syn Fluid Cells4 Normal | 0.0 |

TABLE JC
General_screening_panel_v1.4

| | Rel. Exp. (%) | Rel. Exp. (%) | | Rel. Exp. (%) | Rel. Exp. (%) |
|---|---|---|---|---|---|
| | Ag3395, Run | Ag3395, Run | Tissue | Ag3395, Run | Ag3395, Run |
| Tissue Name | 208034252 | 212141064 | Name | 208034252 | 212141064 |
| Adipose | 0.1 | 0.1 | Renal ca. | 0.0 | 0.0 |
| | | | TK-10 | | |
| Melanoma* | 1.2 | 1.9 | Bladder | 0.1 | 0.1 |
| Hs688(A).T | | | | | |
| Melanoma* | 0.3 | 0.5 | Gastric ca. | 0.5 | 0.8 |
| Hs688(B).T | | | (liver met.) | | |
| | | | NCI-N87 | | |
| Melanoma* | 0.1 | 0.1 | Gastric ca. | 0.4 | 0.8 |
| M14 | | | KATO III | | |
| Melanoma* | 3.2 | 6.6 | Colon ca. | 0.0 | 0.0 |
| LOXIMVI | | | SW-948 | | |
| Melanoma* | 0.0 | 0.0 | Colon ca. | 0.0 | 0.0 |
| SK-MEL-5 | | | SW480 | | |
| Squamous cell | 0.0 | 0.1 | Colon ca.* | 0.0 | 0.0 |
| carcinoma | | | (SW480 | | |
| SCC-4 | | | met) SW620 | | |
| Testis Pool | 0.8 | 1.2 | Colon ca. | 0.0 | 0.0 |
| | | | HT29 | | |
| Prostate ca.* | 0.1 | 0.1 | Colon ca. | 0.0 | 0.0 |
| (bone met) | | | HCT-116 | | |
| PC-3 | | | | | |
| Prostate Pool | 0.1 | 0.2 | Colon ca. | 0.1 | 0.1 |
| | | | CaCo-2 | | |
| Placenta | 0.0 | 0.0 | Colon cancer | 30.1 | 37.1 |
| | | | tissue | | |
| Uterus Pool | 0.0 | 0.0 | Colon ca. | 0.0 | 0.0 |
| | | | SW1116 | | |
| Ovarian ca. | 0.0 | 0.1 | Colon ca. | 0.0 | 0.0 |
| OVCAR-3 | | | Colo-205 | | |
| Ovarian ca. | 0.0 | 0.3 | Colon ca. | 0.0 | 0.0 |
| SK-OV-3 | | | SW-48 | | |
| Ovarian ca. | 0.0 | 0.0 | Colon Pool | 0.0 | 0.0 |
| OVCAR-4 | | | | | |
| Ovarian ca. | 0.2 | 0.4 | Small | 0.6 | 1.2 |
| OVCAR-5 | | | Intestine | | |
| | | | Pool | | |
| Ovarian ca. | 0.0 | 0.1 | Stomach Pool | 2.2 | 3.7 |
| IGROV-1 | | | | | |
| Ovarian ca. | 0.0 | 0.0 | Bone Marrow | 0.0 | 0.0 |
| OVCAR-8 | | | Pool | | |
| Ovary | 0.0 | 0.0 | Fetal Heart | 0.0 | 0.0 |
| Breast ca. | 0.0 | 0.0 | Heart Pool | 0.0 | 0.0 |
| MCF-7 | | | | | |
| Breast ca. | 0.0 | 0.0 | Lymph Node | 0.0 | 0.0 |
| MDA-MB-231 | | | Pool | | |
| Breast ca. BT | 0.1 | 0.2 | Fetal Skeletal | 0.0 | 0.0 |
| 549 | | | Muscle | | |
| Breast ca. T47D | 0.1 | 0.3 | Skeletal | 0.1 | 0.2 |
| | | | Muscle Pool | | |
| Breast ca. | 0.1 | 0.2 | Spleen Pool | 0.1 | 0.1 |
| MDA-N | | | | | |
| Breast Pool | 0.1 | 0.3 | Thymus Pool | 0.0 | 0.1 |
| Trachea | 1.6 | 1.8 | CNS cancer | 100.0 | 100.0 |
| | | | (glio/astro) | | |
| | | | U87-MG | | |
| Lung | 0.0 | 0.0 | CNS cancer | 52.5 | 72.7 |
| | | | (glio/astro) | | |
| | | | U-118-MG | | |
| Fetal Lung | 0.1 | 0.1 | CNS cancer | 0.0 | 0.0 |
| | | | (neuro; met) | | |
| | | | SK-N-AS | | |
| Lung ca. | 0.0 | 0.0 | CNS cancer | 0.1 | 0.2 |
| NCI-N417 | | | (astro) | | |
| | | | SF-539 | | |
| Lung ca. LX-1 | 0.0 | 0.0 | CNS cancer | 0.3 | 0.7 |
| | | | (astro) | | |
| | | | SNB-75 | | |
| Lung ca. | 0.0 | 0.0 | CNS cancer | 0.1 | 0.2 |
| NCI-H146 | | | (glio) SNB-19 | | |
| Lung ca. | 0.0 | 0.0 | CNS cancer | 21.2 | 54.0 |
| SHP-77 | | | (glio) SF-295 | | |
| Lung ca. A549 | 0.0 | 0.0 | Brain | 0.0 | 0.0 |
| | | | (Amygdala) | | |
| | | | Pool | | |
| Lung ca. | 0.0 | 0.0 | Brain | 0.0 | 0.0 |
| NCI-H526 | | | (cerebellum) | | |
| Lung ca. | 0.0 | 0.4 | Brain (fetal) | 0.0 | 0.0 |
| NCI-H23 | | | | | |
| Lung ca. | 0.0 | 0.2 | Brain | 0.1 | 0.2 |
| NCI-H460 | | | (Hippocampus) | | |
| | | | Pool | | |
| Lung ca. | 0.0 | 0.0 | Cerebral | 0.0 | 0.0 |
| HOP-62 | | | Cortex Pool | | |
| Lung ca. | 0.1 | 0.3 | Brain | 0.0 | 0.0 |
| NCI-H522 | | | (Substantia | | |
| | | | nigra) Pool | | |
| Liver | 0.0 | 0.0 | Brain | 0.0 | 0.0 |
| | | | (Thalamus) | | |
| | | | Pool | | |
| Fetal Liver | 0.0 | 0.0 | Brain (whole) | 0.0 | 0.1 |
| Liver ca. | 0.0 | 0.0 | Spinal Cord | 0.0 | 0.0 |
| HepG2 | | | Pool | | |
| Kidney Pool | 0.0 | 0.0 | Adrenal | 0.0 | 0.1 |
| | | | Gland | | |
| Fetal Kidney | 0.3 | 0.5 | Pituitary | 0.0 | 0.0 |
| | | | gland Pool | | |
| Renal ca. 786-0 | 0.0 | 0.0 | Salivary | 0.1 | 0.0 |
| | | | Gland | | |
| Renal ca. A498 | 0.0 | 0.0 | Thyroid | 0.0 | 0.0 |
| | | | (female) | | |
| Renal ca. | 0.0 | 0.8 | Pancreatic ca. | 0.1 | 0.1 |
| ACHN | | | CAPAN2 | | |
| Renal ca. | 0.0 | 0.0 | Pancreas Pool | 0.0 | 0.1 |
| UO-31 | | | | | |

TABLE JD
Panel 1.3D

| | Rel. | Rel. | | Rel. | Rel. |
|---|---|---|---|---|---|
| | Exp. (%) | Exp. (%) | | Exp. (%) | Exp. (%) |
| | Ag3395, | Ag3395, | | Ag3395, | Ag3395, |
| | Run | Run | | Run | Run |
| Tissue Name | 165524931 | 167595399 | Tissue Name | 165524931 | 167595399 |
| Liver adenocarcinoma | 0.0 | 0.0 | Kidney (fetal) | 0.1 | 1.4 |
| Pancreas | 0.0 | 0.0 | Renal ca. 786-0 | 0.0 | 0.0 |
| Pancreatic ca. CAPAN 2 | 0.2 | 0.0 | Renal ca. A498 | 0.2 | 0.2 |
| Adrenal gland | 0.2 | 0.1 | Renal ca. RXF | 0.0 | 1.4 |
| | | | 393 | | |
| Thyroid | 0.0 | 0.0 | Renal ca. ACHN | 0.0 | 0.0 |
| Salivary gland | 0.0 | 0.0 | Renal ca. UO-31 | 0.0 | 0.0 |
| Pituitary gland | 0.0 | 0.0 | Renal ca. TK-10 | 0.0 | 0.1 |
| Brain (fetal) | 0.0 | 0.0 | Liver | 0.0 | 0.0 |
| Brain (whole) | 0.5 | 0.1 | Liver (fetal) | 0.0 | 0.0 |
| Brain (amygdala) | 0.0 | 0.0 | Liver ca. | 0.0 | 0.0 |
| | | | (hepatoblast) | | |
| | | | HepG2 | | |
| Brain (cerebellum) | 0.0 | 0.0 | Lung | 0.0 | 0.0 |
| Brain (hippocampus) | 0.3 | 0.1 | Lung (fetal) | 0.0 | 0.2 |
| Brain (substantia nigra) | 0.0 | 0.0 | Lung ca. (small | 0.0 | 0.0 |
| | | | cell) LX-1 | | |
| Brain (thalamus) | 0.0 | 0.1 | Lung ca. (small | 0.0 | 0.0 |
| | | | cell) NCI-H69 | | |
| Cerebral Cortex | 0.0 | 0.0 | Lung ca. (s.cell | 0.0 | 0.2 |
| | | | var.) SHP-77 | | |
| Spinal cord | 0.0 | 0.0 | Lung ca. (large | 0.2 | 0.0 |
| | | | cell) NCI-H460 | | |
| glio/astro U87-MG | 76.3 | 100.0 | Lung ca. | 0.0 | 0.1 |
| | | | (non-sm. cell) | | |
| | | | A549 | | |
| glio/astro U-118-MG | 100.0 | 69.3 | Lung ca. | 0.0 | 0.0 |
| | | | (non-s.cell) | | |
| | | | NCI-H23 | | |
| astrocytoma SW1783 | 3.3 | 4.5 | Lung ca. | 0.0 | 0.0 |
| | | | (non-s.cell) | | |
| | | | HOP-62 | | |
| neuro*; met SK-N-AS | 0.0 | 0.0 | Lung ca. | 0.0 | 0.5 |
| | | | (non-s.cl) | | |
| | | | NCI-H522 | | |
| astrocytoma SF-539 | 0.0 | 0.0 | Lung ca. | 0.0 | 0.1 |
| | | | (squam.) SW 900 | | |
| astrocytoma SNB-75 | 0.2 | 0.4 | Lung ca. | 0.0 | 0.7 |
| | | | (squam.) | | |
| | | | NCI-H596 | | |
| glioma SNB-19 | 0.0 | 0.0 | Mammary gland | 2.5 | 3.3 |
| glioma U251 | 0.5 | 0.1 | Breast ca.* | 0.0 | 0.0 |
| | | | (pl.ef) MCF-7 | | |
| glioma SF-295 | 13.5 | 42.0 | Breast ca.* | 0.0 | 0.0 |
| | | | (pl.ef) | | |
| | | | MDA-MB-231 | | |
| Heart (fetal) | 0.0 | 0.0 | Breast ca.* | 0.0 | 0.0 |
| | | | (pl.ef) T47D | | |
| Heart | 0.0 | 0.1 | Breast ca. | 1.0 | 0.0 |
| | | | BT-549 | | |
| Skeletal muscle (fetal) | 0.0 | 0.1 | Breast ca. | 0.2 | 0.4 |
| | | | MDA-N | | |
| Skeletal muscle | 3.3 | 1.9 | Ovary | 0.0 | 0.0 |
| Bone marrow | 0.0 | 0.7 | Ovarian ca. | 0.0 | 0.0 |
| | | | OVCAR-3 | | |
| Thymus | 0.0 | 0.0 | Ovarian ca. | 0.0 | 0.0 |
| | | | OVCAR-4 | | |
| Spleen | 0.0 | 0.0 | Ovarian ca. | 0.1 | 0.8 |
| | | | OVCAR-5 | | |
| Lymph node | 0.0 | 0.0 | Ovarian ca. | 0.0 | 0.0 |
| | | | OVCAR-8 | | |
| Colorectal | 0.9 | 1.2 | Ovarian ca. | 0.6 | 0.2 |
| | | | IGROV-1 | | |
| Stomach | 2.2 | 1.5 | Ovarian ca.* | 0.0 | 0.0 |
| | | | (ascites) | | |
| | | | SK-OV-3 | | |
| Small intestine | 1.5 | 1.1 | Uterus | 8.2 | 3.1 |
| Colon ca. SW480 | 0.0 | 0.0 | Placenta | 0.0 | 0.0 |
| Colon ca.* | 0.4 | 0.0 | Prostate | 0.0 | 0.0 |
| SW620(SW480 met) | | | | | |
| Colon ca. HT29 | 0.0 | 0.0 | Prostate ca.* | 0.0 | 0.0 |
| | | | (bone met) PC-3 | | |
| Colon ca. HCT-116 | 0.0 | 0.0 | Testis | 0.2 | 0.4 |
| Colon ca. CaCo-2 | 0.0 | 0.0 | Melanoma | 2.2 | 2.5 |
| | | | Hs688(A).T | | |
| Colon ca. | 42.0 | 28.9 | Melanoma* | 0.0 | 0.2 |
| tissue(ODO3866) | | | (met) | | |
| | | | Hs688(B).T | | |
| Colon ca. HCC-2998 | 0.0 | 0.0 | Melanoma | 0.0 | 0.0 |
| | | | UACC-62 | | |
| Gastric ca.* (liver met) | 5.3 | 1.6 | Melanoma M14 | 0.2 | 0.1 |
| NCI-N87 | | | | | |
| Bladder | 0.0 | 0.2 | Melanoma LOX | 0.2 | 1.7 |
| | | | IMVI | | |
| Trachea | 2.0 | 1.1 | Melanoma* | 0.0 | 0.0 |
| | | | (met) SK-MEL-5 | | |
| Kidney | 0.0 | 0.7 | Adipose | 0.3 | 0.2 |

TABLE JE
Panel 2D

| | Rel. Exp. (%) | | Rel. Exp. (%) |
|---|---|---|---|
| | Ag3395, Run | | Ag3395, Run |
| Tissue Name | 165469036 | Tissue Name | 165469036 |
| Normal Colon | 4.4 | Kidney Margin 8120608 | 0.0 |
| CC Well to Mod Diff | 48.6 | Kidney Cancer 8120613 | 0.0 |
| (ODO3866) | | | |
| CC Margin (ODO3866) | 4.6 | Kidney Margin 8120614 | 0.2 |
| CC Gr.2 rectosigmoid | 9.0 | Kidney Cancer 9010320 | 0.6 |
| (ODO3868) | | | |
| CC Margin (ODO3868) | 0.3 | Kidney Margin 9010321 | 1.1 |
| CC Mod Diff (ODO3920) | 10.9 | Normal Uterus | 0.5 |
| CC Margin (ODO3920) | 1.8 | Uterus Cancer 064011 | 0.9 |
| CC Gr.2 ascend colon | 100.0 | Normal Thyroid | 0.2 |
| (ODO3921) | | | |
| CC Margin (ODO3921) | 3.1 | Thyroid Cancer 064010 | 0.0 |
| CC from Partial Hepatectomy | 1.4 | Thyroid Cancer A302152 | 0.9 |
| (ODO4309) Mets | | | |
| Liver Margin (ODO4309) | 0.3 | Thyroid Margin A302153 | 0.0 |
| Colon mets to lung | 0.1 | Normal Breast | 5.8 |
| (OD04451-01) | | | |
| Lung Margin (OD04451-02) | 0.0 | Breast Cancer (OD04566) | 3.8 |
| Normal Prostate 6546-1 | 1.9 | Breast Cancer (OD04590-01) | 2.7 |
| Prostate Cancer (OD04410) | 0.3 | Breast Cancer Mets | 2.5 |
| | | (OD04590-03) | |
| Prostate Margin (OD04410) | 0.0 | Breast Cancer Metastasis | 0.3 |
| | | (OD04655-05) | |
| Prostate Cancer (OD04720-01) | 0.5 | Breast Cancer 064006 | 17.7 |
| Prostate Margin (OD04720-02) | 0.9 | Breast Cancer 1024 | 4.1 |
| Normal Lung 061010 | 0.4 | Breast Cancer 9100266 | 18.2 |
| Lung Met to Muscle | 0.4 | Breast Margin 9100265 | 30.4 |
| (ODO4286) | | | |
| Muscle Margin (ODO4286) | 9.3 | Breast Cancer A209073 | 16.8 |
| Lung Malignant Cancer | 2.6 | Breast Margin A209073 | 19.3 |
| (OD03126) | | | |
| Lung Margin (OD03126) | 0.3 | Normal Liver | 0.1 |
| Lung Cancer (OD04404) | 25.9 | Liver Cancer 064003 | 0.1 |
| Lung Margin (OD04404) | 0.2 | Liver Cancer 1025 | 0.0 |
| Lung Cancer (OD04565) | 21.9 | Liver Cancer 1026 | 0.0 |
| Lung Margin (OD04565) | 0.4 | Liver Cancer 6004-T | 0.0 |
| Lung Cancer (OD04237-01) | 1.4 | Liver Tissue 6004-N | 1.6 |
| Lung Margin (OD04237-02) | 0.3 | Liver Cancer 6005-T | 0.0 |
| Ocular Mel Met to Liver | 0.1 | Liver Tissue 6005-N | 0.0 |
| (ODO4310) | | | |
| Liver Margin (ODO4310) | 0.1 | Normal Bladder | 0.5 |
| Melanoma Mets to Lung | 0.2 | Bladder Cancer 1023 | 0.6 |
| (OD04321) | | | |
| Lung Margin (OD04321) | 0.3 | Bladder Cancer A302173 | 4.3 |
| Normal Kidney | 1.7 | Bladder Cancer | 13.4 |
| | | (OD04718-01) | |
| Kidney Ca, Nuclear grade 2 | 0.1 | Bladder Normal Adjacent | 35.4 |
| (OD04338) | | (OD04718-03) | |
| Kidney Margin (OD04338) | 1.0 | Normal Ovary | 0.0 |
| Kidney Ca Nuclear grade 1/2 | 0.1 | Ovarian Cancer 064008 | 1.3 |
| (OD04339) | | | |
| Kidney Margin (OD04339) | 1.4 | Ovarian Cancer | 0.0 |
| | | (OD04768-07) | |
| Kidney Ca, Clear cell type | 0.0 | Ovary Margin (OD04768-08) | 1.7 |
| (OD04340) | | | |
| Kidney Margin (OD04340) | 0.5 | Normal Stomach | 1.3 |
| Kidney Ca, Nuclear grade 3 | 0.0 | Gastric Cancer 9060358 | 6.9 |
| (OD04348) | | | |
| Kidney Margin (OD04348) | 1.2 | Stomach Margin 9060359 | 1.4 |
| Kidney Cancer (OD04622-01) | 0.1 | Gastric Cancer 9060395 | 10.2 |
| Kidney Margin (OD04622-03) | 0.3 | Stomach Margin 9060394 | 1.3 |
| Kidney Cancer (OD04450-01) | 0.3 | Gastric Cancer 9060397 | 25.0 |
| Kidney Margin (OD04450-03) | 0.2 | Stomach Margin 9060396 | 1.0 |
| Kidney Cancer 8120607 | 0.5 | Gastric Cancer 064005 | 60.7 |

TABLE JF
Panel 3D

| | Rel. | Rel. | | Rel. | Rel. |
|---|---|---|---|---|---|
| | Exp. (%) | Exp. (%) | | Exp. (%) | Exp. (%) |
| | Ag3395, | Ag3395, | | Ag3395, | Ag3395, |
| | Run | Run | | Run | Run |
| Tissue Name | 165924467 | 167542915 | Tissue Name | 165924467 | 167542915 |
| Daoy- | 0.0 | 0.0 | Ca Ski-Cervical | 0.3 | 0.2 |
| Medulloblastoma | | | epidermoid | | |
| | | | carcinoma | | |
| | | | (metastasis) | | |
| TE671- | 0.0 | 0.0 | ES-2-Ovarian clear | 3.1 | 4.0 |
| Medulloblastoma | | | cell carcinoma | | |
| D283 Med- | 0.0 | 0.0 | Ramos-Stimulated | 0.0 | 0.0 |
| Medulloblastoma | | | with | | |
| | | | PMA/ionomycin 6 h | | |
| PFSK-1-Primitive | 0.0 | 0.0 | Ramos-Stimulated | 0.0 | 0.0 |
| Neuroectodermal | | | with | | |
| | | | PMA/ionomycin | | |
| | | | 14 h | | |
| XF-498-CNS | 0.1 | 0.1 | MEG-01-Chronic | 0.0 | 0.0 |
| | | | myelogenous | | |
| | | | leukemia | | |
| | | | (megokaryoblast) | | |
| SNB-78-Glioma | 0.3 | 0.2 | Raji-Burkitt's | 0.0 | 0.0 |
| | | | lymphoma | | |
| SF-268- | 0.0 | 0.1 | Daudi-Burkitt's | 0.0 | 0.0 |
| Glioblastoma | | | lymphoma | | |
| T98G- | 0.8 | 1.3 | U266-B-cell | 0.0 | 0.0 |
| Glioblastoma | | | plasmacytoma | | |
| SK-N-SH- | 12.9 | 16.7 | CA46-Burkitt's | 0.0 | 0.0 |
| Neuroblastoma | | | lymphoma | | |
| (metastasis) | | | | | |
| SF-295- | 100.0 | 100.0 | RL-non-Hodgkin's | 0.0 | 0.0 |
| Glioblastoma | | | B-cell lymphoma | | |
| Cerebellum | 0.0 | 0.0 | JM1-pre-B-cell | 0.0 | 0.0 |
| | | | lymphoma | | |
| Cerebellum | 0.0 | 0.0 | Jurkat-T cell | 0.0 | 0.0 |
| | | | leukemia | | |
| NCI-H292- | 0.0 | 0.0 | TF-1- | 0.0 | 0.0 |
| Mucoepidermoid | | | Erythroleukemia | | |
| lung carcinoma | | | | | |
| DMS-114-Small | 0.2 | 0.3 | HUT 78-T-cell | 0.0 | 0.0 |
| cell lung cancer | | | lymphoma | | |
| DMS-79-Small cell | 0.0 | 0.0 | U937-Histiocytic | 0.0 | 0.0 |
| lung cancer | | | lymphoma | | |
| NCI-H146-Small | 0.0 | 0.0 | KU-812- | 0.0 | 0.2 |
| cell lung cancer | | | Myelogenous | | |
| | | | leukemia | | |
| NCI-H526-Small | 0.0 | 0.0 | 769-P-Clear cell | 0.0 | 0.0 |
| cell lung cancer | | | renal carcinoma | | |
| NCI-N417-Small | 0.0 | 0.0 | Caki-2-Clear cell | 0.0 | 0.0 |
| cell lung cancer | | | renal carcinoma | | |
| NCI-H82-Small | 0.0 | 0.0 | SW 839-Clear cell | 0.0 | 0.0 |
| cell lung cancer | | | renal carcinoma | | |
| NCI-H157- | 0.0 | 0.0 | G401-Wilms' tumor | 0.0 | 0.0 |
| Squamous cell lung | | | | | |
| cancer (metastasis) | | | | | |
| NCI-H1155-Large | 0.0 | 0.0 | Hs766T-Pancreatic | 0.0 | 0.0 |
| cell lung cancer | | | carcinoma (LN | | |
| | | | metastasis) | | |
| NCI-H1299-Large | 0.0 | 0.0 | CAPAN-1- | 0.0 | 0.0 |
| cell lung cancer | | | Pancreatic | | |
| | | | adenocarcinoma | | |
| | | | (liver metastasis) | | |
| NCI-H727-Lung | 0.0 | 0.0 | SU86.86-Pancreatic | 0.0 | 0.1 |
| carcinoid | | | carcinoma (liver | | |
| | | | metastasis) | | |
| NCI-UMC-11- | 0.1 | 0.0 | BxPC-3-Pancreatic | 0.2 | 0.1 |
| Lung carcinoid | | | adenocarcinoma | | |
| LX-1-Small cell | 0.0 | 0.0 | HPAC-Pancreatic | 0.0 | 0.0 |
| lung cancer | | | adenocarcinoma | | |
| Colo-205-Colon | 0.0 | 0.0 | MIA PaCa-2- | 0.0 | 0.0 |
| cancer | | | Pancreatic carcinoma | | |
| KM12-Colon | 0.0 | 0.0 | CFPAC-1-Pancreatic | 0.0 | 0.1 |
| cancer | | | ductal | | |
| | | | adenocarcinoma | | |
| KM20L2-Colon | 0.0 | 0.0 | PANC-1-Pancreatic | 0.0 | 0.0 |
| cancer | | | epithelioid ductal | | |
| | | | carcinoma | | |
| NCI-H716-Colon | 0.0 | 0.0 | T24-Bladder | 0.0 | 0.0 |
| cancer | | | carcinma (transitional | | |
| | | | cell) | | |
| SW-48-Colon | 0.0 | 0.0 | 5637-Bladder | 0.0 | 0.0 |
| adenocarcinoma | | | carcinoma | | |
| SW1116-Colon | 0.0 | 0.0 | HT-1197-Bladder | 0.0 | 0.1 |
| adenocarcinoma | | | carcinoma | | |
| LS 174T-Colon | 0.0 | 0.0 | UM-UC-3-Bladder | 0.2 | 0.2 |
| adenocarcinoma | | | carcinma (transitional | | |
| | | | cell) | | |
| SW-948-Colon | 0.0 | 0.0 | A204- | 0.2 | 0.2 |
| adenocarcinoma | | | Rhabdomyosarcoma | | |
| SW-480-Colon | 0.0 | 0.0 | HT-1080- | 0.0 | 0.1 |
| adenocarcinoma | | | Fibrosarcoma | | |
| NCI-SNU-5- | 0.0 | 0.0 | MG-63- | 0.3 | 0.5 |
| Gastric carcinoma | | | Osteosarcoma | | |
| KATO III-Gastric | 0.0 | 0.0 | SK-LMS-1- | 23.7 | 38.4 |
| carcinoma | | | Leiomyosarcoma | | |
| | | | (vulva) | | |
| NCI-SNU-16- | 0.1 | 0.2 | SJRH30- | 0.0 | 0.0 |
| Gastric carcinoma | | | Rhabdomyosarcoma | | |
| | | | (met to bone marrow) | | |
| NCI-SNU-1- | 0.0 | 0.0 | A431-Epidermoid | 0.0 | 0.0 |
| Gastric carcinoma | | | carcinoma | | |
| RF-1-Gastric | 0.0 | 0.0 | WM266-4- | 0.0 | 0.0 |
| adenocarcinoma | | | Melanoma | | |
| RF-48-Gastric | 0.0 | 0.0 | DU 145-Prostate | 0.0 | 0.0 |
| adenocarcinoma | | | carcinoma (brain | | |
| | | | metastasis) | | |
| MKN-45-Gastric | 0.0 | 0.0 | MDA-MB-468- | 0.0 | 0.0 |
| carcinoma | | | Breast | | |
| | | | adenocarcinoma | | |
| NCI-N87-Gastric | 0.0 | 0.0 | SCC-4-Squamous | 0.0 | 0.0 |
| carcinoma | | | cell carcinoma of | | |
| | | | tongue | | |
| OVCAR-5- | 0.0 | 0.0 | SCC-9-Squamous | 0.0 | 0.0 |
| Ovarian carcinoma | | | cell carcinoma of | | |
| | | | tongue | | |
| RL95-2-Uterine | 0.0 | 0.0 | SCC-15-Squamous | 0.0 | 0.0 |
| carcinoma | | | cell carcinoma of | | |
| | | | tongue | | |
| HelaS3-Cervical | 0.0 | 0.0 | CAL 27-Squamous | 0.0 | 4.3 |
| adenocarcinoma | | | cell carcinoma of | | |
| | | | tongue | | |

TABLE JG
Panel 4D

| | Rel. Exp. (%) | | Rel. Exp. (%) |
|---|---|---|---|
| | Ag3395, Run | | Ag3395, Run |
| Tissue Name | 165222711 | Tissue Name | 165222711 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 4.0 |
| | | TNF alpha + IL1beta | |
| Primary Th2 rest | 0.0 | Small airway epithelium none | 0.8 |
| Primary Tr1 rest | 0.0 | Small airway epithelium | 7.7 |
| | | TNF alpha + IL-1beta | |
| CD45RA CD4 lymphocyte act | 23.3 | Coronery artery SMC rest | 0.6 |
| CD45RO CD4 lymphocyte act | 0.0 | Coronery artery SMC | 0.8 |
| | | TNF alpha + IL-1beta | |
| CD8 lymphocyte act | 0.0 | Astrocytes rest | 0.0 |
| Secondary CD8 lymphocyte | 0.0 | Astrocytes TNF alpha + | 0.3 |
| rest | | IL-1beta | |
| Secondary CD8 lymphocyte | 0.0 | KU-812 (Basophil) rest | 0.0 |
| act | | | |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) | 0.0 |
| CH11 | | none | |
| LAK cells rest | 0.0 | CCD1106 (Keratinocytes) | 0.1 |
| | | TNF alpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + | 0.0 |
| | | IL-1beta | |
| PBMC rest | 0.0 | Lung fibroblast none | 0.3 |
| PBMC PWM | 0.0 | Lung fibroblast TNF alpha + | 56.6 |
| | | IL-1beta | |
| PBMC PHA-L | 0.0 | Lung fibroblast IL-4 | 0.2 |
| Ramos (B cell) none | 0.0 | Lung fibroblast IL-9 | 1.7 |
| Ramos (B cell) ionomycin | 0.0 | Lung fibroblast IL-13 | 0.1 |
| B lymphocytes PWM | 0.0 | Lung fibroblast IFN gamma | 0.2 |
| B lymphocytes CD40L and | 0.0 | Dermal fibroblast CCD1070 | 16.5 |
| IL-4 | | rest | |
| EOL-1 dbcAMP | 0.0 | Dermal fibroblast CCD1070 | 57.0 |
| | | TNF alpha | |
| EOL-1 dbcAMP | 0.0 | Dermal fibroblast CCD1070 | 100.0 |
| PMA/ionomycin | | IL-1beta | |
| Dendritic cells none | 0.0 | Dermal fibroblast IFN gamma | 1.7 |
| Dendritic cells LPS | 0.0 | Dermal fibroblast IL-4 | 2.9 |
| Dendritic cells anti-CD40 | 0.0 | IBD Colitis 2 | 0.1 |
| Monocytes rest | 0.0 | IBD Crohn's | 0.0 |
| Monocytes LPS | 0.0 | Colon | 0.1 |
| Macrophages rest | 0.0 | Lung | 0.0 |
| Macrophages LPS | 0.0 | Thymus | 0.1 |
| HUVEC none | 0.0 | Kidney | 0.0 |
| HUVEC starved | 0.0 | | |

AI_comprehensive panel_v1.0 Summary: Ag3395 The CG91708-01 transcript is expressed in OA tissue but not in control tissue. The transcript encodes a protein homologous to MMP3 which has been shown to be present in OA joint tissue and may contribute to the pathology of this disease. See, Bluteau G., et al. Biochim Biophys Acta May 3, 2001;1526(2):147-58.

General_screening_panel_v1.4 Summary: Ag3395 Two experiments with the same probe and primer produce results that are in excellent agreement. The expression of this gene appears to be highest in a sample derived a brain cancer cell line (U87-MG) (CTs=22-24). In addition, there appears to be substantial expression in brain cancer cell lines, colon cancer cell lines and melanoma cell lines. Thus, the expression of this gene could be used to distinguish U87-MG cells from other samples in the panel. Moreover, therapeutic modulation of this gene, through the use of small molecule drugs, protein therapeutics or antibodies could be of benefit in the treatment of brain or colon cancer or melanoma.

Among tissues with metabolic function, this gene is expressed at low levels in pancreas, adipose, and fetal skeletal muscle. This expression suggests that this gene product may play a role in normal neuroendocrine and metabolic and that disregulated expression of this gene may contribute to neuroendocrine disorders or metabolic diseases, such as obesity and diabetes.

This gene is also expressed at low but significant levels in the hippocampus, a structure critical for learning and memory. The hippocampus-preferential expression of this gene suggests that it may play a role in learning and memory processes. Agents that modulate the activity and function of CG56633-01 may have utility in treating CNS disorders involving memory deficits, including Alzheimer's disease and aging.

Panel 1.3D Summary: Ag3395 The expression of this gene appears to be highest in samples derived from brain cancer cell lines (U87-MG, U-118-MG). In addition, there appears to be substantial expression in brain cancer cell lines, colon cancer cell lines and gastric cancer cell lines. Thus, the expression of this gene could be used to distinguish U87-MG and U-1118-MG cells from other samples in the panel. Moreover, therapeutic modulation of this gene, through the use of small molecule drugs, protein therapeutics or antibodies could be of benefit in the treatment of brain, colon or gastric cancer.

Panel 2D Summary: Ag3395 The expression of this gene appears to be highest in a sample derived from a colon cancer (CT=26.8). In addition, there appears to be substantial expression in gastric cancer, bladder cancer, breast cancer, lung cancer and colon cancer. Thus, the expression of this gene could be used to distinguish colon cancer cells from other samples in the panel. Moreover, therapeutic modulation of this gene, through the use of small molecule drugs, protein therapeutics or antibodies could be of benefit in the treatment of gastric, bladder, breast, lung or colon cancer.

Panel 3D Summary: Ag3395 Two experiments with two different probes and primers produce results that are in excellent agreement. The expression of this gene appears to be highest in a sample derived from a brain cancer cell line (SF-295) (CTs=24-26). Thus, the expression of this gene could be used to distinguish SF-295 cells from other samples in the panel. Moreover, therapeutic modulation of this gene, through the use of small molecule drugs, protein therapeutics or antibodies could be of benefit in the treatment of brain cancer.

Panel 4D Summary: Ag3395 The CG91708-01 transcript is induced in lung and dermal fibroblasts after treatment with IL-1beta and/or TNF alpha (CTs=21.5-22.5). The protein encoded for by this transcript may facilitate tissue destruction, remodeling and participate in cell:cell interactions that prevent the resolution of the inflammatory response.

Therapeutic targeting of the putative MMP-3 encoded for by this transcript with a human monoclonal antibody may reduce or eliminate inflammation in the skin resulting from psoriasis and allergy, promote wound healing and prevent delayed type hypersensitivity type reactions. In the lung, these therapeutic drugs may reduce or inhibit inflammation and tissue remodeling due to asthma/allergy and emphysema. See, Pilcher B. K., et al. Ann NY Acad Sci Jun. 30, 1999;878:12-24; Dahlen B., et al. Thorax July 1999;54(7):590-6 (PMID: 10377203).

K. NOV15a and NOV15b (CG91729-01 and CG91729-02): MMP13

Expression of gene CG91729-01 and fall length physical clone CG91729-02 was assessed using the primer-probe set Ag3396, described in Table KA. Results of the RTQ-PCR runs are shown in Tables KB, KC, KD, KE, KF and KG.

TABLE KA

Probe Name Ag3396

| | | | Start | SEQ ID |
|---|---|---|---|---|
| Primers | Sequences | Length | Position | No |
| Forward | 5′-ttccctcgaactcttaaatggt-3′ | 22 | 347 | 124 |
| Probe | TET-5′-cctacagaattgtgaattacacccctga-3′-TAMRA | 28 | 384 | 125 |
| Reverse | 5′-aatgccttttcgacttcagaat-3′ | 22 | 420 | 126 |

TABLE KB
AI_comprehensive panel_v1.0

| | Rel. | Rel. | | Rel. | Rel. |
|---|---|---|---|---|---|
| | Exp. (%) | Exp. (%) | | Exp. (%) | Exp. (%) |
| | Ag3396, | Ag3396, | | Ag3396, | Ag3396, |
| | Run | Run | | Run | Run |
| Tissue Name | 211147206 | 212317715 | Tissue Name | 211147206 | 212317715 |
| 110967 | 0.0 | 0.0 | 112427 Match | 0.0 | 0.0 |
| COPD-F | | | Control Psoriasis-F | | |
| 110980 | 0.0 | 0.0 | 112418 Psoriasis-M | 0.0 | 0.0 |
| COPD-F | | | | | |
| 110968 | 0.0 | 0.0 | 112723 Match | 0.0 | 0.0 |
| COPD-M | | | Control Psoriasis-M | | |
| 110977 | 0.0 | 0.0 | 112419 Psoriasis-M | 0.0 | 0.0 |
| COPD-M | | | | | |
| 110989 | 0.0 | 0.0 | 112424 Match | 0.0 | 0.0 |
| Emphysema-F | | | Control Psoriasis-M | | |
| 110992 | 0.0 | 0.0 | 112420 Psoriasis-M | 0.0 | 0.0 |
| Emphysema-F | | | | | |
| 110993 | 0.0 | 0.0 | 112425 Match | 0.0 | 0.0 |
| Emphysema-F | | | Control Psoriasis-M | | |
| 110994 | 0.0 | 0.0 | 104689 (MF) OA | 72.2 | 45.7 |
| Emphysema-F | | | Bone-Backus | | |
| 110995 | 0.0 | 0.0 | 104690 (MF) Adj | 2.0 | 1.6 |
| Emphysema-F | | | “Normal” | | |
| | | | Bone-Backus | | |
| 110996 | 0.0 | 0.0 | 104691 (MF) OA | 0.0 | 0.0 |
| Emphysema-F | | | Synovium-Backus | | |
| 110997 | 0.0 | 0.0 | 104692 (BA) OA | 0.1 | 0.1 |
| Asthma-M | | | Cartilage-Backus | | |
| 111001 | 0.0 | 0.0 | 104694 (BA) OA | 100.0 | 100.0 |
| Asthma-F | | | Bone-Backus | | |
| 111002 | 0.0 | 0.0 | 104695 (BA) Adj | 15.1 | 13.4 |
| Asthma-F | | | “Normal” | | |
| | | | Bone-Backus | | |
| 111003 Atopic | 0.0 | 0.0 | 104696 (BA) OA | 0.4 | 0.2 |
| Asthma-F | | | Synovium-Backus | | |
| 111004 Atopic | 0.0 | 0.0 | 104700 (SS) OA | 1.6 | 2.3 |
| Asthma-F | | | Bone-Backus | | |
| 111005 Atopic | 0.0 | 0.0 | 104701 (SS) Adj | 9.1 | 6.3 |
| Asthma-F | | | “Normal” | | |
| | | | Bone-Backus | | |
| 111006 Atopic | 0.0 | 0.0 | 104702 (SS) OA | 0.0 | 0.0 |
| Asthma-F | | | Synovium- Backus | | |
| 111417 | 0.0 | 0.0 | 117093 OA | 0.0 | 0.0 |
| Allergy-M | | | Cartilage Rep7 | | |
| 112347 | 0.0 | 0.0 | 112672 OA Bone5 | 0.0 | 0.0 |
| Allergy-M | | | | | |
| 112349 Normal | 0.0 | 0.0 | 112673 OA | 0.0 | 0.0 |
| Lung-F | | | Synovium5 | | |
| 112357 Normal | 0.0 | 0.0 | 112674 OA | 0.0 | 0.0 |
| Lung-F | | | Synovial Fluid | | |
| | | | cells5 | | |
| 112354 Normal | 0.0 | 0.0 | 117100 OA | 0.0 | 0.0 |
| Lung-M | | | Cartilage Rep14 | | |
| 112374 | 1.3 | 1.1 | 112756 OA Bone9 | 0.0 | 0.1 |
| Crohns-F | | | | | |
| 112389 Match | 0.0 | 0.0 | 112757 OA | 0.0 | 0.0 |
| Control | | | Synovium9 | | |
| Crohns-F | | | | | |
| 112375 | 1.4 | 1.0 | 112758 OA | 0.0 | 0.0 |
| Crohns-F | | | Synovial Fluid | | |
| | | | Cells9 | | |
| 112732 Match | 0.0 | 0.0 | 117125 RA | 0.0 | 0.0 |
| Control | | | Cartilage Rep2 | | |
| Crohns-F | | | | | |
| 112725 | 0.0 | 0.0 | 113492 Bone2 RA | 0.0 | 0.1 |
| Crohns-M | | | | | |
| 112387 Match | 0.0 | 0.0 | 113493 Synovium2 | 0.0 | 0.0 |
| Control | | | RA | | |
| Crohns-M | | | | | |
| 112378 | 0.0 | 0.0 | 113494 Syn Fluid | 0.0 | 0.0 |
| Crohns-M | | | Cells RA | | |
| 112390 Match | 0.1 | 0.1 | 113499 Cartilage4 RA | 0.0 | 0.0 |
| Control | | | | | |
| Crohns-M | | | | | |
| 112726 | 0.0 | 0.0 | 113500 Bone4 RA | 0.0 | 0.0 |
| Crohns-M | | | | | |
| 112731 Match | 0.0 | 0.0 | 113501 Synovium4 | 0.0 | 0.0 |
| Control | | | RA | | |
| Crohns-M | | | | | |
| 112380 Ulcer | 0.0 | 0.0 | 113502 Syn Fluid | 0.0 | 0.0 |
| Col-F | | | Cells4 RA | | |
| 112734 Match | 0.0 | 0.0 | 113495 Cartilage3 RA | 0.0 | 0.0 |
| Control Ulcer | | | | | |
| Col-F | | | | | |
| 112384 Ulcer | 0.0 | 0.0 | 113496 Bone3 RA | 0.0 | 0.0 |
| Col-F | | | | | |
| 112737 Match | 0.0 | 0.0 | 113497 Synovium3 | 0.0 | 0.0 |
| Control Ulcer | | | RA | | |
| Col-F | | | | | |
| 112386 Ulcer | 0.1 | 0.1 | 113498 Syn Fluid | 0.0 | 0.0 |
| Col-F | | | Cells3 RA | | |
| 112738 Match | 0.0 | 0.0 | 117106 Normal | 0.0 | 0.0 |
| Control Ulcer | | | Cartilage Rep20 | | |
| Col-F | | | | | |
| 112381 Ulcer | 0.0 | 0.0 | 113663 Bone3 | 0.0 | 0.0 |
| Col-M | | | Normal | | |
| 112735 Match | 1.0 | 0.6 | 113664 Synovium3 | 0.0 | 0.0 |
| Control Ulcer | | | Normal | | |
| Col-M | | | | | |
| 112382 Ulcer | 0.0 | 0.0 | 113665 Syn Fluid | 0.0 | 0.0 |
| Col-M | | | Cells3 Normal | | |
| 112394 Match | 0.0 | 0.0 | 117107 Normal | 0.0 | 0.0 |
| Control Ulcer | | | Cartilage Rep22 | | |
| Col-M | | | | | |
| 112383 Ulcer | 0.0 | 0.0 | 113667 Bone4 | 0.0 | 0.0 |
| Col-M | | | Normal | | |
| 112736 Match | 0.0 | 0.0 | 113668 Synovium4 | 0.0 | 0.0 |
| Control User | | | Normal | | |
| Col-M | | | | | |
| 112423 | 0.0 | 0.0 | 113669 Syn Fluid | 0.0 | 0.0 |
| Psoriasis-F | | | Cells4 Normal | | |

TABLE KC
Panel 1.3D

| | Rel. Exp. (%) | Rel. Exp. (%) | | Rel. Exp. (%) | Rel. Exp. (%) |
|---|---|---|---|---|---|
| | Ag3396, Run | Ag3396, Run | | Ag3396, Run | Ag3396, Run |
| Tissue Name | 165524932 | 167595424 | Tissue Name | 165524932 | 167595424 |
| Liver | 0.0 | 0.0 | Kidney (fetal) | 0.0 | 0.1 |
| adenocarcinoma | | | | | |
| Pancreas | 0.0 | 0.0 | Renal ca. 786-0 | 5.6 | 5.9 |
| Pancreatic ca. | 0.7 | 0.3 | Renal ca. A498 | 0.2 | 0.2 |
| CAPAN 2 | | | | | |
| Adrenal gland | 0.0 | 0.0 | Renal ca. RXF | 30.4 | 36.3 |
| | | | 393 | | |
| Thyroid | 0.0 | 0.0 | Renal ca. | 0.0 | 0.0 |
| | | | ACHN | | |
| Salivary gland | 0.0 | 0.0 | Renal ca. UO-31 | 0.0 | 0.0 |
| Pituitary gland | 1.5 | 0.3 | Renal ca. TK-10 | 0.0 | 0.0 |
| Brain (fetal) | 0.0 | 0.0 | Liver | 0.0 | 0.0 |
| Brain (whole) | 0.0 | 0.0 | Liver (fetal) | 0.0 | 0.0 |
| Brain (amygdala) | 0.0 | 0.0 | Liver ca. | 0.0 | 0.0 |
| | | | (hepatoblast) | | |
| | | | HepG2 | | |
| Brain (cerebellum) | 0.0 | 0.0 | Lung | 0.0 | 0.0 |
| Brain | 0.0 | 0.0 | Lung (fetal) | 0.0 | 0.3 |
| (hippocampus) | | | | | |
| Brain (substantia | 0.0 | 0.0 | Lung ca. (small | 0.0 | 0.0 |
| nigra) | | | cell) LX-1 | | |
| Brain (thalamus) | 0.0 | 0.0 | Lung ca. (small | 0.0 | 0.0 |
| | | | cell) NCI-H69 | | |
| Cerebral Cortex | 0.0 | 0.0 | Lung ca. (s.cell | 0.0 | 0.1 |
| | | | var.) SHP-77 | | |
| Spinal cord | 0.0 | 0.0 | Lung ca. (large | 0.0 | 0.0 |
| | | | cell) NCI-H460 | | |
| glio/astro | 0.0 | 0.0 | Lung ca. | 0.0 | 0.0 |
| U87-MG | | | (non-sm. cell) | | |
| | | | A549 | | |
| glio/astro | 0.0 | 0.0 | Lung ca. | 0.0 | 0.0 |
| U-118-MG | | | (non-s.cell) | | |
| | | | NCI-H23 | | |
| astrocytoma | 0.6 | 0.7 | Lung ca. | 0.0 | 0.9 |
| SW1783 | | | (non-s.cell) | | |
| | | | HOP-62 | | |
| neuro*; met | 0.0 | 0.0 | Lung ca. | 0.0 | 0.0 |
| SK-N-AS | | | (non-s.cl) | | |
| | | | NCI-H522 | | |
| astrocytoma | 0.8 | 0.3 | Lung ca. | 100.0 | 100.0 |
| SF-539 | | | (squam.) SW | | |
| | | | 900 | | |
| astrocytoma | 45.7 | 43.5 | Lung ca. | 0.0 | 0.0 |
| SNB-75 | | | (squam.) | | |
| | | | NCI-H596 | | |
| glioma SNB-19 | 0.0 | 0.0 | Mammary gland | 0.9 | 0.7 |
| glioma U251 | 0.4 | 0.0 | Breast ca.* | 0.7 | 2.6 |
| | | | (pl.ef) MCF-7 | | |
| glioma SF-295 | 20.0 | 47.6 | Breast ca.* | 0.0 | 0.1 |
| | | | (pl.ef) | | |
| | | | MDA-MB-231 | | |
| Heart (fetal) | 0.0 | 0.0 | Breast ca.* | 0.0 | 0.3 |
| | | | (pl.ef) T47D | | |
| Heart | 0.0 | 0.0 | Breast ca. | 0.0 | 0.1 |
| | | | BT-549 | | |
| Skeletal muscle | 0.0 | 0.0 | Breast ca. | 0.0 | 0.0 |
| (fetal) | | | MDA-N | | |
| Skeletal muscle | 0.0 | 0.0 | Ovary | 0.0 | 0.0 |
| Bone marrow | 0.9 | 0.7 | Ovarian ca. | 0.0 | 0.0 |
| | | | OVCAR-3 | | |
| Thymus | 0.0 | 0.0 | Ovarian ca. | 0.0 | 0.0 |
| | | | OVCAR-4 | | |
| Spleen | 0.0 | 0.0 | Ovarian ca. | 0.0 | 0.0 |
| | | | OVCAR-5 | | |
| Lymph node | 0.0 | 0.0 | Ovarian ca. | 0.0 | 0.0 |
| | | | OVCAR-8 | | |
| Colorectal | 0.0 | 0.0 | Ovarian ca. | 0.0 | 0.0 |
| | | | IGROV-1 | | |
| Stomach | 0.0 | 0.0 | Ovarian ca.* | 0.6 | 1.4 |
| | | | (ascites) | | |
| | | | SK-OV-3 | | |
| Small intestine | 0.0 | 0.0 | Uterus | 0.0 | 0.0 |
| Colon ca. SW480 | 0.0 | 0.0 | Placenta | 0.0 | 0.0 |
| Colon ca.* | 0.0 | 0.1 | Prostate | 0.0 | 0.1 |
| SW620(SW480 | | | | | |
| met) | | | | | |
| Colon ca. HT29 | 0.0 | 0.0 | Prostate ca.* | 10.4 | 18.6 |
| | | | (bone met) PC-3 | | |
| Colon ca. | 0.0 | 0.0 | Testis | 0.0 | 0.0 |
| HCT-116 | | | | | |
| Colon ca. CaCo-2 | 0.0 | 0.2 | Melanoma | 0.0 | 0.0 |
| | | | Hs688(A).T | | |
| Colon ca. | 7.3 | 4.3 | Melanoma* | 0.0 | 0.0 |
| tissue(ODO3866) | | | (met) | | |
| | | | Hs688(B).T | | |
| Colon ca. | 1.4 | 0.3 | Melanoma | 0.0 | 0.0 |
| HCC-2998 | | | UACC-62 | | |
| Gastric ca.* (liver | 6.3 | 2.5 | Melanoma M14 | 0.0 | 0.0 |
| met) NCI-N87 | | | | | |
| Bladder | 0.3 | 0.0 | Melanoma LOX | 0.0 | 0.0 |
| | | | IMVI | | |
| Trachea | 3.0 | 1.0 | Melanoma* (met) | 0.0 | 0.0 |
| | | | SK-MEL-5 | | |
| Kidney | 0.0 | 0.0 | Adipose | 0.0 | 0.0 |

TABLE KD
Panel 2D

| | Rel. Exp. (%) | | Rel. Exp. (%) |
|---|---|---|---|
| | Ag3396, Run | | Ag3396, Run |
| Tissue Name | 165468498 | Tissue Name | 165468498 |
| Normal Colon | 0.3 | Kidney Margin 8120608 | 0.0 |
| CC Well to Mod Diff (ODO3866) | 25.9 | Kidney Cancer 8120613 | 0.0 |
| CC Margin (ODO3866) | 0.1 | Kidney Margin 8120614 | 0.0 |
| CC Gr.2 rectosigmoid | 1.1 | Kidney Cancer 9010320 | 0.0 |
| (ODO3868) | | | |
| CC Margin (ODO3868) | 0.0 | Kidney Margin 9010321 | 0.0 |
| CC Mod Diff (ODO3920) | 0.0 | Normal Uterus | 0.0 |
| CC Margin (ODO3920) | 0.0 | Uterus Cancer 064011 | 0.1 |
| CC Gr.2 ascend colon | 1.3 | Normal Thyroid | 0.0 |
| (ODO3921) | | | |
| CC Margin (ODO3921) | 0.1 | Thyroid Cancer 064010 | 2.0 |
| CC from Partial Hepatectomy | 0.2 | Thyroid Cancer A302152 | 12.8 |
| (ODO4309) Mets | | | |
| Liver Margin (ODO4309) | 0.0 | Thyroid Margin A302153 | 0.0 |
| Colon mets to lung | 0.0 | Normal Breast | 0.6 |
| (OD04451-01) | | | |
| Lung Margin (OD04451-02) | 0.0 | Breast Cancer (OD04566) | 3.1 |
| Normal Prostate 6546-1 | 0.9 | Breast Cancer | 1.9 |
| | | (OD04590-01) | |
| Prostate Cancer (OD04410) | 0.0 | Breast Cancer Mets | 0.0 |
| | | (OD04590-03) | |
| Prostate Margin (OD04410) | 0.3 | Breast Cancer Metastasis | 0.2 |
| | | (OD04655-05) | |
| Prostate Cancer (OD04720-01) | 2.3 | Breast Cancer 064006 | 26.2 |
| Prostate Margin (OD04720-02) | 1.0 | Breast Cancer 1024 | 0.7 |
| Normal Lung 061010 | 0.0 | Breast Cancer 9100266 | 5.0 |
| Lung Met to Muscle (ODO4286) | 1.3 | Breast Margin 9100265 | 6.7 |
| Muscle Margin (ODO4286) | 0.0 | Breast Cancer A209073 | 7.7 |
| Lung Malignant Cancer | 4.0 | Breast Margin A209073 | 5.2 |
| (OD03126) | | | |
| Lung Margin (OD03126) | 0.5 | Normal Liver | 0.0 |
| Lung Cancer (OD04404) | 6.9 | Liver Cancer 064003 | 0.2 |
| Lung Margin (OD04404) | 3.9 | Liver Cancer 1025 | 0.0 |
| Lung Cancer (OD04565) | 100.0 | Liver Cancer 1026 | 0.0 |
| Lung Margin (OD04565) | 0.3 | Liver Cancer 6004-T | 0.0 |
| Lung Cancer (OD04237-01) | 1.7 | Liver Tissue 6004-N | 0.0 |
| Lung Margin (OD04237-02) | 0.5 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 1.5 |
| Melanoma Mets to Lung | 0.0 | Bladder Cancer 1023 | 1.2 |
| (OD04321) | | | |
| Lung Margin (OD04321) | 0.2 | Bladder Cancer A302173 | 40.3 |
| Normal Kidney | 0.0 | Bladder Cancer | 4.5 |
| | | (OD04718-01) | |
| Kidney Ca, Nuclear grade 2 | 0.6 | Bladder Normal Adjacent | 0.0 |
| (OD04338) | | (OD04718-03) | |
| Kidney Margin (OD04338) | 0.0 | Normal Ovary | 0.0 |
| Kidney Ca Nuclear grade 1/2 | 0.0 | Ovarian Cancer 064008 | 3.1 |
| (OD04339) | | | |
| Kidney Margin (OD04339) | 0.0 | Ovarian Cancer | 0.0 |
| | | (OD04768-07) | |
| Kidney Ca, Clear cell type | 0.0 | Ovary Margin | 0.0 |
| (OD04340) | | (OD04768-08) | |
| Kidney Margin (OD04340) | 0.0 | Normal Stomach | 0.0 |
| Kidney Ca, Nuclear grade 3 | 0.6 | Gastric Cancer 9060358 | 0.0 |
| (OD04348) | | | |
| Kidney Margin (OD04348) | 0.0 | Stomach Margin 9060359 | 0.0 |
| Kidney Cancer (OD04622-01) | 0.3 | Gastric Cancer 9060395 | 0.0 |
| Kidney Margin (OD04622-03) | 0.0 | Stomach Margin 9060394 | 0.0 |
| Kidney Cancer (OD04450-01) | 0.0 | Gastric Cancer 9060397 | 2.6 |
| Kidney Margin (OD04450-03) | 0.0 | Stomach Margin 9060396 | 0.0 |
| Kidney Cancer 8120607 | 7.0 | Gastric Cancer 064005 | 0.2 |

TABLE KE
Panel 3D

| | Rel. | Rel. | | Rel. | Rel. |
|---|---|---|---|---|---|
| | Exp. (%) | Exp. (%) | | Exp. (%) | Exp. (%) |
| | Ag3396, | Ag3396, | | Ag3396, | Ag3396, |
| | Run | Run | | Run | Run |
| Tissue Name | 165924635 | 167542917 | Tissue Name | 165924635 | 167542917 |
| Daoy- | 0.1 | 0.1 | Ca Ski-Cervical | 2.0 | 2.2 |
| Medulloblastoma | | | epidermoid | | |
| | | | carcinoma | | |
| | | | (metastasis) | | |
| TE671- | 0.0 | 0.0 | ES-2-Ovarian clear | 0.0 | 0.1 |
| Medulloblastoma | | | cell carcinoma | | |
| D283 Med- | 0.0 | 0.0 | Ramos-Stimulated | 0.0 | 0.0 |
| Medulloblastoma | | | with | | |
| | | | PMA/ionomycin 6 h | | |
| PFSK-1-Primitive | 0.3 | 0.3 | Ramos-Stimulated | 0.0 | 0.0 |
| Neuroectodermal | | | with | | |
| | | | PMA/ionomycin | | |
| | | | 14 h | | |
| XF-498-CNS | 0.0 | 0.0 | MEG-01-Chronic | 0.0 | 0.0 |
| | | | myelogenous | | |
| | | | leukemia | | |
| | | | (megokaryoblast) | | |
| SNB-78-Glioma | 0.0 | 0.0 | Raji-Burkitt's | 0.0 | 0.0 |
| | | | lymphoma | | |
| SF-268- | 0.2 | 0.1 | Daudi-Burkitt's | 0.0 | 0.0 |
| Glioblastoma | | | lymphoma | | |
| T98G- | 9.8 | 19.2 | U266-B-cell | 0.0 | 0.1 |
| Glioblastoma | | | plasmacytoma | | |
| SK-N-SH- | 0.0 | 0.1 | CA46-Burkitt's | 0.0 | 0.0 |
| Neuroblastoma | | | lymphoma | | |
| (metastasis) | | | | | |
| SF-295- | 100.0 | 100.0 | RL-non-Hodgkin's | 0.0 | 0.0 |
| Glioblastoma | | | B-cell lymphoma | | |
| Cerebellum | 0.0 | 0.0 | JM1-pre-B-cell | 0.0 | 0.0 |
| | | | lymphoma | | |
| Cerebellum | 0.0 | 0.0 | Jurkat-T cell | 0.0 | 0.0 |
| | | | leukemia | | |
| NCI-H292- | 0.4 | 0.7 | TF-1- | 0.0 | 0.0 |
| Mucoepidermoid | | | Erythroleukemia | | |
| lung carcinoma | | | | | |
| DMS-114-Small | 0.0 | 0.0 | HUT 78-T-cell | 0.0 | 0.0 |
| cell lung cancer | | | lymphoma | | |
| DMS-79-Small | 0.0 | 0.4 | U937-Histiocytic | 0.0 | 0.0 |
| cell lung cancer | | | lymphoma | | |
| NCI-H146-Small | 0.0 | 0.0 | KU-812- | 0.0 | 0.0 |
| cell lung cancer | | | Myelogenous | | |
| | | | leukemia | | |
| NCI-H526-Small | 0.0 | 0.0 | 769-P-Clear cell | 0.0 | 0.0 |
| cell lung cancer | | | renal carcinoma | | |
| NCI-N417-Small | 0.0 | 0.0 | Caki-2-Clear cell | 0.0 | 0.0 |
| cell lung cancer | | | renal carcinoma | | |
| NCI-H82-Small | 0.0 | 0.0 | SW 839-Clear cell | 0.0 | 0.0 |
| cell lung cancer | | | renal carcinoma | | |
| NCI-H157- | 0.0 | 0.1 | G401-Wilms' | 0.0 | 0.0 |
| Squamous cell | | | tumor | | |
| lung cancer | | | | | |
| (metastasis) | | | | | |
| NCI-H1155-Large | 0.0 | 0.0 | Hs766T-Pancreatic | 0.0 | 0.1 |
| cell lung cancer | | | carcinoma (LN | | |
| | | | metastasis) | | |
| NCI-H1299-Large | 0.5 | 1.2 | CAPAN-1- | 0.0 | 0.6 |
| cell lung cancer | | | Pancreatic | | |
| | | | adenocarcinoma | | |
| | | | (liver metastasis) | | |
| NCI-H727-Lung | 24.5 | 40.3 | SU86.86-Pancreatic | 0.3 | 0.1 |
| carcinoid | | | carcinoma (liver | | |
| | | | metastasis) | | |
| NCI-UMC-11- | 0.4 | 0.7 | BxPC-3-Pancreatic | 0.7 | 1.0 |
| Lung carcinoid | | | adenocarcinoma | | |
| LX-1-Small cell | 0.0 | 0.0 | HPAC-Pancreatic | 0.0 | 0.1 |
| lung cancer | | | adenocarcinoma | | |
| Colo-205-Colon | 0.0 | 0.0 | MIA PaCa-2- | 0.0 | 0.0 |
| cancer | | | Pancreatic carcinoma | | |
| KM12-Colon | 0.0 | 0.0 | CFPAC-1-Pancreatic | 1.8 | 3.5 |
| cancer | | | ductal | | |
| | | | adenocarcinoma | | |
| KM20L2-Colon | 0.0 | 0.0 | PANC-1-Pancreatic | 0.0 | 0.0 |
| cancer | | | epithelioid ductal | | |
| | | | carcinoma | | |
| NCI-H716-Colon | 0.0 | 0.2 | T24-Bladder | 0.3 | 0.2 |
| cancer | | | carcinma (transitional | | |
| | | | cell) | | |
| SW-48-Colon | 0.0 | 0.0 | 5637-Bladder | 0.3 | 0.4 |
| adenocarcinoma | | | carcinoma | | |
| SW1116-Colon | 0.0 | 0.0 | HT-1197-Bladder | 1.2 | 1.8 |
| adenocarcinoma | | | carcinoma | | |
| LS 174T-Colon | 0.0 | 0.0 | UM-UC-3-Bladder | 0.0 | 0.0 |
| adenocarcinoma | | | carcinma (transitional | | |
| | | | cell) | | |
| SW-948-Colon | 0.0 | 0.0 | A204- | 0.0 | 0.0 |
| adenocarcinoma | | | Rhabdomyosarcoma | | |
| SW-480-Colon | 0.0 | 0.0 | HT-1080- | 0.1 | 0.2 |
| adenocarcinoma | | | Fibrosarcoma | | |
| NCI-SNU-5- | 0.0 | 0.0 | MG-63- | 0.0 | 0.0 |
| Gastric carcinoma | | | Osteosarcoma | | |
| KATO III-Gastric | 0.0 | 0.0 | SK-LMS-1- | 0.3 | 0.8 |
| carcinoma | | | Leiomyosarcoma | | |
| | | | (vulva) | | |
| NCI-SNU-16- | 0.0 | 0.0 | SJRH30- | 0.0 | 0.0 |
| Gastric carcinoma | | | Rhabdomyosarcoma | | |
| | | | (met to bone marrow) | | |
| NCI-SNU-1- | 0.0 | 0.0 | A431-Epidermoid | 1.0 | 4.3 |
| Gastric carcinoma | | | carcinoma | | |
| RF-1-Gastric | 0.0 | 0.0 | WM266-4- | 0.0 | 0.0 |
| adenocarcinoma | | | Melanoma | | |
| RF-48-Gastric | 0.0 | 0.0 | DU 145-Prostate | 0.0 | 0.0 |
| adenocarcinoma | | | carcinoma (brain | | |
| | | | metastasis) | | |
| MKN-45-Gastric | 0.2 | 0.2 | MDA-MB-468- | 0.0 | 0.0 |
| carcinoma | | | Breast | | |
| | | | adenocarcinoma | | |
| NCI-N87-Gastric | 0.0 | 0.1 | SCC-4-Squamous | 0.0 | 0.0 |
| carcinoma | | | cell carcinoma of | | |
| | | | tongue | | |
| OVCAR-5- | 0.0 | 0.0 | SCC-9-Squamous | 0.0 | 0.0 |
| Ovarian carcinoma | | | cell carcinoma of | | |
| | | | tongue | | |
| RL95-2-Uterine | 0.0 | 0.0 | SCC-15-Squamous | 0.2 | 2.3 |
| carcinoma | | | cell carcinoma of | | |
| | | | tongue | | |
| HelaS3-Cervical | 0.0 | 0.0 | CAL 27-Squamous | 6.0 | 11.6 |
| adenocarcinoma | | | cell carcinoma of | | |
| | | | tongue | | |

TABLE KF
Panel 4.1D

| | Rel. Exp. (%) | | Rel. Exp. (%) |
|---|---|---|---|
| | Ag3396, Run | | Ag3396, Run |
| Tissue Name | 169838993 | Tissue Name | 169838993 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.3 |
| | | TNF alpha + IL-1beta | |
| Primary Th2 act | 0.0 | Microvascular Dermal EC | 0.1 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 100.0 |
| | | TNF alpha + IL1beta | |
| Primary Th2 rest | 0.0 | Small airway epithelium none | 0.3 |
| Primary Tr1 rest | 0.0 | Small airway epithelium | 29.5 |
| | | TNF alpha + IL-1beta | |
| CD45RA CD4 lymphocyte act | 0.2 | Coronery artery SMC rest | 0.0 |
| CD45RO CD4 lymphocyte act | 0.0 | Coronery artery SMC | 0.0 |
| | | TNF alpha + IL-1beta | |
| CD8 lymphocyte act | 0.0 | Astrocytes rest | 0.2 |
| Secondary CD8 lymphocyte | 0.0 | Astrocytes TNF alpha + | 9.0 |
| rest | | IL-1beta | |
| Secondary CD8 lymphocyte | 0.0 | KU-812 (Basophil) rest | 0.0 |
| act | | | |
| CD4 lymphocyte none | 0.0 | KU-812 (Basophil) | 0.3 |
| | | PMA/ionomycin | |
| 2ry Th1/Th2/Tr1_anti-CD95 | 0.0 | CCD1106 (Keratinocytes) | 1.3 |
| CH11 | | none | |
| LAK cells rest | 0.0 | CCD1106 (Keratinocytes) | 17.7 |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 1.8 |
| LAK cells IL-2 + IFN gamma | 0.0 | NCI-H292 IL-4 | 1.4 |
| LAK cells IL-2 + IL-18 | 0.0 | NCI-H292 IL-9 | 1.9 |
| LAK cells PMA/ionomycin | 0.0 | NCI-H292 IL-13 | 0.9 |
| NK Cells IL-2 rest | 0.0 | NCI-H292 IFN gamma | 0.6 |
| Two Way MLR 3 day | 0.0 | HPAEC none | 0.0 |
| Two Way MLR 5 day | 0.0 | HPAEC TNF alpha + | 0.0 |
| | | IL-1beta | |
| Two Way MLR 7 day | 0.0 | Lung fibroblast none | 0.1 |
| PBMC rest | 0.0 | Lung fibroblast TNF alpha + | 5.4 |
| | | IL-1beta | |
| PBMC PWM | 0.0 | Lung fibroblast IL-4 | 0.1 |
| PBMC PHA-L | 0.0 | Lung fibroblast IL-9 | 0.6 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 | 0.1 |
| | | rest | |
| B lymphocytes CD40L and | 0.0 | Dermal fibroblast CCD1070 | 0.0 |
| IL-4 | | TNF alpha | |
| EOL-1 dbcAMP | 0.0 | Dermal fibroblast CCD1070 | 0.1 |
| | | IL-1beta | |
| EOL-1 dbcAMP | 0.1 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.0 |
| Macrophages rest | 0.0 | Lung | 0.5 |
| Macrophages LPS | 0.0 | Thymus | 0.0 |
| HUVEC none | 0.0 | Kidney | 0.0 |
| HUVEC starved | 0.0 | | |

AI_comprehensive panel v1.0 Summary: Ag3396 The CG91729-01 transcript is expressed in OA tissue but not in control tissue in two experiments with the same probe and primer set (CTs=24-26). The transcript encodes a putative MMP13 which has been shown to be present in OA joint tissue and may contribute to the pathology of this disease. See, Bluteau G., et al. Biochim BiophysActa May 3, 2001;1526(2):147-58.

Panel 1.3D Summary: Ag3396 The expression of this gene appears to be highest in a sample derived from a lung cancer cell line (SW-900) in two experiments with the same probe and primer set (CTs=27-29). In addition, there appears to be substantial expression in prostate cancer cell lines, renal cancer cell lines and brain cancer cell lines. Thus, the expression of this gene could be used to distinguish SW-900 cells from other samples in the panel. Moreover, therapeutic modulation of this gene, through the use of small molecule drugs, protein therapeutics or antibodies could be of benefit in the treatment of lung, prostate, renal or brain cancer.

Panel 2D Summary: Ag3396 The expression of this gene appears to be highest in a sample derived from a lung cancer (CT=27.7). In addition, there appears to be substantial expression in bladder cancer, breast cancer, thyroid cancer and lung cancer. Thus, the expression of this gene could be used to distinguish lung cancer cells from other samples in the panel. Moreover, therapeutic modulation of this gene, through the use of small molecule drugs, protein therapeutics or antibodies could be of benefit in the treatment of bladder, breast, thyroid or lung cancer.

Panel 3D Summary: Ag3396 Two experiments with the same probe and primer set show the expression of this gene highest in a sample derived from a brain cancer cell line (SF-295) (CTs=26.5-27.5). In addition, there appears to be substantial expression in brain cancer cell lines and lung cancer cell lines. Thus, the expression of this gene could be used to distinguish SF-295 cells from other samples in the panel. Moreover, therapeutic modulation of this gene, through the use of small molecule drugs, protein therapeutics or antibodies could be of benefit in the treatment of brain or lung cancer.

Panels 4D and 4.1D Summary: Ag3396 The CG91729-01 transcript is induced in TNFalpha and IL-1beta treated fibroblasts, keratinocytes, and epithelium (CTs—29-31.5). The transcript encodes a putative MMP-13, collagenase 3, which is involved in OA and in wound repair in general. See, Wu N, et al. Matrix Biol March 2002;21(2): 149-61). Human monoclonal antibodies against this protein could be used to treat OA and other conditions such as psoriasis and emphysema in which aberrant wound healing contribute to the pathology.

L. NOV16a (CG92489-01): BCG Induced Integral Membrane Protein

Expression of gene CG92489-01 was assessed using the primer-probe set Ag2558, described in Table LA. Results of the RTQ-PCR runs are shown in Tables LB, LC, LD and LE.

TABLE LA

Probe Name Ag2558

| | | | Start | SEQ ID |
|---|---|---|---|---|
| Primers | Sequences | Length | Position | No |
| Forward | 5′-atgattcagaatgctggaatgt-3′ | 22 | 1588 | 127 |
| Probe | TET-5′-aactggattcacagccattctactca-3′-TAMRA | 26 | 1611 | 128 |
| Reverse | 5′-attcgatttctcctgcatacaa-3′ | 22 | 1642 | 129 |

TABLE LB
AI_comprehensive panel_v1.0

| | Rel. | Rel. | | Rel. | Rel. |
|---|---|---|---|---|---|
| | Exp. (%) | Exp. (%) | | Exp. (%) | Exp. (%) |
| | Ag2558, | Ag2558, | | Ag2558, | Ag2558, |
| | Run | Run | | Run | Run |
| Tissue Name | 228059678 | 229393909 | Tissue Name | 228059678 | 229393909 |
| 110967 | 1.2 | 0.9 | 112427 Match | 6.7 | 3.0 |
| COPD-F | | | Control Psoriasis-F | | |
| 110980 | 2.6 | 2.3 | 112418 Psoriasis-M | 1.3 | 0.9 |
| COPD-F | | | | | |
| 110968 | 1.6 | 0.9 | 112723 Match | 0.9 | 0.7 |
| COPD-M | | | Control Psoriasis-M | | |
| 110977 | 4.6 | 3.7 | 112419 Psoriasis-M | 2.7 | 2.5 |
| COPD-M | | | | | |
| 110989 | 7.1 | 5.8 | 112424 Match | 1.3 | 1.3 |
| Emphysema-F | | | Control Psoriasis-M | | |
| 110992 | 3.8 | 4.4 | 112420 Psoriasis-M | 8.5 | 6.7 |
| Emphysema-F | | | | | |
| 110993 | 0.8 | 0.9 | 112425 Match | 5.6 | 2.3 |
| Emphysema-F | | | Control Psoriasis-M | | |
| 110994 | 0.7 | 0.5 | 104689 (MF) OA | 17.8 | 14.9 |
| Emphysema-F | | | Bone-Backus | | |
| 110995 | 9.7 | 8.2 | 104690 (MF) Adj | 10.3 | 9.3 |
| Emphysema-F | | | “Normal” | | |
| | | | Bone-Backus | | |
| 110996 | 3.0 | 3.0 | 104691 (MF) OA | 6.0 | 5.3 |
| Emphysema-F | | | Synovium-Backus | | |
| 110997 | 2.6 | 2.7 | 104692 (BA) OA | 48.3 | 35.1 |
| Asthma-M | | | Cartilage-Backus | | |
| 111001 | 3.6 | 2.4 | 104694 (BA) OA | 12.9 | 12.2 |
| Asthma-F | | | Bone-Backus | | |
| 111002 | 3.8 | 4.4 | 104695 (BA) Adj | 14.3 | 12.3 |
| Asthma-F | | | “Normal” | | |
| | | | Bone-Backus | | |
| 111003 Atopic | 6.6 | 4.7 | 104696 (BA) OA | 9.7 | 8.9 |
| Asthma-F | | | Synovium-Backus | | |
| 111004 Atopic | 11.5 | 8.8 | 104700 (SS) OA | 9.3 | 9.2 |
| Asthma-F | | | Bone-Backus | | |
| 111005 Atopic | 7.6 | 5.2 | 104701 (SS) Adj | 10.5 | 11.8 |
| Asthma-F | | | “Normal” | | |
| | | | Bone-Backus | | |
| 111006 Atopic | 1.8 | 1.1 | 104702 (SS) OA | 12.8 | 10.6 |
| Asthma-F | | | Synovium-Backus | | |
| 111417 | 5.1 | 2.0 | 117093 OA | 2.8 | 2.7 |
| Allergy-M | | | Cartilage Rep7 | | |
| 112347 | 0.0 | 0.1 | 112672 OA Bone5 | 2.7 | 2.1 |
| Allergy-M | | | | | |
| 112349 Normal | 0.0 | 0.1 | 112673 OA | 1.2 | 1.3 |
| Lung-F | | | Synovium5 | | |
| 112357 Normal | 9.5 | 9.1 | 112674 OA | 1.5 | 1.6 |
| Lung-F | | | Synovial Fluid | | |
| | | | cells5 | | |
| 112354 Normal | 3.9 | 2.6 | 117100 OA | 0.7 | 0.4 |
| Lung-M | | | Cartilage Rep14 | | |
| 112374 | 1.9 | 1.6 | 112756 OA Bone9 | 5.6 | 4.8 |
| Crohns-F | | | | | |
| 112389 Match | 1.6 | 1.3 | 112757 OA | 0.3 | 0.2 |
| Control | | | Synovium9 | | |
| Crohns-F | | | | | |
| 112375 | 1.4 | 1.5 | 112758 OA | 0.8 | 1.4 |
| Crohns-F | | | Synovial Fluid | | |
| | | | Cells9 | | |
| 112732 Match | 5.1 | 4.5 | 117125 RA | 0.4 | 0.4 |
| Control | | | Cartilage Rep2 | | |
| Crohns-F | | | | | |
| 112725 | 2.1 | 2.4 | 113492 Bone2 RA | 65.5 | 70.7 |
| Crohns-M | | | | | |
| 112387 Match | 0.5 | 0.4 | 113493 Synovium2 | 18.9 | 19.2 |
| Control | | | RA | | |
| Crohns-M | | | | | |
| 112378 | 0.2 | 0.2 | 113494 Syn Fluid | 36.1 | 44.1 |
| Crohns-M | | | Cells RA | | |
| 112390 Match | 6.4 | 2.9 | 113499 Cartilage4 | 78.5 | 82.4 |
| Control | | | RA | | |
| Crohns-M | | | | | |
| 112726 | 6.3 | 7.6 | 113500 Bone4 RA | 100.0 | 100.0 |
| Crohns-M | | | | | |
| 112731 Match | 7.9 | 6.5 | 113501 Synovium4 | 70.7 | 72.2 |
| Control | | | RA | | |
| Crohns-M | | | | | |
| 112380 Ulcer | 4.9 | 4.2 | 113502 Syn Fluid | 45.7 | 46.3 |
| Col-F | | | Cells4 RA | | |
| 112734 Match | 13.8 | 9.5 | 113495 Cartilage3 | 39.2 | 45.1 |
| Control Ulcer | | | RA | | |
| Col-F | | | | | |
| 112384 Ulcer | 5.8 | 5.2 | 113496 Bone3 RA | 30.4 | 55.1 |
| Col-F | | | | | |
| 112737 Match | 5.5 | 4.0 | 113497 Synovium3 | 30.1 | 31.0 |
| Control Ulcer | | | RA | | |
| Col-F | | | | | |
| 112386 Ulcer | 0.9 | 0.2 | 113498 Syn Fluid | 79.0 | 72.7 |
| Col-F | | | Cells3 RA | | |
| 112738 Match | 9.5 | 7.5 | 117106 Normal | 0.1 | 0.1 |
| Control Ulcer | | | Cartilage Rep20 | | |
| Col-F | | | | | |
| 112381 Ulcer | 0.4 | 0.3 | 113663 Bone3 | 0.0 | 0.5 |
| Col-M | | | Normal | | |
| 112735 Match | 3.5 | 2.3 | 113664 Synovium3 | 0.0 | 0.0 |
| Control Ulcer | | | Normal | | |
| Col-M | | | | | |
| 112382 Ulcer | 2.3 | 1.7 | 113665 Syn Fluid | 0.2 | 0.2 |
| Col-M | | | Cells3 Normal | | |
| 112394 Match | 0.3 | 0.2 | 117107 Normal | 0.7 | 0.6 |
| Control Ulcer | | | Cartilage Rep22 | | |
| Col-M | | | | | |
| 112383 Ulcer | 7.5 | 6.1 | 113667 Bone4 | 0.8 | 0.8 |
| Col-M | | | Normal | | |
| 112736 Match | 1.1 | 1.1 | 113668 Synovium4 | 1.3 | 1.6 |
| Control Ulcer | | | Normal | | |
| Col-M | | | | | |
| 112423 | 3.8 | 2.0 | 113669 Syn Fluid | 2.0 | 1.8 |
| Psoriasis-F | | | Cells4 Normal | | |

TABLE LC
Panel 1.3D

| | Rel. Exp. (%) | | Rel. Exp. (%) |
|---|---|---|---|
| | Ag2558, Run | | Ag2558, Run |
| Tissue Name | 161905853 | Tissue Name | 161905853 |
| Liver adenocarcinoma | 5.3 | Kidney (fetal) | 10.2 |
| Pancreas | 3.9 | Renal ca. 786-0 | 11.0 |
| Pancreatic ca. CAPAN 2 | 7.1 | Renal ca. A498 | 3.2 |
| Adrenal gland | 0.9 | Renal ca. RXF 393 | 3.7 |
| Thyroid | 3.5 | Renal ca. ACHN | 3.6 |
| Salivary gland | 18.7 | Renal ca. UO-31 | 4.0 |
| Pituitary gland | 3.2 | Renal ca. TK-10 | 1.3 |
| Brain (fetal) | 1.0 | Liver | 2.5 |
| Brain (whole) | 2.0 | Liver (fetal) | 7.2 |
| Brain (amygdala) | 1.5 | Liver ca. (hepatoblast) | 0.1 |
| | | HepG2 | |
| Brain (cerebellum) | 2.7 | Lung | 53.6 |
| Brain (hippocampus) | 2.6 | Lung (fetal) | 12.2 |
| Brain (substantia nigra) | 1.4 | Lung ca. (small cell) LX-1 | 2.0 |
| Brain (thalamus) | 2.0 | Lung ca. (small cell) | 0.9 |
| | | NCI-H69 | |
| Cerebral Cortex | 4.5 | Lung ca. (s.cell var.) SHP-77 | 7.7 |
| Spinal cord | 10.4 | Lung ca. (large | 100.0 |
| | | cell)NCI-H460 | |
| glio/astro U87-MG | 13.6 | Lung ca. (non-sm. cell) A549 | 2.0 |
| glio/astro U-118-MG | 3.5 | Lung ca. (non-s.cell) | 2.9 |
| | | NCI-H23 | |
| astrocytoma SW1783 | 10.1 | Lung ca. (non-s.cell) | 8.4 |
| | | HOP-62 | |
| neuro*; met SK-N-AS | 4.3 | Lung ca. (non-s.cl) | 0.1 |
| | | NCI-H522 | |
| astrocytoma SF-539 | 7.0 | Lung ca. (squam.) SW 900 | 2.8 |
| astrocytoma SNB-75 | 2.9 | Lung ca. (squam.) NCI-H596 | 0.2 |
| glioma SNB-19 | 2.5 | Mammary gland | 4.8 |
| glioma U251 | 2.9 | Breast ca.* (pl.ef) MCF-7 | 7.9 |
| glioma SF-295 | 1.2 | Breast ca.* (pl.ef) | 4.5 |
| | | MDA-MB-231 | |
| Heart (fetal) | 0.8 | Breast ca.* (pl.ef) T47D | 4.1 |
| Heart | 3.6 | Breast ca. BT-549 | 14.8 |
| Skeletal muscle (fetal) | 1.4 | Breast ca. MDA-N | 7.9 |
| Skeletal muscle | 0.8 | Ovary | 3.2 |
| Bone marrow | 4.3 | Ovarian ca. OVCAR-3 | 5.5 |
| Thymus | 17.0 | Ovarian ca. OVCAR-4 | 1.4 |
| Spleen | 3.2 | Ovarian ca. OVCAR-5 | 2.0 |
| Lymph node | 2.1 | Ovarian ca. OVCAR-8 | 2.2 |
| Colorectal | 11.4 | Ovarian ca. IGROV-1 | 0.9 |
| Stomach | 1.4 | Ovarian ca.* (ascites) | 14.7 |
| | | SK-OV-3 | |
| Small intestine | 2.3 | Uterus | 1.2 |
| Colon ca. SW480 | 3.4 | Placenta | 36.3 |
| Colon ca.* SW620(SW480 | 1.4 | Prostate | 1.9 |
| met) | | | |
| Colon ca. HT29 | 5.6 | Prostate ca.* (bone met)PC-3 | 1.8 |
| Colon ca. HCT-116 | 6.6 | Testis | 1.5 |
| Colon ca. CaCo-2 | 2.3 | Melanoma Hs688(A).T | 0.2 |
| Colon ca. tissue(ODO3866) | 14.8 | Melanoma* (met) | 0.5 |
| | | Hs688(B).T | |
| Colon ca. HCC-2998 | 3.0 | Melanoma UACC-62 | 0.2 |
| Gastric ca.* (liver met) | 6.3 | Melanoma M14 | 1.0 |
| NCI-N87 | | | |
| Bladder | 23.7 | Melanoma LOX IMVI | 0.4 |
| Trachea | 13.7 | Melanoma* (met) | 2.8 |
| | | SK-MEL-5 | |
| Kidney | 13.6 | Adipose | 15.6 |

TABLE LD
Panel 2D

| | Rel. Exp. (%) | | Rel. Exp. (%) |
|---|---|---|---|
| | Ag2558, Run | | Ag2558, Run |
| Tissue Name | 161905854 | Tissue Name | 161905854 |
| Normal Colon | 23.3 | Kidney Margin 8120608 | 0.4 |
| CC Well to Mod Diff | 4.4 | Kidney Cancer 8120613 | 0.4 |
| (ODO3866) | | | |
| CC Margin (ODO3866) | 4.7 | Kidney Margin 8120614 | 0.9 |
| CC Gr.2 rectosigmoid | 2.7 | Kidney Cancer 9010320 | 1.7 |
| (ODO3868) | | | |
| CC Margin (ODO3868) | 0.3 | Kidney Margin 9010321 | 1.1 |
| CC Mod Diff (ODO3920) | 15.7 | Normal Uterus | 0.5 |
| CC Margin (ODO3920) | 4.8 | Uterus Cancer 064011 | 4.3 |
| CC Gr.2 ascend colon | 18.0 | Normal Thyroid | 3.1 |
| (ODO3921) | | | |
| CC Margin (ODO3921) | 4.5 | Thyroid Cancer 064010 | 3.8 |
| CC from Partial Hepatectomy | 3.8 | Thyroid Cancer A302152 | 3.0 |
| (ODO4309) Mets | | | |
| Liver Margin (ODO4309) | 8.7 | Thyroid Margin A302153 | 3.6 |
| Colon mets to lung | 8.7 | Normal Breast | 2.3 |
| (OD04451-01) | | | |
| Lung Margin (OD04451-02) | 34.6 | Breast Cancer (OD04566) | 1.6 |
| Normal Prostate 6546-1 | 1.4 | Breast Cancer | 7.5 |
| | | (OD04590-01) | |
| Prostate Cancer (OD04410) | 2.0 | Breast Cancer Mets | 6.5 |
| | | (OD04590-03) | |
| Prostate Margin (OD04410) | 3.1 | Breast Cancer Metastasis | 1.8 |
| | | (OD04655-05) | |
| Prostate Cancer (OD04720-01) | 1.5 | Breast Cancer 064006 | 2.0 |
| Prostate Margin (OD04720-02) | 4.5 | Breast Cancer 1024 | 5.7 |
| Normal Lung 061010 | 70.2 | Breast Cancer 9100266 | 1.4 |
| Lung Met to Muscle | 8.2 | Breast Margin 9100265 | 1.1 |
| (ODO4286) | | | |
| Muscle Margin (ODO4286) | 1.7 | Breast Cancer A209073 | 5.4 |
| Lung Malignant Cancer | 15.5 | Breast Margin A209073 | 3.2 |
| (OD03126) | | | |
| Lung Margin (OD03126) | 100.0 | Normal Liver | 4.2 |
| Lung Cancer (OD04404) | 17.7 | Liver Cancer 064003 | 1.5 |
| Lung Margin (OD04404) | 34.2 | Liver Cancer 1025 | 1.8 |
| Lung Cancer (OD04565) | 0.8 | Liver Cancer 1026 | 0.8 |
| Lung Margin (OD04565) | 27.2 | Liver Cancer 6004-T | 2.4 |
| Lung Cancer (OD04237-01) | 9.3 | Liver Tissue 6004-N | 1.2 |
| Lung Margin (OD04237-02) | 41.5 | Liver Cancer 6005-T | 0.6 |
| Ocular Mel Met to Liver | 0.1 | Liver Tissue 6005-N | 0.5 |
| (ODO4310) | | | |
| Liver Margin (ODO4310) | 4.0 | Normal Bladder | 13.4 |
| Melanoma Mets to Lung | 4.0 | Bladder Cancer 1023 | 1.1 |
| (OD04321) | | | |
| Lung Margin (OD04321) | 80.7 | Bladder Cancer A302173 | 2.4 |
| Normal Kidney | 8.7 | Bladder Cancer | 9.3 |
| | | (OD04718-01) | |
| Kidney Ca, Nuclear grade 2 | 15.9 | Bladder Normal Adjacent | 4.9 |
| (OD04338) | | (OD04718-03) | |
| Kidney Margin (OD04338) | 5.5 | Normal Ovary | 0.5 |
| Kidney Ca Nuclear grade 1/2 | 25.3 | Ovarian Cancer 064008 | 5.6 |
| (OD04339) | | | |
| Kidney Margin (OD04339) | 7.6 | Ovarian Cancer | 5.1 |
| | | (OD04768-07) | |
| Kidney Ca, Clear cell type | 7.1 | Ovary Margin | 3.9 |
| (OD04340) | | (OD04768-08) | |
| Kidney Margin (OD04340) | 3.5 | Normal Stomach | 0.9 |
| Kidney Ca, Nuclear grade 3 | 0.5 | Gastric Cancer 9060358 | 0.4 |
| (OD04348) | | | |
| Kidney Margin (OD04348) | 2.2 | Stomach Margin 9060359 | 0.9 |
| Kidney Cancer (OD04622-01) | 2.2 | Gastric Cancer 9060395 | 1.1 |
| Kidney Margin (OD04622-03) | 0.6 | Stomach Margin 9060394 | 3.0 |
| Kidney Cancer (OD04450-01) | 3.2 | Gastric Cancer 9060397 | 5.7 |
| Kidney Margin (OD04450-03) | 3.5 | Stomach Margin 9060396 | 0.7 |
| Kidney Cancer 8120607 | 0.2 | Gastric Cancer 064005 | 3.2 |

TABLE LE
Panel 4D

| | Rel. Exp. (%) | | Rel. Exp. (%) |
|---|---|---|---|
| | Ag2558, Run | | Ag2558, Run |
| Tissue Name | 161905855 | Tissue Name | 161905855 |
| Secondary Th1 act | 11.6 | HUVEC IL-1beta | 0.2 |
| Secondary Th2 act | 14.4 | HUVEC IFN gamma | 0.4 |
| Secondary Tr1 act | 16.6 | HUVEC TNF alpha + IFN | 0.3 |
| | | gamma | |
| Secondary Th1 rest | 1.2 | HUVEC TNF alpha + IL4 | 2.2 |
| Secondary Th2 rest | 2.3 | HUVEC IL-11 | 0.2 |
| Secondary Tr1 rest | 2.3 | Lung Microvascular EC none | 0.2 |
| Primary Th1 act | 9.2 | Lung Microvascular EC | 0.4 |
| | | TNF alpha + IL-1beta | |
| Primary Th2 act | 12.8 | Microvascular Dermal EC | 0.4 |
| | | none | |
| Primary Tr1 act | 15.9 | Microsvasular Dermal EC | 0.3 |
| | | TNF alpha + IL-1beta | |
| Primary Th1 rest | 12.1 | Bronchial epithelium | 3.2 |
| | | TNF alpha + IL1beta | |
| Primary Th2 rest | 6.9 | Small airway epithelium none | 0.9 |
| Primary Tr1 rest | 9.5 | Small airway epithelium | 7.9 |
| | | TNF alpha + IL-1beta | |
| CD45RA CD4 lymphocyte act | 3.1 | Coronery artery SMC rest | 1.3 |
| CD45RO CD4 lymphocyte act | 8.2 | Coronery artery SMC | 1.0 |
| | | TNF alpha + IL-1beta | |
| CD8 lymphocyte act | 8.2 | Astrocytes rest | 0.5 |
| Secondary CD8 lymphocyte | 9.6 | Astrocytes TNF alpha + | 1.4 |
| rest | | IL-1beta | |
| Secondary CD8 lymphocyte act | 10.8 | KU-812 (Basophil) rest | 33.9 |
| CD4 lymphocyte none | 0.7 | KU-812 (Basophil) | 68.3 |
| | | PMA/ionomycin | |
| 2ry Th1/Th2/Tr1_anti-CD95 | 2.6 | CCD1106 (Keratinocytes) | 2.4 |
| CH11 | | none | |
| LAK cells rest | 9.2 | CCD1106 (Keratinocytes) | 0.8 |
| | | TNF alpha + IL-1beta | |
| LAK cells IL-2 | 6.7 | Liver cirrhosis | 0.3 |
| LAK cells IL-2 + IL-12 | 9.2 | Lupus kidney | 0.1 |
| LAK cells IL-2 + IFN gamma | 12.8 | NCI-H292 none | 17.1 |
| LAK cells IL-2 + IL-18 | 15.4 | NCI-H292 IL-4 | 36.9 |
| LAK cells PMA/ionomycin | 8.7 | NCI-H292 IL-9 | 21.3 |
| NK Cells IL-2 rest | 5.3 | NCI-H292 IL-13 | 15.5 |
| Two Way MLR 3 day | 16.2 | NCI-H292 IFN gamma | 11.5 |
| Two Way MLR 5 day | 10.5 | HPAEC none | 0.5 |
| Two Way MLR 7 day | 2.8 | HPAEC TNF alpha + | 0.6 |
| | | IL-1beta | |
| PBMC rest | 0.8 | Lung fibroblast none | 0.1 |
| PBMC PWM | 39.8 | Lung fibroblast TNF alpha + | 4.1 |
| | | IL-1beta | |
| PBMC PHA-L | 19.6 | Lung fibroblast IL-4 | 0.6 |
| Ramos (B cell) none | 8.3 | Lung fibroblast IL-9 | 0.3 |
| Ramos (B cell) ionomycin | 54.7 | Lung fibroblast IL-13 | 0.2 |
| B lymphocytes PWM | 35.4 | Lung fibroblast IFN gamma | 0.3 |
| B lymphocytes CD40L and IL-4 | 12.1 | Dermal fibroblast CCD1070 | 0.2 |
| | | rest | |
| EOL-1 dbcAMP | 7.2 | Dermal fibroblast CCD1070 | 12.2 |
| | | TNF alpha | |
| EOL-1 dbcAMP | 9.2 | Dermal fibroblast CCD1070 | 0.7 |
| PMA/ionomycin | | IL-1beta | |
| Dendritic cells none | 4.2 | Dermal fibroblast IFN gamma | 0.7 |
| Dendritic cells LPS | 21.0 | Dermal fibroblast IL-4 | 3.0 |
| Dendritic cells anti-CD40 | 2.6 | IBD Colitis 2 | 0.1 |
| Monocytes rest | 0.3 | IBD Crohn's | 0.0 |
| Monocytes LPS | 100.0 | Colon | 1.4 |
| Macrophages rest | 13.3 | Lung | 20.4 |
| Macrophages LPS | 38.2 | Thymus | 4.6 |
| HUVEC none | 0.9 | Kidney | 3.2 |
| HUVEC starved | 1.0 | | |

AI_comprehensive panel_v1.0 Summary: Ag2558 Two experiments with the same probe and primer produce results that are in excellent agreement. The transcript is induced in rheumatoid (CTs=27-29) and osteoarthritic (CTs=26-28) joint tissue as compared to normal control joint. The transcript is expressed at lower levels in several other tissues. This gene encodes a protein with a putative ZIP Zinc Transporter domain. Therapeutic modulation of the expression or function of this protein may be useful in the treatment of arthritis. See, Lioumi M., et al., Genomics Dec. 1, 1999;62(2):272-80 (PMID: 10610721).

Panel 1.3D Summary: Ag2558 Highest expression of the CG92489-01 gene is seen in a lung cancer cell line (CT=27.4). Thus, expression of this gene could be used to differentiate between this sample and other samples on this panel and as a marker for lung cancer. Furthermore, therapeutic modulation of the expression or function of this gene may be useful in the treatment of lung cancer.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed at low levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 2D Summary: Ag2558 Highest expression of the CG92489-01 gene is seen in normal lung tissue adjacent to a tumor (CT=25.6). In addition, expression of this gene appears to be higher in normal lung tissue than in matched tumor tissue in four out of five matched tissue pairs. Thus, expression of this gene could be used to differentiate between this sample and other samples on this panel and as a marker for lung cancer. Furthermore, therapeutic modulation of the expression or function of this gene may be useful in the treatment of lung cancer.

Panel 4D Summary: Ag2558: The transcript is expressed in activated macrophages, monocyte, and T cells as well as TNFalpha treated dermal fibroblasts, with highest expression in LPS treated monocytes (CT=25). It is expressed in normal lung (possibly as a result of the presence of normal macrophages which express the transcript). The transcript encodes a putative Zinc transporter that may be important in leukocyte and fibroblast activation. Humanized antibodies that antagonize the function of this molecule may be important in the treatment of OA and RA (see A/I panel).

M. NOV18a and NOV18b and NOV18c (CG93252-01 and CG93252-02 and CG93252-03): Cathepsin L Precursor

Expression of gene CG93252-01 and variants CG93252-02 and CG93252-03 was assessed using the primer-probe sets Ag1081 and Ag1304b, described in Tables MA and MB. Please note that the probe and primer set Ag1304b is specific to CG93252-03 only.

TABLE MA

Probe Name Ag1081

| | | | Start | SEQ ID |
|---|---|---|---|---|
| Primers | Sequences | Length | Position | No |
| Forward | 5′-tcactcctccttgctgtcttt-3′ | 21 | 25 | 130 |
| Probe | TET-5′-tgcctgagattagcctcagctagtct-3′-TAMRA | 26 | 46 | 131 |
| Reverse | 5′-tgccttccactgatctaaactg-3′ | 22 | 84 | 132 |

TABLE MB

Probe Name Ag1304b

| | | | Start | SEQ ID |
|---|---|---|---|---|
| Primers | Sequences | Length | Position | No |
| Forward | 5′-ctctaggcttcaaggcaatgtt-3′ | 22 | 459 | 133 |
| Probe | TET-5′-tttggagaaccattattttgcttcca-3′-TAMRA | 26 | 490 | 134 |
| Reverse | 5′-ctccattgtcggcaacatac-3′ | 20 | 516 | 135 |

General_screening_panel_v1.4 Summary: Ag1081 Expression of the CG93252-01 gene is low/undetectable in all samples on this panel (CTs>35).

Panel 4D Summary: Ag1081/Ag1304b Expression of the CG93252-01 gene is low/undetectable in all samples on this panel (CTs>35).

N. NOV19 (CG93285-01): Matrix Metalloprotease

Expression of gene CG93285-01 was assessed using the primer-probe set Ag3849 described in Table NA. Results of the RTQ-PCR runs are shown in Table NB.

TABLE NA
Probe Name Ag3849

| Primers | Sequences | Length | Start Position | SEQ ID NO |
|---|---|---|---|---|
| Forward | 5′-ctgggaaagcctttgaactct-3′ | 21 | 428 | 136 |
| Probe | TET-5′-agtaaggcctcggccctgacctt-3′-TAMRA | 23 | 451 | 137 |
| Reverse | 5′-atgtccccttcactctcaaagt-3′ | 22 | 482 | 138 |

TABLE NB
General_screening_panel_v1.4

| | Rel. Exp. (%) | | Rel. Exp. (%) |
|---|---|---|---|
| | Ag3849, Run | | Ag3849, Run |
| Tissue Name | 218998428 | Tissue Name | 218998428 |
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) | 100.0 |
| | | NCI-N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 2.0 |
| Squamous cell carcinoma | 5.2 | Colon ca.* (SW480 met) | 0.7 |
| SCC-4 | | SW620 | |
| Testis Pool | 0.0 | Colon ca. HT29 | 0.0 |
| Prostate ca.* (bone met) | 0.0 | Colon HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.8 | Colon Pool | 0.0 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) | 0.0 |
| | | U87-MG | |
| Lung | 0.0 | CNS cancer (glio/astro) | 0.0 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 2.7 | CNS cancer (astro) SNB-75 | 0.0 |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 2.7 | Brain (Hippocampus) Pool | 0.0 |
| Lung ca. HOP-62 | 0.0 | Cerebral Cortex Pool | 0.0 |
| Lung ca. NCI-H522 | 2.0 | Brain (Substantia nigra) Pool | 0.0 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 2.7 |
| Renal ca. UO-31 | 0.0 | Pancreas Pool | 0.0 |

AI_comprehensive panel_v1.0 Summary: Ag3849 Expression of the CG93285-01 gene is low/undetectable in all samples on this panel (CTs>35).

CNS_neurodegeneration_v1.0 Summary: Ag3849 Expression of the CG93285-01 gene is low/undetectable in all samples on this panel (CTs>35).

General_screening_panel_v1.4 Summary: Ag3849 Expression of the CG93285-01 gene is restricted to a sample derived from a gastric cancer cell line (CT=32.4). Thus, expression of this gene could be used to differentiate between this sample and other samples on this panel and as a marker to detect the presence of gastric cancer. Furthermore, therapeutic modulation of the expression or function of this gene may be effective in the treatment of gastric cancer.

O. NOV20a and NOV20b (CG93387-01 and CG93387-02): Fibropellin I Precursor

Expression of gene CG93387-01 and variant CG93387-02 was assessed using the primer-probe sets Ag1143, Ag1921, Ag3082, Ag752, Ag923, Ag345 and Ag558, described in Tables OA, OB, OC, OD, OE, OF and OG. Results of the RTQ-PCR runs are shown in Tables OH, OI, OJ, OK, OL and OM.

TABLE OA
Probe Name Ag1143

| | | | Start | SEQ ID |
|---|---|---|---|---|
| Primers | Sequences | Length | Position | NO |
| Forward | 5′-gagatggatgtggacgactg-3′ | 20 | 964 | 139 |
| Probe | TET-5′-cctgaatggaggctcttgtgttgacc-3′-TAMRA | 26 | 999 | 140 |
| Reverse | 5′-acaagcaggtgtaattcccc-3′ | 20 | 1029 | 141 |

TABLE OB
Probe Name Ag1921

| | | | Start | SEQ ID |
|---|---|---|---|---|
| Primers | Sequences | Length | Position | NO |
| Forward | 5′-acacattccagactgtgctcat-3′ | 22 | 317 | 142 |
| Probe | TET-5′-acggcaagctctccttcaccatctt-3′-TAMRA | 25 | 344 | 143 |
| Reverse | 5′-tccacacgatggactcatagtt-3′ | 22 | 370 | 144 |

TABLE OC
Probe Name Ag3082

| | | | Start | SEQ ID |
|---|---|---|---|---|
| Primers | Sequences | Length | Position | NO |
| Forward | 5′-cagacgcagaggtagctcc-3′ | 19 | 1385 | 145 |
| Probe | TET-5′-catctgggcactgtgtgttcatgttg-3′-TAMRA | 26 | 1335 | 146 |
| Reverse | 5′-atttgaaatcacagccatgc-3′ | 20 | 1311 | 147 |

TABLE OD
Probe Name Ag752

| | | | Start | SEQ ID |
|---|---|---|---|---|
| Primers | Sequences | Length | Position | NO |
| Forward | 5′-ggaggacgtcaggcactact-3′ | 20 | 204 | 148 |
| Probe | TET-5′-ctggacttcaatgccacctgggtttt-3′-TAMRA | 26 | 235 | 149 |
| Reverse | 5′-gaactgcctccaaagaaggt-3′ | 20 | 283 | 150 |

TABLE OE
Probe Name Ag923

| | | | Start | SEQ ID |
|---|---|---|---|---|
| Primers | Sequences | Length | Position | NO |
| Forward | 5′-tcctgtctcacagcgaagtc-3′ | 20 | 1067 | 151 |
| Probe | TET-5′-cacacaagcaggtgtaattccccact-3′-TAMRA | 26 | 1026 | 152 |
| Reverse | 5′-aatggaggctcttgtgttgac-3′ | 21 | 1003 | 153 |

TABLE OF
Probe Name Ag345

| Primers | Sequences | Length | Start Position | SEQ ID NO |
|---|---|---|---|---|
| Forward | 5′-cagcctgcgagatggatgt-3′ | 19 | 956 | 154 |
| Probe | TET-5′-acgactgcagccctgacccctg-3′-TAMRA | 22 | 977 | 155 |
| Reverse | 5′-tccccactaggtcaacacaaga-3′ | 22 | 1012 | 156 |

TABLE OG
Probe Name Ag558

| Primers | Sequences | Length | Start Position | SEQ ID NO |
|---|---|---|---|---|
| Forward | 5′-gccacctggacgtgaacg-3′ | 18 | 734 | 157 |
| Probe | TET-5′-tgtgcctcccagccctgtcaga-3′-TAMRA | 22 | 754 | 158 |
| Reverse | 5′-aaactgttgatgccgtgagtaca-3′ | 23 | 787 | 159 |

TABLE OH
Panel 1

| | Rel. Exp. (%) | | Rel. Exp. (%) |
|---|---|---|---|
| | Ag345, Run | | Ag345, Run |
| Tissue Name | 87584724 | Tissue Name | 87584724 |
| Endothelial cells | 6.8 | Renal ca. 786-0 | 0.0 |
| Endothelial cells (treated) | 2.1 | Renal ca. A498 | 0.0 |
| Pancreas | 13.9 | Renal ca. RXF 393 | 0.0 |
| Pancreatic ca. CAPAN 2 | 0.2 | Renal ca. ACHN | 0.8 |
| Adrenal gland | 23.7 | Renal ca. UO-31 | 0.6 |
| Thyroid | 26.1 | Renal ca. TK-10 | 0.0 |
| Salivary gland | 4.4 | Liver | 15.2 |
| Pituitary gland | 12.0 | Liver (fetal) | 1.0 |
| Brain (fetal) | 2.3 | Liver ca. (hepatoblast) HepG2 | 0.2 |
| Brain (whole) | 25.7 | Lung | 23.0 |
| Brain (amygdala) | 8.9 | Lung (fetal) | 24.5 |
| Brain (cerebellum) | 100.0 | Lung ca. (small cell) LX-1 | 4.1 |
| Brain (hippocampus) | 11.3 | Lung ca. (small cell) NCI-H69 | 0.8 |
| Brain (substantia nigra) | 6.1 | Lung ca. (s.cell var.) SHP-77 | 11.6 |
| Brain (thalamus) | 6.7 | Lung ca. (large cell) NCI-H460 | 4.8 |
| Brain (hypothalamus) | 11.0 | Lung ca. (non-sm. cell) A549 | 2.2 |
| Spinal cord | 11.9 | Lung ca. (non-s.cell) NCI-H23 | 3.1 |
| glio/astro U87-MG | 4.0 | Lung ca. (non-s.cell) HOP-62 | 3.7 |
| glio/astro U-118-MG | 31.9 | Lung ca. (non-s.cl) NCI-H522 | 1.3 |
| astrocytoma SW1783 | 5.6 | Lung ca. (squam.) SW 900 | 11.3 |
| neuro*; met SK-N-AS | 3.2 | Lung ca. (squam.) NCI-H596 | 0.7 |
| astrocytoma SF-539 | 7.7 | Mammary gland | 46.7 |
| astrocytoma SNB-75 | 12.8 | Breast ca.* (pl.ef) MCF-7 | 1.8 |
| glioma SNB-19 | 13.4 | Breast ca.* (pl.ef) | 1.4 |
| | | MDA-MB-231 | |
| glioma U251 | 2.9 | Breast ca.* (pl. ef) T47D | 5.4 |
| glioma SF-295 | 51.1 | Breast ca. BT-549 | 18.9 |
| Heart | 9.3 | Breast ca. MDA-N | 2.9 |
| Skeletal muscle | 8.4 | Ovary | 49.3 |
| Bone marrow | 3.1 | Ovarian ca. OVCAR-3 | 0.3 |
| Thymus | 34.4 | Ovarian ca. OVCAR-4 | 0.6 |
| Spleen | 15.8 | Ovarian ca. OVCAR-5 | 23.7 |
| Lymph node | 14.7 | Ovarian ca. OVCAR-8 | 1.4 |
| Colon (ascending) | 13.5 | Ovarian ca. IGROV-1 | 0.3 |
| Stomach | 20.0 | Ovarian ca. (ascites) SK-OV-3 | 9.9 |
| Small intestine | 11.6 | Uterus | 13.2 |
| Colon ca. SW480 | 0.2 | Placenta | 3.1 |
| Colon ca.* SW620 (SW480 | 0.4 | Prostate | 23.5 |
| met) | | | |
| Colon ca. HT29 | 0.0 | Prostate ca.* (bone met) PC-3 | 1.3 |
| Colon ca. HCT-116 | 3.4 | Testis | 98.6 |
| Colon ca. CaCo-2 | 0.1 | Melanoma Hs688(A).T | 6.4 |
| Colon ca. HCT-15 | 1.1 | Melanoma* (met) Hs688(B).T | 0.0 |
| Colon ca. HCC-2998 | 0.7 | Melanoma UACC-62 | 2.8 |
| Gastric ca.* (liver met) | 3.1 | Melanoma M14 | 18.2 |
| NCI-N87 | | | |
| Bladder | 23.8 | Melanoma LOX IMVI | 0.3 |
| Trachea | 12.6 | Melanoma* (met) SK-MEL-5 | 0.2 |
| Kidney | 10.4 | Melanoma SK-MEL-28 | 5.5 |
| Kidney (fetal) | 17.7 | | |

TABLE OI
Panel 1.1

| | Rel. Exp. (%) | | Rel. Exp. (%) |
|---|---|---|---|
| | Ag558, Run | | Ag558, Run |
| Tissue Name | 109666712 | Tissue Name | 109666712 |
| Adrenal gland | 19.1 | Renal ca. UO-31 | 0.0 |
| Bladder | 34.4 | Renal ca. RXF 393 | 0.0 |
| Brain (amygdala) | 3.8 | Liver | 17.3 |
| Brain (cerebellum) | 51.1 | Liver (fetal) | 0.3 |
| Brain (hippocampus) | 17.0 | Liver ca. (hepatoblast) | 0.0 |
| | | HepG2 | |
| Brain (substantia nigra) | 24.5 | Lung | 8.8 |
| Brain (thalamus) | 5.4 | Lung (fetal) | 29.5 |
| Cerebral Cortex | 21.0 | Lung ca. (non-s.cell) | 20.9 |
| | | HOP-62 | |
| Brain (fetal) | 5.4 | Lung ca. (large | 2.6 |
| | | cell) NCI-H460 | |
| Brain (whole) | 19.8 | Lung ca. (non-s.cell) | 7.0 |
| | | NCI-H23 | |
| glio/astro U-118-MG | 42.6 | Lung ca. (non-s.cl) | 0.0 |
| | | NCI-H522 | |
| astrocytoma SF-539 | 14.3 | Lung ca. (non-sm. cell) | 0.3 |
| | | A549 | |
| astrocytoma SNB-75 | 8.9 | Lung ca. (s.cell var.) | 4.1 |
| | | SHP-77 | |
| astrocytoma SW1783 | 3.6 | Lung ca. (small cell) LX-1 | 9.4 |
| glioma U251 | 1.3 | Lung ca. (small cell) | 0.5 |
| | | NCI-H69 | |
| glioma SF-295 | 100.0 | Lung ca. (squam.) SW 900 | 6.2 |
| glioma SNB-19 | 15.4 | Lung ca. (squam.) | 0.5 |
| | | NCI-H596 | |
| glio/astro U87-MG | 5.1 | Lymph node | 16.3 |
| neuro*; met SK-N-AS | 7.4 | Spleen | 0.0 |
| Mammary gland | 21.3 | Thymus | 4.2 |
| Breast ca. BT-549 | 4.8 | Ovary | 53.2 |
| Breast ca. MDA-N | 3.0 | Ovarian ca. IGROV-1 | 0.0 |
| Breast ca.* (pl.ef) T47D | 4.0 | Ovarian ca. OVCAR-3 | 0.0 |
| Breast ca.* (pl.ef) MCF-7 | 0.9 | Ovarian ca. OVCAR-4 | 0.4 |
| Breast ca.* (pl.ef) | 0.3 | Ovarian ca. OVCAR-5 | 57.8 |
| MDA-MB-231 | | | |
| Small intestine | 15.5 | Ovarian ca. OVCAR-8 | 0.0 |
| Colorectal | 1.9 | Ovarian ca.* (ascites) | 23.3 |
| | | SK-OV-3 | |
| Colon ca. HT29 | 0.0 | Pancreas | 44.1 |
| Colon ca. CaCo-2 | 0.0 | Pancreatic ca. CAPAN 2 | 0.0 |
| Colon ca. HCT-15 | 0.1 | Pituitary gland | 18.3 |
| Colon ca. HCT-116 | 2.5 | Placenta | 1.1 |
| Colon ca. HCC-2998 | 0.2 | Prostate | 20.7 |
| Colon ca. SW480 | 0.1 | Prostate ca.* (bone met) | 0.0 |
| | | PC-3 | |
| Colon ca.* SW620 (SW480 | 0.4 | Salivary gland | 11.0 |
| met) | | | |
| Stomach | 15.8 | Trachea | 9.7 |
| Gastric ca. (liver met) NCI-N87 | 1.3 | Spinal cord | 16.0 |
| Heart | 36.6 | Testis | 22.7 |
| Skeletal muscle (Fetal) | 12.2 | Thyroid | 62.4 |
| Skeletal muscle | 57.8 | Uterus | 18.3 |
| Endothelial cells | 3.1 | Melanoma M14 | 17.2 |
| Heart (Fetal) | 2.0 | Melanoma LOX IMVI | 0.0 |
| Kidney | 27.0 | Melanoma UACC-62 | 9.0 |
| Kidney (fetal) | 14.1 | Melanoma SK-MEL-28 | 12.5 |
| Renal ca. 786-0 | 0.0 | Melanoma* (met) | 0.0 |
| | | SK-MEL-5 | |
| Renal ca. A498 | 0.0 | Melanoma Hs688(A).T | 2.2 |
| Renal ca. ACHN | 0.0 | Melanoma* (met) | 0.1 |
| | | Hs688(B).T | |
| Renal ca. TK-10 | 0.0 | | |

TABLE OJ
Panel 1.2

| | Rel. Exp. (%) | | Rel. Exp. (%) |
|---|---|---|---|
| | Ag752, Run | | Ag752, Run |
| Tissue Name | 119778329 | Tissue Name | 119778329 |
| Endothelial cells | 29.5 | Renal ca. 786-0 | 0.0 |
| Heart (Fetal) | 3.2 | Renal ca. A498 | 0.0 |
| Pancreas | 50.0 | Renal ca. RXF 393 | 0.0 |
| Pancreatic ca. CAPAN 2 | 0.0 | Renal ca. ACHN | 0.9 |
| Adrenal Gland | 51.8 | Renal ca. UO-31 | 0.2 |
| Thyroid | 100.0 | Renal ca. TK-10 | 0.0 |
| Salivary gland | 19.9 | Liver | 32.1 |
| Pituitary gland | 30.1 | Liver (fetal) | 2.2 |
| Brain (fetal) | 8.4 | Liver ca. (hepatoblast) | 0.0 |
| | | HepG2 | |
| Brain (whole) | 31.6 | Lung | 20.7 |
| Brain (amygdala) | 14.4 | Lung (fetal) | 37.6 |
| Brain (cerebellum) | 27.9 | Lung ca. (small cell) LX-1 | 5.4 |
| Brain (hippocampus) | 24.1 | Lung ca. (small cell) | 1.2 |
| | | NCI-H69 | |
| Brain (thalamus) | 7.5 | Lung ca. (s.cell var.) SHP-77 | 2.6 |
| Cerebral Cortex | 32.5 | Lung ca. (large | 6.1 |
| | | cell) NCI-H460 | |
| Spinal cord | 12.8 | Lung ca. (non-sm. cell) | 3.2 |
| | | A549 | |
| glio/astro U87-MG | 6.0 | Lung ca. (non-s.cell) | 4.8 |
| | | NCI-H23 | |
| glio/astro U-118-MG | 38.2 | Lung ca. (non-s.cell) | 10.7 |
| | | HOP-62 | |
| astrocytoma SW1783 | 5.9 | Lung ca. (non-s.cl) | 3.1 |
| | | NCI-H522 | |
| neuro*; met SK-N-AS | 10.0 | Lung ca. (squam.) SW 900 | 7.5 |
| astrocytoma SF-539 | 13.5 | Lung ca. (squam.) NCI-H596 | 1.8 |
| astrocytoma SNB-75 | 2.7 | Mammary gland | 50.0 |
| glioma SNB-19 | 14.7 | Breast ca.* (pl.ef) MCF-7 | 1.0 |
| glioma U251 | 2.9 | Breast ca.* (pl.ef) | 1.7 |
| | | MDA-MB-231 | |
| glioma SF-295 | 56.6 | Breast ca.* (pl. ef) T47D | 7.5 |
| Heart | 31.2 | Breast ca. BT-549 | 11.2 |
| Skeletal Muscle | 75.3 | Breast ca. MDA-N | 4.0 |
| Bone marrow | 1.8 | Ovary | 54.7 |
| Thymus | 1.0 | Ovarian ca. OVCAR-3 | 0.2 |
| Spleen | 18.6 | Ovarian ca. OVCAR-4 | 1.1 |
| Lymph node | 16.3 | Ovarian ca. OVCAR-5 | 38.2 |
| Colorectal Tissue | 5.4 | Ovarian ca. OVCAR-8 | 0.8 |
| Stomach | 19.1 | Ovarian ca. IGROV-1 | 0.4 |
| Small intestine | 21.9 | Ovarian ca. (ascites) | 24.8 |
| | | SK-OV-3 | |
| Colon ca. SW480 | 0.3 | Uterus | 24.3 |
| Colon ca.* SW620 (SW480 | 0.4 | Placenta | 6.0 |
| met) | | | |
| Colon ca. HT29 | 0.0 | Prostate | 39.0 |
| Colon ca. HCT-116 | 3.1 | Prostate ca.* (bone met) | 1.6 |
| | | PC-3 | |
| Colon ca. CaCo-2 | 0.0 | Testis | 31.9 |
| Colon ca. Tissue (ODO3866) | 1.9 | Melanoma Hs688(A).T | 6.3 |
| Colon ca. HCC-2998 | 1.9 | Melanoma* (met) | 3.4 |
| | | Hs688(B).T | |
| Gastric ca.* (liver met) | 5.4 | Melanoma UACC-62 | 12.0 |
| NCI-N87 | | | |
| Bladder | 81.2 | Melanoma M14 | 12.7 |
| Trachea | 10.9 | Melanoma LOX IMVI | 0.0 |
| Kidney | 14.9 | Melanoma* (met) | 0.4 |
| | | SK-MEL-5 | |
| Kidney (fetal) | 26.6 | | |

TABLE OK
Panel 1.3D

| | Rel. Exp. (%) | | Rel. Exp. (%) |
|---|---|---|---|
| | Ag3082, Run | | Ag3082, Run |
| Tissue Name | 165673172 | Tissue Name | 165673172 |
| Liver adenocarcinoma | 31.2 | Kidney (fetal) | 3.4 |
| Pancreas | 2.5 | Renal ca. 786-0 | 0.2 |
| Pancreatic ca. CAPAN 2 | 0.2 | Renal ca. A498 | 12.4 |
| Adrenal gland | 12.3 | Renal ca. RXF 393 | 0.0 |
| Thyroid | 19.5 | Renal ca. ACHN | 0.0 |
| Salivary gland | 2.3 | Renal ca. UO-31 | 0.2 |
| Pituitary gland | 10.2 | Renal ca. TK-10 | 0.1 |
| Brain (fetal) | 2.1 | Liver | 2.4 |
| Brain (whole) | 10.8 | Liver (fetal) | 1.6 |
| Brain (amygdala) | 12.3 | Liver ca. (hepatoblast) | 0.0 |
| | | HepG2 | |
| Brain (cerebellum) | 21.0 | Lung | 9.0 |
| Brain (hippocampus) | 21.8 | Lung (fetal) | 6.4 |
| Brain (substantia nigra) | 3.9 | Lung ca. (small cell) LX-1 | 2.9 |
| Brain (thalamus) | 8.7 | Lung ca. (small cell) | 0.3 |
| | | NCI-H69 | |
| Cerebral Cortex | 8.7 | Lung ca. (s.cell var.) | 4.0 |
| | | SHP-77 | |
| Spinal cord | 12.8 | Lung ca. (large | 4.4 |
| | | cell) NCI-H460 | |
| glio/astro U87-MG | 3.0 | Lung ca. (non-sm. cell) | 1.3 |
| | | A549 | |
| glio/astro U-118-MG | 100.0 | Lung ca. (non-s.cell) | 2.9 |
| | | NCI-H23 | |
| astrocytoma SW1783 | 6.9 | Lung ca. (non-s.cell) | 2.7 |
| | | HOP-62 | |
| neuro*; met SK-N-AS | 3.1 | Lung ca. (non-s.cl) | 0.0 |
| | | NCI-H522 | |
| astrocytoma SF-539 | 12.6 | Lung ca. (squam.) SW 900 | 6.0 |
| astrocytoma SNB-75 | 25.9 | Lung ca. (squam.) | 0.8 |
| | | NCI-H596 | |
| glioma SNB-19 | 8.9 | Mammary gland | 12.5 |
| glioma U251 | 4.9 | Breast ca.* (pl.ef) MCF-7 | 0.3 |
| glioma SF-295 | 33.7 | Breast ca.* (pl.ef) | 1.7 |
| | | MDA-MB-231 | |
| Heart (fetal) | 1.6 | Breast ca.* (pl.ef) T47D | 1.0 |
| Heart | 5.8 | Breast ca. BT-549 | 15.1 |
| Skeletal muscle (fetal) | 5.8 | Breast ca. MDA-N | 0.5 |
| Skeletal muscle | 14.3 | Ovary | 13.4 |
| Bone marrow | 1.0 | Ovarian ca. OVCAR-3 | 1.1 |
| Thymus | 5.1 | Ovarian ca. OVCAR-4 | 0. 0 |
| Spleen | 22.5 | Ovarian ca. OVCAR-5 | 12.2 |
| Lymph node | 28.5 | Ovarian ca. OVCAR-8 | 0.5 |
| Colorectal | 3.6 | Ovarian ca. IGROV-1 | 0.0 |
| Stomach | 11.8 | Ovarian ca.* (ascites) | 7.2 |
| | | SK-OV-3 | |
| Small intestine | 14.5 | Uterus | 32.8 |
| Colon ca. SW480 | 0.8 | Placenta | 1.1 |
| Colon ca.* SW620(SW480 | 0.3 | Prostate | 12.4 |
| met) | | | |
| Colon ca. HT29 | 0.0 | Prostate ca.* (bone | 0.0 |
| | | met)PC-3 | |
| Colon ca. HCT-116 | 1.1 | Testis | 8.1 |
| Colon ca. CaCo-2 | 0.0 | Melanoma Hs688(A).T | 2.6 |
| Colon ca. tissue(ODO3866) | 3.1 | Melanoma* (met) | 0.9 |
| | | Hs688(B).T | |
| Colon ca. HCC-2998 | 0.4 | Melanoma UACC-62 | 4.8 |
| Gastric ca.* (liver met) | 3.5 | Melanoma M14 | 17.8 |
| NCI-N87 | | | |
| Bladder | 8.8 | Melanoma LOX IMVI | 0.2 |
| Trachea | 7.5 | Melanoma* (met) | 0.2 |
| | | SK-MEL-5 | |
| Kidney | 3.5 | Adipose | 17.2 |

TABLE OL
Panel 2.2

| | Rel. Exp. (%) | | Rel. Exp. (%) |
|---|---|---|---|
| | Ag3082, Run | | Ag3082, Run |
| Tissue Name | 174284798 | Tissue Name | 174284798 |
| Normal Colon | 20.3 | Kidney Margin (OD04348) | 54.0 |
| Colon cancer (OD06064) | 8.5 | Kidney malignant cancer | 0.6 |
| | | (OD06204B) | |
| Colon Margin (OD06064) | 6.4 | Kidney normal adjacent | 8.5 |
| | | tissue (OD06204E) | |
| Colon cancer (OD06159) | 1.4 | Kidney Cancer | 0.5 |
| | | (OD04450-01) | |
| Colon Margin (OD06159) | 9.6 | Kidney Margin | 16.4 |
| | | (OD04450-03) | |
| Colon cancer (OD06297-04) | 1.1 | Kidney Cancer 8120613 | 0.0 |
| Colon Margin (OD06297-05) | 5.7 | Kidney Margin 8120614 | 11.0 |
| CC Gr.2 ascend colon | 5.9 | Kidney Cancer 9010320 | 1.5 |
| (ODO3921) | | | |
| CC Margin (ODO3921) | 2.9 | Kidney Margin 9010321 | 7.1 |
| Colon cancer metastasis | 1.3 | Kidney Cancer 8120607 | 6.3 |
| (OD06104) | | | |
| Lung Margin (OD06104) | 0.8 | Kidney Margin 8120608 | 5.9 |
| Colon mets to lung | 17.3 | Normal Uterus | 52.1 |
| (OD04451-01) | | | |
| Lung Margin (OD04451-02) | 17.2 | Uterine Cancer 064011 | 37.6 |
| Normal Prostate | 20.3 | Normal Thyroid | 10.2 |
| Prostate Cancer (OD04410) | 13.1 | Thyroid Cancer 064010 | 4.0 |
| Prostate Margin (OD04410) | 10.1 | Thyroid Cancer A302152 | 21.0 |
| Normal Ovary | 52.5 | Thyroid Margin A302153 | 29.5 |
| Ovarian cancer | 8.6 | Normal Breast | 52.1 |
| (OD06283-03) | | | |
| Ovarian Margin | 13.1 | Breast Cancer (OD04566) | 2.7 |
| (OD06283-07) | | | |
| Ovarian Cancer 064008 | 23.2 | Breast Cancer 1024 | 43.5 |
| Ovarian cancer (OD06145) | 6.4 | Breast Cancer | 59.5 |
| | | (OD04590-01) | |
| Ovarian Margin (OD06145) | 52.5 | Breast Cancer Mets | 35.4 |
| | | (OD04590-03) | |
| Ovarian cancer | 13.6 | Breast Cancer Metastasis | 44.1 |
| (OD06455-03) | | (OD04655-05) | |
| Ovarian Margin | 33.2 | Breast Cancer 064006 | 17.9 |
| (OD06455-07) | | | |
| Normal Lung | 19.6 | Breast Cancer 9100266 | 16.6 |
| Invasive poor diff. lung adeno | 7.7 | Breast Margin 9100265 | 26.1 |
| (ODO4945-01) | | | |
| Lung Margin (ODO4945-03) | 48.3 | Breast Cancer A209073 | 9.8 |
| Lung Malignant Cancer | 11.0 | Breast Margin A2090734 | 28.3 |
| (OD03126) | | | |
| Lung Margin (OD03126) | 6.5 | Breast cancer (OD06083) | 63.7 |
| Lung Cancer (OD05014A) | 11.8 | Breast cancer node | 100.0 |
| | | metastasis (OD06083) | |
| Lung Margin (OD05014B) | 49.0 | Normal Liver | 39.8 |
| Lung cancer (OD06081) | 17.6 | Liver Cancer 1026 | 17.2 |
| Lung Margin (OD06081) | 37.9 | Liver Cancer 1025 | 40.3 |
| Lung Cancer (OD04237-01) | 1.2 | Liver Cancer 6004-T | 33.7 |
| Lung Margin (OD04237-02) | 23.7 | Liver Tissue 6004-N | 2.2 |
| Ocular Melanoma Metastasis | 44.1 | Liver Cancer 6005-T | 42.3 |
| Ocular Melanoma Margin | 14.9 | Liver Tissue 6005-N | 44.8 |
| (Liver) | | | |
| Melanoma Metastasis | 0.0 | Liver Cancer 064003 | 4.2 |
| Melanoma Margin (Lung) | 16.3 | Normal Bladder | 17.6 |
| Normal Kidney | 9.8 | Bladder Cancer 1023 | 8.4 |
| Kidney Ca, Nuclear grade 2 | 26.6 | Bladder Cancer A302173 | 3.2 |
| (OD04338) | | | |
| Kidney Margin (OD04338) | 1.6 | Normal Stomach | 25.2 |
| Kidney Ca Nuclear grade 1/2 | 20.2 | Gastric Cancer 9060397 | 0.7 |
| (OD04339) | | | |
| Kidney Margin (OD04339) | 10.2 | Stomach Margin 9060396 | 4.7 |
| Kidney Ca, Clear cell type | 20.9 | Gastric Cancer 9060395 | 8.1 |
| (OD04340) | | | |
| Kidney Margin (OD04340) | 13.3 | Stomach Margin 9060394 | 12.2 |
| Kidney Ca, Nuclear grade 3 | 4.4 | Gastric Cancer 064005 | 7.2 |
| (OD04348) | | | |

TABLE OM
Panel 4D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1143, | Ag1921, | Ag3082, | | Ag1143, | Ag1921, | Ag3082, |
| Tissue | Run | Run | Run | Tissue | Run | Run | Run |
| Name | 139943479 | 164629443 | 164681898 | Name | 139943479 | 164629443 | 164681898 |
| Secondary | 1.3 | 0.0 | 1.4 | HUVEC | 0.9 | 1.6 | 3.3 |
| Th1 act | | | | IL-1beta | | | |
| Secondary | 6.3 | 0.5 | 5.8 | HUVEC | 23.0 | 13.6 | 13.6 |
| Th2 act | | | | IFN | | | |
| | | | | gamma | | | |
| Secondary | 5.5 | 0.7 | 3.8 | HUVEC | 2.1 | 3.5 | 7.5 |
| Tr1 act | | | | TNF | | | |
| | | | | alpha + | | | |
| | | | | IFN | | | |
| | | | | gamma | | | |
| Secondary | 6.3 | 1.9 | 12.8 | HUVEC | 6.2 | 4.2 | 3.2 |
| Th1 rest | | | | TNF | | | |
| | | | | alpha + | | | |
| | | | | IL4 | | | |
| Secondary | 17.0 | 2.0 | 19.2 | HUVEC | 12.1 | 8.7 | 8.1 |
| Th2 rest | | | | IL-11 | | | |
| Secondary | 12.9 | 0.8 | 18.8 | Lung | 27.7 | 24.3 | 21.5 |
| Tr1 rest | | | | Microvascular | | | |
| | | | | EC | | | |
| | | | | none | | | |
| Primary | 0.9 | 0.0 | 0.8 | Lung | 8.7 | 14.5 | 16.6 |
| Th1 act | | | | Microvascular | | | |
| | | | | EC | | | |
| | | | | TNF alpha + | | | |
| | | | | IL-1beta | | | |
| Primary | 0.7 | 0.0 | 2.3 | Microvascular | 19.5 | 19.3 | 20.3 |
| Th2 act | | | | Dermal | | | |
| | | | | EC none | | | |
| Primary | 1.5 | 0.1 | 2.3 | Microsvasular | 10.1 | 11.3 | 10.3 |
| Tr1 act | | | | Dermal | | | |
| | | | | EC | | | |
| | | | | TNF alpha + | | | |
| | | | | IL-1beta | | | |
| Primary | 21.0 | 5.8 | 50.7 | Bronchial | 0.3 | 0.4 | 1.0 |
| Th1 rest | | | | epithelium | | | |
| | | | | TNF alpha + | | | |
| | | | | IL1beta | | | |
| Primary | 32.5 | 7.5 | 0.0 | Small | 2.0 | 4.9 | 2.6 |
| Th2 rest | | | | airway | | | |
| | | | | epithelium | | | |
| | | | | none | | | |
| Primary | 2.3 | 0.3 | 6.3 | Small | 0.7 | 0.2 | 2.1 |
| Tr1 rest | | | | airway | | | |
| | | | | epithelium | | | |
| | | | | TNF alpha + | | | |
| | | | | IL-1beta | | | |
| CD45RA | 2.5 | 1.4 | 2.3 | Coronery | 0.7 | 3.6 | 2.9 |
| CD4 | | | | artery | | | |
| lymphocyte | | | | SMC rest | | | |
| act | | | | | | | |
| CD45RO | 2.7 | 1.1 | 1.8 | Coronery | 6.7 | 1.4 | 3.7 |
| CD4 | | | | artery | | | |
| lymphocyte | | | | SMC | | | |
| act | | | | TNF alpha + | | | |
| | | | | IL-1beta | | | |
| CD8 | 2.1 | 0.6 | 2.9 | Astrocytes | 3.7 | 3.0 | 3.7 |
| lymphocyte | | | | rest | | | |
| act | | | | | | | |
| Secondary | 2.9 | 0.5 | 4.2 | Astrocytes | 3.7 | 3.4 | 4.0 |
| CD8 | | | | TNF alpha + | | | |
| lymphocyte | | | | IL-1beta | | | |
| rest | | | | | | | |
| Secondary | 5.4 | 0.0 | 3.1 | KU-812 | 0.0 | 0.0 | 0.0 |
| CD8 | | | | (Basophil) | | | |
| lymphocyte | | | | rest | | | |
| act | | | | | | | |
| CD4 | 11.7 | 3.7 | 10.8 | KU-812 | 0.0 | 0.1 | 0.1 |
| lymphocyte | | | | (Basophil) | | | |
| none | | | | PMA/ | | | |
| | | | | ionomycin | | | |
| 2ry | 27.5 | 1.3 | 27.0 | CCD1106 | 0.0 | 0.0 | 0.1 |
| Th1/Th2/ | | | | (Keratinocytes) | | | |
| Tr1_anti- | | | | none | | | |
| CD95 | | | | | | | |
| CH11 | | | | | | | |
| LAK cells | 4.5 | 1.1 | 4.4 | CCD1106 | 0.0 | 0.0 | 0.2 |
| rest | | | | (Keratinocytes) | | | |
| | | | | TNF alpha + | | | |
| | | | | IL-1beta | | | |
| LAK cells | 6.8 | 3.7 | 11.3 | Liver | 3.5 | 4.5 | 5.9 |
| IL-2 | | | | cirrhosis | | | |
| LAK cells | 1.8 | 0.3 | 2.8 | Lupus | 15.0 | 6.1 | 10.2 |
| IL-2 + IL-12 | | | | kidney | | | |
| LAK cells | 3.2 | 1.1 | 6.4 | NCI-H292 | 0.4 | 1.6 | 3.5 |
| IL-2 + IFN | | | | none | | | |
| gamma | | | | | | | |
| LAK cells | 1.7 | 0.5 | 7.6 | NCI-H292 | 0.6 | 1.3 | 3.4 |
| IL-2 + | | | | IL-4 | | | |
| IL-18 | | | | | | | |
| LAK cells | 1.6 | 0.1 | 1.1 | NCI-H292 | 1.0 | 2.2 | 3.0 |
| PMA/ionomycin | | | | IL-9 | | | |
| NK Cells | 2.2 | 0.8 | 3.2 | NCI-H292 | 3.5 | 0.4 | 0.8 |
| IL-2 rest | | | | IL-13 | | | |
| Two Way | 5.6 | 2.3 | 6.0 | NCI-H292 | 2.3 | 1.0 | 1.3 |
| MLR 3 | | | | IFN | | | |
| day | | | | gamma | | | |
| Two Way | 1.1 | 0.4 | 1.4 | HPAEC | 58.6 | 32.5 | 32.1 |
| MLR 5 | | | | none | | | |
| day | | | | | | | |
| Two Way | 0.9 | 0.5 | 1.4 | HPAEC | 18.6 | 16.8 | 22.4 |
| MLR 7 | | | | TNF | | | |
| day | | | | alpha + | | | |
| | | | | IL-1beta | | | |
| PBMC | 7.1 | 1.7 | 4.6 | Lung | 78.5 | 79.6 | 90.1 |
| rest | | | | fibroblast | | | |
| | | | | none | | | |
| PBMC | 0.9 | 1.0 | 3.8 | Lung | 20.0 | 18.0 | 24.0 |
| PWM | | | | fibroblast | | | |
| | | | | TNF | | | |
| | | | | alpha + | | | |
| | | | | IL-1beta | | | |
| PBMC | 3.0 | 0.4 | 2.6 | Lung | 48.3 | 100.0 | 100.0 |
| PHA-L | | | | fibroblast | | | |
| | | | | IL-4 | | | |
| Ramos (B | 0.0 | 0.0 | 0.0 | Lung | 35.4 | 56.6 | 49.7 |
| cell) none | | | | fibroblast | | | |
| | | | | IL-9 | | | |
| Ramos (B | 0.0 | 0.0 | 0.2 | Lung | 100.0 | 64.2 | 77.4 |
| cell) | | | | fibroblast | | | |
| ionomycin | | | | IL-13 | | | |
| B | 5.3 | 2.0 | 22.8 | Lung | 56.6 | 96.6 | 82.4 |
| lymphocytes | | | | fibroblast | | | |
| PWM | | | | IFN | | | |
| | | | | gamma | | | |
| B | 18.0 | 3.6 | 57.8 | Dermal | 5.7 | 9.3 | 17.2 |
| lymphocytes | | | | fibroblast | | | |
| CD40L | | | | CCD1070 | | | |
| and IL-4 | | | | rest | | | |
| EOL-1 | 0.0 | 0.1 | 0.2 | Dermal | 11.3 | 6.0 | 25.5 |
| dbcAMP | | | | fibroblast | | | |
| | | | | CCD1070 | | | |
| | | | | TNF | | | |
| | | | | alpha | | | |
| EOL-1 | 0.0 | 0.0 | 0.0 | Dermal | 20.3 | 4.0 | 6.5 |
| dbcAMP | | | | fibroblast | | | |
| PMA/ionomycin | | | | CCD1070 | | | |
| | | | | IL-1beta | | | |
| Dendritic | 3.3 | 4.2 | 5.1 | Dermal | 30.8 | 9.2 | 18.7 |
| cells none | | | | fibroblast | | | |
| | | | | IFN | | | |
| | | | | gamma | | | |
| Dendritic | 0.8 | 1.5 | 1.3 | Dermal | 35.1 | 17.8 | 26.4 |
| cells LPS | | | | fibroblast | | | |
| | | | | IL-4 | | | |
| Dendritic | 4.3 | 4.6 | 5.4 | IBD | 4.1 | 2.8 | 5.5 |
| cells | | | | Colitis 2 | | | |
| anti-CD40 | | | | | | | |
| Monocytes | 2.4 | 8.6 | 6.3 | IBD | 1.7 | 1.4 | 2.2 |
| rest | | | | Crohn's | | | |
| Monocytes | 0.0 | 0.2 | 1.0 | Colon | 6.2 | 7.4 | 8.5 |
| LPS | | | | | | | |
| Macrophages | 1.8 | 2.9 | 1.7 | Lung | 13.9 | 19.1 | 20.3 |
| rest | | | | | | | |
| Macrophages | 0.7 | 0.5 | 0.5 | Thymus | 25.2 | 16.4 | 14.7 |
| LPS | | | | | | | |
| HUVEC | 6.2 | 12.1 | 10.4 | Kidney | 13.8 | 8.6 | 31.0 |
| none | | | | | | | |
| HUVEC | 4.4 | 11.9 | 12.9 | | | | |
| starved | | | | | | | |

Panel 1 Summary: Ag345 Highest expression of the CG93887-01 gene is seen in the cerebellum (CT=24). High levels of expression are also seen in all regions of the CNS examined, including pituitary, amygdala, hypothalamus, thalamus, substantia nigra, and hippocampus. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Among tissues with metabolic function, this gene is expressed at high levels in pituitary, adrenal gland, pancreas, thyroid, skeletal muscle, heart, and adult and fetal liver. This widespread expression among these tissues suggests that this gene product may play a role in normal neuroendocrine and metabolic and that disregulated expression of this gene may contribute to neuroendocrine disorders or metabolic diseases, such as obesity and diabetes.

High levels of expression are also seen in cell lines derived from ovarian, breast, lung, brain and melanoma cancers. Therefore, therapeutic modulation of the expression or function of this gene may be effective in the treatment of these cancers.

In addition, this gene is expressed at much higher levels in liver tissue (CT=27) when compared to expression in the fetal counterpart (CTs=31). Thus, expression of this gene may be used to differentiate between the fetal and adult source of this tissue.

Panel 1.1 Summary: Ag558 Highest expression of the CG93387-01 gene is seen in a brain cancer cell line (CT=23.8). High levels of expression are also seen in cell lines derived from melanoma, ovarian, and lung cancers. Thus, expression of this gene could be used to differentiate between the brain cancer cell line sample and other samples on this panel and as a marker for brain cancers. Furthermore, therapeutic modulation of the expression or function of this gene may be effective in the treatment of ovarian, lung, brain, and melanoma cancers.

Among tissues with metabolic function, this gene is expressed at high to moderate levels in pituitary, adrenal gland, pancreas, thyroid, and adult and fetal liver, heart, and skeletal muscle. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at much higher levels in heart and liver tissue (CTs=25-26) when compared to expression in the fetal counterpart (CTs=29-32). Thus, expression of this gene may be used to differentiate between the fetal and adult source of these tissues.

High levels of expression are also seen in all regions of the CNS examined, including pituitary, amygdala, thalamus, substantia nigra, cerebral cortex, and hippocampus. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 1.2 Summary: Ag752 Highest expression of the CG93387-01 gene is seen in the thyroid (CT=25). High levels of expression are also seen among other metabolic tissues, including pancreas, adrenal, pituitary, skeletal muscle and adult and fetal heart and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at much higher levels in heart and liver tissue (CTs=26.8) when compared to expression in the fetal counterpart (CTs=30-31). Thus, expression of this gene may be used to differentiate between the fetal and adult source of these tissues.

High levels of expression are also seen in all regions of the CNS examined, including pituitary, amygdala, thalamus, cerebral cortex, and hippocampus. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Overall, expression of this gene appears to be more highly associated with normal tissues than cancer cell lines. High levels of expression are seen, however, in brain and ovarian cancer cell lines. Thus, this gene product may be involved in cancer of these tissues.

Panel 1.3D Summary: Ag3082 Highest expression of the CG93387-01 gene is seen in a brain cancer cell line (CT=27.3). Significant levels of expression are also seen in a cluster of samples derived from ovarian, breast, melanoma and brain cancer cell lines. Thus, expression of this gene could be used to differentiate between the brain cancer samples and other samples on this panel and as a marker to detect the presence of these cancers. This gene encodes a protien that is homologous to an epidermal growth factor related protein (fibropellin like). Fibropellins are a family of extracellular sea urchin matrix proteins that have been implicated in cell adhestion. Therefore, therapeutic modulation of the expression or function of this gene may be effective in the treatment of ovarian, breast, melanoma and brain cancers.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

Moderate to low levels of expression are also seen in all regions of the CNS examined, including pituitary, amygdala, thalamus, substantia nigra, cerebral cortex, and hippocampus. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 2.2 Summary: Ag3082 Highest expression of the CG93387-01 gene is seen in a breast cancer metastasis (CT=28.3). Significant levels of expression are also seen in a cluster of breast cancer samples. Conversely, expressoin appears to be higher in normal ovary and lung tissue when compared to expression in the normal adjacent tissue. Thus, therapeutic modulation of the expression or function of this gene may be effective in the treatment of breast, ovarian and lung cancers.

Panel 4D Summary: Ag1143/Ag1921/Ag3082 Three experiments with three different probe and primer sets produce results that are in very good agreement, with highest experession of the CG93387-01 gene in treated lung fibroblasts (CTs=27-29). Moderate levels of expression are also seen in treated dermal fibroblasts, and lung and dermal microvasculature, and HUVECs. Thus, expression of this gene could be used as a marker of fibroblasts or vasculature. The putative protein encoded by the transcript may also play an important role in the normal homeostasis of these tissues. Therefore, therapeutics designed with this gene product could be important for maintaining or restoring normal function to these organs during inflammation associated with asthma, psoriasis, and emphysema.

P. NOV21 (CG93702-01): Interleukin Receptor

Expression of gene CG93702-01 was assessed using the primer-probe sets Ag3878, Ag4529 and Ag4733, described in Tables PA, PB and PC. Results of the RTQ-PCR runs are shown in Table PD.

TABLE PA
Probe Name Ag3878

| | | | Start | SEQ ID |
|---|---|---|---|---|
| Primers | Sequences | Length | Position | NO |
| Forward | 5′-ccagagacaaggtcctctgat-3′ | 21 | 885 | 160 |
| Probe | TET-5′-ccaggcaacacctttgttgctgtg-3′-TAMRA | 24 | 922 | 161 |
| Reverse | 5′-agtcagcaggagaaagatgga-3′ | 21 | 946 | 162 |

TABLE PB
Probe Name Ag4529

| Primers | Sequences | Length | Start Position | SEQ ID NO |
|---|---|---|---|---|
| Forward | 5′-agatggagatccactgagcact-3′ | 22 | 52 | 163 |
| Probe | TET-5′-gctggaccttggagagtgaggcc-3′-TAMRA | 23 | 152 | 164 |
| Reverse | 5′-cctgagaatgttgttggtgagg-3′ | 22 | 294 | 165 |

TABLE PC
Probe Name Ag4733

| | | | | |
|---|---|---|---|---|
| | | | Start | SEQ ID |
| Primers | Sequences | Length | Position | NO |

| | | | | |
|---|---|---|---|---|
| Forward | 5′-tactcccttccgtccttagc-3′ | 20 | 1378 | 166 |
| Probe | TET-5′-aggcttggtcctaatcccagcacttt-3′-TAMRA | 26 | 1399 | 167 |
| Reverse | 5′-ctgatctgcaggttgcaaag-3′ | 20 | 1458 | 168 |

TABLE PD
Panel 4.1D

| | Rel. Exp. (%) | | Rel. Exp. (%) |
|---|---|---|---|
| | Ag3878, Run | | Ag3878, Run |
| Tissue Name | 170129734 | Tissue Name | 170129734 |
| Secondary Th1 act | 3.6 | HUVEC IL-1beta | 0.0 |
| Secondary Th2 act | 100.0 | HUVEC IFN gamma | 0.0 |
| Secondary Tr1 act | 71.2 | HUVEC TNF alpha + IFN | 0.0 |
| | | gamma | |
| Secondary Th1 rest | 5.2 | HUVEC TNF alpha + IL4 | 0.0 |
| Secondary Th2 rest | 45.1 | HUVEC IL-11 | 0.0 |
| Secondary Tr1 rest | 36.1 | Lung Microvascular EC none | 0.0 |
| Primary Th1 act | 0.0 | Lung Microvascular EC | 0.0 |
| | | TNF alpha + IL-1beta | |
| Primary Th2 act | 22.8 | Microvascular Dermal EC | 0.0 |
| | | none | |
| Primary Tr1 act | 4.9 | Microsvasular Dermal EC | 0.0 |
| | | TNF alpha + IL-1beta | |
| Primary Th1 rest | 10.4 | Bronchial epithelium | 0.0 |
| | | TNF alpha + IL1beta | |
| Primary Th2 rest | 17.7 | Small airway epithelium none | 0.2 |
| Primary Tr1 rest | 31.2 | Small airway epithelium | 0.4 |
| | | TNF alpha + IL-1beta | |
| CD45RA CD4 lymphocyte act | 1.2 | Coronery artery SMC rest | 0.0 |
| CD45RO CD4 lymphocyte act | 3.5 | Coronery artery SMC | 0.0 |
| | | TNF alpha + IL-1beta | |
| CD8 lymphocyte act | 2.2 | Astrocytes rest | 0.0 |
| Secondary CD8 lymphocyte | 1.6 | Astrocytes TNF alpha + | 0.0 |
| rest | | IL-1beta | 0.0 |
| Secondary CD8 lymphocyte act | 29.5 | KU-812 (Basophil) rest | 5.0 |
| CD4 lymphocyte none | 0.2 | KU-812 (Basophil) | 8.6 |
| | | PMA/ionomycin | |
| 2ry Th1/Th2/Tr1_anti-CD95 | 64.2 | CCD1106 (Keratinocytes) | 0.4 |
| CH11 | | none | |
| LAK cells rest | 2.2 | CCD1106 (Keratinocytes) | 0.3 |
| | | TNF alpha + IL-1beta | |
| LAK cells IL-2 | 8.3 | Liver cirrhosis | 0.0 |
| LAK cells IL-2 + IL-12 | 3.2 | NCI-H292 none | 0.0 |
| LAK cells IL-2 + IFN gamma | 2.7 | NCI-H292 IL-4 | 0.0 |
| LAK cells IL-2 + IL-18 | 4.9 | NCI-H292 IL-9 | 0.0 |
| LAK cells PMA/ionomycin | 6.0 | NCI-H292 IL-13 | 0.0 |
| NK Cells IL-2 rest | 8.1 | NCI-H292 IFN gamma | 0.3 |
| Two Way MLR 3 day | 0.6 | HPAEC none | 0.0 |
| Two Way MLR 5 day | 0.4 | HPAEC TNF alpha + | 0.0 |
| | | IL-1beta | |
| Two Way MLR 7 day | 1.9 | Lung fibroblast none | 0.0 |
| PBMC rest | 0.4 | Lung fibroblast TNF alpha + | 0.0 |
| | | IL-1beta | |
| PBMC PWM | 1.1 | Lung fibroblast IL-4 | 0.0 |
| PBMC PHA-L | 2.9 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 1.5 | Dermal fibroblast CCD1070 | 0.0 |
| | | rest | |
| B lymphocytes CD40L and IL-4 | 2.6 | Dermal fibroblast CCD1070 | 62.4 |
| | | TNF alpha | |
| EOL-1 dbcAMP | 0.2 | Dermal fibroblast CCD1070 | 0.0 |
| | | IL-1beta | |
| EOL-1 dbcAMP | 0.4 | Dermal fibroblast IFN gamma | 0.0 |
| PMA/ionomycin | | | |
| Dendritic cells none | 0.3 | Dermal fibroblast IL-4 | 0.1 |
| Dendritic cells LPS | 0.0 | Dermal fibroblasts rest | 0.0 |
| Dendritic cells anti-CD40 | 0.0 | Neutrophils TNFa + LPS | 0.3 |
| Monocytes rest | 0.0 | Neutrophils rest | 0.0 |
| Monocytes LPS | 0.0 | Colon | 0.1 |
| Macrophages rest | 0.0 | Lung | 0.4 |
| Macrophages LPS | 0.0 | Thymus | 6.0 |
| HUVEC none | 0.0 | Kidney | 0.0 |
| HUVEC starved | 0.0 | | |

CNS_neurodegeneration v1.0 Summary: Ag3878/Ag4529/Ag4733 Expression of the CG93702-01 gene is low/undetectable (CTs>35) across all of the samples on this panel.

General_screening_panel_v1.4 Summary: Ag3878 Results from one experiment with the CG93702-01 gene are not included. The amp plot indicates that there were experimental difficulties with this run.

Panel 4.1D Summary: Ag3878 Highest expression of the CG93702-01 gene is detected in activated secondary Th2 (CT=27.6). In addition high expression of this gene is also seen in resting and activated primary and secondary Th1, Th2, Tr1 cells, CD45RA CD4 lymphocyte, secondary CD8 lymphocyte, resting and lymphokine activated killer (LAK) cells. Since these cells play an important role in lung pathology, inflammatory bowel disease and autoimmune disorders, including rheumatoid arthritis, antibody or small molecule therapies designed with the protein encoded by this gene may block or inhibit inflammation and tissue resulting from asthma, allergies, hypersensitivity reactions, inflammatory bowel disease, viral infections and autoimmune diseases.

Interestingly, expression of this gene is also stimulated in TNF alpha treated dermal fibroblast CCD1070 cells (CT=28) as compared to the resting cells (CT=40). Thus expression of this gene can be used to distinguish between these two samples. In addition, expression in TNF alpha treated dermal fibroblasts suggests that this gene product may be involved in skin disorders, including psoriasis.

Expression of this gene is also detected in basophils (KU-812 cells) (CTs=31). Therefore, antibody or small molecule therapies designed with the protein encoded for by this gene could block or inhibit inflammation or tissue damage due to basophil activation in response to asthma, allergies, hypersensitivity reactions, psoriasis, and viral infections.

Ag4529/Ag4733 Expression of this gene is low/undetectable (CTs>35) across all of the samples on this panel.

Q. NOV23 and NOV22 (CG94013-01 and CG93792-01): Ig, TSP and EGF Domain-Containing Protein

Expression of gene CG94013-01 and variant CG93792-01 was assessed using the primer-probe sets Ag1315b, Ag1316b, Ag1924, Ag3108, Ag900, Ag3899, Ag3960, Ag4338 and Ag343, described in Tables QA, QB, QC, QD, QE, QF, QG, QH and QI. Results of the RTQ-PCR runs are shown in Tables QJ, QK, QL, QM, QN, QO and QP. Please note that the probe and primer sets Ag3108 and Ag3899 are specific to CG94013-01.

TABLE QA

Probe Name Ag1315b

| | | | Start | SEQ ID |
|---|---|---|---|---|
| Primers | Sequences | Length | Position | NO |
| Forward | 5′-catcagaggttcttcgaaagc-3′ | 21 | 4844 | 169 |
| Probe | TET-5′-cacaacggaccacacagcgataagat-3′-TAMRA | 26 | 4812 | 170 |
| Reverse | 5′-aggactgtgacaatacgattgg-3′ | 22 | 4790 | 171 |

TABLE QB

Probe Name Ag1316b

| | | | Start | SEQ ID |
|---|---|---|---|---|
| Primers | Sequences | Length | Position | NO |
| Forward | 5′-aatgccatggggacttactact-3′ | 22 | 4672 | 172 |
| Probe | TET-5′-cctaaaggcctcaccatagctgcaga-3′-TAMRA | 26 | 4702 | 173 |
| Reverse | 5′-cccaaagcacactcatcaatat-3′ | 22 | 4745 | 174 |

TABLE QC

Probe Name Ag1924

| | | | Start | SEQ ID |
|---|---|---|---|---|
| Primers | Sequences | Length | Position | NO |
| Forward | 5′-ctatgggagcagggattcc-3′ | 19 | 4646 | 175 |
| Probe | TET-5′-ctgcacattcatcctcatcagcacaa-3′-TAMRA | 26 | 4617 | 176 |
| Reverse | 5′-ccgggtttaccttagactcagt-3′ | 22 | 4586 | 177 |

TABLE QD

Probe Name Ag3108

| | | | Start | SEQ ID |
|---|---|---|---|---|
| Primers | Sequences | Length | Position | NO |
| Forward | 5′-attccattgcccaaattaaca-3′ | 21 | 2101 | 178 |
| Probe | TET-5′-ccttcaataacaatattattccagccca-3′-TAMRA | 28 | 2126 | 179 |
| Reverse | 5′-actgtgtccattcacactgtca-3′ | 22 | 2157 | 180 |

TABLE QE

Probe Name Ag900

| | | | Start | SEQ ID |
|---|---|---|---|---|
| Primers | Sequences | Length | Position | NO |
| Forward | 5′-aatgccatggggacttactact-3′ | 22 | 4672 | 181 |
| Probe | TET-5′-cctaaaggcctcaccatagctgcaga-3′-TAMRA | 26 | 4702 | 182 |
| Reverse | 5′-cccaaagcacactcatcaatat-3′ | 22 | 4745 | 183 |

TABLE QF

Probe Name Ag3899

| | | | Start | SEQ ID |
|---|---|---|---|---|
| Primers | Sequences | Length | Position | NO |
| Forward | 5′-ccattgcccaaattaacatg-3′ | 20 | 2104 | 184 |
| Probe | TET-5′-ccttcaataacaatattattccagccca-3′-TAMRA | 28 | 2126 | 185 |
| Reverse | 5′-actgtgtccattcacactgtca-3′ | 22 | 2157 | 186 |

TABLE QG

Probe Name Ag3960

| | | | Start | SEQ ID |
|---|---|---|---|---|
| Primers | Sequences | Length | Position | NO |
| Forward | 5′-aaacacttcatgcatcctctgt-3′ | 22 | 4475 | 187 |
| Probe | TET-5′-cactgggttttaaaattcatgcttca-3′-TAMRA | 26 | 4526 | 188 |
| Reverse | 5′-ttactgcgatctcctttggata-3′ | 22 | 4553 | 189 |

TABLE QH

Probe Name Ag4338

| | | | Start | SEQ ID |
|---|---|---|---|---|
| Primers | Sequences | Length | Position | NO |
| Forward | 5′-tcatgcatcctctgtggaat-3′ | 20 | 4482 | 190 |
| Probe | TET-5′-cactgggttttaaaattcatgcttca-3′-TAMRA | 26 | 4526 | 191 |
| Reverse | 5′-ctgattactgcgatctcctttg-3′ | 22 | 4557 | 192 |

TABLE QI

Probe Name Ag343

| Primers | Sequences | Length | Start Position | SEQ ID NO |
|---|---|---|---|---|
| Forward | 5′-attgcacctggtcacctgagt-3′ | 21 | 3877 | 193 |
| Probe | TET-5′-tggccgtccctgtcccgga-3′-TAMRA | 19 | 3852 | 194 |
| Reverse | 5′-gctgtgcgaccatcctgtg-3′ | 19 | 3822 | 195 |

TABLE QJ
CNS_neurodegeneration_v1.0

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag3899, | Ag3960, | Ag4338, | | Ag3899, | Ag3960, | Ag4338, |
| Tissue | Run | Run | Run | Tissue | Run | Run | Run |
| Name | 212247977 | 212347483 | 224349481 | Name | 212247977 | 212347483 | 224349481 |
| AD 1 | 0.0 | 5.6 | 0.0 | Control | 0.0 | 9.3 | 0.0 |
| Hippo | | | | (Path) 3 | | | |
| | | | | Temporal | | | |
| | | | | Ctx | | | |
| AD 2 | 0.0 | 6.2 | 0.0 | Control | 32.3 | 33.9 | 35.6 |
| Hippo | | | | (Path) 4 | | | |
| | | | | Temporal | | | |
| | | | | Ctx | | | |
| AD 3 | 0.0 | 8.1 | 0.0 | AD 1 | 0.0 | 30.4 | 25.0 |
| Hippo | | | | Occipital | | | |
| | | | | Ctx | | | |
| AD 4 | 0.0 | 0.0 | 0.0 | AD 2 | 0.0 | 0.0 | 0.0 |
| Hippo | | | | Occipital | | | |
| | | | | Ctx | | | |
| | | | | (Missing) | | | |
| AD 5 | 13.3 | 53.6 | 49.3 | AD 3 | 0.0 | 6.3 | 0.0 |
| hippo | | | | Occipital | | | |
| | | | | Ctx | | | |
| AD 6 | 24.1 | 39.8 | 0.0 | AD 4 | 0.0 | 14.1 | 0.0 |
| Hippo | | | | Occipital | | | |
| | | | | Ctx | | | |
| Control 2 | 0.0 | 9.4 | 0.0 | AD 5 | 6.2 | 0.0 | 0.0 |
| Hippo | | | | Occipital | | | |
| | | | | Ctx | | | |
| Control 4 | 9.9 | 8.3 | 0.0 | AD 6 | 0.0 | 9.5 | 51.8 |
| Hippo | | | | Occipital | | | |
| | | | | Ctx | | | |
| Control | 0.0 | 0.0 | 0.0 | Control 1 | 17.4 | 25.9 | 55.9 |
| (Path) 3 | | | | Occipital | | | |
| Hippo | | | | Ctx | | | |
| AD 1 | 0.0 | 15.8 | 18.6 | Control 2 | 0.0 | 11.2 | 0.0 |
| Temporal | | | | Occipital | | | |
| Ctx | | | | Ctx | | | |
| AD 2 | 0.0 | 10.1 | 0.0 | Control 3 | 0.0 | 11.1 | 0.0 |
| Temporal | | | | Occipital | | | |
| Ctx | | | | Ctx | | | |
| AD 3 | 0.0 | 5.4 | 0.0 | Control 4 | 0.0 | 3.1 | 0.0 |
| Temporal | | | | Occipital | | | |
| Ctx | | | | Ctx | | | |
| AD 4 | 0.0 | 11.2 | 0.0 | Control | 100.0 | 49.3 | 100.0 |
| Temporal | | | | (Path) 1 | | | |
| Ctx | | | | Occipital | | | |
| | | | | Ctx | | | |
| AD 5 Inf | 15.3 | 11.8 | 0.0 | Control | 0.0 | 9.0 | 0.0 |
| Temporal | | | | (Path) 2 | | | |
| Ctx | | | | Occipital | | | |
| | | | | Ctx | | | |
| AD 5 | 0.0 | 10.2 | 0.0 | Control | 0.0 | 15.8 | 53.6 |
| SupTemporal | | | | (Path) 3 | | | |
| Ctx | | | | Occipital | | | |
| | | | | Ctx | | | |
| AD 6 Inf | 28.9 | 90.1 | 37.9 | Control | 0.0 | 27.4 | 0.0 |
| Temporal | | | | (Path) 4 | | | |
| Ctx | | | | Occipital | | | |
| | | | | Ctx | | | |
| AD 6 | 21.3 | 41.5 | 65.5 | Control 1 | 0.0 | 21.6 | 0.0 |
| Sup | | | | Parietal | | | |
| Temporal | | | | Ctx | | | |
| Ctx | | | | | | | |
| Control 1 | 0.0 | 0.0 | 36.6 | Control 2 | 0.0 | 8.5 | 0.0 |
| Temporal | | | | Parietal | | | |
| Ctx | | | | Ctx | | | |
| Control 2 | 0.0 | 0.0 | 29.9 | Control 3 | 0.0 | 3.7 | 0.0 |
| Temporal | | | | Parietal | | | |
| Ctx | | | | Ctx | | | |
| Control 3 | 0.0 | 6.1 | 0.0 | Control | 10.6 | 39.2 | 66.0 |
| Temporal | | | | (Path) 1 | | | |
| Ctx | | | | Parietal | | | |
| | | | | Ctx | | | |
| Control 4 | 0.0 | 17.4 | 0.0 | Control | 0.0 | 57.0 | 21.2 |
| Temporal | | | | (Path) 2 | | | |
| Ctx | | | | Parietal | | | |
| | | | | Ctx | | | |
| Control | 52.1 | 38.2 | 0.0 | Control | 0.0 | 15.1 | 29.7 |
| (Path) 1 | | | | (Path) 3 | | | |
| Temporal | | | | Parietal | | | |
| Ctx | | | | Ctx | | | |
| Control | 0.0 | 35.8 | 24.0 | Control | 0.0 | 100.0 | 71.7 |
| (Path) 2 | | | | (Path) 4 | | | |
| Temporal | | | | Parietal | | | |
| Ctx | | | | Ctx | | | |

TABLE QK
General_screening_panel_v1.4

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag3899, | Ag3960, | Ag4338, | | Ag3899, | Ag3960, | Ag4338, |
| Tissue | Run | Run | Run | | Run | Run | Run |
| Name | 219166475 | 217310662 | 222550860 | Tissue Name | 219166475 | 217310662 | 222550860 |
| Adipose | 1.0 | 1.9 | 2.6 | Renal ca. | 0.0 | 0.0 | 0.0 |
| | | | | TK-10 | | | |
| Melanoma* | 33.9 | 72.7 | 79.0 | Bladder | 0.6 | 1.2 | 1.1 |
| Hs688(A).T | | | | | | | |
| Melanoma* | 8.4 | 22.4 | 28.9 | Gastric ca. | 0.0 | 0.0 | 0.1 |
| Hs688(B).T | | | | (liver met.) | | | |
| | | | | NCI-N87 | | | |
| Melanoma* | 12.9 | 24.0 | 25.3 | Gastric ca. | 0.0 | 0.1 | 0.1 |
| M14 | | | | KATO III | | | |
| Melanoma* | 0.1 | 0.2 | 0.4 | Colon ca. | 0.0 | 0.0 | 0.0 |
| LOXIMVI | | | | SW-948 | | | |
| Melanoma* | 58.6 | 58.2 | 77.4 | Colon ca. | 0.0 | 0.1 | 0.2 |
| SK-MEL-5 | | | | SW480 | | | |
| Squamous | 0.0 | 0.0 | 0.1 | Colon ca.* | 0.0 | 0.0 | 0.0 |
| cell | | | | (SW480 met) | | | |
| carcinoma | | | | SW620 | | | |
| SCC-4 | | | | | | | |
| Testis Pool | 0.6 | 0.9 | 0.9 | Colon ca. | 0.0 | 0.0 | 0.0 |
| | | | | HT29 | | | |
| Prostate ca.* | 0.2 | 0.6 | 0.8 | Colon ca. | 0.0 | 0.1 | 0.1 |
| (bone met) | | | | HCT-116 | | | |
| PC-3 | | | | | | | |
| Prostate | 0.4 | 1.4 | 2.1 | Colon ca. | 0.0 | 0.0 | 0.1 |
| Pool | | | | CaCo-2 | | | |
| Placenta | 0.1 | 0.3 | 0.5 | Colon cancer | 1.2 | 2.1 | 3.8 |
| | | | | tissue | | | |
| Uterus Pool | 0.1 | 0.2 | 0.6 | Colon ca. | 0.0 | 0.0 | 0.0 |
| | | | | SW1116 | | | |
| Ovarian ca. | 0.4 | 1.2 | 1.2 | Colon ca. | 0.0 | 0.0 | 0.0 |
| OVCAR-3 | | | | Colo-205 | | | |
| Ovarian ca. | 0.1 | 0.8 | 0.5 | Colon ca. | 0.0 | 0.0 | 0.0 |
| SK-OV-3 | | | | SW-48 | | | |
| Ovarian ca. | 0.1 | 0.1 | 0.2 | Colon Pool | 0.2 | 1.5 | 1.8 |
| OVCAR-4 | | | | | | | |
| Ovarian ca. | 0.2 | 0.4 | 0.6 | Small Intestine | 0.2 | 1.2 | 1.0 |
| OVCAR-5 | | | | Pool | | | |
| Ovarian ca. | 0.1 | 0.1 | 0.0 | Stomach Pool | 0.1 | 0.9 | 0.8 |
| IGROV-1 | | | | | | | |
| Ovarian ca. | 0.1 | 0.2 | 0.1 | Bone Marrow | 0.2 | 0.4 | 0.6 |
| OVCAR-8 | | | | Pool | | | |
| Ovary | 3.6 | 4.3 | 5.6 | Fetal Heart | 1.0 | 1.3 | 1.9 |
| Breast ca. | 0.5 | 2.0 | 2.7 | Heart Pool | 0.3 | 0.8 | 0.7 |
| MCF-7 | | | | | | | |
| Breast ca. | 0.1 | 0.2 | 0.1 | Lymph Node | 0.4 | 1.8 | 2.2 |
| MDA-MB-231 | | | | Pool | | | |
| Breast ca. | 2.6 | 10.0 | 7.1 | Fetal Skeletal | 0.1 | 0.5 | 0.7 |
| BT 549 | | | | Muscle | | | |
| Breast ca. | 0.2 | 0.4 | 0.7 | Skeletal | 0.2 | 0.8 | 0.6 |
| T47D | | | | Muscle Pool | | | |
| Breast ca. | 2.2 | 15.1 | 20.3 | Spleen Pool | 1.1 | 2.3 | 2.8 |
| MDA-N | | | | | | | |
| Breast Pool | 0.1 | 1.1 | 1.9 | Thymus Pool | 0.6 | 1.0 | 1.3 |
| Trachea | 1.0 | 2.8 | 2.9 | CNS cancer | 0.8 | 1.9 | 2.4 |
| | | | | (glio/astro) | | | |
| | | | | U87-MG | | | |
| Lung | 0.0 | 0.5 | 0.7 | CNS cancer | 3.0 | 10.0 | 10.5 |
| | | | | (glio/astro) | | | |
| | | | | U-118-MG | | | |
| Fetal Lung | 5.6 | 21.9 | 23.7 | CNS cancer | 0.0 | 0.0 | 0.0 |
| | | | | (neuro; met) | | | |
| | | | | SK-N-AS | | | |
| Lung ca. | 0.0 | 0.1 | 0.1 | CNS cancer | 18.8 | 37.1 | 37.1 |
| NCI-N417 | | | | (astro) SF-539 | | | |
| Lung ca. | 0.0 | 0.0 | 0.0 | CNS cancer | 100.0 | 100.0 | 100.0 |
| LX-1 | | | | (astro) SNB-75 | | | |
| Lung ca. | 0.0 | 0.1 | 0.1 | CNS cancer | 0.0 | 0.1 | 0.0 |
| NCI-H146 | | | | (glio) SNB-19 | | | |
| Lung ca. | 0.0 | 0.0 | 0.0 | CNS cancer | 0.8 | 2.4 | 3.1 |
| SHP-77 | | | | (glio) SF-295 | | | |
| Lung ca. | 0.0 | 0.0 | 0.0 | Brain | 0.0 | 0.0 | 0.0 |
| A549 | | | | (Amygdala) | | | |
| | | | | Pool | | | |
| Lung ca. | 0.0 | 0.0 | 0.0 | Brain | 0.0 | 0.0 | 0.0 |
| NCI-H526 | | | | (cerebellum) | | | |
| Lung ca. | 0.3 | 0.2 | 0.3 | Brain (fetal) | 0.0 | 0.2 | 0.3 |
| NCI-H23 | | | | | | | |
| Lung ca. | 0.1 | 2.3 | 1.3 | Brain | 0.0 | 0.1 | 0.3 |
| NCI-H460 | | | | (Hippocampus) | | | |
| | | | | Pool | | | |
| Lung ca. | 0.6 | 1.7 | 2.6 | Cerebral | 0.0 | 0.1 | 0.1 |
| HOP-62 | | | | Cortex Pool | | | |
| Lung ca. | 0.0 | 0.1 | 0.0 | Brain | 0.0 | 0.1 | 0.1 |
| NCI-H522 | | | | (Substantia | | | |
| | | | | nigra) Pool | | | |
| Liver | 0.0 | 0.1 | 0.2 | Brain | 0.0 | 0.2 | 0.2 |
| | | | | (Thalamus) | | | |
| | | | | Pool | | | |
| Fetal Liver | 1.3 | 1.7 | 2.4 | Brain (whole) | 0.0 | 0.2 | 0.2 |
| Liver ca. | 0.0 | 0.0 | 0.0 | Spinal Cord | 0.1 | 0.3 | 0.2 |
| HepG2 | | | | Pool | | | |
| Kidney Pool | 0.2 | 0.7 | 0.6 | Adrenal Gland | 0.1 | 0.4 | 0.4 |
| Fetal | 1.4 | 2.4 | 3.6 | Pituitary gland | 0.1 | 0.2 | 0.5 |
| Kidney | | | | Pool | | | |
| Renal ca. | 0.2 | 0.8 | 0.4 | Salivary Gland | 0.2 | 0.6 | 0.7 |
| 786-0 | | | | | | | |
| Renal ca. | 0.0 | 0.2 | 0.2 | Thyroid | 0.1 | 0.2 | 0.7 |
| A498 | | | | (female) | | | |
| Renal ca. | 0.0 | 0.0 | 0.0 | Pancreatic ca. | 0.0 | 0.0 | 0.0 |
| ACHN | | | | CAPAN2 | | | |
| Renal ca. | 4.6 | 4.8 | 1.3 | Pancreas Pool | 0.4 | 1.4 | 1.4 |
| UO-31 | | | | | | | |

TABLE QL
Panel 1

| | Rel. Exp. (%) | | Rel. Exp. (%) |
|---|---|---|---|
| | Ag343, Run | | Ag343, Run |
| Tissue Name | 87586142 | Tissue Name | 87586142 |
| Endothelial cells | 0.0 | Renal ca. 786-0 | 0.9 |
| Endothelial cells (treated) | 0.0 | Renal ca. A498 | 0.0 |
| Pancreas | 0.3 | Renal ca. RXF 393 | 0.0 |
| Pancreatic ca. CAPAN 2 | 0.0 | Renal ca. ACHN | 0.0 |
| Adrenal gland | 1.3 | Renal ca. UO-31 | 4.3 |
| Thyroid | 4.2 | Renal ca. TK-10 | 0.0 |
| Salivary gland | 6.1 | Liver | 14.6 |
| Pituitary gland | 2.6 | Liver (fetal) | 3.7 |
| Brain (fetal) | 0.0 | Liver ca. (hepatoblast) HepG2 | 0.0 |
| Brain (whole) | 0.0 | Lung | 12.4 |
| Brain (amygdala) | 0.0 | Lung (fetal) | 29.1 |
| Brain (cerebellum) | 0.2 | Lung ca. (small cell) LX-1 | 0.0 |
| Brain (hippocampus) | 0.0 | Lung ca. (small cell) NCI-H69 | 0.0 |
| Brain (substantia nigra) | 0.0 | Lung ca. (s.cell var.) SHP-77 | 0.0 |
| Brain (thalamus) | 0.0 | Lung ca. (large cell) NCI-H460 | 15.7 |
| Brain (hypothalamus) | 6.5 | Lung ca. (non-sm. cell) A549 | 0.0 |
| Spinal cord | 2.9 | Lung ca. (non-s.cell) NCI-H23 | 0.0 |
| glio/astro U87-MG | 6.3 | Lung ca. (non-s.cell) HOP-62 | 7.2 |
| glio/astro U-118-MG | 10.6 | Lung ca. (non-s.cl) NCI-H522 | 0.0 |
| astrocytoma SW1783 | 1.6 | Lung ca. (squam.) SW 900 | 9.2 |
| neuro*; met SK-N-AS | 0.0 | Lung ca. (squam.) NCI-H596 | 0.0 |
| astrocytoma SF-539 | 54.7 | Mammary gland | 72.2 |
| astrocytoma SNB-75 | 29.7 | Breast ca.* (pl.ef) MCF-7 | 13.7 |
| glioma SNB-19 | 0.0 | Breast ca.* (pl.ef) | 0.0 |
| | | MDA-MB-231 | |
| glioma U251 | 0.6 | Breast ca.* (pl. ef) T47D | 0.0 |
| glioma SF-295 | 1.8 | Breast ca. BT-549 | 2.6 |
| Heart | 18.4 | Breast ca. MDA-N | 100.0 |
| Skeletal muscle | 1.7 | Ovary | 24.0 |
| Bone marrow | 0.0 | Ovarian ca. OVCAR-3 | 0.0 |
| Thymus | 7.1 | Ovarian ca. OVCAR-4 | 0.0 |
| Spleen | 20.3 | Ovarian ca. OVCAR-5 | 0.6 |
| Lymph node | 8.8 | Ovarian ca. OVCAR-8 | 0.0 |
| Colon (ascending) | 7.9 | Ovarian ca. IGROV-1 | 0.0 |
| Stomach | 20.3 | Ovarian ca* (ascites) SK-OV-3 | 0.0 |
| Small intestine | 13.7 | Uterus | 10.3 |
| Colon ca. SW480 | 0.0 | Placenta | 10.7 |
| Colon ca.* SW620 (SW480 | 0.0 | Prostate | 7.4 |
| met) | | | |
| Colon ca. HT29 | 0.0 | Prostate ca.* (bone met) PC-3 | 3.0 |
| Colon ca. HCT-116 | 0.0 | Testis | 45.7 |
| Colon ca. CaCo-2 | 0.0 | Melanoma Hs688(A).T | 45.7 |
| Colon ca. HCT-15 | 0.0 | Melanoma* (met) Hs688(B).T | 62.9 |
| Colon ca. HCC-2998 | 0.0 | Melanoma UACC-62 | 97.3 |
| Gastric ca.* (liver met) | 0.0 | Melanoma M14 | 90.1 |
| NCI-N87 | | | |
| Bladder | 5.0 | Melanoma LOX IMVI | 0.5 |
| Trachea | 10.6 | Melanoma* (met) SK-MEL-5 | 95.9 |
| Kidney | 7.2 | Melanoma SK-MEL-28 | 72.7 |
| Kidney (fetal) | 29.9 | | |

TABLE QM
Panel 1.3D

| | Rel. Exp. (%) | | Rel. Exp. (%) |
|---|---|---|---|
| | Ag3108, Run | | Ag3108, Run |
| Tissue Name | 167985250 | Tissue Name | 167985250 |
| Liver adenocarcinoma | 0.2 | Kidney (fetal) | 4.2 |
| Pancreas | 0.1 | Renal ca. 786-0 | 0.5 |
| Pancreatic ca. CAPAN 2 | 0.0 | Renal ca. A498 | 7.7 |
| Adrenal gland | 0.0 | Renal ca. RXF 393 | 0.5 |
| Thyroid | 0.3 | Renal ca. ACHN | 0.0 |
| Salivary gland | 0.0 | Renal ca. UO-31 | 7.9 |
| Pituitary gland | 0.3 | Renal ca. TK-10 | 0.0 |
| Brain (fetal) | 0.1 | Liver | 0.2 |
| Brain (whole) | 0.3 | Liver (fetal) | 0.7 |
| Brain (amygdala) | 0.0 | Liver ca. (hepatoblast) HepG2 | 0.0 |
| Brain (cerebellum) | 0.0 | Lung | 0.4 |
| Brain (hippocampus) | 0.0 | Lung (fetal) | 5.7 |
| Brain (substantia nigra) | 0.2 | Lung ca. (small cell) LX-1 | 0.0 |
| Brain (thalamus) | 0.0 | Lung ca. (small cell) NCI-H69 | 0.1 |
| Cerebral Cortex | 0.0 | Lung ca. (s.cell var.) SHP-77 | 0.1 |
| Spinal cord | 0.5 | Lung ca. (large cell) NCI-H460 | 0.6 |
| glio/astro U87-MG | 1.2 | Lung ca. (non-sm. cell) A549 | 0.0 |
| glio/astro U-118-MG | 3.1 | Lung ca. (non-s.cell) NCI-H23 | 0.4 |
| astrocytoma SW1783 | 1.4 | Lung ca. (non-s.cell) HOP-62 | 1.9 |
| neuro*; met SK-N-AS | 0.0 | Lung ca. (non-s.cl) NCI-H522 | 0.1 |
| astrocytoma SF-539 | 25.2 | Lung ca. (squam.) SW 900 | 1.7 |
| astrocytoma SNB-75 | 30.8 | Lung ca. (squam.) NCI-H596 | 0.3 |
| glioma SNB-19 | 0.0 | Mammary gland | 1.2 |
| glioma U251 | 2.4 | Breast ca.* (pl.ef) MCF-7 | 1.0 |
| glioma SF-295 | 1.1 | Breast ca.* (pl.ef) | 0.0 |
| | | MDA-MB-231 | |
| Heart (fetal) | 0.8 | Breast ca.* (pl.ef) T47D | 0.1 |
| Heart | 1.2 | Breast ca. BT-549 | 0.2 |
| Skeletal muscle (fetal) | 0.1 | Breast ca. MDA-N | 28.7 |
| Skeletal muscle | 0.7 | Ovary | 1.0 |
| Bone marrow | 0.0 | Ovarian ca. OVCAR-3 | 0.8 |
| Thymus | 0.1 | Ovarian ca. OVCAR-4 | 0.1 |
| Spleen | 0.6 | Ovarian ca. OVCAR-5 | 0.8 |
| Lymph node | 0.2 | Ovarian ca. OVCAR-8 | 0.0 |
| Colorectal | 0.0 | Ovarian ca. IGROV-1 | 0.2 |
| Stomach | 0.2 | Ovarian ca.* (ascites) | 0.5 |
| | | SK-OV-3 | |
| Small intestine | 0.4 | Uterus | 0.4 |
| Colon ca. SW480 | 0.0 | Placenta | 0.2 |
| Colon ca.* SW620(SW480 | 0.0 | Prostate | 0.2 |
| met) | | | |
| Colon ca. HT29 | 0.0 | Prostate ca.* (bone met) PC-3 | 0.7 |
| Colon ca. HCT-116 | 0.0 | Testis | 0.3 |
| Colon ca. CaCo-2 | 0.0 | Melanoma Hs688(A).T | 12.4 |
| Colon ca. tissue(ODO3866) | 4.2 | Melanoma* (met) Hs688(B).T | 2.2 |
| Colon ca. HCC-2998 | 0.0 | Melanoma UACC-62 | 100.0 |
| Gastric ca.* (liver met) | 0.0 | Melanoma M14 | 14.6 |
| NCI-N87 | | | |
| Bladder | 0.3 | Melanoma LOX IMVI | 0.2 |
| Trachea | 0.4 | Melanoma* (met) SK-MEL-5 | 20.3 |
| Kidney | 0.4 | Adipose | 3.3 |

TABLE QN
Panel 2.1

| | Rel. Exp. (%) | | Rel. Exp. (%) |
|---|---|---|---|
| | Ag3108, Run | | Ag3108, Run |
| Tissue Name | 170686074 | Tissue Name | 170686074 |
| Normal Colon | 0.7 | Kidney Cancer 9010320 | 0.9 |
| Colon cancer (OD06064) | 1.3 | Kidney margin 9010321 | 9.5 |
| Colon cancer margin (OD06064) | 0.0 | Kidney Cancer 8120607 | 0.6 |
| Colon cancer (OD06159) | 0.5 | Kidney margin 8120608 | 0.7 |
| Colon cancer margin (OD06159) | 1.8 | Normal Uterus | 1.7 |
| Colon cancer (OD06298-08) | 1.6 | Uterus Cancer | 1.2 |
| Colon cancer margin | 0.3 | Normal Thyroid | 0.1 |
| (OD06298-018) | | | |
| Colon Cancer Gr.2 ascend colon | 1.6 | Thyroid Cancer | 0.9 |
| (ODO3921) | | | |
| Colon Cancer margin (ODO3921) | 4.6 | Thyroid Cancer A302152 | 1.2 |
| Colon cancer metastasis | 2.1 | Thyroid margin A302153 | 0.9 |
| (OD06104) | | | |
| Lung margin (OD06104) | 2.8 | Normal Breast | 12.4 |
| Colon mets to lung (OD04451-01) | 4.5 | Breast Cancer | 0.9 |
| Lung margin (OD04451-02) | 10.7 | Breast Cancer | 4.3 |
| Normal Prostate | 0.8 | Breast Cancer | 0.6 |
| | | (OD04590-01) | |
| Prostate Cancer (OD04410) | 0.7 | Breast Cancer Mets | 6.6 |
| | | (OD04590-03) | |
| Prostate margin (OD04410) | 13.6 | Breast Cancer Metastasis | 2.1 |
| Normal Lung | 34.2 | Breast Cancer | 3.3 |
| Invasive poor diff. lung adeno 1 | 9.2 | Breast Cancer 9100266 | 4.6 |
| (ODO4945-01) | | | |
| Lung margin (ODO4945-03) | 6.2 | Breast margin 9100265 | 1.5 |
| Lung Malignant Cancer | 11.1 | Breast Cancer A209073 | 2.5 |
| (OD03126) | | | |
| Lung margin (OD03126) | 34.9 | Breast margin A2090734 | 9.9 |
| Lung Cancer (OD05014A) | 25.2 | Normal Liver | 4.2 |
| Lung margin (OD05014B) | 5.6 | Liver Cancer 1026 | 1.8 |
| Lung Cancer (OD04237-01) | 1.5 | Liver Cancer 1025 | 6.1 |
| Lung margin (OD04237-02) | 63.3 | Liver Cancer 6004-T | 3.5 |
| Ocular Mel Met to Liver | 24.3 | Liver Tissue 6004-N | 0.8 |
| (ODO4310) | | | |
| Liver margin (ODO4310) | 7.6 | Liver Cancer 6005-T | 14.2 |
| Melanoma Mets to Lung | 100.0 | Liver Tissue 6005-N | 14.8 |
| (OD04321) | | | |
| Lung margin (OD04321) | 20.2 | Liver Cancer | 1.4 |
| Normal Kidney | 3.6 | Normal Bladder | 1.7 |
| Kidney Ca, Nuclear grade 2 | 6.9 | Bladder Cancer | 1.8 |
| (OD04338) | | | |
| Kidney margin (OD04338) | 2.1 | Bladder Cancer | 2.4 |
| Kidney Ca Nuclear grade 1/2 | 1.1 | Normal Ovary | 7.7 |
| (OD04339) | | | |
| Kidney margin (OD04339) | 0.2 | Ovarian Cancer | 13.6 |
| Kidney Ca, Clear cell type | 8.8 | Ovarian cancer (OD06145) | 0.6 |
| (OD04340) | | | |
| Kidney margin (OD04340) | 4.5 | Ovarian cancer margin | 2.2 |
| | | (OD06145) | |
| Kidney Ca, Nuclear grade 3 | 1.3 | Normal Stomach | 4.1 |
| (OD04348) | | | |
| Kidney margin (OD04348) | 1.8 | Gastric Cancer 9060397 | 1.2 |
| Kidney Cancer (OD04450-01) | 0.6 | Stomach margin 9060396 | 0.5 |
| Kidney margin (OD04450-03) | 4.6 | Gastric Cancer 9060395 | 7.4 |
| Kidney Cancer 8120613 | 0.3 | Stomach margin 9060394 | 2.6 |
| Kidney margin 8120614 | 0.5 | Gastric Cancer 064005 | 4.3 |

TABLE QO
Panel 4.1D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag3899, | Ag3960, | Ag4338, | | Ag3899, | Ag3960, | Ag4338, |
| Tissue | Run | Run | Run | Tissue | Run | Run | Run |
| Name | 170120166 | 170739794 | 184798156 | Name | 170120166 | 170739794 | 184798156 |
| Secondary | 0.0 | 0.0 | 0.0 | HUVEC | 4.1 | 3.9 | 7.4 |
| Th1 act | | | | IL-1beta | | | |
| Secondary | 0.0 | 0.0 | 0.0 | HUVEC | 15.8 | 22.8 | 22.4 |
| Th2 act | | | | IFN | | | |
| | | | | gamma | | | |
| Secondary | 0.0 | 0.0 | 0.0 | HUVEC | 1.0 | 8.0 | 8.8 |
| Tr1 act | | | | TNF alpha + | | | |
| | | | | IFN | | | |
| | | | | gamma | | | |
| Secondary | 0.0 | 0.0 | 0.0 | HUVEC | 2.9 | 4.7 | 8.0 |
| Th1 rest | | | | TNF alpha + | | | |
| | | | | IL4 | | | |
| Secondary | 0.0 | 0.0 | 0.6 | HUVEC | 4.2 | 10.2 | 10.4 |
| Th2 rest | | | | IL-11 | | | |
| Secondary | 0.0 | 0.0 | 0.0 | Lung | 1.5 | 8.1 | 8.4 |
| Tr1 rest | | | | Microvascular | | | |
| | | | | EC | | | |
| | | | | none | | | |
| Primary | 0.0 | 0.0 | 0.0 | Lung | 0.0 | 2.7 | 3.3 |
| Th1 act | | | | Microvascular | | | |
| | | | | EC | | | |
| | | | | TNF alpha + | | | |
| | | | | IL-1beta | | | |
| Primary | 0.0 | 0.0 | 0.0 | Microvascular | 0.0 | 1.0 | 1.6 |
| Th2 act | | | | Dermal | | | |
| | | | | EC none | | | |
| Primary | 0.0 | 0.0 | 0.0 | Microsvascular | 0.0 | 0.0 | 1.5 |
| Tr1 act | | | | Dermal | | | |
| | | | | EC | | | |
| | | | | TNF alpha + | | | |
| | | | | IL-1beta | | | |
| Primary | 0.0 | 0.0 | 0.0 | Bronchial | 0.4 | 7.7 | 5.0 |
| Th1 rest | | | | epithelium | | | |
| | | | | TNF alpha + | | | |
| | | | | IL1beta | | | |
| Primary | 0.0 | 0.0 | 0.0 | Small | 0.0 | 0.0 | 0.6 |
| Th2 rest | | | | airway | | | |
| | | | | epithelium | | | |
| | | | | none | | | |
| Primary | 0.0 | 0.4 | 0.6 | Small | 0.0 | 0.5 | 0.0 |
| Tr1 rest | | | | airway | | | |
| | | | | epithelium | | | |
| | | | | TNF alpha + | | | |
| | | | | IL-1beta | | | |
| CD45RA | 0.3 | 2.2 | 2.4 | Coronery | 8.5 | 12.7 | 8.2 |
| CD4 | | | | artery | | | |
| lymphocyte | | | | SMC rest | | | |
| act | | | | | | | |
| CD45RO | 0.0 | 0.0 | 0.0 | Coronery | 1.8 | 10.6 | 9.8 |
| CD4 | | | | artery | | | |
| lymphocyte | | | | SMC | | | |
| act | | | | TNF alpha + | | | |
| | | | | IL-1beta | | | |
| CD8 | 0.0 | 0.0 | 0.0 | Astrocytes | 0.0 | 0.5 | 0.8 |
| lymphocyte | | | | rest | | | |
| act | | | | | | | |
| Secondary | 0.0 | 0.0 | 0.0 | Astrocytes | 0.5 | 1.3 | 2.3 |
| CD8 | | | | TNF alpha + | | | |
| lymphocyte | | | | IL-1beta | | | |
| rest | | | | | | | |
| Secondary | 0.0 | 0.0 | 0.0 | KU-812 | 1.0 | 3.1 | 3.4 |
| CD8 | | | | (Basophil) | | | |
| lymphocyte | | | | rest | | | |
| act | | | | | | | |
| CD4 | 0.0 | 0.4 | 0.0 | KU-812 | 8.0 | 27.9 | 28.9 |
| lymphocyte | | | | (Basophil) | | | |
| none | | | | PMA/ | | | |
| | | | | ionomycin | | | |
| 2ry | 0.0 | 0.0 | 1.1 | CCD1106 | 0.0 | 1.6 | 4.0 |
| Th1/Th2/Tr1_{—} | | | | (Keratinocytes) | | | |
| anti-C | | | | none | | | |
| D95 CH11 | | | | | | | |
| LAK cells | 0.0 | 0.0 | 0.0 | CCD1106 | 0.0 | 1.1 | 2.0 |
| rest | | | | (Keratinocytes) | | | |
| | | | | TNF alpha + | | | |
| | | | | IL-1beta | | | |
| LAK cells | 0.0 | 0.0 | 0.0 | Liver | 7.6 | 18.6 | 14.2 |
| IL-2 | | | | cirrhosis | | | |
| LAK cells | 0.0 | 0.4 | 0.0 | NCI-H292 | 0.0 | 0.0 | 0.0 |
| IL-2 + IL-12 | | | | none | | | |
| LAK cells | 0.0 | 0.0 | 0.0 | NCI-H292 | 0.0 | 0.0 | 0.0 |
| IL-2 + IFN | | | | IL-4 | | | |
| gamma | | | | | | | |
| LAK cells | 0.0 | 0.0 | 0.0 | NCI-H292 | 0.0 | 0.0 | 0.0 |
| IL-2 + | | | | IL-9 | | | |
| IL-18 | | | | | | | |
| LAK cells | 0.0 | 0.0 | 0.0 | NCI-H292 | 0.0 | 0.5 | 0.5 |
| PMA/ionomycin | | | | IL-13 | | | |
| NK Cells | 0.0 | 0.0 | 0.0 | NCI-H292 | 0.0 | 0.0 | 0.0 |
| IL-2 rest | | | | IFN | | | |
| | | | | gamma | | | |
| Two Way | 0.0 | 0.0 | 0.0 | HPAEC | 17.9 | 21.8 | 13.2 |
| MLR 3 | | | | none | | | |
| day | | | | | | | |
| Two Way | 0.0 | 0.0 | 0.0 | HPAEC | 11.3 | 14.6 | 13.4 |
| MLR 5 | | | | TNF alpha + | | | |
| day | | | | IL-1 | | | |
| | | | | beta | | | |
| Two Way | 0.0 | 0.0 | 0.0 | Lung | 3.4 | 3.3 | 5.8 |
| MLR 7 | | | | fibroblast | | | |
| day | | | | none | | | |
| PBMC rest | 0.0 | 0.0 | 0.0 | Lung | 2.7 | 2.0 | 5.3 |
| | | | | fibroblast | | | |
| | | | | TNF alpha + | | | |
| | | | | IL-1 | | | |
| | | | | beta | | | |
| PBMC | 0.0 | 0.0 | 1.9 | Lung | 4.4 | 1.8 | 7.1 |
| PWM | | | | fibroblast | | | |
| | | | | IL-4 | | | |
| PBMC | 0.0 | 0.0 | 0.0 | Lung | 2.2 | 3.6 | 5.2 |
| PHA-L | | | | fibroblast | | | |
| | | | | IL-9 | | | |
| Ramos (B | 0.0 | 0.0 | 0.0 | Lung | 3.9 | 6.4 | 6.4 |
| cell) none | | | | fibroblast | | | |
| | | | | IL-13 | | | |
| Ramos (B | 0.0 | 0.0 | 0.0 | Lung | 7.2 | 6.5 | 7.8 |
| cell) | | | | fibroblast | | | |
| ionomycin | | | | IFN | | | |
| | | | | gamma | | | |
| B | 0.0 | 0.0 | 0.7 | Dermal | 5.5 | 11.4 | 9.3 |
| lymphocytes | | | | fibroblast | | | |
| PWM | | | | CCD1070 | | | |
| | | | | rest | | | |
| B | 0.0 | 0.0 | 0.9 | Dermal | 1.9 | 8.4 | 9.5 |
| lymphocytes | | | | fibroblast | | | |
| CD40L | | | | CCD1070 | | | |
| and IL-4 | | | | TNF alpha | | | |
| EOL-1 | 0.0 | 0.0 | 0.0 | Dermal | 1.5 | 6.7 | 6.8 |
| dbcAMP | | | | fibroblast | | | |
| | | | | CCD1070 | | | |
| | | | | IL-1beta | | | |
| EOL-1 | 0.0 | 0.0 | 0.0 | Dermal | 29.5 | 41.8 | 17.7 |
| dbcAMP | | | | fibroblast | | | |
| PMA/ionomycin | | | | IFN | | | |
| | | | | gamma | | | |
| Dendritic | 0.0 | 0.0 | 0.0 | Dermal | 75.8 | 69.3 | 51.8 |
| cells none | | | | fibroblast | | | |
| | | | | IL-4 | | | |
| Dendritic | 0.0 | 0.0 | 0.0 | Dermal | 21.5 | 36.9 | 29.5 |
| cells LPS | | | | Fibroblasts | | | |
| | | | | rest | | | |
| Dendritic | 0.0 | 0.0 | 0.0 | Neutrophils | 0.0 | 2.2 | 0.0 |
| cells | | | | TNFa + LPS | | | |
| anti-CD40 | | | | | | | |
| Monocytes | 0.0 | 0.0 | 0.0 | Neutrophils | 0.0 | 6.6 | 0.4 |
| rest | | | | rest | | | |
| Monocytes | 0.0 | 0.0 | 0.0 | Colon | 2.0 | 5.6 | 2.3 |
| LPS | | | | | | | |
| Macrophages | 0.0 | 0.0 | 0.0 | Lung | 100.0 | 100.0 | 100.0 |
| rest | | | | | | | |
| Macrophages | 0.0 | 0.0 | 0.0 | Thymus | 0.5 | 4.4 | 4.5 |
| LPS | | | | | | | |
| HUVEC | 3.2 | 7.8 | 10.5 | Kidney | 3.4 | 8.4 | 8.8 |
| none | | | | | | | |
| HUVEC | 8.1 | 15.4 | 14.6 | | | | |
| starved | | | | | | | |

TABLE QP
Panel 4D

| | Rel. Exp. (%) | | Rel. Exp. (%) |
|---|---|---|---|
| | Ag3108, Run | | Ag3108, Run |
| Tissue Name | 164529436 | Tissue Name | 164529436 |
| Secondary Th1 act | 0.0 | HUVEC IL-1beta | 3.1 |
| Secondary Th2 act | 0.0 | HUVEC IFN gamma | 7.9 |
| Secondary Tr1 act | 0.0 | HUVEC TNF alpha + IFN | 3.5 |
| | | gamma | |
| Secondary Th1 rest | 0.0 | HUVEC TNF alpha + IL4 | 7.1 |
| Secondary Th2 rest | 0.2 | HUVEC IL-11 | 4.6 |
| Secondary Tr1 rest | 0.3 | Lung Microvascular EC none | 2.3 |
| Primary Th1 act | 0.0 | Lung Microvascular EC | 0.3 |
| | | TNF alpha + IL-1beta | |
| Primary Th2 act | 0.0 | Microvascular Dermal EC | 1.2 |
| | | none | |
| Primary Tr1 act | 0.0 | Microsvasular Dermal EC | 0.6 |
| | | TNF alpha + IL-1beta | |
| Primary Th1 rest | 0.0 | Bronchial epithelium | 3.2 |
| | | TNF alpha + IL1beta | |
| Primary Th2 rest | 0.3 | Small airway epithelium none | 0.2 |
| Primary Tr1 rest | 0.0 | Small airway epithelium | 0.3 |
| | | TNF alpha + IL-1beta | |
| CD45RA CD4 lymphocyte act | 1.5 | Coronery artery SMC rest | 11.7 |
| CD45RO CD4 lymphocyte act | 0.0 | Coronery artery SMC | 3.6 |
| | | TNF alpha + IL-1beta | |
| CD8 lymphocyte act | 0.0 | Astrocytes rest | 0.2 |
| Secondary CD8 lymphocyte | 0.0 | Astrocytes TNF alpha + | 3.7 |
| rest | | IL-1beta | |
| Secondary CD8 lymphocyte act | 0.0 | KU-812 (Basophil) rest | 0.6 |
| CD4 lymphocyte none | 0.0 | KU-812 (Basophil) | 25.7 |
| | | PMA/ionomycin | |
| 2ry Th1/Th2/Tr1_anti-CD95 | 0.0 | CCD1106 (Keratinocytes) | 0.6 |
| CH11 | | none | |
| LAK cells rest | 0.1 | CCD1106 (Keratinocytes) | 0.4 |
| | | TNF alpha + IL-1beta | |
| LAK cells IL-2 | 0.3 | Liver cirrhosis | 12.2 |
| LAK cells IL-2 + IL-12 | 0.0 | Lupus kidney | 0.2 |
| LAK cells IL-2 + IFN gamma | 0.0 | NCI-H292 none | 0.3 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.2 | NCI-H292 IFN gamma | 0.0 |
| Two Way MLR 5 day | 0.0 | HPAEC none | 11.2 |
| Two Way MLR 7 day | 0.0 | HPAEC TNF alpha + IL-1 | 6.3 |
| | | beta | |
| PBMC rest | 0.0 | Lung fibroblast none | 1.1 |
| PBMC PWM | 0.9 | Lung fibroblast TNF alpha + | 3.0 |
| | | IL-1beta | |
| PBMC PHA-L | 0.0 | Lung fibroblast IL-4 | 4.2 |
| Ramos (B cell) none | 0.0 | Lung fibroblast IL-9 | 3.5 |
| Ramos (B cell) ionomycin | 0.0 | Lung fibroblast IL-13 | 5.0 |
| B lymphocytes PWM | 0.5 | Lung fibroblast IFN gamma | 6.9 |
| B lymphocytes CD40L and IL-4 | 0.0 | Dermal fibroblast CCD1070 | 9.0 |
| | | rest | |
| EOL-1 dbcAMP | 0.0 | Dermal fibroblast CCD1070 | 10.9 |
| | | TNF alpha | |
| EOL-1 dbcAMP | 0.0 | Dermal fibroblast CCD1070 | 3.6 |
| PMA/ionomycin | | IL-1beta | |
| Dendritic cells none | 0.0 | Dermal fibroblast IFN gamma | 22.8 |
| Dendritic cells LPS | 0.0 | Dermal fibroblast IL-4 | 34.2 |
| Dendritic cells anti-CD40 | 0.0 | IBD Colitis 2 | 0.2 |
| Monocytes rest | 0.0 | IBD Crohn's | 3.2 |
| Monocytes LPS | 0.0 | Colon | 13.0 |
| Macrophages rest | 0.0 | Lung | 100.0 |
| Macrophages LPS | 0.0 | Thymus | 16.2 |
| HUVEC none | 6.0 | Kidney | 3.7 |
| HUVEC starved | 19.3 | | |

CNS_neurodegeneration_v1.0 Summary: Ag3899/Ag3960/Ag4338 Expression of the CG94013-01 gene is low/undetectable (CTs>34) across all of the samples on this panel.

General_screening_panel_v1.4 Summary: Ag3899/Ag3960/Ag4338 Results of three experiments with two different primer and probe sets are in excellent agreement, with highest expression of the CG94013-01 gene in CNS cancer (astro) SNB-75 cell line (CTs=23-26). In addition, high expression of this gene is seen in CNS cancer cell lines, colon cancer tissue, renal cancer cell line UO-31, breast cancer and melanoma cell lines. Therefore, expression of this gene can be used to distinguish these samples from other samples in the panel and also as marker for detection of these cancers. In addition, therapeutic modulation of the activity of this gene or its protein product, through the use of small molecule drugs, protein therapeutics or antibodies, might be beneficial in the treatment of these cancers.

Among tissues with metabolic or endocrine function, this gene is expressed at low to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Interestingly, this gene is expressed at much higher levels in fetal liver (CTs=31-32) and lung (CTs-28) when compared to corresponding adult tissue (CTs=33-35). This observation suggests that expression of this gene can be used to distinguish these fetal tissues from corresponding adult tissues.

Panel 1 Summary: Ag343 Highest expression of the CG94013-01 gene is detected in breast cancer MDA-N cell line (CTs=26). In addition high expression of this gene is also observed in melanoma, astrocytoma, and lung cance cell lines. Please see panel 1.4 for the utility of this gene.

Panel 1.3D Summary: Ag3108 Highest expression of the CG94013-01 gene is detected in melanoma (met) Hs688(B).T cell line (CT=27). In addition, expression of this gene is also seen in melanoma, breast cancer, lung cancer, astrocytoma cell lines and colon cancer well to moderately differentiated (OD03866) tissue. Please see panel 1.4 for the utility of this gene.

Panel 2.1 Summary: Ag3108 Highest expression of the CG94013-01 gene is detected in melanoma metastasis sample (CT=29). In addition, expression of this gene is higher in metastasis breast cancer (OD04590-03) (CT=33) as compared to breast cancer (OD04590-01) (CT=36.7). Thus, expression of this gene can be used to distinguish these two samples from each other and also as marker for cancer metastasis. Please see panel 1.4 for further utility of this gene.

Panel 4.1D Summary: Ag3899/Ag3960/Ag4338 Results of three experiments with two different primer and probe sets are in excellent agreement, with highest expression of the CG94013-01 gene in lung (CT=30-31). In addition, significant expression of this gene is seen in HUVEC cells, lung fibroblast and dermal fibroblasts. Therefore, antibody or small molecule therapies designed with the protein encoded for by this gene could be important in the treatment of inflammatory lung disorders such as chronic obstructive pulmonary disease, asthma, allergy and emphysema and skin disorders including psoriasis.

In addition, low expression of this gene is also seen in kidney. Therefore, antibody or small molecule therapies designed with the protein encoded for by this gene could modulate kidney function and be important in the treatment of inflammatory or autoimmune diseases that affect the kidney, including lupus and glomerulonephritis.

Panel 4D Summary: Ag3108 Highest expression of the CG94013-01 gene in lung (CT=28.6). In addition, significant expression of this gene is seen in HPAEC cells, HUVEC cells, lung fibroblast, TNFalpha+IL1beta treated bronchial epithelium and dermal fibroblasts. Therefore, antibody or small molecule therapies designed with the protein encoded for by this gene could be important in the treatment of inflammatory lung disorders such as chronic obstructive pulmonary disease, asthma, allergy and emphysema and skin disorders including psoriasis.

In addition, low expression of this gene is also seen in kidney and colon. Therefore, antibody or small molecule therapies designed with the protein encoded for by this gene be important in the treatment of inflammatory or autoimmune diseases that affect the kidney, including lupus and glomerulonephritis, as well as, inflammatory bowel diseases such as Crohns.

Interestingly, expression of this gene is stimulated in PMA/ionomycin treated basophils (CT=30) as compared to resting basophils (CT=36). Basophils release histamines and other biological modifiers in reponse to allergens and play an important role in the pathology of asthma and hypersensitivity reactions. Therefore, therapeutics designed against the putative protein encoded by this gene may reduce or inhibit inflammation by blocking basophil function in these diseases. In addition, these cells are a reasonable model for the inflammatory cells that take part in various inflammatory lung and bowel diseases, such as asthma, Crohn's disease, and ulcerative colitis. Therefore, therapeutics that modulate the function of this gene product may reduce or eliminate the symptoms of patients suffering from asthma, Crohn's disease, and ulcerative colitis.

Ag1924 Results from one experiment with the CG94013-01 gene are not included. The amp plot indicates that there were experimental difficulties with this run.

R. NOV24 (CG94442-01): Carboxylesterase Precursor

Expression of gene CG94442-01 was assessed using the primer-probe set Ag3908, described in Table RA.

TABLE RA
Probe Name Ag3908

| | | | Start | SEQ ID |
|---|---|---|---|---|
| Primers | Sequences | Length | Position | NO |
| Forward | 5′-gctgaaggacaaggaagtagct-3′ | 22 | 1602 | 196 |
| Probe | TET-5′-tctggaccaaactcttcgccaagaag-3′-TAMRA | 26 | 1625 | 197 |
| Reverse | 5′-tcagctccatggcttagttcta-3′ | 22 | 1673 | 198 |

CNS_neurodegeneration_v1.0 Summary: Ag3908 Expression of the CG94442-01 gene is low/undetectable in all samples on this panel (CTs>35).

General_screening_panel_v1.4 Summary: Ag3908 Expression of the CG94442-01 gene is low/undetectable in all samples on this panel (CTs>35).

Panel 4.1D Summary: Ag3908 Expression of the CG94442-01 gene is low/undetectable in all samples on this panel (CTs>35).

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

We claim:

1. An isolated polypeptide comprising an amino acid sequence selected from the group consisting of:
a) a mature form of the amino acid sequence selected from the group consisting of SEQ ID NO:2n, wherein n is an integer between 1 and 45;
b) a variant of a mature form of the amino acid sequence selected from the group consisting of SEQ ID NO:2n, wherein n is an integer between 1 and 45, wherein any amino acid in the mature form is changed to a different amino acid, provided that no more than 15% of the amino acid residues in the sequence of the mature form are so changed;
c) the amino acid sequence selected from the group consisting of SEQ ID NO:2n, wherein n is an integer between 1 and 45;
d) a variant of the amino acid sequence selected from the group consisting of SEQ ID NO:2n, wherein n is an integer between 1 and 45, wherein any amino acid specified in the chosen sequence is changed to a different amino acid, provided that no more than 15% of the amino acid residues in the sequence are so changed; and
e) a fragment of any of a) through d).

2. The polypeptide of claim 1 that is a naturally occurring allelic variant of the sequence selected from the group consisting of SEQ ID NO:2n, wherein n is an integer between 1 and 45.

3. The polypeptide of claim 2, wherein the allelic variant comprises an amino acid sequence that is the translation of a nucleic acid sequence differing by a single nucleotide from a nucleic acid sequence selected from the group consisting of SEQ ID NOS: 2n, wherein n is an integer between 1 and 45.

4. The polypeptide of claim 1 that is a variant polypeptide described therein, wherein any amino acid specified in the chosen sequence is changed to provide a conservative substitution.

5. A pharmaceutical composition comprising the polypeptide of claim 1 and a pharmaceutically acceptable carrier.

6. A kit comprising in one or more containers, the pharmaceutical composition of claim 5.

7. The use of a therapeutic in the manufacture of a medicament for treating a syndrome associated with a human disease, the disease selected from a pathology associated with the polypeptide of claim 1, wherein the therapeutic is the polypeptide of claim 1.

8. A method for determining the presence or amount of the polypeptide of claim 1 in a sample, the method comprising:
(a) providing the sample;
(b) introducing the sample to an antibody that binds immunospecifically to the polypeptide; and
(c) determining the presence or amount of antibody bound to the polypeptide, thereby determining the presence or amount of polypeptide in the sample.

9. A method for determining the presence of or predisposition to a disease associated with altered levels of the polypeptide of claim 1 in a first mammalian subject, the method comprising:
a) measuring the level of expression of the polypeptide in a sample from the first mammalian subject; and
b) comparing the amount of the polypeptide in the sample of step (a) to the amount of the polypeptide present in a control sample from a second mammalian subject known not to have, or not to be predisposed to, the disease,
wherein an alteration in the expression level of the polypeptide in the first subject as compared to the control sample indicates the presence of or predisposition to the disease.

10. A method of identifying an agent that binds to the polypeptide of claim 1, the method comprising:
(a) introducing the polypeptide to the agent; and
(b) determining whether the agent binds to the polypeptide.

11. The method of claim 10 wherein the agent is a cellular receptor or a downstream effector.

12. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition devoid of the substance, the substance is identified as a potential therapeutic agent.

13. A method for screening for a modulator of activity or of latency or predisposition to a pathology associated with the polypeptide of claim 1, the method comprising:
a) administering a test compound to a test animal at increased risk for a pathology associated with the polypeptide of claim 1, wherein the test animal recombinantly expresses the polypeptide of claim 1;
b) measuring the activity of the polypeptide in the test animal after administering the compound of step (a); and
c) comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of claim 1.

14. The method of claim 13, wherein the test animal is a recombinant test animal that expresses a test protein transgene or expresses the transgene under the control of a promoter at an increased level relative to a wild-type test animal, and wherein the promoter is not the native gene promoter of the transgene.

15. A method for modulating the activity of the polypeptide of claim 1, the method comprising introducing a cell sample expressing the polypeptide of the claim with a compound that binds to the polypeptide in an amount sufficient to modulate the activity of the polypeptide.

16. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

17. The method of claim 16, wherein the subject is a human.

18. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 45, or a biologically active fragment thereof.

19. An isolated nucleic acid molecule comprising a nucleic acid sequence encoding a polypeptide comprising an amino acid sequence selected from the group consisting of:
a) a mature form of the amino acid sequence given SEQ ID NO:2n, wherein n is an integer between 1 and 45;
b) a variant of a mature form of the amino acid sequence selected from the group consisting of SEQ ID NO:2n, wherein n is an integer between 1 and 45, wherein any amino acid in the mature form of the chosen sequence is changed to a different amino acid, provided that no more than 15% of the amino acid residues in the sequence of the mature form are so changed;
c) the amino acid sequence selected from the group consisting of SEQ ID NO:2n, wherein n is an integer between 1 and 45;
d) a variant of the amino acid sequence selected from the group consisting of SEQ ID NO:2n, wherein n is an integer between 1 and 45, in which any amino acid specified in the chosen sequence is changed to a different amino acid, provided that no more than 15% of the amino acid residues in the sequence are so changed;
e) a nucleic acid fragment encoding at least a portion of a polypeptide comprising the amino acid sequence selected from the group consisting of SEQ ID NO:2n, wherein n is an integer between 1 and 45, or any variant of the polypeptide wherein any amino acid of the chosen sequence is changed to a different amino acid, provided that no more than 10% of the amino acid residues in the sequence are so changed; and
f) the complement of any of the nucleic acid molecules.

20. The nucleic acid molecule of claim 19, wherein the nucleic acid molecule comprises the nucleotide sequence of a naturally occurring allelic nucleic acid variant.

21. The nucleic acid molecule of claim 19 that encodes a variant polypeptide, wherein the variant polypeptide has the polypeptide sequence of a naturally occurring polypeptide variant.

22. The nucleic acid molecule of claim 19, wherein the nucleic acid molecule differs by a single nucleotide from a nucleic acid sequence selected from the group consisting of SEQ ID NOS: 2n-1, wherein n is an integer between 1 and 45.

23. The nucleic acid molecule of claim 19, wherein the nucleic acid molecule comprises a nucleotide sequence selected from the group consisting of
a) the nucleotide sequence selected from the group consisting of SEQ ID NO:2n−1, wherein n is an integer between 1 and 45;
b) a nucleotide sequence wherein one or more nucleotides in the nucleotide sequence selected from the group consisting of SEQ ID NO:2n-1, wherein n is an integer between 1 and 45, is changed from that selected from the group consisting of the chosen sequence to a different nucleotide provided that no more than 15% of the nucleotides are so changed;
c) a nucleic acid fragment of the sequence selected from the group consisting of SEQ ID NO:2n-1, wherein n is an integer between 1 and 45; and
d) a nucleic acid fragment wherein one or more nucleotides in the nucleotide sequence selected from the group consisting of SEQ ID NO:2n-1, wherein n is an integer between 1 and 45, is changed from that selected from the group consisting of the chosen sequence to a different nucleotide provided that no more than 15% of the nucleotides are so changed.

24. The nucleic acid molecule of claim 19, wherein the nucleic acid molecule hybridizes under stringent conditions to the nucleotide sequence selected from the group consisting of SEQ ID NO:2n-1, wherein n is an integer between 1 and 45, or a complement of the nucleotide sequence.

25. The nucleic acid molecule of claim 19, wherein the nucleic acid molecule comprises a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

26. A vector comprising the nucleic acid molecule of claim 19.

27. The vector of claim 26, further comprising a promoter operably linked to the nucleic acid molecule.

28. A cell comprising the vector of claim 27.

29. A method for determining the presence or amount of the nucleic acid molecule of claim 19 in a sample, the method comprising:
(a) providing the sample;
(b) introducing the sample to a probe that binds to the nucleic acid molecule; and
(c) determining the presence or amount of the probe bound to the nucleic acid molecule,
thereby determining the presence or amount of the nucleic acid molecule in the sample.

30. The method of claim 29 wherein presence or amount of the nucleic acid molecule is used as a marker for cell or tissue type.

31. The method of claim 30 wherein the cell or tissue type is cancerous.

32. A method for determining the presence of or predisposition to a disease associated with altered levels of the nucleic acid molecule of claim 19 in a first mammalian subject, the method comprising:
a) measuring the amount of the nucleic acid in a sample from the first mammalian subject; and
b) comparing the amount of the nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.
